(19) United States
(12) Patent Application Publication
(10) Pub. No.: US 2021/0284638 A1
(43) Pub. Date: Sep. 16, 2021

(54) ALPHAvBETA1 INTEGRIN ANTAGONISTS

(71) Applicants: Saint Louis University, St. Louis, MO (US); Indalo Therapeutics, Inc., St. Louis, MO (US)

(72) Inventors: Peter G. RUMINSKI, St. Louis, MO (US); David W. GRIGGS, St. Louis, MO (US); Scott SEIWERT, St. Louis, MO (US)

(73) Assignees: Saint Louis University, St. Louis, MO (US); Indalo Therapeutics, Inc., St. Louis, MO (US)

(21) Appl. No.: 17/257,432

(22) PCT Filed: Jun. 27, 2019
(86) PCT No.: PCT/US2019/039430

§ 371 Date: Dec. 31, 2020

Related U.S. Application Data
(60) Provisional application No. 62/693,827, filed on Jul. 3, 2018.

(51) Int. Cl. C07D 471/04

(52) U.S. Cl. CPC C07D 471/04

(57) ABSTRACT

The present disclosure provides pharmaceutical agents, including those of the formula: (I) wherein the variables are defined herein. Also provided are pharmaceutical compositions, kits and articles of manufacture comprising such pharmaceutical agents. Methods of using the pharmaceutical agents are also provided. The compounds may be used for the inhibition or antagonism of integrins ανβ₁ and/or α₅β₁. In some embodiments, the compounds provided herein exhibit reduced inhibitory or antagonistic activity of integrins ανβ₃, ανβ₅, ανβ₆, ανβ₈, and/or α_{IIb}β₃.

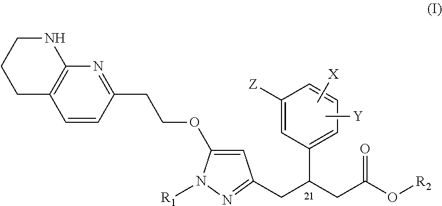

### BACKGROUND OF THE INVENTION

### Field of the Invention

The present disclosure relates to the fields of pharmaceuticals, medicine and cell biology. More specifically, it relates to pharmaceutical agents (compounds) which are useful as integrin antagonists

### Description of the Related Art

Integrins are a family of integral cytoplasmic membrane proteins that mediate cell interactions with other cells and with the extracellular matrix. Recently, integrin α_{V}β₁ was identified to play a role in a variety of fibrotic conditions. Other integrins, such as α_{V}β₃ and α_{V}β₅, are also associated with fibrotic conditions and compounds which inhibit these two integrins may be useful in the treatment of these conditions.

Integrin α₅β₁ is believed to bind to fibronectin in a region that incorporates the ninth and tenth type III fibronectin repeats, the latter of which is believed to contain the RGD motif for integrin binding. In addition to fibronectin, α₅β₁ has been reported to interact with other RGD-containing extracellular matrix proteins including fibrinogen, denatured collagen, and fibrillin-1 (Bax et al., J. Biol. Chem., 278(36):34605-34616, 2003, 2003; Perdih, Curr. Med. Chem., 17(22):2371-2392, 2010; Suehiro et al., J. Biochem., 128(4):705-710, 2000). These ligands are generally classified as components of the provisional matrix that is laid down by cells as part of the wound healing response in tissues. Components of this response are angiogenesis and fibrosis.

In contrast, inhibition of some other integrins, such as α_{V}β₆ and α_{V}β₈, has been associated with a variety of undesired, inflammation-related side effects (Huang, et al., 1996; Lacy-Hulbert, et al., 2007; Travis, et al., 2007; Worthington, et al., 2015). Selective inhibition of α_{V}β₁, α_{V}β₃, α_{V}β₅, and/or α₅β₁ is desirable for some indications.

Integrin α_{IIb}β_{III} (also known as glycoprotein IIb/IIIa or GPIIb/IIIa) is an integrin complex found on platelets. Integrin α_{IIb}β_{III} inhibition is associated with disruption of platelet aggregation, which is associated with toxicity and/or contraindicated when treating certain disease or disorders (King et al., 2016; Bennet, 2005; Giordano et al, 2016; Cook et al., 1997).

### SUMMARY

The present disclosure provides novel integrin receptor antagonists, pharmaceutical compositions, and methods for their manufacture, and methods for their use.

In some aspects, the present disclosure provides compounds of the formula:

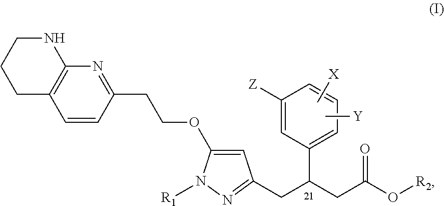

or a pharmaceutically acceptable salt, solvate or tautomer of the above formula, wherein: R₁, R₂, X, Y and Z have any of the values described herein.

In some embodiments, the compounds are further defined as:

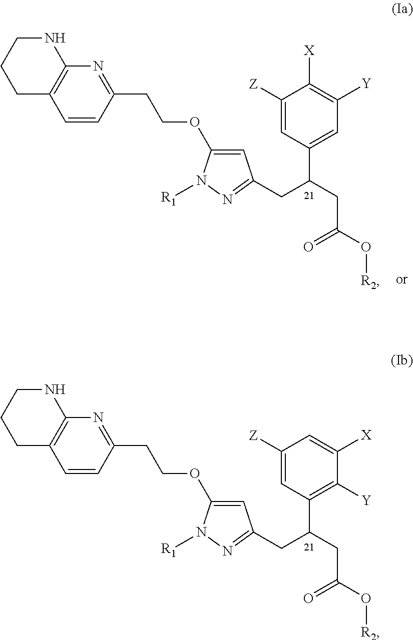

or a pharmaceutically acceptable salt, solvate or tautomer thereof, wherein: R₁, R₂, X, Y and Z have any of the values described herein.

In some embodiments, the compounds are further defined as:

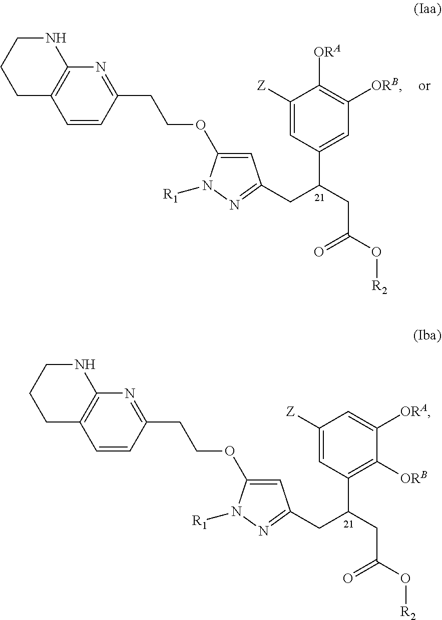

or a pharmaceutically acceptable salt, solvate or tautomer thereof, wherein: R₁, R₂, R^{A}, R^{B}, X and Z have any of the values described herein.

In some embodiments of Formula (I), (Ia), and (Ib), R₁ may be hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, or substituted C₇₋₁₂aralkyl;

R₂ may be hydrogen, unsubstituted C₁₋₈alkyl, or substituted C₁₋₈alkyl;
i) X and Y are each independently —OR^{X}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryl-CH₂O—, substituted C_{6 or 10}aryl-CH₂O—, unsubstituted C_{6 or 10}aryloxy, substituted C_{6 or 10}aryloxy, unsubstituted C₂₋₁₂acyloxy, substituted C₂₋₁₂acyloxy,

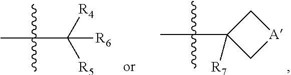

R₄ and R₅ are each independently unsubstituted C₁₋₈alkyl or substituted C₁₋₈alkyl, and

R₆ is hydrogen, —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,

A′ is —CF₂—, —O—, C₁₋₆alkanediyl, C₁₋₈alkoxydiyl, C₂₋₈alkylalkyoxy, or a covalent bond, thereby forming a cyclopropane ring, and

R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and

R^{X} is —(CH₂CH₂O)ᵣH or —(CH₂CH₂O)ᵣC₁₋₆alkyl, where r is an integer from 1-6; or
ii) X is —OR^{A} and Y is —OR^{B}, where R^{A} and R^{B} together are —(CR₁₂)ₙ—,

each R₁₂ is independently hydrogen or unsubstituted C₁₋₆alkyl, and

n is 1 or 2;

Z is —OR^{Z}, t-butyl, unsubstituted 3-12 membered cycloalkyl, substituted 3-12 membered cycloalkyl, or

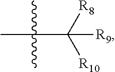

R₈ and R₉ are each independently unsubstituted C₁₋₈alkyl or substituted C₁₋₈alkyl,

R₁₀ is hydrogen, —OH, —CN, —NH₂, —CF₃, —CF₂H, —CFH₂, —CO₂H, —CO₂—C₁₋₈alkyl, —C(═O)NH₂, —CH₂OH, CH₂O—C₁₋₈alkyl, or C₁₋₈alkoxy, and

R^{Z} is —O(CH₂CH₂O)ₛH or —O(CH₂CH₂O)ₛC₁₋₆alkyl, where s is an integer from 1-6,

or

Z is

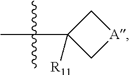

where A″ is —CF₂—, —O—, or C₁₋₈alkoxydiyl; and R₁₁ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy.

In some embodiments of Formula (I), (Ia), (Ib), (Iaa), and (Iba), R₁ is unsubstituted C₁₋₈alkyl, substituted C₁₋₈alkyl, unsubstituted C_{6 or 10}aryl, substituted C_{6 or 10}aryl, unsubstituted C₇₋₁₀aralkyl, or substituted C₇₋₁₀aralkyl; and R₂ is hydrogen, unsubstituted C₁₋₆alkyl, or substituted C₁₋₆alkyl.

In some embodiments of Formula (I), (Ia), (Ib), (Iaa), and (Iba), R₁ is unsubstituted C₁₋₈alkyl, substituted C₁₋₈alkyl, unsubstituted C_{6 or 10}aryl, substituted C_{6 or 10}aryl, unsubstituted C₇₋₁₀aralkyl, or substituted C₇₋₁₀aralkyl; and R₂ is hydrogen, unsubstituted C₁₋₆alkyl, or substituted C₁₋₆alkyl. In some embodiments of Formula (I), (Ia), (Ib), (Iaa), and (Iba), R₁ is unsubstituted C₁₋₈alkyl. In some embodiments of Formula (I), (Ia), (Ib), (Iaa), and (Iba), R₁ is methyl. In some embodiments of Formula (I), (Ia), (Ib), (Iaa), and (Iba), R₂ is hydrogen. In some embodiments of Formula (I), (Ia), (Ib), (Iaa), and (Iba), R^{A} and R^{B} together are —(CH₂)— or —(CH₂CH₂)—. In some embodiments of Formula (I), (Ia), (Ib), (Iaa), and (Iba), Z is t-butyl or adamantyl. In some embodiments of Formula (I), (Ia), (Ib), (Iaa), and (Iba), Z is t-butyl. In some embodiments of Formula (I), (Ia), (Ib), (Iaa), and (Iba), Z is adamantly. In some embodiments of Formula (I), (Ia), (Ib), (Iaa), and (Iba), R^{A} and R^{B} together are —(CH₂)— or —(CH₂CH₂)—.

In some embodiments of Formula (I), (Ia), and (Ib), X is halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl-CH₂O—, substituted C_{6 or 10}aryl-CH₂O—, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, or substituted C₂₋₁₂acyloxy. In some embodiments of Formula (I), (Ia), and (Ib), X is halo, cyano, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, unsubstituted C_{6 or 10}aryl-CH₂O—, substituted C_{6 or 10}aryl-CH₂O—, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy or

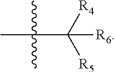

In some embodiments of Formula (I), (Ia), and (Ib), X is halo. In some embodiments of Formula (I), (Ia), and (Ib), X is bromo, fluoro, or chloro. In some embodiments of Formula (I), (Ia), and (Ib), X is —CF₃. In some embodiments of Formula (I), (Ia), and (Ib), X is —OH or cyano. In some embodiments of Formula (I), (Ia), and (Ib), X is unsubstituted C₁₋₈alkyl. In some embodiments of Formula (I), (Ia), and (Ib), X is unsubstituted C₃₋₆alkyl. In some embodiments of Formula (I), (Ia), and (Ib), X is unsubstituted C₁₋₈alkoxy. In some embodiments of Formula (I), (Ia), and (Ib), X is methoxy or isopropoxy. In some embodiments of Formula (I), (Ia), and (Ib), X is t-butyl. In some embodiments of Formula (I), (Ia), (Ib), (Iaa), and (Iba), X is —OR^{A} and Y is —OR^{B} and R^{A} and R^{B} together are —(CH₂)— or —(CH₂CH₂)—. In some embodiments of Formula (I), (Ia), and (Ib), Y is t-butyl.

In some embodiments of Formula (I), (Ia), and (Ib), Y is

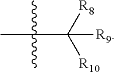

In some embodiments of Formula (I), (Ia), and (Ib), R₈ and R₉ are each independently unsubstituted C₂₋₈alkyl, or R₈ is methyl and R₉ is unsubstituted C₂₋₈alkyl, or R₈ and R₉ are each —CH₃. In some embodiments of Formula (I), (Ia), and (Ib), R₁₀ is —CF₃, —CF₂H, or —CFH₂. In some embodiments of Formula (I), (Ia), and (Ib), R₁₀ is hydrogen or —CF₃. In some embodiments of Formula (I), (Ia), and (Ib), R₁₀ is —CF₃.

In some embodiments of Formula (I), (Ia), and (Ib), Y is

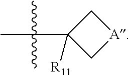

In some embodiments of Formula (I), (Ia), and (Ib), A″ is C₁₋₃alkanediyl, C₁₋₄alkoxydiyl, or a covalent bond, thereby forming a cyclopropane ring. In some embodiments of Formula (I), (Ia), and (Ib), A″ is a covalent bond, thereby forming a cyclopropane ring. In some embodiments of Formula (I), (Ia), and (Ib), R₁₁ is —CF₃, —CF₂H, —CH₂F, —CH₂O—C₁₋₆alkyl, C₁₋₆alkyl or C₁₋₈alkoxy. In some embodiments of Formula (I), (Ia), and (Ib), R₁₁ is —CF₃, —CF₂H, —CH₂F, C₁₋₆alkyl or C₁₋₆alkoxy. In some embodiments of Formula (I), (Ia), and (Ib), R₁₁ is —CF₃, —CF₂H or methoxy. In some embodiments of Formula (I), (Ia), and (Ib), R₁₁ is —CF₃ or —CF₂H. In some embodiments of Formula (I), (Ia), and (Ib), R₁₁ is —CH₂O—CH₃.

In some embodiments of Formula (I), (Ia), and (Ib), Y is halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl-CH₂O—, substituted C_{6 or 10}aryl-CH₂O—, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, or substituted C₂₋₁₂acyloxy. In some embodiments of Formula (I), (Ia), and (Ib), Y is halo, cyano, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, unsubstituted C_{6 or 10}aryl-CH₂O—, substituted C_{6 or 10}aryl-CH₂O—, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy or

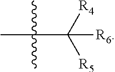

In some embodiments of Formula (I), (Ia), and (Ib), Y is halo. In some embodiments of Formula (I), (Ia), and (Ib), Y is bromo, fluoro, or chloro. In some embodiments of Formula (I), (Ia), and (Ib), Y is —CF₃. In some embodiments of Formula (I), (Ia), and (Ib), Y is —OH or cyano. In some embodiments of Formula (I), (Ia), and (Ib), Y is unsubstituted C₁₋₈alkyl. In some embodiments of Formula (I), (Ia), and (Ib), Y is unsubstituted C₃₋₆alkyl. In some embodiments of Formula (I), (Ia), and (Ib), Y is t-butyl. In some embodiments of Formula (I), (Ia), and (Ib), Y is unsubstituted C₁₋₈alkoxy. In some embodiments of Formula (I), (Ia), and (Ib), the compound is not the compound of Example 11. In some embodiments of Formula (I), (Ia), and (Ib), Z is t-butyl, X is t-butyl, Y is Y is halo, cyano, unsubstituted C₁₋₁₂alkyl, substituted C₁₋₁₂alkyl, unsubstituted C₂₋₁₂alkoxy, substituted C₁₋₁₂alkoxy, unsubstituted C_{6 or 10}aryl-CH₂O—, substituted C_{6 or 10}aryl-CH₂O—, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, or substituted C₂₋₁₂acyloxy.

In some aspects, the present disclosure provides compounds of the formula:

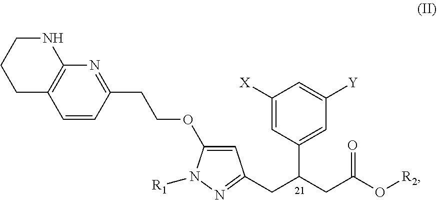

or a pharmaceutically acceptable salt, solvate or tautomer of the above formula, wherein: R₁, R₂, X and Y have any of the values described herein.

In some embodiments of Formula (II), R₁ may be hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, or substituted C₇₋₁₂aralkyl;

R₂ is hydrogen, unsubstituted C₁₋₈alkyl, or substituted C₁₋₈alkyl;

i) X is —OR^{X}, or halo;

Y is —OH, —OR^{Z}, —SF₅,

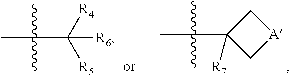

R₄ and R₅ are each independently unsubstituted C₁₋₈alkyl or substituted C₁₋₈alkyl, and

R₆ is —OH, —CN, —NH₂, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,

A′ is —CF₂—, —O—, C₁₋₆alkanediyl, or a covalent bond, thereby forming a cyclopropane ring, and

R₇ is —OH, —CN, —NH₂, —CO₂H, —CO₂—C₁₋₈alkyl, —C(═O)NH₂, —CF₃, —CH₂F, —CH₂OH, —CH₂O—C₁₋₈alkyl, C₁₋₈alkyl or C₁₋₈alkoxy,

where R^{X} is —(CH₂CH₂O)ᵣH or —(CH₂CH₂O)ᵣC₁₋₆alkyl, where r is an integer from 1-6, and

where R^{Z} is —(CH₂CH₂O)ₛH or —(CH₂CH₂O)ₛC₁₋₆alkyl, where s is an integer from 1-6;

ii) X is i-propyl, t-butyl,

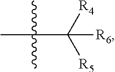

Y is i-propyl,

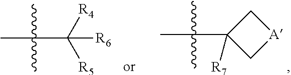

each R₄ and each R₅ are independently unsubstituted C₁₋₈alkyl or substituted C₁₋₈alkyl, and

each R₆ is independently —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,

A′ is —CF₂—, —O—, C₁₋₆alkanediyl, C₁₋₈alkoxydiyl, C₂₋₈alkylalkoxydiyl, or a covalent bond, thereby forming a cyclopropane ring, and

R₇ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy; or

iii) X is cyano,
Y is —OR^{Z}, —SF₅,

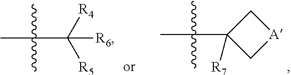

R₄ and R₅ are each independently unsubstituted C₁₋₈alkyl or substituted C₁₋₈alkyl, and
R₆ is —H, —OH, —CN, —NH₂, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
A′ is —CF₂—, —O—, C₁₋₆alkanediyl, C₁₋₈alkoxydiyl, or a covalent bond, thereby forming a cyclopropane ring,
R₇ is —OH, —CN, —NH₂, —CO₂H, —CO₂—C₁₋₈alkyl, —C(═O)NH₂, —CF₃, —CH₂F, —CH₂OH, —CH₂O—C₁₋₈alkyl, C₁₋₈alkyl or C₁₋₈alkoxy,
where R^{X} is —(CH₂CH₂O)ᵣH or —(CH₂CH₂O)ᵣC₁₋₆alkyl, where r is an integer from 1-6, and

where R^{Z} is —(CH₂CH₂O)ₛH or —(CH₂CH₂O)ₛC₁₋₆alkyl, where s is an integer from 1-6.

In some embodiments of Formula (II), R₁ may be unsubstituted C₁₋₈alkyl. In some embodiments of Formula (II), R₁ may be methyl. In some embodiments of Formula (II), R₂ may be hydrogen. In some embodiments of Formula (II), Y may be —OR^{Z}. In some embodiments of Formula (II), Y may be —SF₅. In some embodiments of Formula (II), Y may be i-propyl. In some embodiments of Formula (II), Y may be —OH. In some embodiments of Formula (II), Y may be i-propyl, or

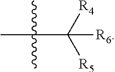

In some embodiments of Formula (II), Y may be

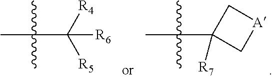

In some embodiments of Formula (II), Y may be

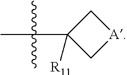

In some aspects, the present disclosure provides compounds of the formula:

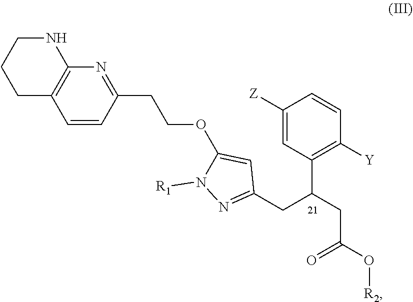

or a pharmaceutically acceptable salt, solvate or tautomer of the above formula, wherein: R₁, R₂, Y and Z have any of the values described herein.

In some embodiments of Formula (III), R₁ may be hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, or substituted C₇₋₁₂aralkyl;

R₂ is hydrogen, unsubstituted C₁₋₈alkyl, or substituted C₁₋₈alkyl;

i) Y is bromo, fluoro, cyano or substituted C₁₋₁₂alkoxy,

Z is t-butyl, —SF₅, or

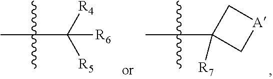

R₄ and R₅ are each independently unsubstituted C₁₋₈alkyl or substituted C₁₋₈alkyl, and

R₆ is —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,

A′ is —CF₂—, —O—, C₁₋₆alkanediyl, C₁₋₈alkoxydiyl, or a covalent bond, thereby forming a cyclopropane ring, and

R₇ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy; or

ii) Y is —OR^{X} or C₁₋₈alkoxy,

Z is-OR^{Z}, —SF₅, unsubstituted 3-10 membered heterocycloalkyl,

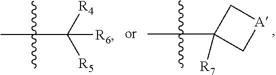

R₄ and R₅ are each independently unsubstituted C₁₋₈alkyl or substituted C₁₋₈alkyl, and

R₆ is —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,

A′ is —CF₂—, —O—, C₁₋₆alkanediyl, or a covalent bond, thereby forming a cyclopropane ring,

R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and

R^{X} is —(CH₂CH₂O)ᵣH or —(CH₂CH₂O)ᵣC₁₋₆alkyl, where r is an integer from 1-6, and

R^{Z} is —(CH₂CH₂O)ₛH or —(CH₂CH₂O)ₛC₁₋₆alkyl, where s is an integer from 1-6.

In some embodiments of Formula (III), R₁ may be unsubstituted C₁₋₈alkyl. In some embodiments of Formula (III), R₁ may be methyl. In some embodiments of Formula (III), R₂ may be hydrogen. In some embodiments of Formula (III), Y may be bromo, fluoro, cyano or substituted C₁₋₂alkoxy. In some embodiments of Formula (III), Y may be —OCF₃. In some embodiments of Formula (III), Y may be C₁₋₃lkoxy. In some embodiments of Formula (III), Y may be —OCH₃. In some embodiments of Formula (III), Z may be t-butyl. In some embodiments of Formula (III),

Z may be

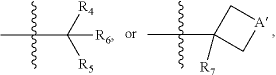

R₄ and R₅ are each —CH₃, and R₆ is —OH, —CN, —CF₃, —CF₂H, —CH₂F, —CH₂OH or C₁₋₈alkoxy, A′ is —CF₂—, —O—, or a covalent bond, thereby forming a cyclopropane ring, and R₇ is hydrogen, —OH, —CN, —CF₃, —CF₂H, —CH₂F, —CH₂OH, —CH₂O—C₁₋₈alkyl, C₁₋₃alkyl or C₁₋₃alkoxy. In some embodiments of Formula (III), Z may be

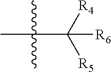

where R₆ is —OH, or —CH₂OH. In some embodiments of Formula (III), Z may be

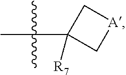

A′ is —O—, or a covalent bond, thereby forming a cyclopropane ring, and R₇ is hydrogen, —CF₃, —CF₂H, —CH₂F or —OCH₃.

In some aspects, the present disclosure provides compounds of the formula:

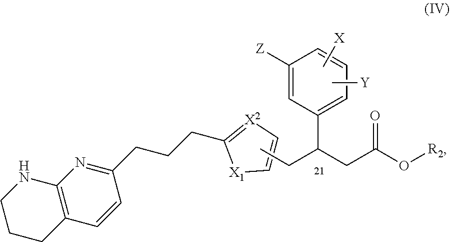

or a pharmaceutically acceptable salt, solvate or tautomer of the above formula, wherein: R₁, R₂, X, X₁, X₂, Y and Z have any of the values described herein.

In some embodiments of Formula (IV), R₁ may be hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, or substituted C₇₋₁₂aralkyl;

X₁ is O (oxygen), S (sulfur), or —NR^{1A}—;

R^{1A} is hydrogen, unsubstituted C₁₋₈alkyl or substituted C₁₋₈alkyl;

X₂ is N (nitrogen);
i) X and Y are each independently hydrogen, —OR^{X}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryl-CH₂O—, substituted C_{6 or 10}aryl-CH₂O—, unsubstituted C_{6 or 10}aryloxy, substituted C_{6 or 10}aryloxy, unsubstituted C₂₋₁₂acyloxy, substituted C₂₋₁₂acyloxy,

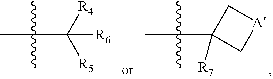

where R₄ and R₅ are each independently unsubstituted C₁₋₈alkyl or substituted C₁₋₈alkyl, and

R₆ is hydrogen, —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,

where A′ is —CF₂—, —O—, C₁₋₆alkanediyl, C₁₋₈alkoxydiyl, C₂₋₈alkylalkoxydiyl, or a covalent bond, thereby forming a cyclopropane ring, and

R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and

R^{X} is —O(CH₂CH₂O)ᵣH or —O(CH₂CH₂O)ᵣC₁₋₆alkyl, where r is an integer from 1-6; or
ii) X is —OR^{A} and Y is —OR^{B}, where R^{A} and R^{B} together are —(CR₁₂)ₙ—,

each R₁₂ is independently hydrogen or unsubstituted C₁₋₆alkyl, and

n is 1 or 2;

Z is —OR^{Z}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy, or

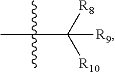

where R₈ and R₉ are each independently unsubstituted C₁₋₈alkyl or substituted C₁₋₈alkyl,

R₁₀ is hydrogen, —OH, —CN, —NH₂, —CF₃, —CF₂H, —CFH₂, —CO₂H, —CO₂—C₁₋₈alkyl, —C(═O)NH₂, —CH₂OH, CH₂O—C₁₋₈alkyl, or C₁₋₈alkoxy, and

R^{Z} is —O(CH₂CH₂O)ₛH or —O(CH₂CH₂O)ₛC₁₋₆alkyl, where s is an integer from 1-6, or

### Z is

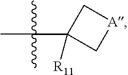

where A″ is —CF₂—, —O—, C₁₋₆alkanediyl, C₁₋₈alkoxydiyl, or C₂₋₈alkylalkoxydiyl, where R₁₁ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy.

In some embodiments, the compounds are further defined as:

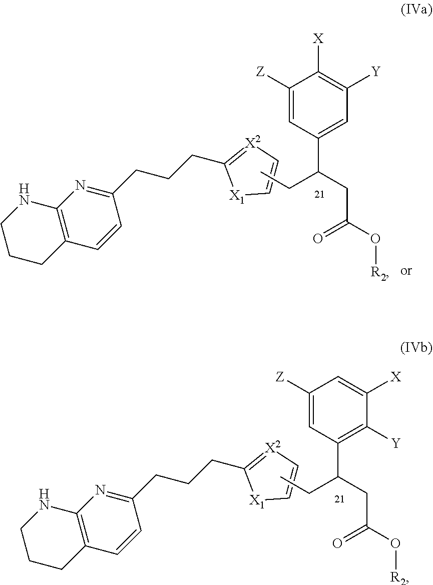

or a pharmaceutically acceptable salt, solvate or tautomer thereof, wherein: R₁, R₂, X, X₁, X₂, Y and Z have any of the values described herein.

In some embodiments, the compounds are further defined as:

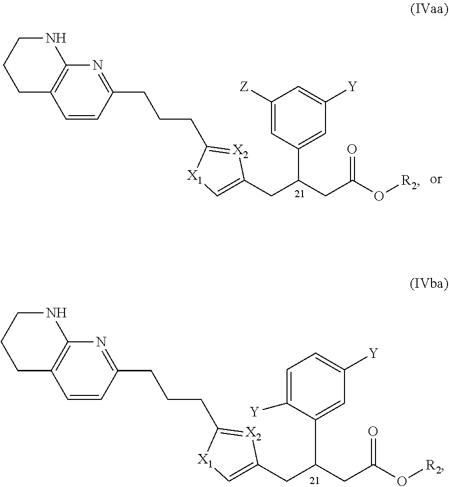

or a pharmaceutically acceptable salt, solvate or tautomer thereof, wherein: R₁, R₂, X₁, X₂, Y and Z have any of the values described herein.

In some embodiments, the compounds are further defined as:

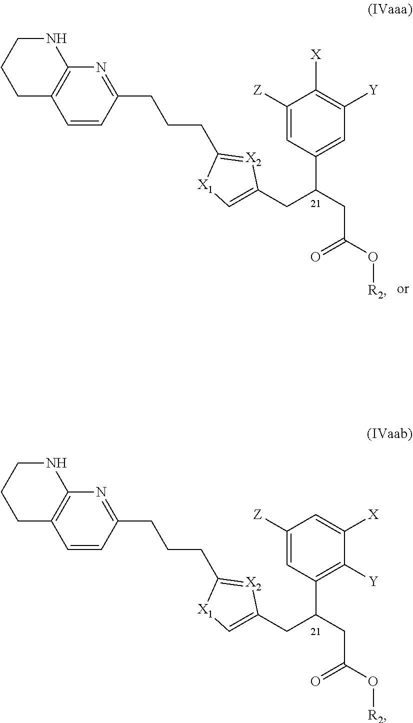

or a pharmaceutically acceptable salt, solvate or tautomer thereof, wherein: R₁, R₂, X, X₁, X₂, Y and Z have any of the values described herein.

In some embodiments of Formula (IV), (IVa), (IVb), (IVaa), (IVba), (IVaaa), and (IVaab), X₁ is O (oxygen), or S (sulfur). In some embodiments of Formula (IV), (IVa), (IVb), (IVaa), (IVba), (IVaaa), and (IVaab), X₁ is S (sulfur). In some embodiments of Formula (IV), (IVa), (IVb), (IVaa), (IVba), (IVaaa), and (IVaab), R₂ is hydrogen. In some embodiments of Formula (IV), (IVa), (IVb), (IVaa), (IVba), (IVaaa), and (IVaab), X is hydrogen, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, or substituted C₂₋₁₂acyloxy.

In some aspects, the present disclosure provides compounds of the formula:

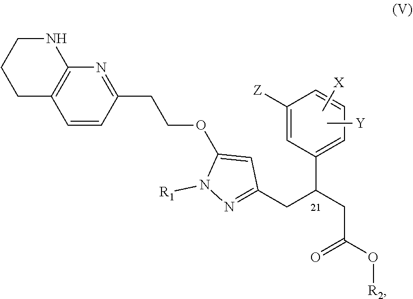

or a pharmaceutically acceptable salt, solvate or tautomer of the above formula, wherein: R₁, R₂, X, Y and Z have any of the values described herein.

In some embodiments of Formula (V), R₁ may be unsubstituted C₂₋₈alkyl, substituted C₁₋₈alkyl, unsubstituted C_{6 or 10}aryl, substituted C_{6 or 10}aryl, unsubstituted C₇₋₁₂aralkyl, or substituted C₇₋₁₂aralkyl;

R₂ may be hydrogen, unsubstituted C₁₋₈alkyl, or substituted C₁₋₈alkyl;
i) X and Y are each independently hydrogen, —OR^{X}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryl-CH₂O—, substituted C_{6 or 10}aryl-CH₂O—, unsubstituted C_{6 or 10}aryloxy, substituted C_{6 or 10}aryloxy, unsubstituted C₂₋₁₂acyloxy, substituted C₂₋₁₂acyloxy,

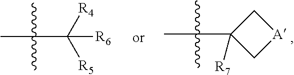

where R₄ and R₅ are each independently unsubstituted C₁₋₈alkyl or substituted C₁₋₈alkyl, and
R₆ is hydrogen, —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
where A′ is —CF₂—, —O—, C₁₋₆alkanediyl, C₁₋₈alkoxydiyl, C₂₋₈alkylalkoxydiyl, or a covalent bond, thereby forming a cyclopropane ring, and
R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and
R^{X} is —(CH₂CH₂O)ᵣH or —(CH₂CH₂O)ᵣC₁₋₆alkyl, where r is an integer from 1-6; or
ii) X is —OR^{A} and Y is —OR^{B}, where R^{A} and R^{B} together are —(CR₁₂)ₙ—,
   each R₁₂ is independently hydrogen or unsubstituted C₁₋₆alkyl, and
   n is 1 or 2; and
Z is —OR^{Z}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy, or

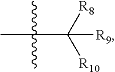

where R₈ and R₉ are each independently unsubstituted C₁₋₈alkyl or substituted C₁₋₈alkyl,
R₁₀ is hydrogen, —OH, —CN, —NH₂, —CF₃, —CF₂H, —CFH₂, —CO₂H, —CO₂— C₁₋₈alkyl, —C(═O)NH₂, —CH₂OH, CH₂O—C₁₋₈alkyl, or C₁₋₈alkoxy, and
R^{Z} is —(CH₂CH₂O)ₛH or —(CH₂CH₂O)ₛC₁₋₆alkyl, where s is an integer from 1-6, or

Z is

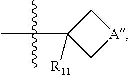

where A″ is —CF₂—, —O—, C₁₋₆alkanediyl, C₁₋₈alkoxydiyl, or C₂₋₈alkylalkoxydiyl, where R₁₁ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy.

In some embodiments, the compounds are further defined as:

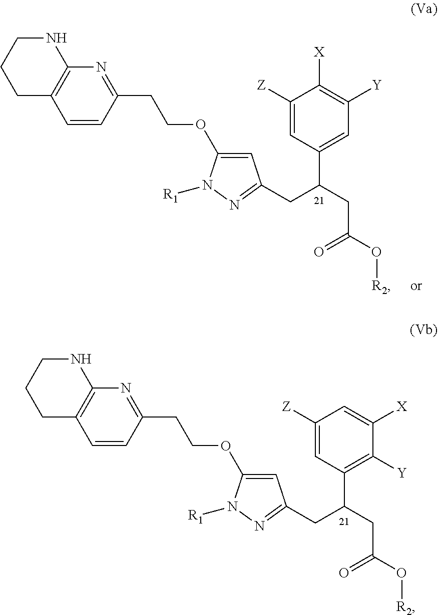

or a pharmaceutically acceptable salt, solvate or tautomer thereof, wherein: R₁, R₂, X, Y and Z have any of the values described herein.

In some embodiments, the compounds are further defined as:

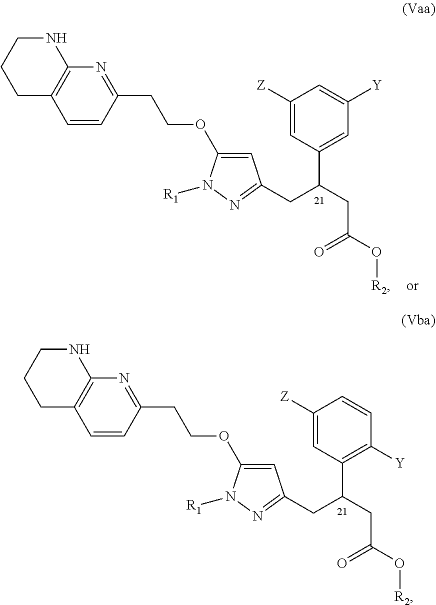

or a pharmaceutically acceptable salt, solvate or tautomer thereof, wherein: R₁, R₂, Y and Z have any of the values described herein.

In some aspects, the present disclosure provides compounds of the formula:

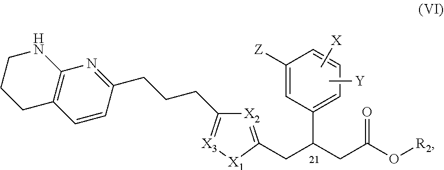

or a pharmaceutically acceptable salt, solvate or tautomer of the above formula, wherein: R₁, R₂, X, X₁, X₂, X₃, Y and Z have any of the values described herein.

In some embodiments of Formula (VI),
R₂ may be hydrogen, unsubstituted C₁₋₈alkyl, or substituted C₁₋₈alkyl;
X₁ may be O (oxygen), S (sulfur), or —NR^{1A}—;
X₂ may be CR^{1A} or N (nitrogen);
X₃ may be CR^{1A} or N (nitrogen);
each R^{1A} may independently be hydrogen, unsubstituted C₁₋₈alkyl or substituted C₁₋₈alkyl;
   i) X and Y are each independently hydrogen, —OR^{X}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryl-CH₂O—, substituted C_{6 or 10}aryl-CH₂O—, unsubstituted C_{6 or 10}aryloxy, substituted C_{6 or 10}aryloxy, unsubstituted C₂₋₁₂acyloxy, substituted C₂₋₁₂acyloxy,

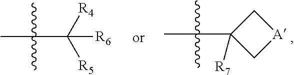

where R₄ and R₅ are each independently unsubstituted C₁₋₈alkyl or substituted C₁₋₈alkyl, and
R₆ is hydrogen, —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
where A′ is —CF₂—, —O—, C₁₋₆alkanediyl, C₁₋₈alkoxydiyl, C₂₋₈alkylalkoxydiyl, or a covalent bond, thereby forming a cyclopropane ring, and
R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and
R^{X} is —(CH₂CH₂O)ᵣH or —(CH₂CH₂O)ᵣC₁₋₆alkyl, where r is an integer from 1-6; or
ii) X is —OR^{A} and Y is —OR^{B}, where R^{A} and R^{B} together are —(CR₁₂)ₙ—,
   each R₁₂ is independently hydrogen or unsubstituted C₁₋₆alkyl, and
   n is 1 or 2; and
Z may be —OR^{Z}, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy, or

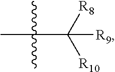

where R₈ and R₉ are each independently unsubstituted C₁₋₈alkyl or substituted C₁₋₈alkyl,
R₁₀ is hydrogen, —OH, —CN, —NH₂, —CF₃, —CF₂H, —CFH₂, —CO₂H, —CO₂— C₁₋₈alkyl, —C(═O)NH₂, —CH₂OH, CH₂O—C₁₋₈alkyl, or C₁₋₈alkoxy, and
R^{Z} is —(CH₂CH₂O)ₛH or —(CH₂CH₂O)ₛC₁₋₆alkyl, where s is an integer from 1-6, or

Z may be

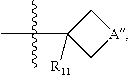

where A″ is —CF₂—, —O—, C₁₋₆alkanediyl, C₁₋₈alkoxydiyl, or C₂₋₈alkylalkoxydiyl, where R₁₁ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy. In some embodiments, X₂ may be N (nitrogen). In some embodiments, X₃ may be N (nitrogen).

In some embodiments of Formula (IV), (IVa), (IVb), (IVaa), (IVba), (IVaaa), (IVaab), (V), (Va), (Vb), (Vaa), (Vba), and (VI), X is hydrogen, halo, cyano, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy or

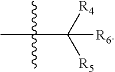

In some embodiments of Formula (IV), (IVa), (IVb), (IVaa), (IVba), (IVaaa), (IVaab), (V), (Va), (Vb), (Vaa), (Vba), and (VI), X is halo. In some embodiments of Formula (IV), (IVa), (IVb), (IVaa), (IVba), (IVaaa), (IVaab), (V), (Va), (Vb), (Vaa), (Vba), and (VI), X is bromo, fluoro, or chloro. In some embodiments of Formula (IV), (IVa), (IVb), (IVaa), (IVba), (IVaaa), (IVaab), (V), (Va), (Vb), (Vaa), (Vba), and (VI), X is —CF₃. In some embodiments of Formula (IV), (IVa), (IVb), (IVaa), (IVba), (IVaaa), (IVaab), (V), (Va), (Vb), (Vaa), (Vba), and (VI), X is —OH or cyano. In some embodiments of Formula (IV), (IVa), (IVb), (IVaa), (IVba), (IVaaa), (IVaab), (V), (Va), (Vb), (Vaa), (Vba), and (VI), X is unsubstituted C₁₋₈alkyl. In some embodiments of Formula (IV), (IVa), (IVb), (IVaa), (IVba), (IVaaa), (IVaab), (V), (Va), (Vb), (Vaa), (Vba), and (VI), X is unsubstituted C₃₋₆alkyl. In some embodiments of Formula (IV), (IVa), (IVb), (IVaa), (IVba), (IVaaa), (IVaab), (V), (Va), (Vb), (Vaa), (Vba), and (VI), X is unsubstituted C₁₋₈alkoxy. In some embodiments of Formula (IV), (IVa), (IVb), (IVaa), (IVba), (IVaaa), (IVaab), (V), (Va), (Vb), (Vaa), (Vba), and (VI), X is methoxy or isopropoxy.

In some embodiments of Formula (IV), (IVa), (IVb), (IVaa), (IVba), (IVaaa), (IVaab), (V), (Va), (Vb), (Vaa), (Vba), and (VI), Y or Z is t-butyl. In some embodiments of Formula (IV), (IVa), (IVb), (IVaa), (IVba), (IVaaa), (IVaab), (V), (Va), (Vb), (Vaa), (Vba), and (VI), Y or Z is

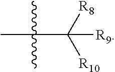

In some embodiments of Formula (IV), (IVa), (IVb), (IVaa), (IVba), (IVaaa), (IVaab), (V), (Va), (Vb), (Vaa), (Vba), and (VI), Y or Z is

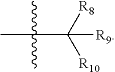

In some embodiments of Formula (IV), (IVa), (IVb), (IVaa), (IVba), (IVaaa), (IVaab), (V), (Va), (Vb), (Vaa), (Vba), and (VI), R₈ and R₉ are each independently unsubstituted C₂₋₈alkyl. In some embodiments of Formula (IV), (IVa), (IVb), (IVaa), (IVba), (IVaaa), (IVaab), (V), (Va), (Vb), (Vaa), (Vba), and (VI), R₈ and R₉ are each —CH₃. In some embodiments of Formula (IV), (IVa), (IVb), (IVaa), (IVba), (IVaaa), (IVaab), (V), (Va), (Vb), (Vaa), (Vba), and (VI), R₁₀ is —CF₃, —CF₂H, or —CFH₂. In some embodiments of Formula (IV), (IVa), (IVb), (IVaa), (IVba), (IVaaa), (IVaab), (V), (Va), (Vb), (Vaa), (Vba), and (VI), R₁₀ is —CF₃. In some embodiments of Formula (IV), (IVa), (IVb), (IVaa), (IVba), (IVaaa), (IVaab), (V), (Va), (Vb), (Vaa), (Vba), and (VI), R₁₀ is hydrogen or —CH₃.

In some embodiments of Formula (IV), (IVa), (IVb), (IVaa), (IVba), (IVaaa), (IVaab), (V), (Va), (Vb), (Vaa), (Vba), and (VI), Y or Z is

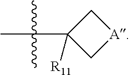

In some embodiments of Formula (IV), (IVa), (IVb), (IVaa), (IVba), (IVaaa), (IVaab), (V), (Va), (Vb), (Vaa), (Vba), and (VI), A″ is C₁₋₃alkanediyl, C₁₋₄alkoxydiyl, or a covalent bond, thereby forming a cyclopropane ring. In some embodiments of Formula (IV), (IVa), (IVb), (IVaa), (IVba), (IVaaa), (IVaab), (V), (Va), (Vb), (Vaa), (Vba), and (VI), A″ is a covalent bond, thereby forming a cyclopropane ring. In some embodiments of Formula (IV), (IVa), (IVb), (IVaa), (IVba), (IVaaa), (IVaab), (V), (Va), (Vb), (Vaa), (Vba), and (VI), R₁₁ is —CF₃, —CF₂H, —CH₂F, —CH₂O—C₁₋₆alkyl, C₁₋₆alkyl or C₁₋₈alkoxy. In some embodiments of Formula (IV), (IVa), (IVb), (IVaa), (IVba), (IVaaa), (IVaab), (V), (Va), (Vb), (Vaa), (Vba), and (VI), R₁₁ is —CF₃, —CF₂H, —CH₂F, C₁₋₆alkyl or C₁₋₆alkoxy. In some embodiments of Formula (IV), (IVa), (IVb), (IVaa), (IVba), (IVaaa), (IVaab), (V), (Va), (Vb), (Vaa), (Vba), and (VI), R₁₁ is —CF₃, —CF₂H or methoxy. In some embodiments of Formula (IV), (IVa), (IVb), (IVaa), (IVba), (IVaaa), (IVaab), (V), (Va), (Vb), (Vaa), (Vba), and (VI), R₁₁ is —CF₃ or —CF₂H. In some embodiments of Formula (IV), (IVa), (IVb), (IVaa), (IVba), (IVaaa), (IVaab), (V), (Va), (Vb), (Vaa), (Vba), and (VI), R₁₁ is —CH₂O—CH₃.

In some embodiments, the compound may be an integrin antagonist. In some embodiments, the integrin may be an α₅β₁ integrin antagonist. In some embodiments, the compound exhibits an IC₅₀ value for the α₅β₁ integrin of less than 50 nM, 40 nM, 30 nM, 20 nM, 15 nm or 1 nM, or a range defined by any of the preceding as measured by a solid phase receptor assay for α₅β₁ integrin function. In some embodiments, the integrin is an α_{V}β₁ integrin antagonist. In some embodiments, the compound exhibits an IC₅₀ value for the α_{V}β₁ integrin of less than 15 nM as measured by a solid phase receptor assay for α_{V}β₁ integrin function. In some embodiments, the compound exhibits an IC₅₀ value for an α_{V}β₃ integrin of less than 10 nM as measured by a solid phase receptor assay for α_{V}β₃ integrin function. In some embodiments, the compound exhibits an IC₅₀ value for an α_{V}β₅ integrin of less than 10 nM as measured by a solid phase receptor assay for α_{V}β₅ integrin function. In some embodiments, the compound exhibits an IC₅₀ value for the α_{V}β₁, α_{V}β₃, and α_{V}β₅ integrins of less than 10 nM as measured by a solid phase receptor assays for α_{V}β₁, α_{V}β₃, and α_{V}β₅ integrin function. In some embodiments, the compound exhibits an IC₅₀ value for an α_{V}β₆ integrin of greater than 10 nM as measured by a solid phase receptor assay for α_{V}β₆ integrin function. In some embodiments, the compound exhibits an IC₅₀ value for an α_{V}β₈ integrin of greater than 10 nM as measured by a solid phase receptor assay for α_{V}β₈ integrin function. In some embodiments, the compound exhibits an IC₅₀ value for the α_{V}β₆ and α_{V}β₈ integrins of greater than 10 nM as measured by solid phase receptor assays for α_{V}β₆ and α_{V}β₈ integrin function.

In some embodiments, the compound is an integrin antagonist such as an α_{V}β₁ integrin antagonist. In some embodiments, the compound exhibits an IC₅₀ value for the α_{V}β₁ integrin of less than 15 nM as measured by a solid phase receptor assay for α_{V}β₁ integrin function. In some embodiments, the compound exhibits an IC₅₀ value for an α_{V}β₃ integrin of less than 10 nM as measured by a solid phase receptor assay for α_{V}β₆ integrin function. In some embodiments, the compound exhibits an IC₅₀ value for an α_{V}β₅ integrin of less than 10 nM as measured by a solid phase receptor assay for α_{V}β₅ integrin function. In some embodiments, the compound exhibits an IC₅₀ value for the α_{V}β₁, α_{V}β₃, and α_{V}β₅ integrins of less than 10 nM as measured by a solid phase receptor assays for α_{V}β₁, α_{V}β₃, and α_{V}β₅ integrin function. In some embodiments, the compound exhibits an IC₅₀ value for an α_{V}β₆ integrin of greater than 10 nM as measured by a solid phase receptor assay for α_{V}β₁ integrin function. In some embodiments, the compound exhibits an IC₅₀ value for an α_{V}β₈ integrin of greater than 10 nM as measured by a solid phase receptor assay for α_{V}β₁ integrin function. In some embodiments, the compound exhibits an IC₅₀ value for the α_{V}β₆ and α_{V}β₈ integrins of greater than 10 nM as measured by solid phase receptor assays for α_{V}β₆ and α_{V}β₈ integrin function.

In some embodiments, the compounds are further defined as:

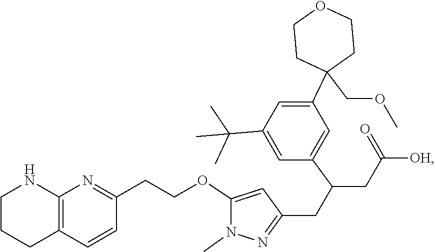 [SMILES: COCC1(c2cc(C(CC(=O)O)Cc3cc(OCCc4ccc5c(n4)NCCC5)n(C)n3)cc(C(C)(C)C)c2)CCOCC1]

or a pharmaceutically acceptable salt thereof. In some embodiments, the compounds are further defined as:

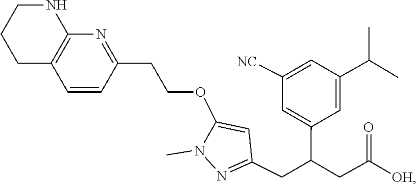 [SMILES: CC(C)c1cc(C#N)cc(C(CC(=O)O)Cc2cc(OCCc3ccc4c(n3)NCCC4)n(C)n2)c1]

or a pharmaceutically acceptable salt thereof.

In some embodiments, the compounds are further defined as:

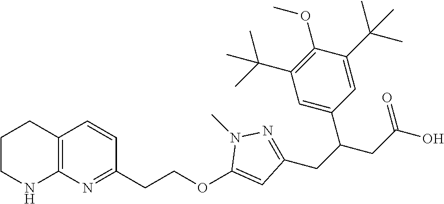 [SMILES: COc1c(C(C)(C)C)cc(C(CC(=O)O)Cc2cc(OCCc3ccc4c(n3)NCCC4)n(C)n2)cc1C(C)(C)C]

or a pharmaceutically acceptable salt, solvate or tautomer thereof.

In some embodiments, the compounds are further defined as:

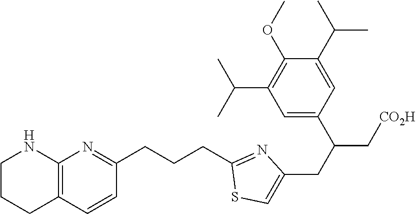 [SMILES: COc1c(C(C)C)cc(C(CC(=O)O)Cc2csc(CCCc3ccc4c(n3)NCCC4)n2)cc1C(C)C]

or a pharmaceutically acceptable salt, solvate or tautomer thereof.

In still yet another aspect, the present disclosure provides pharmaceutical compositions comprising:
a) a compound as disclosed and described herein; and
b) an excipient.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. The pharmaceutical composition may be formulated for oral, topical, intravenous, or intravitreal administration. In some embodiments, the pharmaceutical composition is formulated as a unit dose.

In yet another aspect, the present disclosure provides methods of treating and/or preventing a disease or a disorder in a patient in need thereof, comprising administering to the patient a compound or composition described herein in an amount sufficient to treat and/or prevent the disease or disorder. In some embodiments, the disease or disorder is associated with fibrosis. The disease or disorder may be scleroderma or fibrosis of the lungs, liver, kidneys, heart, skin, or pancreas. In some embodiments, the disease or disorder is fibrosis of the lungs. In other embodiments, the disease or disorder is fibrosis of the liver. In other embodiments, the disease or disorder is fibrosis of the heart. In other embodiments, the disease or disorder is fibrosis of the kidneys. In other embodiments, the disease or disorder is fibrosis of the pancreas. In other embodiments, the disease or disorder is fibrosis of the skin. In some embodiments, the disease or disorder is scleroderma.

In some embodiments, the patient is a human, monkey, cow, horse, sheep, goat, dog, cat, mouse, rat, guinea pig, or transgenic species thereof. The patient may be a monkey, cow, horse, sheep, goat, dog, cat, mouse, rat, or guinea pig. Alternatively, the patient may be a human.

In still yet another aspect, the present disclosure provides methods of inhibiting the binding of an integrin comprising contacting the integrin with a compound or composition described herein. The integrin may be α₅β₁, α_{V}β₁, α_{V}β₃, or α_{V}β₅. In some embodiments, the integrin is α₅β₁. In some further embodiments, the integrin is α_{V}β₁. In some embodiments, the method is performed in vitro. In other embodiments, the method is performed ex vivo or in vivo. In some embodiments, the inhibition of binding is sufficient to treat or prevent a disease or disorder in a patient.

Some embodiments provide a method of treating and/or preventing a disease or a disorder in a patient in need thereof, comprising administering to the patient a compound or composition as disclosed and described herein in an amount sufficient to treat and/or prevent the disease or disorder. In some embodiments, the disease or disorder is associated with fibrosis. In some embodiments, the disease or disorder is scleroderma or fibrosis of the lungs, liver, kidneys, heart, skin, or pancreas. In some embodiments, the disease or disorder is fibrosis of the lungs. In some embodiments, the disease or disorder is fibrosis of the liver. In some embodiments, the disease or disorder is fibrosis of the heart. In some embodiments, the disease or disorder is fibrosis of the kidneys. In some embodiments, the disease or disorder is fibrosis of the pancreas. In some embodiments, the disease or disorder is fibrosis of the skin. In some embodiments, the disease or disorder is scleroderma. In some embodiments, the patient is a human, monkey, cow, horse, sheep, goat, dog, cat, mouse, rat, guinea pig, or transgenic species thereof. In some embodiments, the patient is a monkey, cow, horse, sheep, goat, dog, cat, mouse, rat, or guinea pig. In some embodiments, the patient is a human.

Some embodiments provide a method of inhibiting the binding of an integrin comprising contacting the integrin with a compound or composition as disclosed and described herein. In some embodiments, the integrin is α5β1, αVβ1, αVβ3, or αVβ5. In some embodiments, the integrin is αVβ1. In some embodiments, the integrin is α5β1. In some embodiments, the method is performed in vitro. In some embodiments, the method is performed ex vivo or in vivo. In some embodiments, the inhibition of binding is sufficient to treat or prevent a disease or disorder in a patient.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### DETAILED DESCRIPTION

Disclosed herein are new compounds and compositions which may act as α₅β₁, or αᵥβ₁ integrin antagonist, methods for their manufacture, and methods for their use, including for the treatment and/or prevention of diseases or disorders mediated by integrins. In some embodiments, the compounds provided herein may be used for the selective inhibition or antagonism of integrins α₅β₁, α_{V}β₁, α_{V}β₃ and/or α_{V}β₅. In some embodiments, the compounds provided herein exhibit reduced inhibitory or antagonistic activity of integrins α_{V}β₆, α_{V}β₈, and/or α_{IIb}β₃.

### I. COMPOUNDS AND SYNTHETIC METHODS

The compounds provided by the present disclosure may be made using the methods outlined below and further described in the Examples section. Those with skill in the art will readily understand that known variations of the conditions and processes described in the Examples can be used to synthesize the compounds of the present disclosure. Starting materials and equipment employed were either commercially available prepared by methods previously reported and readily duplicated by those skilled in the art. Such principles and techniques are taught, for example, in March's Advanced Organic Chemistry: Reactions, Mechanisms, and Structure (2007), which is incorporated by reference herein.

In some embodiments, the compounds of the present disclosure include the compounds described in the Examples and claims listed below. Some embodiments include compounds active as inhibitors of integrin α_{V}β1 and α₅β₁, such as compounds listed in Table 1 below (compounds of Formula (II) which contain X and Y substituent groups, one of which is a bulky group). Some embodiments include compounds active as inhibitors of integrin α_{V}β1, that also in general have increased activity as inhibitors of integrin α₅β₁ as compared with compounds that do not contain a bulky substituent.

TABLE 1
Example Compounds of the Present Disclosure

| Example Number | Compound Structure |
|---|---|
| Example 1 | 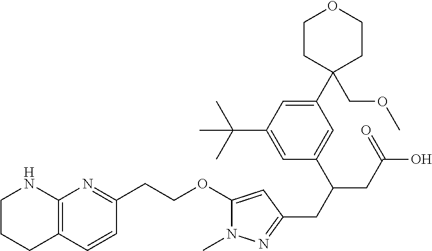 [SMILES: COCC1(c2cc(C(CC(=O)O)Cc3cc(OCCc4ccc5c(n4)NCCC5)n(C)n3)cc(C(C)(C)C)c2)CCOCC1] |
| Example 2 | 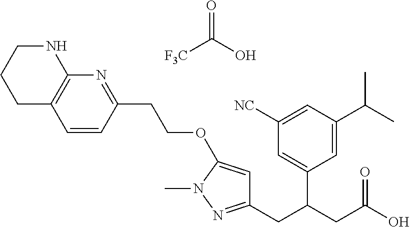 [SMILES: CC(C)c1cc(C#N)cc(C(CC(=O)O)Cc2cc(OCCc3ccc4c(n3)NCCC4)n(C)n2)c1 ; O=C(O)C(F)(F)F] |
| Example 3 | 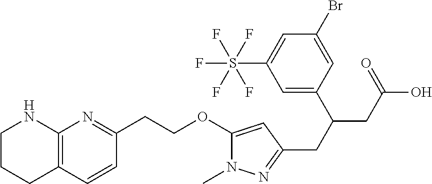 [SMILES: Cn1nc(CC(CC(=O)O)c2cc(Br)cc(S(F)(F)(F)(F)F)c2)cc1OCCc1ccc2c(n1)NCCC2] |
| Example 4 | 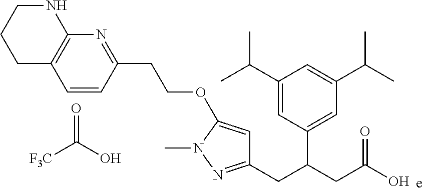 |
| Example 5 | 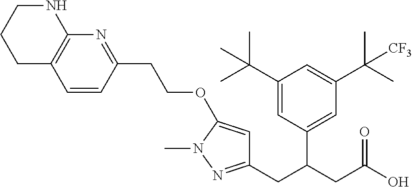 [SMILES: Cn1nc(CC(CC(=O)O)c2cc(C(C)(C)C)cc(C(C)(C)C(F)(F)F)c2)cc1OCCc1ccc2c(n1)NCCC2] |
| Example 6 | 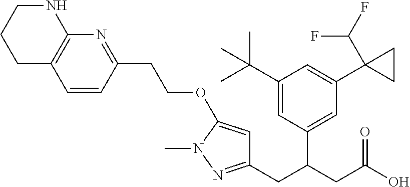 [SMILES: Cn1nc(CC(CC(=O)O)c2cc(C(C)(C)C)cc(C3(C(F)F)CC3)c2)cc1OCCc1ccc2c(n1)NCCC2] |
| Example 7 | 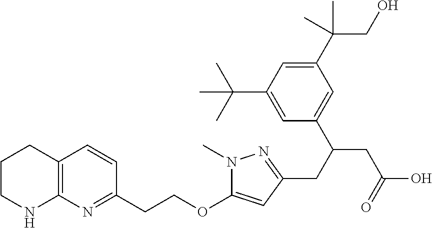 [SMILES: Cn1nc(CC(CC(=O)O)c2cc(C(C)(C)C)cc(C(C)(C)CO)c2)cc1OCCc1ccc2c(n1)NCCC2] |
| Example 8 | 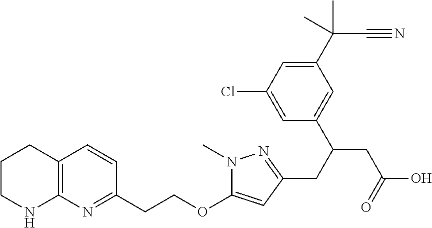 [SMILES: Cn1nc(CC(CC(=O)O)c2cc(Cl)cc(C(C)(C)C#N)c2)cc1OCCc1ccc2c(n1)NCCC2] |
| Example 9 | 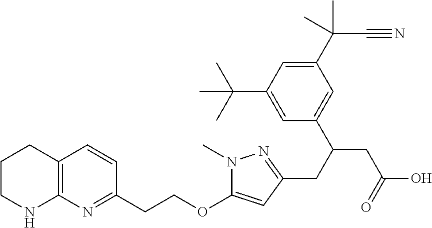 [SMILES: Cn1nc(CC(CC(=O)O)c2cc(C(C)(C)C)cc(C(C)(C)C#N)c2)cc1OCCc1ccc2c(n1)NCCC2] |
| Example 40 | 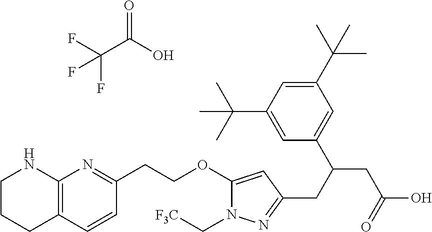 [SMILES: CC(C)(C)c1cc(C(CC(=O)O)Cc2cc(OCCc3ccc4c(n3)NCCC4)n(CC(F)(F)F)n2)cc(C(C)(C)C)c1 ; O=C(O)C(F)(F)F] |
| Example 41 | 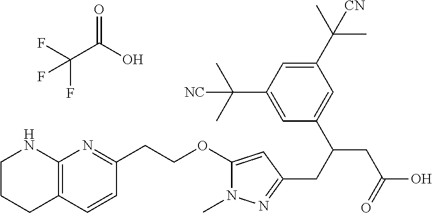 [SMILES: Cn1nc(CC(CC(=O)O)c2cc(C(C)(C)C#N)cc(C(C)(C)C#N)c2)cc1OCCc1ccc2c(n1)NCCC2 ; O=C(O)C(F)(F)F] |

Some embodiments include compounds active as inhibitors of integrin α_{V}β1, such as compounds listed in Table 2 below (compounds of Formula (I), which contain X, Y and Z substituent groups). Some embodiments include compounds active as inhibitors of integrin α_{V}β1 and integrin α₅β₁.

TABLE 2
Example Compounds of the Present Disclosure

| Example Number | Compound Structure |
|---|---|
| Example 10 | 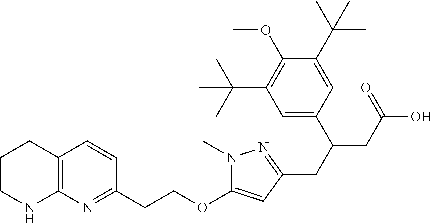 [SMILES: COc1c(C(C)(C)C)cc(C(CC(=O)O)Cc2cc(OCCc3ccc4c(n3)NCCC4)n(C)n2)cc1C(C)(C)C] |
| Example 11 | 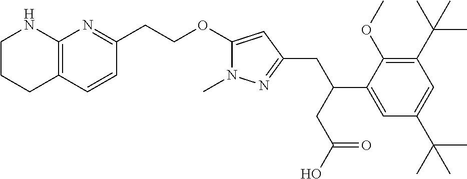 [SMILES: COc1c(C(CC(=O)O)Cc2cc(OCCc3ccc4c(n3)NCCC4)n(C)n2)cc(C(C)(C)C)cc1C(C)(C)C] |
| Example 12 | 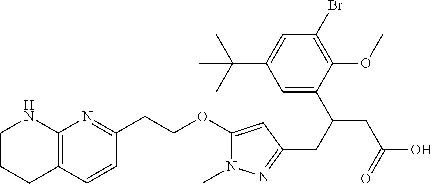 [SMILES: COc1c(Br)cc(C(C)(C)C)cc1C(CC(=O)O)Cc1cc(OCCc2ccc3c(n2)NCCC3)n(C)n1] |
| Example 13 | 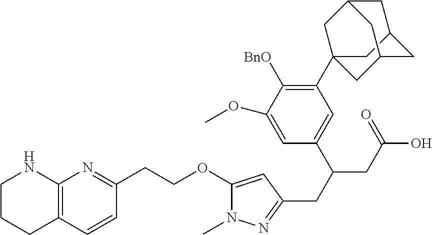 [SMILES: COc1cc(C(CC(=O)O)Cc2cc(OCCc3ccc4c(n3)NCCC4)n(C)n2)cc(C23CC4CC(CC(C4)C2)C3)c1OCc1ccccc1] |
| Example 14 | 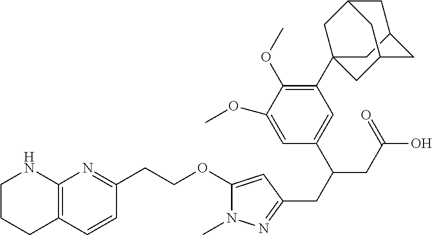 [SMILES: COc1cc(C(CC(=O)O)Cc2cc(OCCc3ccc4c(n3)NCCC4)n(C)n2)cc(C23CC4CC(CC(C4)C2)C3)c1OC] |
| Example 15 | 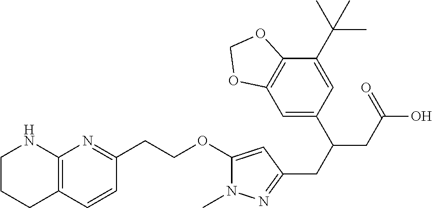 [SMILES: Cn1nc(CC(CC(=O)O)c2cc3c(c(C(C)(C)C)c2)OCO3)cc1OCCc1ccc2c(n1)NCCC2] |
| Example 16 | 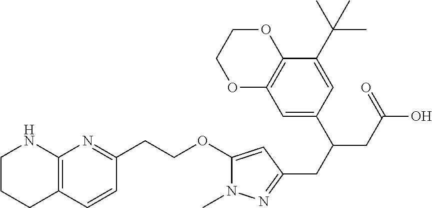 [SMILES: Cn1nc(CC(CC(=O)O)c2cc3c(c(C(C)(C)C)c2)OCCO3)cc1OCCc1ccc2c(n1)NCCC2] |
| Example 25 | 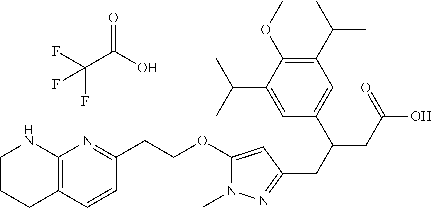 |
| Example 47 | 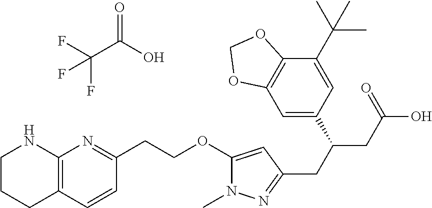 [SMILES: Cn1nc(C[C@@H](CC(=O)O)c2cc3c(c(C(C)(C)C)c2)OCO3)cc1OCCc1ccc2c(n1)NCCC2 ; O=C(O)C(F)(F)F] |

Some embodiments include compounds active as inhibitors of integrin α_{V}β1 listed in Table 3 below (compounds of Formula (III), which contain Z and Y substituent groups, where Z is a bulky group). Some embodiments include compounds active as inhibitors of integrin α_{V}β1 and integrin α₅β₁.

TABLE 3
Example Compounds of the Present Disclosure

| Example Number | Compound Structure |
|---|---|
| Example 17 | 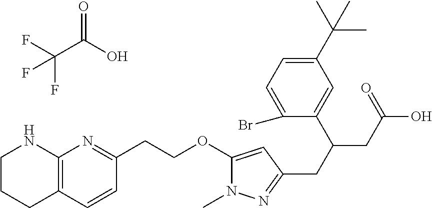 [SMILES: Cn1nc(CC(CC(=O)O)c2cc(C(C)(C)C)ccc2Br)cc1OCCc1ccc2c(n1)NCCC2 ; O=C(O)C(F)(F)F] |
| Example 18 | 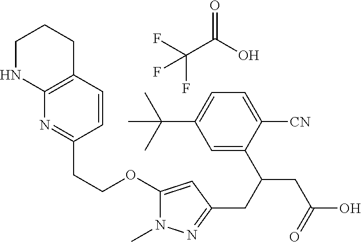 |
| Example 19 | 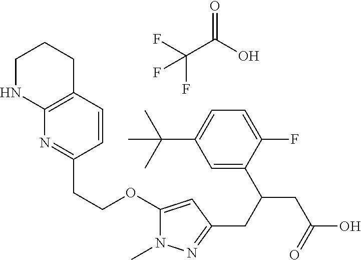 |
| Example 20 | 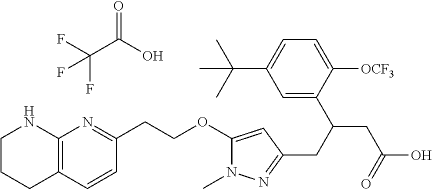 |
| Example 21 | 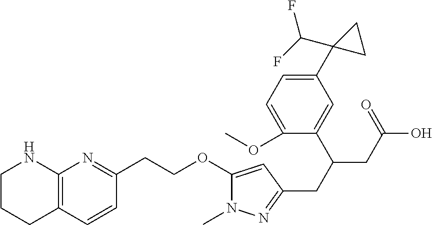 [SMILES: COc1ccc(C2(C(F)F)CC2)cc1C(CC(=O)O)Cc1cc(OCCc2ccc3c(n2)NCCC3)n(C)n1] |
| Example 22 | 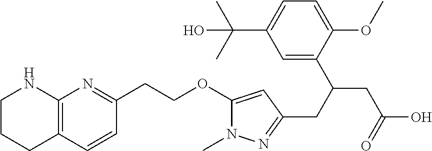 [SMILES: COc1ccc(C(C)(C)O)cc1C(CC(=O)O)Cc1cc(OCCc2ccc3c(n2)NCCC3)n(C)n1] |
| Example 23 | 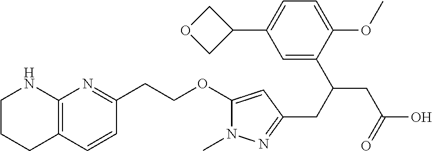 [SMILES: COc1ccc(C2COC2)cc1C(CC(=O)O)Cc1cc(OCCc2ccc3c(n2)NCCC3)n(C)n1] |
| Example 24 | 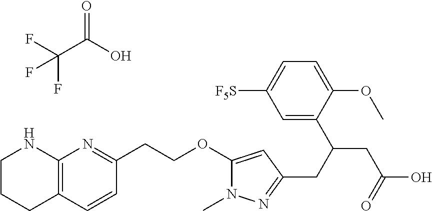 |
| Example 39 | 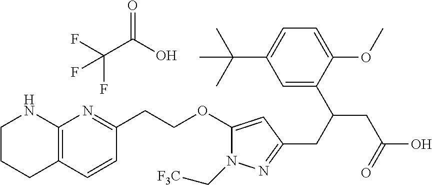 |
| Example 42 | 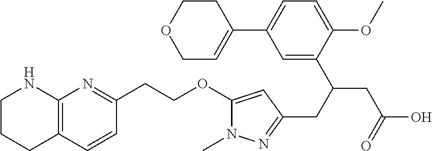 [SMILES: COc1ccc(C2=CCOCC2)cc1C(CC(=O)O)Cc1cc(OCCc2ccc3c(n2)NCCC3)n(C)n1] |
| Example 45 | 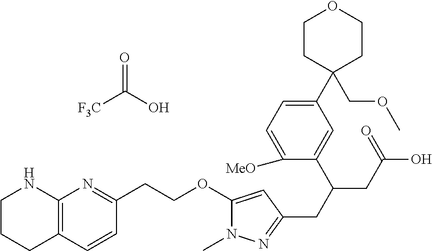 [SMILES: COCC1(c2ccc(OC)c(C(CC(=O)O)Cc3cc(OCCc4ccc5c(n4)NCCC5)n(C)n3)c2)CCOCC1 ; O=C(O)C(F)(F)F] |
| Example 46 | 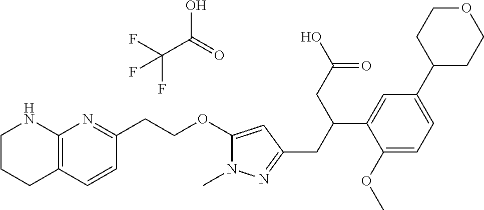 |
| Example 48 | 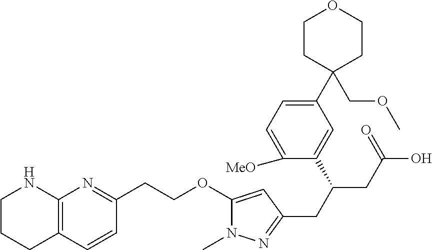 [SMILES: COCC1(c2ccc(OC)c([C@H](CC(=O)O)Cc3cc(OCCc4ccc5c(n4)NCCC5)n(C)n3)c2)CCOCC1] |

Some embodiments include compounds active as inhibitors of integrin α_{V}β1, such as compounds listed in Table 4 below (compounds of Formula (IV)).

TABLE 4
Example Compounds of the Present Disclosure

| Example Number | Compound Structure |
|---|---|
| Example 26 | 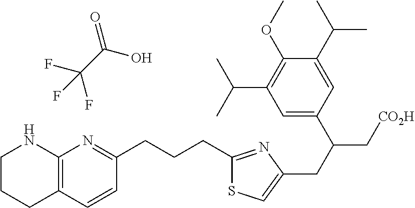 |
| Example 27 | 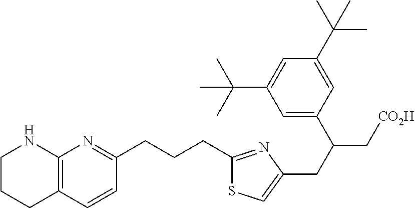 [SMILES: CC(C)(C)c1cc(C(CC(=O)O)Cc2csc(CCCc3ccc4c(n3)NCCC4)n2)cc(C(C)(C)C)c1] |
| Example 28 | 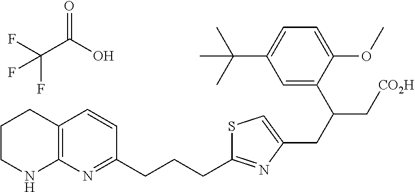 |
| Example 29 | 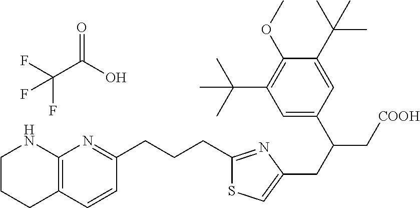 |
| Example 30 | 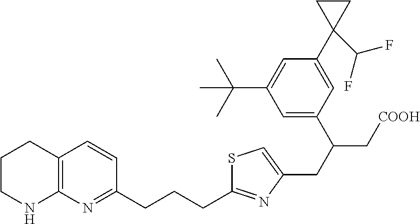 [SMILES: CC(C)(C)c1cc(C(CC(=O)O)Cc2csc(CCCc3ccc4c(n3)NCCC4)n2)cc(C2(C(F)F)CC2)c1] |
| Example 31 | 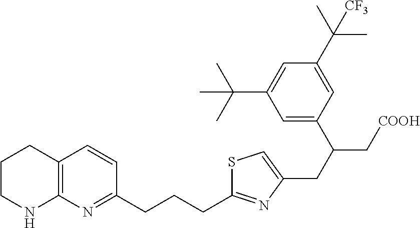 [SMILES: CC(C)(C)c1cc(C(CC(=O)O)Cc2csc(CCCc3ccc4c(n3)NCCC4)n2)cc(C(C)(C)C(F)(F)F)c1] |
| Example 32 | 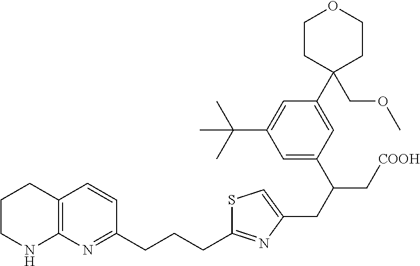 [SMILES: COCC1(c2cc(C(CC(=O)O)Cc3csc(CCCc4ccc5c(n4)NCCC5)n3)cc(C(C)(C)C)c2)CCOCC1] |
| Example 33 | 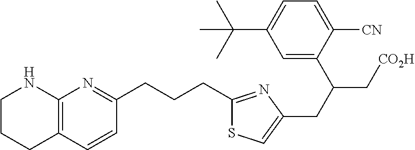 [SMILES: CC(C)(C)c1ccc(C#N)c(C(CC(=O)O)Cc2csc(CCCc3ccc4c(n3)NCCC4)n2)c1] |
| Example 34 | 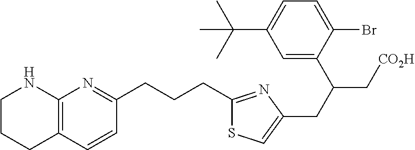 [SMILES: CC(C)(C)c1ccc(Br)c(C(CC(=O)O)Cc2csc(CCCc3ccc4c(n3)NCCC4)n2)c1] |
| Example 35 | 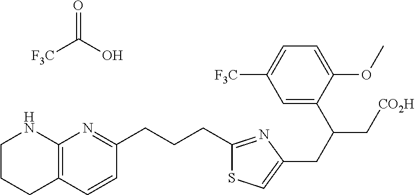 [SMILES: COc1ccc(C(F)(F)F)cc1C(CC(=O)O)Cc1csc(CCCc2ccc3c(n2)NCCC3)n1 ; O=C(O)C(F)(F)F] |
| Example 36 | 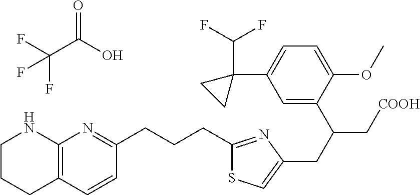 |
| Example 37 | 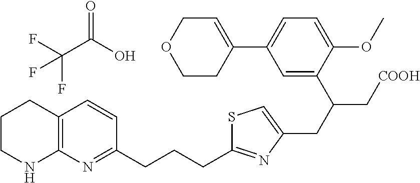 |
| Example 38 | 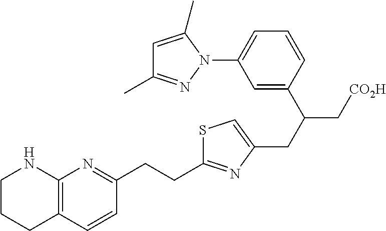 [SMILES: Cc1cc(C)n(-c2cccc(C(CC(=O)O)Cc3csc(CCc4ccc5c(n4)NCCC5)n3)c2)n1] |
| Example 43 | 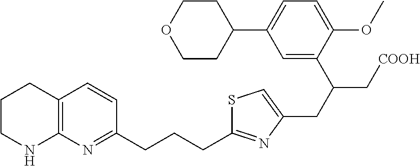 [SMILES: COc1ccc(C2CCOCC2)cc1C(CC(=O)O)Cc1csc(CCCc2ccc3c(n2)NCCC3)n1] |

All of the compounds of the present disclosure may be useful for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all of the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds employed in methods of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration, as defined by the IUPAC 1974 Recommendations. In some embodiments, the compounds of the present disclosure are in the S configuration. For example, mixtures of stereoisomers may be separated using the techniques taught in the Examples section below, as well as modifications thereof. Tautomeric forms are also included as well as pharmaceutically acceptable salts of such isomers and tautomers.

Atoms making up the compounds of the present disclosure are intended to include all isotopic forms of such atoms. Compounds of the present disclosure include those with one or more atoms that have been isotopically modified or enriched, in particular those with pharmaceutically acceptable isotopes or those useful for pharmaceutically research. Isotopes, as used herein, include those atoms having the same atomic number but different mass numbers. By way of general example and without limitation, isotopes of hydrogen include deuterium and tritium, and isotopes of carbon include ¹³C and ¹⁴C. Similarly, it is contemplated that one or more carbon atom(s) of a compound of the present disclosure may be replaced by a silicon atom(s). Furthermore, it is contemplated that one or more oxygen atom(s) of a compound of the present disclosure may be replaced by a sulfur or selenium atom(s).

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It should be further recognized that the compounds of the present disclosure include those that have been further modified to comprise substituents that are convertible to hydrogen in vivo. This includes those groups that may be convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃, BOC), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), lie (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃, Boc), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), ten-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trltyl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

### II. BIOLOGICAL ACTIVITY

It is another object of the disclosure to provide new compounds and compositions which may act as αᵥβ₁ and/or α₅β₁ integrin antagonist, methods for their manufacture, and methods for their use, including for the treatment and/or prevention of diseases or disorders mediated by integrins. In some embodiments, the compounds may be used for the selective inhibition or antagonism of integrins α₅β₁, α_{V}β₁, α_{V}β₃, and/or α_{V}β₅. In some embodiments, the compounds provided herein exhibit reduced inhibitory or antagonistic activity of integrins α_{V}β₃ α_{V}β₅, α_{V}β₆, α_{V}β₅, and/or α_{IIb}βₛ. In some further embodiments, the compounds provided herein exhibit reduced inhibitory or antagonistic activity of integrins α_{V}β₃, and/or α_{V}β₅.

Such compounds and compositions are useful in inhibiting or antagonizing integrins, and therefore in another embodiment, the present disclosure provides methods for inhibiting or antagonizing the α₅β₁, α_{V}μ1, α_{V}β, and/or α_{V}β5 integrins.

While not being bound by any particular theory, it has been unexpectedly discovered that compounds having at least one bulky substituent at substituent X, Y, and/or Z exhibit significantly increased activity against integrin α5β1. Examples of bulky substituents include unsubstituted alkyl groups, for example branched alkyl groups; substituted alkyl groups; cyclic groups, for example, cycloalkyl; and heterocycloalkyl groups. In some embodiments, at least one bulky substituent is at the meta position on the phenyl ring. Prior compounds lacking such a bulky substituents primarily acted on other integrin receptors, while activity against α5β1 was relatively low. In some embodiments, a compound having a bulky group at X, Y, or Z exhibits increased activity against integrin α5β1 compared to a structurally related compound lacking such a bulky substituent, for example, comparing compounds having a bulky group with the comparator compounds of Table 5 below.

TABLE 5
Comparator Compounds

| Comparator Number | Compound Structure |
|---|---|
| Comparator 1 (CC1) | 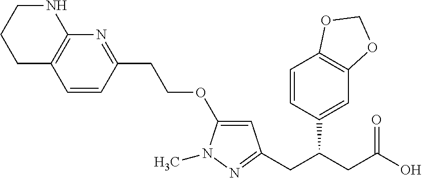 [SMILES: Cn1nc(C[C@@H](CC(=O)O)c2ccc3c(c2)OCO3)cc1OCCc1ccc2c(n1)NCCC2] |
| Comparator 2 (CC2) | 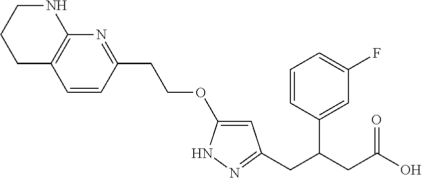 [SMILES: O=C(O)CC(Cc1cc(OCCc2ccc3c(n2)NCCC3)[nH]n1)c1cccc(F)c1] |

Accordingly, compounds having at least one bulky group at substituent X, Y, and/or Z may be used in treating conditions involving integrin α5β1 activity. Cells expressing α5β1 are believed to bind to fibronectin in a region that incorporates the ninth and tenth type III fibronectin repeats, the latter of which is believed to contain the RGD motif for integrin binding. In addition to fibronectin, α5β1 has been reported to interact with other RGD-containing extracellular matrix proteins including fibrinogen, denatured collagen, and fibrillin-1 (Bax et al, J. Biol. Chem., 278(36):34605-34616, 2003, 2003; Perdih, Curr. Med. Chem., 17(22):2371-2392, 2010; Suehiro et al., J. Biochem., 128(4):705-710, 2000). These ligands are generally classified as components of the provisional matrix that is laid down by cells as part of the wound healing response in tissues. Components of this response are angiogenesis (new blood vessel formation) and fibrosis (scar formation) which are beneficial for healing of acute injuries, but can be deleterious in many disease contexts. The important role of α5β1 in angiogenesis is supported by numerous studies. For example, mice lacking this integrin exhibit embryonic lethality at day 10-11 with a phenotype that includes defects in both the embryonic and extraembryonic vasculature (Yang et al., Development, 119(4): 1093-1105, 1993). Angiogenic cytokines such as bFGF, IL-8, TGFβ, and TNFα are believed to upregulate α5β1 expression on endothelial cells in vitro and in vivo, and immunohistochemistry shows coordinated increases in both α5β1 and fibronectin staining in blood vessels from various types of human tumor biopsies and xenograft tumors in animals (Collo, J. Cell Sci., 112(Pt 4):569-578, 1999; Kim et al., Am. J. Pathol., 156(4):1345-1362, 2000). Monoclonal antibodies that specifically inhibit α5β1, and compounds that have been described as α5β1 inhibitors, have been observed to significantly reduce angiogenesis in some experimental models (Kim et al., Am. J. Pathol., 156(4):1345-1362, 2000; Bhaskar et al., J. Transl. Med., 5:61, 2007; Livant et al., J. Clin. Invest., 105(11): 1537-1545, 2000; Zahn et al., Arch. Ophthalmol, 127(10): 1329-1335, 2009).

α5β1 expression is not confined to the endothelium, and it may have other functional roles in addition to angiogenesis. α5β1 is expressed to varying degrees in many cell types including fibroblasts, hematopoietic and immune cells, smooth muscle cells, epithelial cells, and tumor cells. Expression on tumor cells has been implicated in the progression of tumor growth and metastasis (Adachi et al., Clin. Cancer Res., 6(1):96-101, 2000, 2000; Blase et al., Int. J. Cancer, 60(6):860-866, 1995; Danen et al., Histopathology, 24(3):249-256, 1994; Edward, Curr. Opin. Oncol., 7(2): 185-191, 1995). In human fibroblasts, α5β1 was found to promote motility and survival (Lobert et al., Dev. Cell, 19(1): 148-159, 2010). In pancreatic stellate cells, α5β1 interacts with connective tissue growth factor to stimulate adhesion, migration, and fibrogenesis (Gao and Brigstock, Gut, 55:856-862, 2006). It has been shown that pharmacologic antagonism of α5β1 inhibits the attachment migration, and proliferation of human retinal epithelial cells in vitro, and reduces retinal cell proliferation and scarring when administered intravitreally to rabbits with retinal detachment (Li et al., Invest. Ophthalmol. Vis. Sci., 50(12):5988-5996, 2009; Zahn et al., Invest. Ophthalmol. Vis. Sci., 51(2):1028-1035, 2010).

In some embodiments, a compound described herein may be useful in the treatment of angiogenesis, and/or a related condition. Such related conditions include fibrosis, for example, fibroid growth, and/or a disease of cellular proliferation, for example, cancer. Some embodiments include using a compound described herein in the treatment or prevention of both fibrosis and angiogenesis. In some embodiments, a compound described herein is administered to a patient suffering from cancer. In further embodiments, a compound described herein is administered to a patient suffering from a fibrotic growth. In still further embodiments, a compound described herein slows the growth of a fibroid, halts the growth of a fibroid, or reverses the growth of a fibroid. In further embodiments, the fibroid is a tumor.

The term “tumor” is used broadly herein to mean any non-congenital, pathological, localized tissue growth. The tumor can be benign, for example, a hemangioma, glioma, teratoma, and the like, or can be malignant, for example, a carcinoma, sarcoma, glioblastoma, astrocytoma, neuroblastoma, retinoblastoma, and the like. The tumor may or may not be metastatic. The term “cancer” is used generally to refer to a disease that accompanies the appearance of a malignant tumor. The tumor can be a carcinoma of, for example, lung cancer, breast cancer, prostate cancer, cervical cancer, pancreatic cancer, colon cancer or ovarian cancer, or a sarcoma, for example, osteosarcoma or Kaposi's sarcoma.

In further embodiments, the fibroid is a fibroma. The fibroma may be, for example, a hard fibroma or a soft fibroma. The fibroma may be, for further example, an angiofibroma, a cystic fibroma, a myxofibroma, a cemento-ossifying fibroma, a chondromyxoid fibroma, a desmoplasmic fibroma, a nonossifying fibroma, an ossifying fibroma, a nuchal fibroma, a collagenous fibroma, a fibroma of tendon sheath, a perifollicular fibroma, a pleomorphic fibroma, a uterine fibroma, a neurofibroma, or an ovarian fibroma.

The integrin α_{V}β1 is expressed on the surface of the principal cellular mediators of organ fibrosis, activated myofibroblasts (Henderson, et al., 2013). Furthermore, a recent study showed cellular-expressed α_{V}β1 directly binds and activates the pro-fibrotic growth factor, transforming growth factor-β1 (TGFβ1), in vitro (Reed, et al., 2015). This same study also showed that therapeutic treatment with a selective small molecule inhibitor of α_{V}β1 could attenuate injury-induced fibrosis in the lungs or livers of mice. Altogether, these data provide evidence for a critical in vivo role for α_{V}β1 in tissue fibrosis.

Like α_{V}β1, the integrins α_{V}β3 and α_{V}β5 are also capable of binding and activating latent TGFβ in vitro (Tatler, et al., 2011; Wipff, et al, 2007). Specific blockade of α_{V}β3 reduces TGFβ signaling and can normalize pro-fibrotic gene expression patterns in cells (Wipff, et al., 2007; Asano, et al., 2005a; Patsenker, et al., 2007). Mice that are deficient in beta-3 subunit expression, and thus lack α_{V}β3 expression, show attenuated CCL18-driven pulmonary collagen accumulation (Luzina, et al., 2009), and are protected in a mouse model of human “stiff skin syndrome”, a form of scleroderma (Gerber, et al., 2013). Modulation of the level of integrin α_{V}β5 expression on cells affects the nuclear localization of components of the TGFβ signaling pathway, and alters expression of fibrosis markers such as alpha smooth muscle actin and collagen (Luzina, et al, 2009; Asano, et al, 2005b; Scotton, et al., 2009).

Integrins α_{V}β3 and α_{V}β5 have been implicated in promoting angiogenesis (Avraamides et al., 2008), so that their antagonism in addition to other integrins may be predicted to provide superior blockade of this process. Integrin α_{V}β3 is also known to play a role in tumor cell metastasis, and in the elevated bone resorption associated with osteoporosis and some cancers (Nakamura, et al., 2007; Schneider, et al., 2011).

Additionally, in some aspects, the antagonists of the present disclosure show reduced activity for other integrins such as α_{V}β6 and α_{V}β8. Loss or excessive inhibition of these specific integrins has been associated with inflammation-related side effects or development of autoimmunity in mice (Huang, et al., 1996; Lacy-Hulbert, et al., 2007; Travis, et al., 2007; Worthington, et al., 2015).

Additionally, in some embodiments, the compounds of the present disclosure show reduced inhibitory or antagonistic activity for integrin α_{IIb}β_{III}, which is an integrin complex found on platelets. Integrin α_{IIb}β_{III} inhibition is associated with disruption of platelet aggregation, which is associated with toxicity and/or contraindicated when treating certain disease or disorders. In some embodiments, the compounds provided herein exhibit increased specificity for integrins α_{V}β₁ and α₅β₁ relative to an untargeted integrin, e.g., integrin α_{IIb}β_{III}. In some embodiments, the compounds provided herein may be used as anti-fibrotic agents that minimize the potential for toxicities associated with bleeding disorders.

While not being bound by any particular theory, it has been unexpectedly discovered that certain compounds described herein exhibit inhibitory activity for α_{V}β₁ and α₅β₁ while sparing α_{V}β₃, α_{V}β₅, α_{V}β₆, and/or α_{V}β₈.

There are many types of integrin, and many cells have multiple types on their surface. Integrins are of vital importance to all animals and have been found in all animals investigated, from sponges to mammals. As such compounds, which target integrins have found numerous uses in different animals including companion animals, livestock animals, zoo animals as well as wild animals. Integrins have been extensively studied in humans. Integrins work alongside other proteins such as cadherins, immunoglobulin superfamily cell adhesion molecules, selectins and syndecans to mediate cell-cell and cell-matrix interaction and communication. Integrins bind cell surface and ECM components such as fibronectin, vitronectin, collagen, and laminin.

Each integrin is formed by the non-covalent heterodimerization of alpha and beta glycoprotein subunits, the combination of which conveys distinct biological activities such as cell attachment, migration, proliferation, differentiation, and survival. Currently, 24 integrins have been described in mammals that are formed by pairing of 18 α subunits and 8 β subunits and are listed in Table 4:

TABLE 4
Integrins

| Gene | Protein | Synonym | Type |
|---|---|---|---|
| ITGA1 | CD49a | VLA1 | Alpha |
| ITGA2 | CD49b | VLA2 | Alpha |
| ITGA3 | CD49c | VLA3 | Alpha |
| ITGA4 | CD49d | VLA4 | Alpha |
| ITGA5 | CD49e | VLA5 | Alpha |
| ITGA6 | CD49f | VLA6 | Alpha |
| ITGA7 | ITGA7 | FEJ25220 | Alpha |
| ITGA8 | ITGA8 | | Alpha |
| ITGA9 | ITGA9 | REC | Alpha |
| ITGA10 | ITGA10 | | Alpha |
| ITGA11 | ITGA11 | HsT18964 | Alpha |
| ITGAD | CD11D | FEJ39841 | Alpha |
| ITGAE | CD103 | HUMINAE | Alpha |
| ITGAL | CD11a | EFA1A | Alpha |
| ITGAM | CD11b | MAC-1 | Alpha |
| ITGAV | CD51 | VNRA, MSK8 | Alpha |
| ITGAW | ITGAW | | Alpha |
| ITGAX | CD11c | | Alpha |
| ITGB1 | CD29 | FNRB, MSK12, MDF2 | Beta |
| ITGB2 | CD18 | EFA-1, MAC-1, MFI7 | Beta |
| ITGB3 | CD61 | GP3A, GPIIIa | Beta |
| ITGB4 | CD104 | | Beta |
| ITGB5 | ITGB5 | FEJ26658 | Beta |
| ITGB6 | ITGB6 | | Beta |
| ITGB7 | ITGB7 | | Beta |
| ITGB8 | ITGB8 | | Beta |

In addition, variants of some of the subunits are formed by differential splicing; for example, four variants of the beta-1 subunit exist. Through different combinations of these α and β subunits, some 24 unique integrins are generated, although the number varies according to different studies.

In some embodiments, the compound is an integrin antagonist such as an α₅β₁ integrin antagonist. In some embodiments, the compound exhibits an IC₅₀ value for the α₅β₁ integrin of less than 20 nM, less than 15 nM, or less than 10 nM as measured by a solid phase receptor assay for α₅β₁ integrin function. In some embodiments, the compound is an integrin antagonist such as an α_{V}β₁ integrin antagonist. In some embodiments, the compound exhibits an IC₅₀ value for the α_{V}β₁ integrin of less than 15 nM as measured by a solid phase receptor assay for α_{V}β₁ integrin function. In some embodiments, the compound exhibits an IC₅₀ value for an α_{V}β₃ integrin of less than 10 nM as measured by a solid phase receptor assay for α_{V}β₃ integrin function. In some embodiments, the compound exhibits an IC₅₀ value for an α_{V}β₅ integrin of less than 10 nM as measured by a solid phase receptor assay for α_{V}β₅ integrin function. In some embodiments, the compound exhibits an IC₅₀ value for the α_{V}β₁, α_{V}β₃, and α_{V}β₅ integrins of each less than 10 nM as measured by a solid phase receptor assays for α_{V}β₁, α_{V}β₃, and α_{V}β₅ integrin function, respectively. In some embodiments, the compound exhibits an IC₅₀ value for an α_{V}β₆ integrin of greater than 10 nM as measured by a solid phase receptor assay for α_{V}β₆ integrin function. In some embodiments, the compound exhibits an IC₅₀ value for an α_{V}β₈ integrin of greater than 10 nM as measured by a solid phase receptor assay for α_{V}β₈ integrin function. In some embodiments, the compound exhibits an IC₅₀ value for each of the α_{V}β₆ and α_{V}β₈ integrins of greater than 10 nM as measured by solid phase receptor assays for α_{V}β₆ and α_{V}β₈ integrin function, respectively. In some embodiments, the compound exhibits an IC₅₀ value for an α_{IIb}β₃ integrin of greater than 2,000 nM as measured by a solid phase receptor assay for α_{IIb}β₃ integrin function. In some embodiments, the compound exhibits an IC₅₀ value for an α_{IIb}β₃ integrin of greater than 5,000 nM as measured by a solid phase receptor assay for α_{IIb}β₃ integrin function.

### III. THERAPEUTIC METHODS

The present disclosure relates to the fields of pharmaceuticals, medicine and cell biology. More specifically, it relates to pharmaceutical agents (compounds) and pharmaceutical compositions thereof which may be used as antagonists of one or more specific integrins, such as antagonist of the α₅β₁, α_{V}β1, α_{V}β3, and/or α_{V}β5 integrins. As such, these compounds may be used in pharmaceutical compositions and in methods for treating conditions mediated by one or more of such integrins, for example, by inhibiting or antagonizing one or more of these integrins. In several aspects of the present disclosure, the compounds provided herein may be used in a variety of biological, prophylactic or therapeutic areas which involves one of these integrins. In some aspects of the present disclosure, the compounds described herein may also show reduced activity in other integrins, such as α_{V}β6 and α_{V}β8, which have been implicated in inflammatory side effects (Huang, et al., 1996; Lacy-Hulbert, et al., 2007; Travis, et al., 2007; Worthington, et al., 2015).

In another aspect, this disclosure provides methods of inhibiting or antagonizing one or more of the α₅β₁, α_{V}β1, α_{V}β3, and/or α_{V}β5 integrins using one or more of the compounds disclosed herein, as well as pharmaceutical compositions thereof. Such pharmaceutical compositions further comprise one or more non-toxic, pharmaceutically acceptable carriers and/or diluents and/or adjuvants (collectively referred to herein as “carrier” materials) and if desired other active ingredients. In some embodiments, the compound is administered as part of a pharmaceutical composition further comprising a pharmaceutically acceptable carrier. In some embodiments, the compounds and/or pharmaceutical compositions thereof may be administered orally, parenterally, or by inhalation spray, or topically in unit dosage formulations containing conventional pharmaceutically acceptable carriers, adjuvants and vehicles. The term parenteral as used herein includes, for example, subcutaneous, intravenous, intravitreal, intramuscular, intrasternal, infusion techniques or intraperitoneally. In some embodiments, the compounds of the present disclosure are administered by any suitable route in the form of a pharmaceutical composition adapted to such a route, and in a dose effective for the treatment intended. Therapeutically effective doses of the compounds required to prevent or arrest the progress of or to treat a medical condition are readily ascertained by one of ordinary skill in the art using preclinical and clinical approaches familiar to the medicinal arts.

Based upon standard laboratory experimental techniques and procedures well known and appreciated by those skilled in the art, as well as comparisons with compounds of known usefulness, the compounds described above can be used in the treatment of patients suffering from the above pathological conditions. One skilled in the art will recognize that selection of the most appropriate compound of the disclosure is within the ability of one with ordinary skill in the art and will depend on a variety of factors including assessment of results obtained in standard assay and animal models.

In several aspects of the present disclosure, the compounds provided herein may be used in a variety of biological, prophylactic or therapeutic areas, including those in which one or more the α₅β₁, α_{V}β1, α_{V}β3, and/or α_{V}β5 integrins plays a role.

The disclosure further involves treating or inhibiting pathological conditions associated therewith fibrosis and fibrotic diseases such as pulmonary fibrosis, renal, cardiac, muscle, and liver fibrosis, scleroderma, scarring, such as retinal, comeal and dermal scarring. Additionally, such integrin antagonists may be useful for treatment of conditions characterized by increased or excessive bone loss including, but not limited to, osteoporosis, osteogenenesis imperfecta, Paget's disease, humoral hypercalcemia of malignancy, primary and metastatic cancer of bone, and arthritis including rheumatoid arthritis. Further, such pharmaceutical agents may be useful for reduction of pathological angiogenesis and fibrosis associated with diseases that such as cancer, macular degeneration, vitreoretinopathy, and diabetic retinopathy.

### IV. PHARMACEUTICAL FORMULATIONS AND ROUTES OF ADMINISTRATION

For administration to an animal especially a mammal in need of such treatment, the compounds in a therapeutically effective amount are ordinarily combined with one or more excipients appropriate to the indicated route of administration. The compounds of the present disclosure are contemplated to be formulated in a manner amenable to treatment of a veterinary patient as well as a human patient. In some embodiments, the veterinary patient may be a companion animal, livestock animals, zoo animals, and wild animals The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art and may be adapted to the type of animal being treated.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The compounds of the present disclosure may be administered by a variety of methods, e.g., orally or by injection (e.g. subcutaneous, intravenous, intraperitoneal, etc.). Depending on the route of administration, the active compounds may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The therapeutic compound may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions may be suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it may be useful to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the methods of preparation include vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically or by injection to the skin, eye, or mucosa. Alternatively, if local delivery to the lungs is desired the therapeutic compound may be administered by inhalation in a dry-powder or aerosol formulation.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

An effective amount typically will vary from about 0.001 mg/kg to about 1000 mg/kg, from about 0.01 mg/kg to about 750 mg/kg, from about 100 mg/kg to about 500 mg/kg, from about 1.0 mg/kg to about 250 mg/kg, from about 10.0 mg/kg to about 150 mg/kg in one or more dose administrations daily, for one or several days (depending of course of the mode of administration and the factors discussed above).

Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day. For example, regarding treatment of diabetic patients, the unit dosage may be an amount that reduces blood glucose by at least 40% as compared to an untreated subject. In another embodiment, the unit dosage is an amount that reduces blood glucose to a level that is ±10% of the blood glucose level of a non-diabetic subject.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present disclosure may comprise, for example, at least about 0.1% of a compound of the present disclosure. In other embodiments, the compound of the present disclosure may comprise between about 1% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may taken orally and that the timing of which is or is not dependent upon food intake. Thus, for example, the agent can be taken every morning and/or every evening, regardless of when the subject has eaten or will eat.

### V. COMBINATION THERAPY

In addition to being used as a monotherapy, the compounds of the present disclosure may also find use in combination therapies. Effective combination therapy may be achieved with a single composition or pharmacological formulation that includes both agents, or with two distinct compositions or formulations, administered at the same time, wherein one composition includes a compound of this disclosure, and the other includes the second agent(s). Alternatively, the therapy may precede or follow the other agent treatment by intervals ranging from minutes to months.

Non-limiting examples of such combination therapy include combination of one or more compounds of the disclosure with another agent, for example, an anti-inflammatory agent, a chemotherapeutic agent, radiation therapy, an antidepressant, an antipsychotic agent, an anticonvulsant, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders. Compounds of the disclosure may be combined with agents designed to improve a patient's immune response to cancer, including (but not limited to) cancer vaccines.

### VI. DEFINITIONS

When used in the context of a chemical group: “hydrogen” means —H; “hydroxy” means —OH; “halo” means independently —F, —Cl, —Br or —I.

In the context of chemical formulas, the symbol “ [symbol] ” means a single bond, “ [symbol] ” means a double bond, and “ [symbol] ” means triple bond. Furthermore, it is noted that the covalent bond symbol “ [symbol] ”, when connecting one or two stereogenic atoms, does not indicate any preferred stereochemistry. Instead, it covers all stereoisomers as well as mixtures thereof.

When a group “R” is depicted as a “floating group” on a ring system, for example, in the formula:

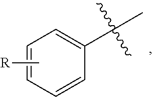

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

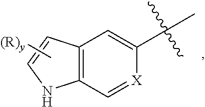

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to the alkyl moiety substituted with at least one halo group. Examples of haloalkyl groups include, but are not limited to, —CF₃, —CHF₂, —CH₂F, —CH₂CF₃, —CH₂CHF₂, —CH₂CH₂F, —CH₂CH₂Cl, or —CH₂CF₂CF₃.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “heterocycloalkyl” means a non-aromatic cyclic ring or ring system containing at least one heteroatom in the ring backbone. Heterocyclyls may be joined together in a fused, bridged or spiro-connected fashion. Heterocycloalkyls may have any degree of saturation provided that at least one ring in the ring system is not aromatic. The heteroatom(s) may be present in either a non-aromatic or aromatic ring in the ring system. The heterocycloalkyl group may have 3 to 20 ring members (i.e., the number of atoms making up the ring backbone, including carbon atoms and heteroatoms), although the present definition also covers the occurrence of the term “heterocycloalkyl” where no numerical range is designated. The heterocycloalkyl group may also be a medium size heterocycloalkyl having 3 to 10 ring members. The hetero heterocycloalkyl cyclyl group could also be a heterocycloalkyl having 3 to 6 ring members. The heterocycloalkyl group may be designated as “3-6 membered heterocycloalkyl” or similar designations. In preferred six membered monocyclic heterocycloalkyls, the heteroatom(s) are selected from one up to three of O, N or S, and in preferred five membered monocyclic heterocycloalkyls, the heteroatom(s) are selected from one or two heteroatoms selected from O, N, or S. Examples of heterocycloalkyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “cycloalkyl” means a fully saturated carbocyclyl ring or ring system. Examples include cyclopropyl, cyclobutyl, cyclopentyl, cyclohexyl, and adamantyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “haloalkoxy” refers to the formula —OR wherein R is a haloalkyl as defined above, such as —CF₃, —CHF₂, —CH₂F, —CH₂CF₃, —CH₂CHF₂, —CH₂CH₂F, —CH₂CH₂Cl, or —CH₂CF₂CF₃.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from halo, cyano, hydroxy, C₁-C₆ alkoxy, C₁-C₆ alkoxy(C₁-C₆)alkyl, aryloxy, C₁-C₆ alkyl, C₂-C₆ alkenyl, C₂-C₆ alkynyl, C₁-C₆ haloalkyl, C₁-C₆ haloalkoxy, C₃-C₇ cycloalkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, acyloxy, cyanato, isocyanato, thiocyanato, isothiocyanato, sulfinyl, sulfonyl, and oxo (═O). Wherever a group is described as “optionally substituted” that group can be substituted with the above substituents.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. Non-limiting examples include formyl, acetyl, propanoyl, benzoyl, and acryl.

As used herein, “acyloxy” refers to —OR wherein R is acyl as defined above.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})C(═O)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})C(═S)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, trifluoroacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical agent, pharmaceutical preparation, pharmaceutical composition, pharmaceutical formulation, pharmaceutical product, medicinal product, medicine, medication, medicament, or simply a drug) is a drug used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-P-hydroxynaphthoate, gentisates, isethionates, di-p-toluoyl tartrates, methane-sulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers.

In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

Other abbreviations used herein are as follows: ¹H NMR is proton nuclear magnetic resonance, AcOH is acetic acid, Ac₂O is acetic anhydride, ACN or CH₃CN is acetonitrile, br is broad, d is doublet, DCM is dichloromethane, DIAD is diisopropyl azodicarboxylate, DMA is A A-di methyl acetamide, DMF is N,N-dimethylformamide, DMSO is dimethylsulfoxide, EtOAc or EA is ethyl acetate, EtOH is ethanol, FAB MS is fast atom bombardment mass spectroscopy, g is gram(s), GC-MS is gas chromatograph mass spectroscopy, HOBT is 1-hydroxybenzotriazole hydrate, HPLC is high performance liquid chromatography, L is liter, LAH is lithium aluminum hydride, LC-MS is liquid chromatograph mass spectroscopy, LDA is lithium diisopropylamide, LiHMDS is lithium bis(trimethylsilyl)amide, m is multiplet, MeOH is methanol, mg is milligram, ml is milliliter, mL is milliliter, MS is mass spectroscopy, N is normal, N₂ is nitrogen, Na₂SO₄ is sodium sulfate, NMR is nuclear magnetic resonance, PE is petroleum ether, q is quintet, rt is retention time, t is triplet, THF is tetrahydrofuran, TLC is thin layer chromatography, and Δ signifies heating the reaction mixture.

The above definitions supersede any conflicting definition in any of the reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### I. Instrumentation and General Methods.

Analytical HPLC analyses were performed on an Agilent 1100 system and LC-MS analyses were conducted on Agilent 1100 Series LC/MSD (G1946C) electrospray mass spectrometer system. Reverse-phase preparative HPLC purifications were performed either on a Biotage SP4 HPFC system or on a CombiFlashRf (Teledyne Isco) system using a variable dual wavelength UV detector on a Biotage KP-C18-HS 120 g SNAP column and on Redisep Rf Gold C18 cartridges using acetonitrile/water gradient containing 0.05% TFA. Normal phase preparative HPLC purifications were performed either on a Biotage SP4 HPFC system or on a CombiFlashRf (Teledyne Isco) system using a variable dual wavelength UV detector on Biotage KP-SIL SNAP cartidges and on Redisep Rf silica gel (Isco) cartridges.

All final compounds were analyzed by analytical HPLC using a C18 analytical column with a diode array detector and peaks were monitored at 210, 254 and 280 nm for their purity. ¹H and ¹⁹F NMR spectra were recorded in deuterated solvents (DMSO-d₆, CD₃OD and CDCl₃) on a Bruker Avance-IIF400 MHz spectrometer equipped with a Broad Band NMR probe. The signal of the deuterated solvent was used as an internal reference. The chemical shifts are expressed in ppm (δ) and coupling constants (J) are reported in hertz (Hz). Reactions were performed under an atmosphere of dry nitrogen unless otherwise stated.

The starting materials were obtained from commercial sources and used without further purification after verifying their purities by LC-MS analysis. Solvents were analytical grade and used as supplied. Non commercially available starting materials were synthesized following the literature procedures and used after further purification and verifying their purities by ¹H NMR and LC-MS analysis.

### II. Preparation of Compounds

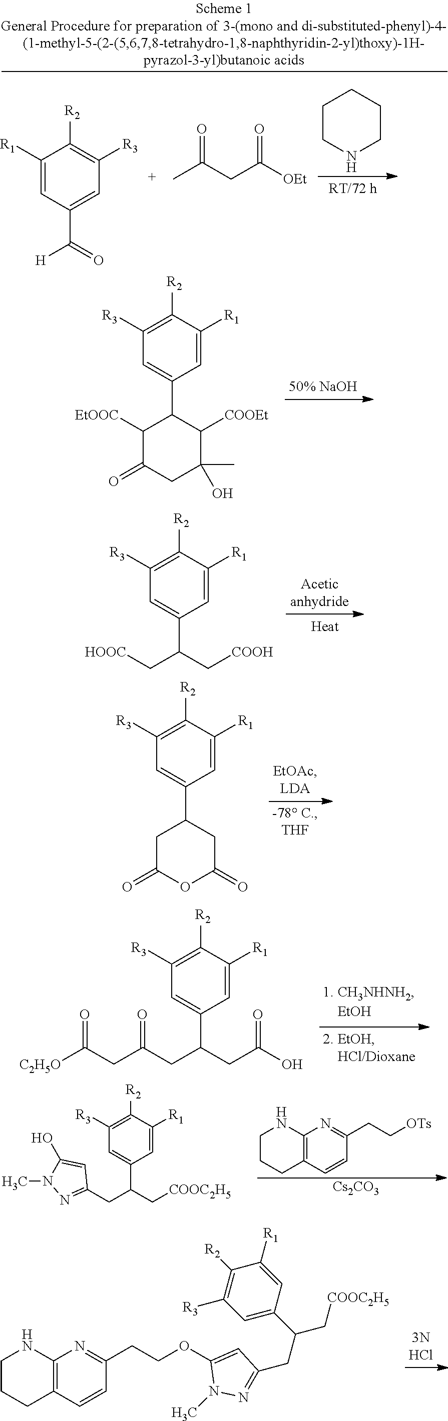

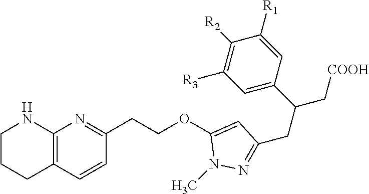

### Example A

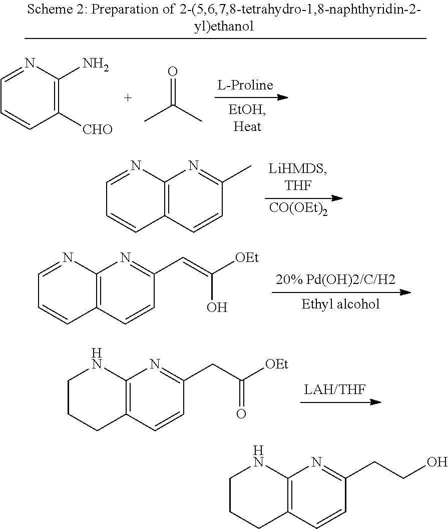

### Step 1. Preparation of 2-methyl-1,8-naphthyridine

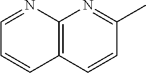

A mixture of 2-aminopyridine-3-carboxyaldehyde (5.125 g, 42.0 mmol), acetone (9.5 mL, 126.0 mmol) and L-proline (5.31 g, 46.2 mmol) in absolute ethyl alcohol (70 mL) was heated at reflux overnight (15 h) under nitrogen atmosphere. The solvent was evaporated in vacuo to afford a canary yellow solid. The solid was dissolved in dichloromethane (50 mL) to give a white precipitate, filtered, washed with dichloromethane and the combined filtrate was evaporated in vacuo to give a yellow-orange residue. The solid was redissolved in dichloromethane (50 mL), washed with water (1×50 mL), the organic layer was separated and the aqueous layer was extracted with dichloromethane (1×25 mL). The combined organic extract was washed with brine (1×50 mL), dried over anhydrous Na₂SO₄, filtered and evaporated in vacuo to afford a dirty yellow solid (6.04 g, yield 99%). GC-MS analysis of the solid shows the desired product's mass: m/z 144 (M⁺); Calculated for C₉H₈N₂:144.17. ¹H NMR (400 MHz, CDCl₃): δ 2.83 (s, 3H), 7.38 (d, J=8.00 Hz, 1H), 7.45 (dd, 1H), 8.09 (d, J=8.00 Hz, 1H), 8.16 (d, J=8.00 Hz, 1H), 9.08 (s, 1H). ¹H NMR spectrum of the sample was consistent with the suggested structure of the product.

### Step 2. Preparation of (E)-1-ethoxy-2-(1,8-naphthridin-2-yl)ethanol

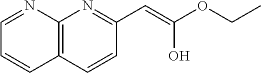 [SMILES: CCO/C(O)=C/c1ccc2cccnc2n1]

To a solution of 2-methyl-1,8-naphthyridine (6.024 g, 41.8 mmol) (from step 1) in anhydrous THF (140 mL) at −40° C. under nitrogen atmosphere was added a 1.0 M solution of lithium bis(trimethylsilyl)amide in THF (88.0 mL) and the reaction mixture was stirred at −40° C. for 30 min to give a blood-red solution. After stirring for 30 min at −40° C., neat diethyl carbonate (5.60 mL) was added drop wise to above solution in 5 min and the reaction mixture was warmed up to 0° C. (ice-bath) and stirred at that temperature for 2 h to give a dark reddish-orange solution. The reaction mixture was quenched with saturated aqueous ammonium chloride solution (60.0 mL) to give an orange-red solution and the THF was removed in vacuo to give an orange-red mixture. The resulting mixture was extracted with ethyl acetate (3×50 mL). The organic layers were combined, washed with brine, dried over anhydrous Na₂SO₄/MgSO₄, filtered and evaporated in vacuo to afford a dark orange-red crystalline solid (8.65 g). The crude residue was purified by Silica-gel flash chromatography using a Varian SF-40-120 g Super Flash silica gel column and elution with 10-100% ethyl acetate in n-heptane to afford the desired product as a yellow-orange crystalline solid (7.76 g, yield 85%). LC-MS analysis of the solid shows the desired product's mass: m/z 217 (M+H) and m/z 239 (M+Na); Calculated for C₁₂H₁₂N₂O₂: 216.23. ¹H NMR (400 MHz, DMSO-d₆): δ 1.21 (t, J=7.0 Hz, 3H), 4.10 (q, 2H), 4.89 (s, 1H), 6.77 (d, J=9.38 Hz, 1H), 7.14 (m, 1H), 7.46 (d, J=9.36 Hz, 1H), 7.89 (d, 1H), 8.36 (d, 1H), 11.80 (brs, 1H, —OH). ¹H NMR of the isolated product was superimposable with that of an authentic sample of the product.

### Step 3. Preparation of Ethyl 5,6,7,8-tetrahydro-1,8-naphthyridin-2-ylacetate

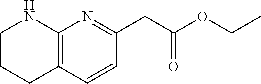 [SMILES: CCOC(=O)Cc1ccc2c(n1)NCCC2]

To a degassed solution of (E)-1-ethoxy-2-(1,8-naphthyridin-2-yl)ethanol (5.18 g, 23.98 mmol) (from step 2) in absolute ethanol (100 mL) was added palladium hydroxide on activated charcoal (1.44 g) and the reaction mixture was stirred at room temperature under a balloon of hydrogen gas overnight (16 h). The reaction mixture was filtered through a pad of Celite® to remove the Pd(OH)₂/C. The residue was washed with absolute ethanol (2×25 mL) and the filtrate was evaporated in vacuo to afford a yellow viscous liquid, crystallized slowly to a pale yellow solid (5.30 g, yield 98%). LC-MS analysis of the product shows the desired product's mass: m/z 221 (M+H); Calculated for C₁₂H₁₆N₂O₂: 220.26. The product will be used such for the next step.

### Step 4. Preparation of 2-(5,6,7,8-tetrahydro-1,8-naphthyridin-2-yl)ethanol

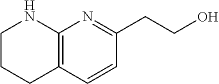 [SMILES: OCCc1ccc2c(n1)NCCC2]

To anhydrous THF (95.0 mL) under nitrogen gas atmosphere at room temperature was added a 1.0 M solution of lithium aluminum hydride in THF (95.0 mL) with stirring. The temperature of the reaction mixture was lowered to 15° C. (water-ice bath) and a solution of ethyl 2-(5,6,7,8-tetrahydro-1,8-naphthyridin-2-yl)acetate (from step 3) in anhydrous THF (50.0 mL) was added drop wise over 30 min to give a yellow solution. The resulting reaction mixture was stirred at room temperature for 4 h. The reaction mixture was cooled to 0° C. (salt-ice bath) and the reaction was quenched slowly with brine (25.0 mL). Additional THF (30.0 mL) was added during the quench to break-up the emulsions. After complete addition of brine, the reaction mixture was stirred at room temperature overnight. Anhydrous sodium sulfate (25.0 g) was added to above reaction mixture and the mixture was stirred at room temperature for another 30 min and filtered. The solid salts residue was washed with ethyl acetate (3×30 mL). The filtrates were combined and concentrated to about 150 mL, dried again with anhydrous sodium sulfate, filtered and evaporated in vacuo to afford an orange viscous liquid (4.8 g). The crude product was purified by Silica-gel flash chromatography on a SF-40-120 g Super Flash silica gel column and elution with 0-5% methanol in ethyl acetate to afford the desired product as a yellow viscous liquid (3.5 g, yield 82%). LC-MS analysis of the purified liquid shows the desired product's mass: m/z 179 (M+H); Calculated for C₁₀H₁₄N₂O: 178.23. The liquid solidified to a pale yellow waxy/crystalline solid on storing in a refrigerator overnight.

### Example 1

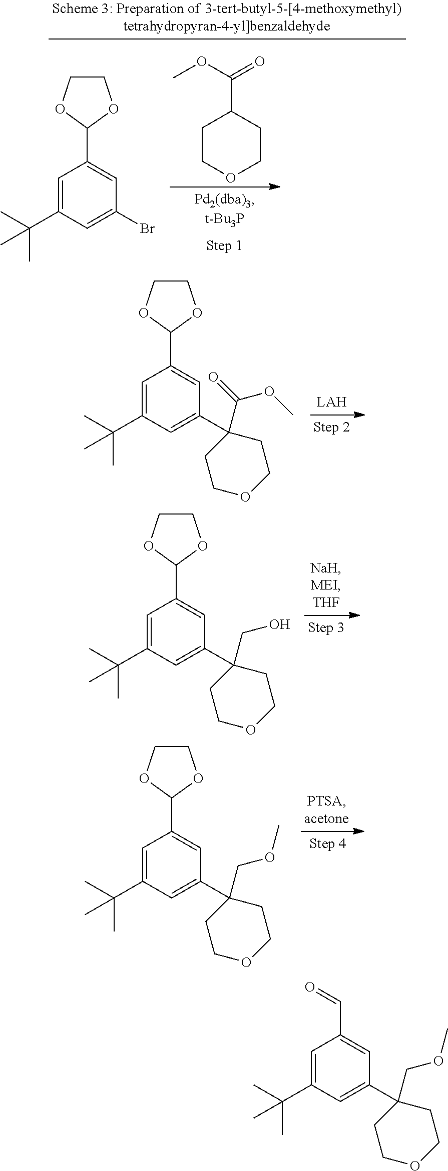

### Step 1. Preparation of methyl 4-[3-tert-butyl-5-(1,3-dioxolan-2-yl)phenyl]tetrahydropyran-4-carboxylate

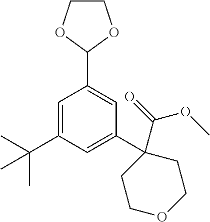 [SMILES: COC(=O)C1(c2cc(C3OCCO3)cc(C(C)(C)C)c2)CCOCC1]

To a mixture of N-cyclohexylcyclohexanamine (827 mg, 4.56 mmol, 908 μL, 1.3 eq) in toluene (10 mL) was added n-BuLi (2.5 M, 1.82 mL, 1.3 eq) at −20° C. under N₂. The mixture warmed to 0° C. and stirred for 20 min, methyl tetrahydropyran-4-carboxylate (506 mg, 3.51 mmol, 468 μL, 1 eq) was added and stirred at 25° C. for 10 min. Then 2-(3-bromo-5-tert-butyl-phenyl)-1,3-dioxolane (1 g, 3.51 mmol, 1 eq), Pd(dba)₂ (60 mg, 105 μmol, 0.03 eq) and t-Bu₃P (213 mg, 105 μmol, 247 μL, 10% purity, 0.03 eq) was added. The mixture was stirred at 25° C. for 12 hr. LCMS showed the desired MS. The mixture was quenched by addition sat. NH₄Cl 20 mL, and then diluted with EtOAc 30 mL and extracted with EtOAc 90 mL (30 mL*3). The combined organic layers were washed with brine (30 mL), dried over Na₂SO₄, filtered and concentrated under reduced pressure to give a residue. The residue was purified by flash silica gel chromatography (ISCO®; 40 g SepaFlash® Silica Flash Column, Eluent of 0-40% Ethyl acetate/Petroleum ether gradient @35 mL/min). The title compound (0.9 g, 2.58 mmol, 74% yield) was obtained as a yellow oil. ¹H NMR (400 MHz, CDCl₃-d₄): δ ppm=7.41-7.36 (m, 2H), 7.27 (s, 1H), 5.75 (s, 1H), 4.16-4.09 (m, 2H), 4.06-3.99 (m, 2H), 3.92 (td, J=3.4, 11.9 Hz, 2H), 3.67-3.62 (m, 3H), 3.53 (dt, J=1.7, 11.6 Hz, 2H), 2.59-2.46 (m, 4H), 1.31-1.25 (m, 9H).

### Step 2. Preparation of [4-[3-tert-butyl-5-(1,3-dioxolan-2-yl)phenyl]tetrahydropyran-4-yl]methanol

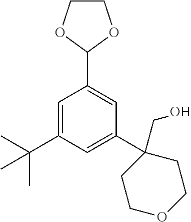 [SMILES: CC(C)(C)c1cc(C2OCCO2)cc(C2(CO)CCOCC2)c1]

A mixture of LAH (196 mg, 5.17 mmol, 2 eq) in THF (4 mL) at 15° C. was treated with methyl 4-[3-tert-butyl-5-(1,3-dioxolan-2-yl)phenyl]tetrahydropyran-4-carboxylate (0.9 g, 2.58 mmol, 1 eq) in THF (22 mL) under N₂ and the resulting mixture was then stirred at 25° C. for 2 hr. The reaction mixture was quenched with H₂O (20 mL) and extracted with ethyl acetate (2*20 mL). The combined organic phase was washed with brine solution (40 mL), dried with anhydrous Na₂SO₄, filtered and evaporated in vacuum. The residue was purified by flash silica gel chromatography (ISCO®; 20 g SepaFlash® Silica Flash Column, Eluent of 0-50% Ethyl acetate/Petroleum ether gradient @ 35 mL/min). The title compound (0.21 g, 655 μmol, 25% yield) was obtained as a colorless oil.

### Step 3. Preparation of 3-[3-tert-butyl-5-[4-(methoxymethyl)tetrahydropyran-4-yl]phenyl]-4-[1-methyl-5-[2-(5,6,7,8-tetrahydro-1,8-naphthyridin-2-yl)ethoxy]pyrazol-3-yl]butanoic acid

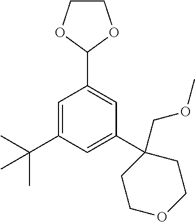 [SMILES: COCC1(c2cc(C3OCCO3)cc(C(C)(C)C)c2)CCOCC1]

Under an argon atmosphere, a solution of [4-[3-tert-butyl-5-(1,3-dioxolan-2-yl)phenyl]tetrahydropyran-4-yl]methanol (1.05 g, 3.28 mmol, 1 eq) in anhydrous THF (10 mL) at 0° C. was treated with NaH (328 mg, 8.2 mmol, 60% purity, 2.5 eq). The resulting mixture was stirred at 0° C. for 30 min. Subsequently, CH₃I (3.51 g, 24.71 mmol, 1.54 mL, 7.54 eq) was added drop wise to the mixture, and the resulting mixture was stirred at 15° C. for 24 hr. LCMS showed the desired MS. The reaction mixture was quenched with brine (50 mL) slowly and then extracted with Ethyl acetate (50 mL*3). The combined organic phase was washed with brine (120 mL), dried over anhydrous Na₂SO₄, filtered and concentrated in vacuum. The residue was purified by flash silica gel chromatography (ISCO®; 12 g SepaFlash® Silica Flash Column, Eluent of 0-21% Ethyl acetate/Petroleum ether gradient @ 30 mL/min). The title compound (775 mg, 2.32 mmol, 70.7% yield) was obtained as a yellow oil.

### Step 4. Preparation of 3-tert-butyl-5-[4-(methoxymethyl)tetrahydropyran-4-yl]benzaldehyde

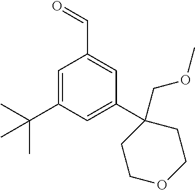 [SMILES: COCC1(c2cc(C=O)cc(C(C)(C)C)c2)CCOCC1]

A mixture of 4-[3-tert-butyl-5-(1,3-dioxolan-2-yl)phenyl]-4-(methoxymethyl)tetrahydropyran (775 mg, 2.32 mmol, 1 eq) and 4-methylbenzenesulfonic acid hydrate (88 mg, 463 μmol, 0.2 eq) in acetone (10 mL) stirred at 15° C. for 12 hours under N₂. LCMS showed the desired MS. The mixture was treated with saturated NaHCO₃ (10 mL) and then extracted with EtOAc (10 mL×2). The combined organic phase was dried over anhydrous Na₂SO₄, filtered and evaporated under vacuum. The residue was purified by flash silica gel chromatography (ISCO®; 12 g SepaFlash® Silica Flash Column, Eluent of 0-12% Ethyl acetate/Petroleum ether gradient @ 30 mL/min). The title compound (545 mg, 1.88 mmol, 81% yield) was obtained as a colorless oil.

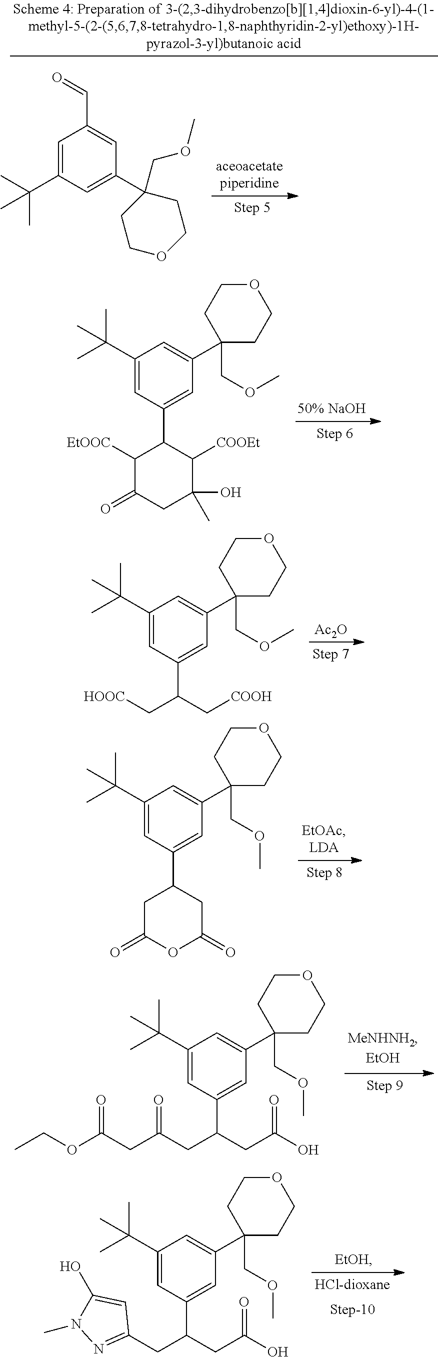

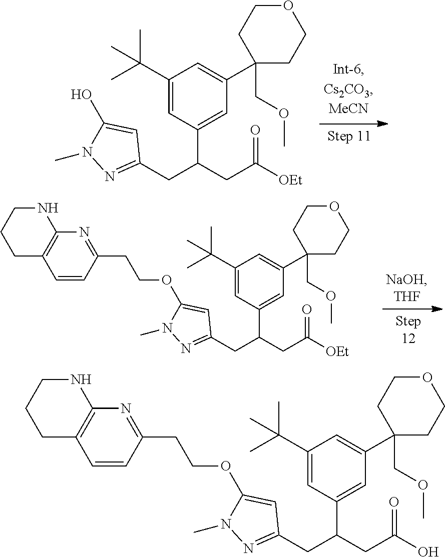

### Step 5. Preparation of 3-tert-butyl-5-[4-(methoxymethyl)tetrahydropyran-4-yl]benzaldehyde

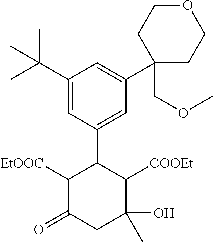 [SMILES: CCOC(=O)C1C(=O)CC(C)(O)C(C(=O)OCC)C1c1cc(C(C)(C)C)cc(C2(COC)CCOCC2)c1]

To a mixture of 3-tert-butyl-5-[4-(methoxymethyl)tetrahydropyran-4-yl]benzaldehyde (545 mg, 1.88 mmol, 1 eq) and piperidine (56 mg, 657 μmol, 65 μL, 0.35 eq) was added ethyl acetoacetate (855 mg, 6.57 mmol, 830 μL, 3.5 eq) in one portion at 15° C. Then the mixture was stirred at 15° C. for 12 hrs. The mixture was concentrated under reduced pressure. The crude compound was directly used in the next step. The title compound (999 mg, crude) was obtained as a yellow oil.

### Step 6. Preparation of 3-[3-tert-butyl-5-[4-(methoxymethyl)tetrahydropyran-4-yl]phenyl]pentanedioic acid

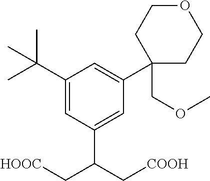 [SMILES: COCC1(c2cc(C(CC(=O)O)CC(=O)O)cc(C(C)(C)C)c2)CCOCC1]

Diethyl 2-[3-tert-butyl-5-[4-(methoxymethyl)tetrahydropyran-4-yl]phenyl]-4-hydroxy-4-methyl-6-oxo-cyclohexane-1,3-dicarboxylate (999 mg, 1.88 mmol, 1 eq) was dissolved in EtOH (4 mL), then NaOH (215 mg, 1.88 mmol, 3 mL, 35% purity, 1 eq) and of H₂O (1 mL) were added. The resulting mixture was heated at 100° C. with stirring for 2.5 h. The mixture was cooled to 15° C. and concentrated under reduced pressure at 60° C. The resulting concentrate was poured into ethyl acetate (20 mL). The organic phase was extracted with water (3×20 mL). The combined water phase was treated with 20% HCl until pH 1 was obtained, and then the mixture was extracted with ethyl acetate (3×20 mL). The combined organic phase was washed with brine solution (2×50 mL), dried with anhydrous Na₂SO₄, filtered and evaporated in vacuum. The crude compound was directly used in the next step. The title compound (670 mg, 1.71 mmol, 90.80% yield) was obtained as a yellow oil.

### Step 7. Preparation of 4-[3-tert-butyl-5-[4-(methoxymethyl)tetrahydropyran-4-yl]phenyl]tetrahydropyran-2,6-dione

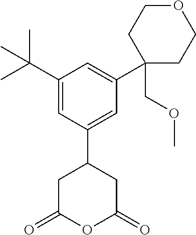 [SMILES: COCC1(c2cc(C3CC(=O)OC(=O)C3)cc(C(C)(C)C)c2)CCOCC1]

A mixture of 3-[3-tert-butyl-5-[4-(methoxymethyl)tetrahydropyran-4-yl]phenyl]pentanedioic acid (670 mg, 1.71 mmol, 1 eq) and Ac₂O (12.2 g, 119.5 mmol, 11.19 mL, 70 eq) was heated with stirring at 140° C. for 2.5 h. The mixture was cooled to 10° C., and then evaporated in vacuum. The crude product was used directly in the next step. The title compound (639 mg, 1.71 mmol, 99.96% yield) was obtained as a yellow oil.

### Step 8. Preparation of 3-[3-tert-butyl-5-[4-(methoxymethyl)tetrahydropyran-4-yl]phenyl]-7-ethoxy-5,7-dioxo-heptanoic acid

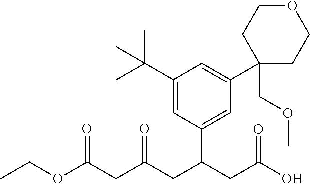 [SMILES: CCOC(=O)CC(=O)CC(CC(=O)O)c1cc(C(C)(C)C)cc(C2(COC)CCOCC2)c1]

A mixture of EtOAc (451 mg, 5.12 mmol, 501 μL, 3 eq) in anhydrous THF (10 mL) at −78° C. under N₂ gas was slowly treated with LDA (2 M, 2.56 mL, 3 eq). The resulting mixture was stirred at −78 C for 25 min and added drop wise via syringe to a solution of the 4-[3-tert-butyl-5-[4-(methoxymethyl)tetrahydropyran-4-yl]phenyl]tetrahydropyran-2,6-dione (639 mg, 1.71 mmol, 1 eq) in anhydrous THF (10 mL) at −78 C under N₂ gas. The resulting mixture was stirred at −78° C. for 1.5 h. The reaction mixture was quenched with 2N HCl in water (20 mL) and allowed to warm up to room temperature. The mixture was diluted with water (20 mL) and then extracted with EtOAc (3×20 mL). The organic layers were combined, washed with brine, dried over Na₂SO₄, and concentrated under reduced pressure. The residue was purified by flash silica gel chromatography (ISCO®; 4 g SepaFlash® Silica Flash Column, Eluent of 0˜100% Ethyl acetate/Petroleum ether gradient @ 30 mL/min). The title compound (500 mg, 1.08 mmol, 63% yield) was obtained as a colorless oil.

### Step 9. Preparation of 3-[3-tert-butyl-5-[4-(methoxymethyl)tetrahydropyran-4-yl]phenyl]-4-(5-hydroxy-1-methyl-pyrazol-3-yl)butanoic acid

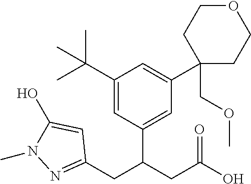 [SMILES: COCC1(c2cc(C(CC(=O)O)Cc3cc(O)n(C)n3)cc(C(C)(C)C)c2)CCOCC1]

To a solution of 3-[3-tert-butyl-5-[4-(methoxymethyl)tetrahydropyran-4-yl]phenyl]-7-ethoxy-5,7-dioxo-heptanoic acid (500 mg, 1.08 mmol, 1 eq) in EtOH (64 mL) was added a solution of methylhydrazine (0.14 g, 1.22 mmol, 160.00 uL, 1.12 eq) in EtOH (64 mL) drop wise at 40° C. over 30 min. The resulting mixture was stirred at 100° C. for 1.5 hr. The mixture was concentrated under reduced pressure to give a product without further purification. The title compound (480 mg, crude) was obtained as a yellow oil.

### Step 10. Preparation of ethyl 3-[3-tert-butyl-5-[4-(methoxymethyl)tetrahydropyran-4-yl]phenyl]-4-(5-hydroxy-1-methyl-pyrazol-3-yl)butanoate

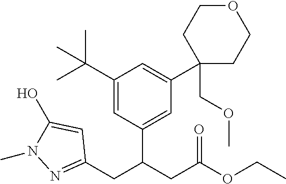 [SMILES: CCOC(=O)CC(Cc1cc(O)n(C)n1)c1cc(C(C)(C)C)cc(C2(COC)CCOCC2)c1]

3-[3-Tert-butyl-5-[4-(methoxymethyl)tetrahydropyran-4-yl]phenyl]-4-(5-hydroxy-1-methyl-pyrazol-3-yl)butanoic acid (480 mg, 1.08 mmol, 1 eq) was dissolved in EtOH (1.52 g, 32.9 mmol, 1.93 mL, 30.5 eq) and then HCl/dioxane (4 M, 3.13 mL, 11.6 eq) was added. The resulting mixture was stirring for 12 h at 15° C. The mixture was concentrated under reduced vacuum. The residue was purified by flash silica gel chromatography (ISCO®; 12 g SepaFlash® Silica Flash Column, Eluent of 100% Ethyl acetate/Petroleum ether @ 30 mL/min). The title compound (220 mg, 465 μmol, 43% yield) was obtained as a yellow oil.

### Step 11. Preparation of ethyl 3-[3-tert-butyl-5-[4-(methoxymethyl)tetrahydropyran-4-yl]phenyl]-4-[1-methyl-5-[2-(5,6,7,8-tetrahydro-1,8-naphthyridin-2-yl)ethoxy]pyrazol-3-yl]butanoate

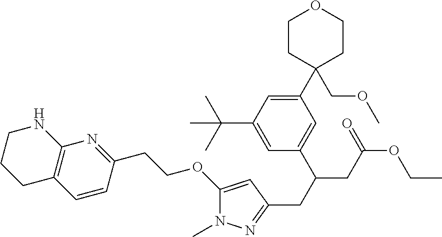 [SMILES: CCOC(=O)CC(Cc1cc(OCCc2ccc3c(n2)NCCC3)n(C)n1)c1cc(C(C)(C)C)cc(C2(COC)CCOCC2)c1]

A mixture of 2-(5,6,7,8-tetrahydro-1,8-naphthyridin-2-yl)ethyl 4-methylbenzenesulfonate (170 mg, 512 μmol, 1.1 eq) and Cs₂CO₃ (379 mg, 1.16 mmol, 2.5 eq) were added to a solution of ethyl 3-[3-tert-butyl-5-[4-(methoxymethyl)tetrahydropyran-4-yl]phenyl]-4-(5-hydroxy-1-methyl-pyrazol-3-yl)butan oate (220 mg, 465.50 μmol, 1 eq) in MeCN (20 mL). The resulting mixture was allowed to stir for 2 h at 80° C. The mixture was then filtered to remove insoluble material and the filtrate was concentrated in vacuum. The residue was purified by flash silica gel chromatography (ISCO®; 4 g SepaFlash® Silica Flash Column, Eluent of 0˜10% Methanol/Ethyl acetate@ 30 mL/min). The title compound (90 mg, 142 μmol, 31% yield) was obtained as a yellow gum.

### Step 12. Preparation of 3-[3-tert-butyl-5-[4-(methoxymethyl)tetrahydropyran-4-yl]phenyl]-4-[1-methyl-5-[2-(5,6,7,8-tetrahydro-1,8-naphthyridin-2-yl)ethoxy]pyrazol-3-yl]butanoic acid

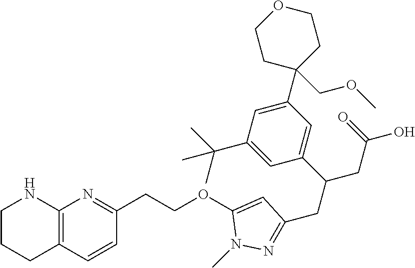 [SMILES: COCC1(c2cc(C(CC(=O)O)Cc3cc(OCCc4ccc5c(n4)NCCC5)n(C)n3)cc(C(C)(C)C)c2)CCOCC1]

A mixture of ethyl 3-[3-tert-butyl-5-[4-(methoxymethyl)tetrahydropyran-4-yl]phenyl]-4-[1-methyl-5-[2-(5,6,7,8-tetrahydro-1,8-naphthyridin-2-yl)ethoxy]pyrazol-3-yl]butanoate (90 mg, 142 μmol, 1 eq) in THF (6 mL) was treated with NaOH (1 M, 142 μL, 1 eq). The resulting mixture was heated at 60° C. for 12 hr. The mixture was allowed to cool to room temperature, then the mixture was acidified with acetic acid to PH=6, and concentrated under reduced pressure. The residue was purified by prep-HPLC (column: Boston Green ODS 150*30 5ρ; mobile phase: [water (0.1% TFA)-ACN]; B %: 30%-56.25%, 7 min). The title compound (44 mg, 62 μmol, 43% yield, 100% purity, TFA) was obtained as a white solid. ¹H NMR (400 MHz, MeOD-d₄): δ ppm=7.59 (d, J=7.3 Hz, 1H), 7.22 (s, 1H), 7.11 (s, 1H), 6.97 (s, 1H), 6.68 (d, J=7.3 Hz, 1H), 5.52 (s, 1H), 4.35-4.27 (m, 2H), 3.70 (tdd, J=4.0, 11.4, 19.3 Hz, 2H), 3.54-3.47 (m, 5H), 3.44-3.37 (m, 2H), 3.32-3.29 (m, 3H), 3.21-3.12 (m, 5H), 2.95-2.88 (m, 1H), 2.84-2.61 (m, 5H), 2.10-2.02 (m, 2H), 2.00-1.89 (m, 4H), 1.28 (s, 9H). ¹⁹F NMR (376 MHz, MeOD-d₄)=−77.35 (s, 1F).

### Example 2

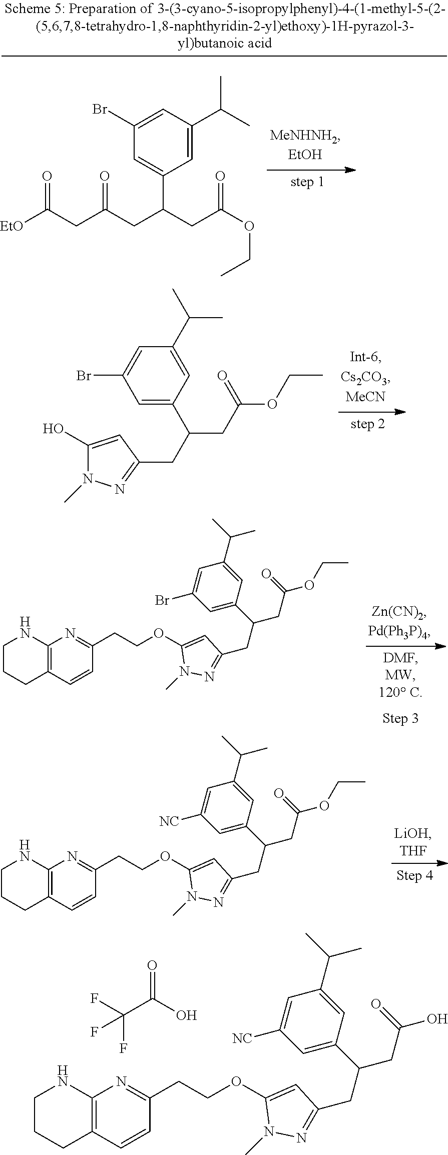

### Step 1. Preparation of ethyl 3-(3-bromo-5-isopropylphenyl)-4-(5-hydroxy-1-methyl-1H-pyrazol-3-yl)butanoate

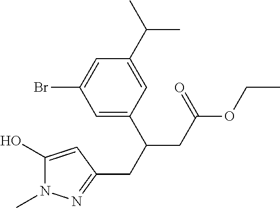 [SMILES: CCOC(=O)CC(Cc1cc(O)n(C)n1)c1cc(Br)cc(C(C)C)c1]

A stirred solution of diethyl 3-(3-bromo-5-isopropyl-phenyl)-5-oxo-heptanedioate (750 mg, 1.76 mmol, 1 eq) in EtOH (75 mL) was treated with HOAc (105 mg, 1.76 mmol, 100 μL, 1 eq) and then CH₃NHNH₂ (222 mg, 1.93 mmol, 254 μL, 1.1 eq) was added in portions at 30° C. for 1 hr. The resulting mixture was stirred at 60° C. for 2 hr. The mixture was cooled to 15° C. and concentrated in vacuo to give residue. The residue was purified by flash silica gel chromatography (ISCO®; 12 g CombiFlash® Silica Flash Column, Eluent of 0˜100% Ethyl acetate/Petroleum ether gradient @ 35 mL/min). The title compound (110 mg, 164 umol, 9% yield, 61% purity) was obtained as a light yellow liquid which was confirmed by LCMS (m/z 410.8 (M+H)).

### Step 2. Preparation of ethyl 3-(3-bromo-5-isopropylphenyl)-4-(1-methyl-5-(2-(5,6,7,8-tetrahydro-1,8-naphthyridin-2-yl)ethoxy)-1H-pyrazol-3-yl)butanoate

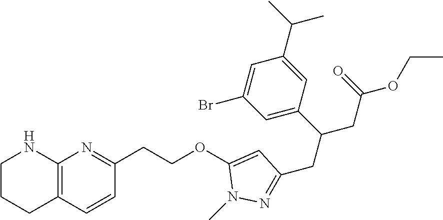 [SMILES: CCOC(=O)CC(Cc1cc(OCCc2ccc3c(n2)NCCC3)n(C)n1)c1cc(Br)cc(C(C)C)c1]

A solution of ethyl 3-(3-bromo-5-isopropyl-phenyl)-4-(5-hydroxy-1-methyl-pyrazol-3-yl)butanoate (110 mg, 269 μmol, 1 eq) in CH₃CN (5 mL) was treated with 2-(5,6,7,8-tetrahydro-1,8-naphthyridin-2-yl)ethyl 4-methylbenzenesulfonate (98 mg, 296 μmol, 1.1 eq) and Cs₂CO₃ (219 mg, 672 μmol, 2.5 eq), and then the resulting mixture was stirred for 2 h at 80° C. The mixture was filtered, and then the filterate was dried under vacuum to give a residue. The residue was purified by flash silica gel chromatography (ISCO®; 12 g CombiFlash® Silica Flash Column, Eluent of 0˜100% Ethyl acetate/Petroleum ether gradient @ 35 mL/min). The title compound (0.07 g, 117 μmol, 44% yield, 95% purity) was obtained as a light yellow liquid which was confirmed by LCMS (m/z 571.1 (M+H)).

### Step 3. Preparation of ethyl 3-(3-cyano-5-isopropylphenyl)-4-(1-methyl-5-(2-(5,6,7,8-tetrahydro-1,8-naphthyridin-2-yl)ethoxy)-1H-pyrazol-3-yl)butanoate

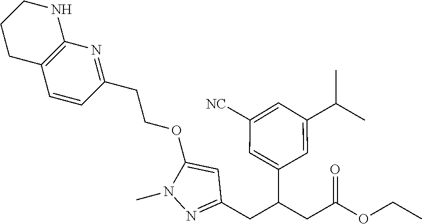 [SMILES: CCOC(=O)CC(Cc1cc(OCCc2ccc3c(n2)NCCC3)n(C)n1)c1cc(C#N)cc(C(C)C)c1]

A microwave vial containing a mixture of ethyl 3-(3-bromo-5-isopropyl-phenyl)-4-[1-methyl-5-[2-(5,6,7,8-tetrahydro-1,8-naphthyridin-2-yl)ethoxy]pyrazol-3-yl]butanoate (70 mg, 123 μmol, 1 eq) and Zn(CN)₂ (43 mg, 369 μmol, 23 μL, 3 eq) in DMF (2 mL) was evacuated and back-filled with N₂ (3×) and then Pd(PPh₃)₄ (14.2 mg, 12.3 μmol, 0.1 eq) was added. The vial was sealed, the mixture was again degassed and back-filled with N₂ (3×), and then stirred at 120° C. for 1.5 hr under micro-wave irradiation. The mixture was poured into water (10 mL), and extracted with EtOAc (3*10 mL). The combined organic layer was washed with brine (20 mL), dried over sodium sulfate, and evaporated to give a residue. The residue was purified by prep-HPLC (column: YMC-Actus Triart C18 100*30 mm*5 μm; mobile phase: [water (0.05% HCl)-ACN]; B %: 40%-63%, 7 min). The title compound (20 mg, 35 umol, 28% yield, 96% purity, HCl) was obtained as a white gum which was confirmed by LCMS (m/z 516.1 (M+H)).

### Step 4. Preparation of 3-(3-cyano-5-isopropylphenyl)-4-(1-methyl-5-(2-(5,6,7,8-tetrahydro-1,8-naphthyridin-2-yl)ethoxy)-1H-pyrazol-3-yl)butanoic acid trifluoroacetic acid

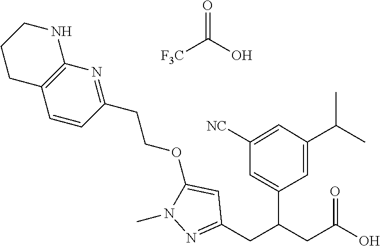 [SMILES: CC(C)c1cc(C#N)cc(C(CC(=O)O)Cc2cc(OCCc3ccc4c(n3)NCCC4)n(C)n2)c1 ; O=C(O)C(F)(F)F]

Ethyl 3-(3-cyano-5-isopropyl-phenyl)-4-[1-methyl-5-[2-(5,6,7,8-tetrahydro-1,8-naphthyridin-2-yl)ethoxy]pyrazol-3-yl]butanoate (20 mg, 39 μmol, 1 eq) in THF (2 mL) was treated with LiOH (aq) (1 M, 465 uL, 12 eq). The resulting mixture was heated at 50° C. for 4 hr. The mixture was allowed to cool to 15° C., then the mixture was acidified with acetic acid adjusting the pH to 3 and extracted with ethyl acetate (10*2 mL). The combined organic layer was dried over sodium sulfate and concentrated under reduced pressure. The residue was purified by prep-HPLC (column: Boston Green ODS 150*30 5μ; mobile phase: [water (0.1% TFA)-ACN]; B %: 20%-50%, 8 min). The title compound (2.2 mg, 3.6 μmol, 9% yield, 99% purity, TFA) was obtained as a white solid. LCMS (m/z 488.0 (M+H)). ¹H NMR (CD₃OD, 400 MHz) 7.60 (d, J=7.3 Hz, 1H), 7.43-7.35 (m, 3H), 6.68 (d, J=7.3 Hz, 1H), 5.47 (s, 1H), 4.36-4.28 (m, 2H), 3.53-3.40 (m, 6H), 3.16 (t, J=6.0 Hz, 2H), 2.99-2.58 (m, 7H), 1.95 (quin, j=5.9 Hz, 2H), 1.21 (dd, j=1.4, 6.9 Hz, 6H). ¹⁹F NMR (CD₃OD, 376 MHz) −77.34 (s, 1F).

### Example 3

### Preparation of 3-[3-bromo-5-(pentafluoro-sulfanyl)phenyl]-4-[1-methyl-5-[2-(5,6,7,8-tetrahydro-1,8-naphthyridin-2-yl)ethoxy]pyrazol-3-yl]butanoic acid

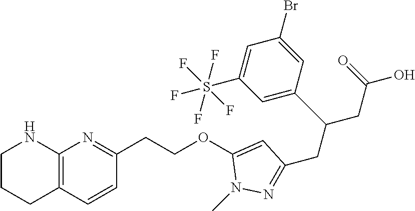 [SMILES: Cn1nc(CC(CC(=O)O)c2cc(Br)cc(S(F)(F)(F)(F)F)c2)cc1OCCc1ccc2c(n1)NCCC2]

Example 3 was prepared in analogous manner to Example 1, using 3-bromo-5-(pentafluoro-sulfanyl)benzaldehyde (obtained according to Example B) as the required benzaldehyde in the reaction Scheme 4. The crude product was purified by reverse-phase preparative HPLC (column: Boston Green ODS 150*30 5μ; mobile phase: [water (0.1% TFA)-ACN]; B %: 20%-55%, 8 min) affording the title compound as a white solid (85 mg, 83 μmol, 45% yield, TFA) was obtained as a white solid. LCMS (m/z 626.9 (M+H)). ¹H NMR (CD₃OD, 400 MHZ) 7.79 (s, 1H), 7.68 (s, 1H), 7.61 (d, J=7.3 Hz, 1H), 7.53 (s, 1H), 6.69 (d, j=7.5 Hz, 1H), 5.46 (s, 1H), 4.31 (t, j=5.9 Hz, 2H), 3.54-3.47 (m, 3H), 3.45 (s, 3H), 3.16 (t, J=6.0 Hz, 2H), 2.93-2.68 (m, 6H), 2.02-1.91 (m, 2H). ¹⁹F NMR (CD₃OD, 376 MHz) 81.89 (quin, J=149.5 Hz, 1F), 61.53 (br d, J=148.8 Hz, 1F), −77.40 (br s, 1F).

### Example B

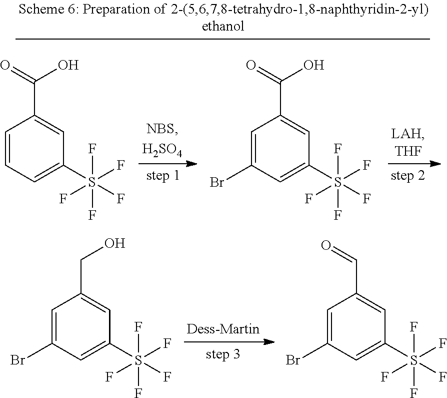

### Step 1. Preparation of 3-bromo-5-(pentafluoro-sulfanyl)benzoic acid

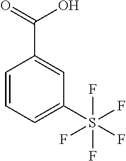 [SMILES: O=C(O)c1cccc(S(F)(F)(F)(F)F)c1]

A mixture of 3-(pentafluoro-sulfanyl)benzoic acid (4 g, 16.12 mmol, 1 eq) and trifluoroacetic acid (12.32 g, 108 mmol, 8 mL, 6.7 eq) was treated with H₂SO₄ (5.52 g, 56.3 mmol, 3 mL, 3.49 eq). The resulting mixture was treated with NBS (4.30 g, 24.2 mmol, 1.5 eq). The resulting mixture was stirred at 50° C. for 12 hr. After cooling to 15° C., the mixture was carefully stirred into about 10 mL of ice water. The precipitated product was filtered off with suction, washed with water and dried under vacuum. The title compound (5 g, 15.3 mmol, 95% yield) was obtained as a white solid. ¹H NMR (CDCl₃, 400 MHz) 8.42-8.32 (m, 2H), 8.26 (t, 7=1.9 Hz, 1H).

### Step 2. Preparation of [3-bromo-5-(pentafluoro-sulfanyl)phenyl]methanol

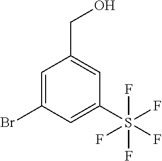 [SMILES: OCc1cc(Br)cc(S(F)(F)(F)(F)F)c1]

A mixture of LAH (1.16 g, 30.6 mmol, 2.0 eq) in THF (50 mL) was treated with 3-bromo-5-(pentafluoro-sulfanyl)benzoic acid (5 g, 15.29 mmol, 1 eq) in THF (50 mL at 15° C. under N₂. The resulting mixture was stirred at 15° C. for 4 hr. The reaction mixture was quenched with H₂O (100 mL) and extracted with ethyl acetate (2*150 mL). The combined organic phase was washed with brine solution (200 mL), dried with anhydrous Na₂SO₄, filtered and evaporated in vacuum. The residue was purified by flash silica gel chromatography (ISCO®; 40 g CombiFlash® Silica Flash Column, Eluent of 0˜10% Ethyl acetate/Petroleum ether gradient @ 40 mL/min). The title compound (1.9 g, 6.07 mmol, 39.7% yield) was obtained as a white solid. ¹H NMR (CDCl₃, 400 MHz) 7.82 (s, 1H), 7.70 (d, J=8.0 Hz, 2H), 4.78 (d, J=5.8 Hz, 2H).

### Step 3. Preparation of 3-bromo-5-(pentafluoro-sulfanyl)benzaldehyde

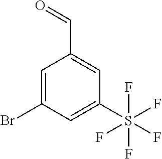 [SMILES: O=Cc1cc(Br)cc(S(F)(F)(F)(F)F)c1]

A mixture of [3-Bromo-5-(pentafluoro-sulfanyl)phenyl]methanol (1.9 g, 6.07 mmol, 1 eq) in DCM (30 mL) was teated with Dess-Martin (3.86 g, 9.1 mmol, 2.82 mL, 1.5 eq) and the resulting mixture was stirred for 2 h at 15° C. The mixture was diluted with DCM (50 mL), washed with saturated Na₂S₂O₃ (100 mL), saturated aqueous NaHCO₃ (100 mL), and brine. Then organic layer was dried over Na₂SO₄ and concentrated under reduced pressure. The residue was purified by flash silica gel chromatography (ISCO®; 12 g CombiFlash® Silica Flash Column, Eluent of 0-5% Ethyl acetate/Petroleum ether gradient @ 30 mL/min). The title compound (1.5 g, 4.82 mmol, 79% yield) was obtained as a colorless liquid. H NMR (CDCl₃, 400 MHz) 10.09-9.96 (m, 1H), 8.25-8.10 (m, 3H).

### Example 4

### Preparation of 3-(3,5-diisopropylphenyl)-4-[1-methyl-5-[2-(5,6,7,8-tetrahydro-1,8-naphthyridin-2-yl)ethoxy]pyrazol-3-yl]butanoic acid

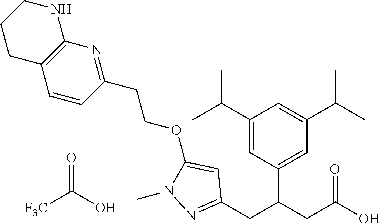

Example 4 was prepared in analogous manner to Example 1, using 3,5-diisopropylbenzaldehyde (obtained according to Example C) as the required benzaldehyde in the reaction Scheme 4. The crude product was purified by prep-HPLC (column: Boston Green ODS 150*30 5μ; mobile phase: [water (0.1% TFA)-ACN]; B %: 35%-61.25%, 7 min) affording the title compound (121 mg, 191 μmol, 46% yield, 98% purity, TFA) was obtained as a white solid. LCMS (m/z 505.2 (M+H)). ¹⁹F NMR (376 MHz, CD₃OD) −77.30 (br s, 3F). ¹H NMR (CD₃OD, 400 MHz) 7.58 (d, J=7.6 Hz, 1H), 6.95-6.84 (m, 3H), 6.67 (d, J=7.6 Hz, 1H), 5.69-5.60 (m, 1H), 4.45-4.29 (m, 2H), 3.53 (s, 3H), 3.52-3.47 (m, 2H), 3.39 (quin, J=7.6 Hz, 1H), 3.17 (t, J=6.0 Hz, 2H), 2.97-2.89 (m, 1H), 2.82 (quin, J=6.8 Hz, 5H), 2.74-2.58 (m, 2H), 1.94 (quin, J=6.0 Hz, 2H), 1.19 (dd, J=2.4, 6.8 Hz, 12H).

### Example C

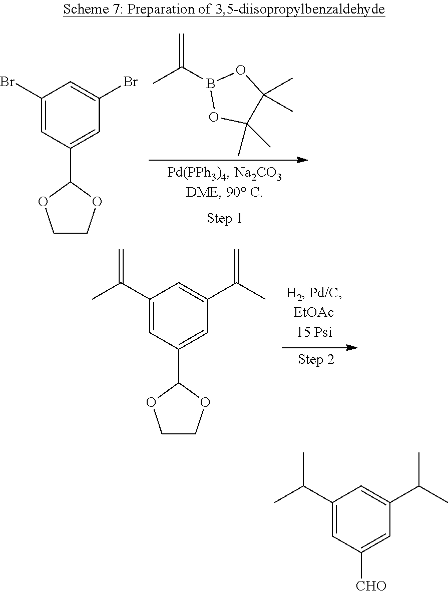

### Step 1. Preparation of 2-(3,5-diisopropenylphenyl)-1,3-dioxolane

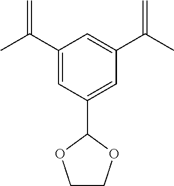 [SMILES: C=C(C)c1cc(C(=C)C)cc(C2OCCO2)c1]

A degassed solution of 2-(3,5-dibromophenyl)-1,3-dioxolane (10 g, 32.5 mmol, 1 eq) in dimethoxyethane (105 mL) was added into a tube charged with a mixture of 2-isopropenyl-4,4,5,5-tetramethyl-[1,3,2]dioxaborolane (10.9 g, 64.9 mmol, 2 eq) and an aqueous solution of Na₂CO₃ (1 M, 58 mL, 1.79 eq), then palladium; triphenylphosphane (760 mg, 658 μmol, 2 eq) is added and the tube is sealed. The mixture was heated to 90° C. and stirred for 12 hr. After the mixture was cooled to 10° C., H₂O (100 mL) was added, the aqueous layer separated and extracted with ethyl acetate (3×150 mL). The combined organic layers were washed by brine, dried over anhydrous Na₂SO₄ and evaporated. The residue was purified by flash silica gel chromatography (ISCO®; 120 g SepaFlash® Silica Flash Column, Eluent of 0˜10% Ethyl acetate/Petroleum ether gradient @ 60 mL/min). The title compound (4.4 g, 19.1 mmol, 59% yield) was obtained as yellow oil. NMR (400 MHz, CDCl₃) 7.62-7.55 (m, 1H), 7.50 (d, J=1.6 Hz, 2H), 5.85 (s, 1H), 5.41 (s, 2H), 5.17-5.10 (m, 2H), 4.19-4.11 (m, 2H), 4.08-4.03 (m, 2H), 2.22-2.12 (m, 6H).

### Step 2. Preparation of 3,5-diisopropylbenzaldehyde

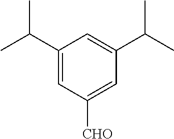

To a solution of 2-(3,5-diisopropenylphenyl)-1,3-dioxolane (3.2 g, 13.89 mmol, 1 eq) in EtOAc (80 mL) was added wet Pd/C (0.8 g, 10% purity) under N₂. The suspension was degassed under vacuum and purged with H₂ 3 times. The mixture was stirred under H₂ (15 psi) at 10° C. for 3 hr. The mixture was filtered and the filterate was concentrated. The residue was purified by flash silica gel chromatography (ISCO®; 12 g SepaFlash® Silica Flash Column, Eluent of 0-5% Ethyl acetate/Petroleum ether gradient @ 20 mL/min). 3,5-Diisopropylbenzaldehyde (2.3 g, 12.1 mmol, 87% yield) was obtained as yellow oil. ¹H NMR (400 MHz, CDCl₃) 10.00 (s, 1H), 7.58 (d, 7=1.6 Hz, 2H), 7.36 (s, 1H), 3.04-2.94 (m, 2H), 1.29 (d, 7=6.8 Hz, 12H).

### Example 5

### Preparation of 3-(3-(ter t-butyl)-5-(1,1,1-trifluoro-2-methylpropan-2-yl)phenyl)-4-(1-methyl-5-(2-(5,6,7,8-tetrahydro-1,8-naphthyridin-2-yl)ethoxy)-1H-pyrazol-3-yl) butanoic acid

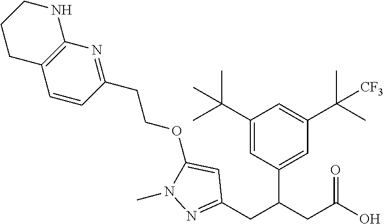 [SMILES: Cn1nc(CC(CC(=O)O)c2cc(C(C)(C)C)cc(C(C)(C)C(F)(F)F)c2)cc1OCCc1ccc2c(n1)NCCC2]

Example 5 was prepared in analogous manner to Example 1, using 3-(tert-butyl)-5-(1,1,1-trifluoro-2-methylpropan-2-yl)benzaldehyde (obtained according to Example D) as the required benzaldehyde in the reaction Scheme 4. The crude product was purified by prep-HPLC (column: Boston Green ODS 150*30 5μ; mobile phase: [water (0.1% TFA)-ACN]; B %: 25%-58.75%, 9 min) affording the title compound (227 mg, 387 μmol, 58% yield, 100% purity as a white solid. LCMS (m/z 587.1 (M+H)). ¹⁹F NMR (376 MHz, CD₃OD) −77.29 (br s, 1F), −77.31 (s, 1F). ¹H NMR (CD₃OD, 400 MHz) δ=7.61 (d, 7=7.3 Hz, 1H), 7.36 (s, 1H), 7.19 (s, 1H), 7.13 (s, 1H), 6.69 (d, 7=7.3 Hz, 1H), 5.38 (d, 7=1.8 Hz, 1H), 4.33-4.23 (m, 2H), 3.53-3.48 (m, 2H), 3.48-3.39 (m, 4H), 3.15 (t, 7=6.0 Hz, 2H), 2.89-2.60 (m, 6H), 1.96 (quin, 7=5.9 Hz, 2H), 1.54 (s, 6H), 1.29 (s, 9H).

### Example D

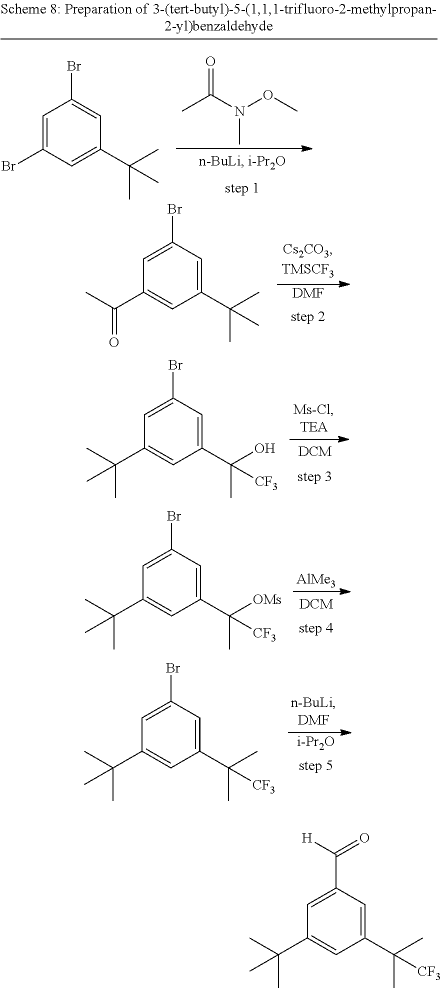

### Step 1. Preparation of 1-(3-bromo-5-(tert-butyl)phenyl)ethanone

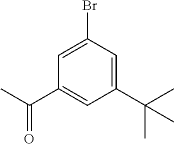

A stirred mixture of 1,3-dibromo-5-tert-butyl-benzene (11 g, 37.67 mmol, 1 eq) in anhydrous i-Pr₂O (160 mL) was cooled to −78° C. under nitrogen atmosphere was treated drop wise with n-BuLi (2.5 M, 15.1 mL, 1 eq). The resulting mixture was stirred at −78° C. for 30 min. After complete addition of n-BuLi, N-methoxy-N-methyl-acetamide (4.66 g, 45.20 mmol, 4.81 mL, 1.2 eq) was added dropwise, while keeping the mixture below −78° C. After addition, the mixture was warmed slowly to 10° C. for 30 min. The reaction mixture was quenched with saturated NH₄Cl (200 mL) then separated and extracted with ethyl acetate (200 mL*2). The combined organic layers were washed with H₂O (200 mL*2) and brine (200 mL), dried over Na₂SO₄, filtered and concentrated under reduced pressure to give a residue. The residue was purified by flash silica gel chromatography (ISCO®; 120 g CombiFlash® Silica Flash Column, Eluent of 0˜10% Ethyl acetate/Petroleum ether gradient @ 85 mL/min). The title was obtained as a light yellow solid (2 batches, 7.3 g, average yield: 64%). ¹H NMR (400 MHz, CDCl₃) δ=7.94-7.90 (m, 1H), 7.88 (t, J=1.7 Hz, 1H), 7.71 (t, J=1.8 Hz, 1H), 2.60 (s, 3H), 1.37-1.31 (m, 9H).

### Step 2. Preparation of 2-(3-bromo-5-(tert-butyl)phenyl)-1,1,1-trifluoropropan-2-ol

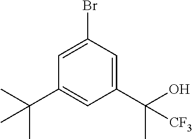 [SMILES: CC(C)(C)c1cc(Br)cc(C(C)(O)C(F)(F)F)c1]

To a solution of 1-(3-bromo-5-tert-butyl-phenyl)ethanone (6.3 g, 24.69 mmol, 1 eq) and TMSCF₃ (7.02 g, 49.38 mmol, 2 eq) in DMF (70 mL) was added Cs₂CO₃ (16.09 g, 49.38 mmol, 2 eq) at 0° C. The resulting mixture was warmed to 10° C. and stirred 16 hr. The reaction mixture was quenched by addition H₂O (100 mL), and then separated and extracted with ethyl acetate (200 mL*2). Combined organic layers were washed with H₂O (300 mL*2) and brine then dried over Na₂SO₄, filtered and concentrated under reduced pressure to give a residue. The residue was purified by flash silica gel chromatography (ISCO®; 120 g CombiFlash® Silica Flash Column, Eluent of 0˜10% Ethyl acetate/Petroleum ether gradient @ 85 mL/min). The title (8 g, 24.6 mmol, 99.6% yield) was obtained as a brown gum. NMR (400 MHz, CDCl₃) δ=7.54 (s, 2H), 7.52-7.50 (m, 1H), 1.32 (s, 12H).

### Step 3. Preparation of Preparation of 2-(3-bromo-5-(tert-butyl)phenyl)-1,1,1-trifluoropropan-2-yl methanesulfonate

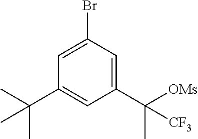 [SMILES: CC(C)(C)c1cc(Br)cc(C(C)(OS(C)(=O)=O)C(F)(F)F)c1]

A stirring mixture of 2-(3-bromo-5-tert-butyl-phenyl)-1,1,1-trifluoro-propan-2-ol (8.5 g, 26.14 mmol, 1 eq) and TEA (7.94 g, 78.42 mmol, 10.92 mL, 3 eq) in DCM (100 mL) under a nitrogen atmosphere was cooled to 0° C. and treated dropwise with MsCl (4.5 g, 39.3 mmol, 3.04 mL, 1.5 eq). The resulting mixture was stirred at 10° C. for 3 h. The reaction mixture was quenched by addition water (100 mL), and then extracted with DCM (150 mL*3). The combined organic layers were washed with brine (200 mL), dried over Na₂SO₄, filtered and concentrated under reduced pressure to give a residue. The residue was purified by flash silica gel chromatography (ISCO®; 120 g CombiLlash® Silica Llash Column, Eluent of 0˜10% Ethyl acetate/Petroleum ether gradient @ 85 mL/min). The title compound (9.1 g, 22.6 mmol, 86% yield) was obtained as a brown gum. NMR (400 MHz, CDCl₃) δ=7.59 (t, 7=1.6 Hz, 1H), 7.56 (s, 1H), 7.46 (s, 1H), 3.15 (s, 3H), 2.28 (s, 3H), 1.33 (s, 9H).

### Step 4. Preparation of 1-bromo-3-(tert-butyl)-5-(1,1,1-trifluoro-2-methylpropan-2-yl (benzene

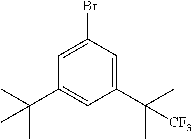 [SMILES: CC(C)(C)c1cc(Br)cc(C(C)(C)C(F)(F)F)c1]

A stirring solution of [1-(3-bromo-5-tert-butyl-phenyl)-2,2,2-trifluoro-1-methyl-ethyl] methanesulfonate (6.1 g, 15.13 mmol, 1 eq) in DCM (70 mL) at −78° C. was treated with Al(CH₃)₃ (1 M, 30.25 mL, 2 eq) by dropwise addition. After addition, the mixture was stirred at −78° C. for 0.5 hr, the mixture was then warmed to 10° C. and stirred for 5.5 hr. The reaction mixture was quenched with saturated NH₄Cl (100 mL) then extracted with DCM (150 mL*3). The combined organic layers were washed with brine (200 mL), dried over Na₂SO₄, filtered and concentrated under reduced pressure to give a residue. The residue was purified by flash silica gel chromatography (ISCO®; 120 g CombiFlash® Silica Flash Column, Eluent of 0˜1% Ethyl acetate/Petroleum ether gradient @ 85 mL/min). The title compound was obtained as a white solid (2 batches, 6.44 g, average yield: 88.34%). ¹H NMR (400 MHz, CDCl₃) δ=7.49-7.46 (m, 1H), 7.44 (s, 2H), 1.57 (s, 6H), 1.32 (s, 9H).

### Step 5. Preparation of 3-(tert-butyl)-5-(1,1,1-trifluoro-2-methylpropan-2-yl)benzaldehyde

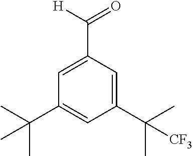 [SMILES: CC(C)(C)c1cc(C=O)cc(C(C)(C)C(F)(F)F)c1]

A stirring solution of 1-bromo-3-tert-butyl-5-(2,2,2-trifluoro-1,1-dimethyl-ethyl)benzene (2.5 g, 7.74 mmol, 1 eq) in anhydrous THF (50 mL) under nitrogen was cooled to −78° C. and treated with n-BuLi (2.5 M, 6.19 mL, 2 eq) by dropwise addition. The resulting mixture was stirred at −78° C. for 30 min. After complete addition of n-BuLi, DMF (1.13 g, 15.47 mmol, 1.19 mL, 2 eq) was added by dropwise addition, keeping the mixture below −78° C. After addition, the resulting mixture was stirred at −78° C. for 1.5 h. The reaction mixture was quenched with saturated NH₄Cl (60 mL) then extracted with EtOAc (100 mL*3). The combined organic layers were washed with water (200 mL) and brine (200 mL), dried over Na₂SO₄, filtered and concentrated under reduced pressure to give a residue. The residue was purified by flash silica gel chromatography (ISCO®; 120 g CombiFlash® Silica Flash Column, Eluent of 0˜5% Ethyl acetate/Petroleum ether gradient @ 85 mL/min). The title was obtained as a white solid (2 batches, 3.4 g, average yield: 73.28%). ¹H NMR (400 MHz, CDCl₃) δ=10.07-10.01 (m, 1H), 7.87 (s, 1H), 7.82 (br d, 7=4.8 Hz, 2H), 1.64 (s, 6H), 1.38 (s, 9H).

### Example 6

### Preparation of 3-(3-(tert-butyl)-5-(1-(difluoromethyl)cyclopropyl)phenyl)-4-(1-methyl-5-(2-(5,6,7,8-tetrahydro-1,8-naphthyridin-2-yl)ethoxy)-1H-pyrazol-3-yl)butanoic acid

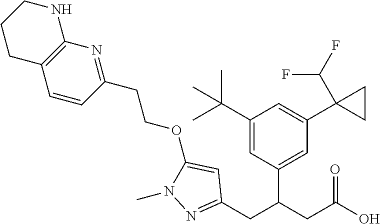 [SMILES: Cn1nc(CC(CC(=O)O)c2cc(C(C)(C)C)cc(C3(C(F)F)CC3)c2)cc1OCCc1ccc2c(n1)NCCC2]

Example 6 was prepared in analogous manner to Example 1, using 3-(tert-butyl)-5-(1-(difluoromethyl)cyclopropyl) benzaldehyde (obtained according to Example E) as the required benzaldehyde in the reaction Scheme 4. The crude product was purified by prep-HPLC (column: Boston Green ODS 150*30 5μ; mobile phase: [water (0.1% TFA)-ACN]; B %: 25%-55%, 8 min) affording the title compound (7.3 mg, 10.7 μmol, 32% yield, 99% purity, TFA) as a white solid. LCMS (m/z 567.1 (M+H)). ¹⁹F NMR (376 MHz, CD₃OD) −77.47 (br s, 3F), −118.13 (br d, J=57.2 Hz, 2F). ¹H NMR (CD₃OD, 400 MHZ) 5 7.60 (d, J=7.6 Hz, 1H), 7.25 (s, 1H), 7.14 (s, 1H), 7.03 (s, 1H), 6.68 (d, J=7.2 Hz, 1H), 5.79-5.47 (m, 1H), 5.46-5.41 (m, 1H), 4.36-4.24 (m, 2H), 3.54-3.46 (m, 5H), 3.45-3.36 (m, 1H), 3.15 (t, J=6.0 Hz, 2H), 2.92-2.75 (m, 4H), 2.75-2.59 (m, 2H), 2.01-1.91 (m, 2H), 1.27 (s, 9H), 1.14-1.06 (m, 2H), 0.90 (br s, 2H).

### Example E

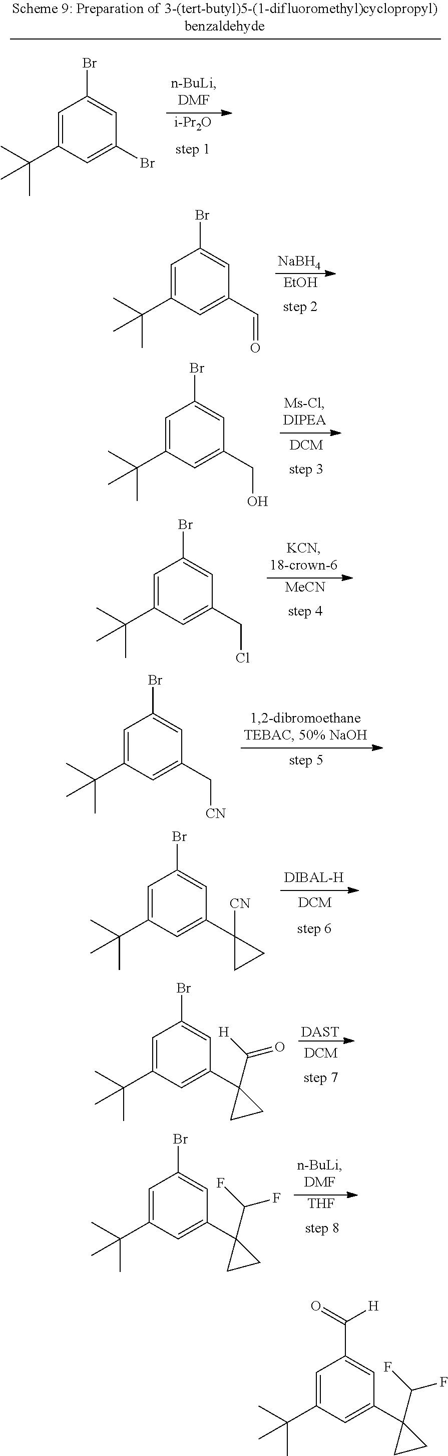

### Step 1. Preparation of 3-bromo-5-(tert-butyl)benzaldehyde

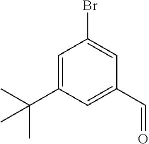

A stirred mixture of 1,3-dibromo-5-tert-butyl-benzene (19 g, 65 mmol, 1 eq) in anhydrous i-Pr₂O (240 mL) was cooled to −78° C. n-BuLi (2.5 M, 26.03 mL, 1 eq) was added dropwise to the mixture and the resulting mixture was stirred at −78° C. for 30 min. After complete addition of n-BuLi, DMF (5.71 g, 78.08 mmol, 6.01 mL, 1.2 eq) was added dropwise, while keeping the mixture below −78° C. After addition, the resulting mixture was stirred at −78° C. for 1.5 h. The reaction mixture was quenched with saturated NH₄Cl (200 mL) and extracted with ethyl acetate (200 mL*2). The combined organic layers were washed with water (200 mL*2) and brine (200 mL), dried over Na₂SO₄, filtered and concentrated under reduced pressure to give a residue. The residue was purified by flash silica gel chromatography (ISCO®; 120 g CombiFlash® Silica Flash Column, Eluent of 0˜10% Ethyl acetate/Petroleum ether gradient @ 85 mL/min). The title compound was obtained as a yellow liquid (2 batches, 15 g the average yield 87%). ¹H NMR (400 MHz, CDCl₃) δ=9.95 (s, 1H), 7.82 (t, J=1.7 Hz, 2H), 7.77 (t, J=1.8 Hz, 1H), 1.35 (s, 9H).

### Step 2. Preparation of (3-bromo-5-(tert-butyl)phenyl)methanol

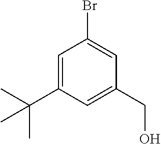

A solution of 3-bromo-5-tert-butyl-benzaldehyde (15 g, 62.21 mmol, 1 eq) in EtOH (150 mL) was treated with NaBH₄ (11.77 g, 311 mmol, 5 eq). The resulting mixture was stirred at 10° C. for 16 hr. The reaction mixture was quenched by addition of water (100 mL), and then extracted with ethyl acetate (200 mL*3). The combined organic layers were washed with brine (300 mL), dried over Na₂SO₄, filtered and concentrated under reduced pressure to give a residue. The residue was purified by flash silica gel chromatography (ISCO®; 120 g CombiFlash® Silica Flash Column, Eluent of 0˜10% Ethyl acetate/Petroleum ether gradient @ 85 mL/min). The title compound (14.7 g, 60 mmol, 97% yield) was obtained as a white solid. ¹H NMR (400 MHz, CDCl₃) δ=7.44 (t, J=1.6 Hz, 1H), 7.34 (s, 1H), 7.30 (s, 1H), 4.66 (br d, J=4.6 Hz, 2H), 1.31 (s, 9H).

### Step 3. Preparation of 1-bromo-3-(tert-butyl)-5-(chloromethyl)benzene

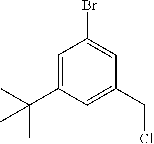

A stirring mixture of (3-bromo-5-tert-butyl-phenyl)methanol (14.7 g, 60.46 mmol, 1 eq) in anhydrous DCM (150 mL) was cooled to 0° C. under nitrogen atmosphere and then treated with DIPEA (15.63 g, 120.92 mmol, 21.06 mL, 2 eq) by drop-wise addition. The resulting mixture was stirred at 0° C. for 0.5 hr. Subsequently, MsCl (12 g, 104.76 mmol, 8.11 mL, 1.73 eq) was added drop-wise to the stirring mixture and the resulting mixture was stirred at 10° C. for 2.5 hr. The reaction mixture was quenched by addition water (150 mL), and then extracted with DCM (200 mL*3). The combined organic layers were washed with sat. NaHCO₃ (300 mL) and brine (300 mL), dried over Na₂SO₄, filtered and concentrated under reduced pressure to give a residue. The residue was purified by flash silica gel chromatography (ISCO®; 120 g CombiFlash® Silica Llash Column, Eluent of 0˜10% Ethyl acetate/Petroleum ether gradient @ 85 mL/min). The title compound (13.6 g, 51.99 mmol, 86% yield) was obtained as a colorless liquid. ¹H NMR (400 MHz, CDCl₃) δ=7.47 (t, J=1.6 Hz, 1H), 7.38 (s, 1H), 7.32 (s, 1H), 4.54 (s, 2H), 1.32 (s, 9H).

### Step 4. Preparation of 2-(3-bromo-5-(tert-butyl)phenyl)acetonitrile

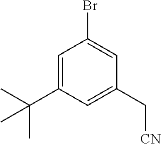

A suspension of 1-bromo-3-tert-butyl-5-(chloromethyl)benzene (13.6 g, 51.99 mmol, 1 eq) 18-CROWN-6 (1.37 g, 5.20 mmol, 0.1 eq) and KCN (17.01 g, 261 mmol, 11.19 mL, 5.02 eq) in CH₃CN (150 mL) was stirred for 16 hr at 10° C. The solvent was evaporated and the remainder was combined with water (200 mL). The aqueous mixture was extracted with DCM (300 mL) and partitioned. The aqueous layer was extracted with DCM (200 mL). The combined organic layers were dried over Na₂SO₄, filtered and concentrated under reduced pressure to give a residue. The residue was purified by flash silica gel chromatography (ISCO®; 120 g CombiFlash® Silica Flash Column, Eluent of 0˜5% Ethyl acetate/Petroleum ether gradient @ 80 mL/min). The title (11.2 g, 44.42 mmol, 85% yield) was obtained as a white solid. ¹H NMR (400 MHz, CDCl₃) δ=7.48 (s, 1H), 7.31 (s, 1H), 7.28-7.24 (m, 1H), 3.73 (s, 2H), 1.32 (s, 9H).

### Step 5. Preparation of 1-(3-bromo-5-(tert-butyl)phenyl)cyclopropanecarbonitrile

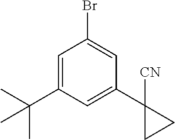 [SMILES: CC(C)(C)c1cc(Br)cc(C2(C#N)CC2)c1]

A mixture of TEBAC (505 mg, 2.22 mmol, 0.05 eq) and aq. NaOH (3.55 g, 44.42 mmol, 100 mL, 50%) were combined with 2-(3-bromo-5-tert-butyl-phenyl)acetonitrile (11.2 g, 44.42 mmol, 1 eq) and 1,2-dibromoethane (25.03 g, 133.25 mmol, 10.05 mL, 3 eq) at 0° C. The resulting mixture was stirred at 10° C. for 5 hrs. The mixture was poured into ice water (200 mL) and extracted with ethyl acetate (3×150 mL). The organic layer was dried over sodium sulfate and concentrated under reduced pressure to give a black brown liquid residue. The residue was purified by flash silica gel chromatography (ISCO®; 120 g CombiFlash® Silica Flash Column, Eluent of 0˜5% Ethyl acetate/Petroleum ether gradient @ 80 mL/min). The title compound (9.3 g, 33.43 mmol, 75% yield) was obtained as a yellow solid. ¹H NMR (400 MHz, CDCl₃) δ=7.44 (t, J=1.7 Hz, 1H), 7.32 (t, J=1.6 Hz, 1H), 7.16 (t, J=1.7 Hz, 1H), 1.77-1.72 (m, 2H), 1.43-1.39 (m, 2H), 1.32 (s, 9H).

### Step 6. Preparation of 1-(3-bromo-5-(tert-butyl)phenyl)cyclopropanecarbaldehyde

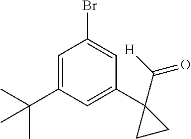 [SMILES: CC(C)(C)c1cc(Br)cc(C2(C=O)CC2)c1]

A solution of 1-(3-bromo-5-tert-butyl-phenyl)cyclopropanecarbonitrile (8.3 g, 29.84 mmol, 1 eq) in DCM (90 mL) was treated with DIBAL-H (1 M, 34.3 mL, 1.15 eq) at −78° C. The resulting mixture was stirred for 2 hr at −78° C. The reaction mixture was quenched with 2N HCl (100 mL) and extracted with DCM (3×100 mL). The combined organic layers were washed with saturated NaHCO₃ (200 mL), followed by brine (300 mL), dried over sodium sulfate and concentrated under reduced pressure to give a residue. The residue was purified by flash silica gel chromatography (ISCO®; 120 g CombiFlash® Silica Flash Column, Eluent of 0˜5% Ethyl acetate/Petroleum ether gradient @ 80 mL/min). The title compound was obtained as a yellow gum (2 batches, 7.9 g, average yield 84%). ¹H NMR (400 MHz, CDCl₃) δ=9.26-9.19 (m, 1H), 7.44 (t, J=1.7 Hz, 1H), 7.27-7.24 (m, 1H), 7.22 (t, J=1.6 Hz, 1H), 1.58-1.53 (m, 2H), 1.40-1.36 (m, 2H), 1.29 (s, 9H).

### Step 7. Preparation of 1-bromo-3-(tert-butyl)-5-(1-(difluoromethyl)cyclopropyl)benzene

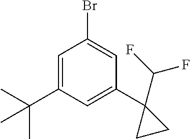 [SMILES: CC(C)(C)c1cc(Br)cc(C2(C(F)F)CC2)c1]

A stirred solution of 1-(3-bromo-5-tert-butyl-phenyl)cyclopropanecarbaldehyde (7.9 g, 28.10 mmol, 1 eq) in DCM (120 mL) was treated with DAST (18.11 g, 112.38 mmol, 14.85 mL, 4 eq) by slow addition at 0° C. The resulting mixture was stirred for 2 hrs at 25° C. The mixture was diluted with DCM (150 mL) and washed with water (150 mL). The organic layer was washed with brine (150 mL), dried over sodium sulfate and concentrated under reduced pressure to give a residue. The residue was purified by flash silica gel chromatography (ISCO®; 120 g CombiFlash® Silica Flash Column, Eluent of 0˜5% Ethyl acetate/Petroleum ether gradient @ 85 mL/min). The title (6.5 g, 21.44 mmol, 76% yield) was obtained as a yellow solid. ¹H NMR (400 MHz, CDCl3) δ=7.45 (t, 7=1.6 Hz, 1H), 7.37 (s, 1H), 7.35 (s, 1H), 5.80-5.46 (m, 1H), 1.31 (s, 9H), 1.19-1.14 (m, 2H), 1.00-0.94 (m, 2H). ¹⁹F NMR (376 MHz, CDCl₃) δ=−117.40 (s, 1F), −127.24-−128.43 (m, 1F).

### Step 8. Preparation of 3-(tert-butyl)-5-(1-(difluoromethyl)cyclopropyl) benzaldehyde

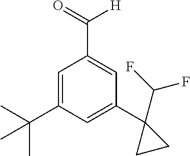 [SMILES: CC(C)(C)c1cc(C=O)cc(C2(C(F)F)CC2)c1]

A stirred solution of 1-bromo-3-tert-butyl-5-[1-(difluoromethyl)cyclopropyl]benzene (2.8 g, 9.24 mmol, 1 eq) in THF (60 mL) at −78° C. was treated with n-BuLi (2.5 M, 7.39 mL, 2 eq) by drop-wise addition. The resulting mixture was stirred for 0.5 h at −78° C. and then treated with DMF (1.35 g, 18.47 mmol, 1.42 mL, 2 eq) at −78 C by drop-wise addition. The resulting mixture was stirred for 1.5 hr. The reaction mixture was quenched with saturated NH₄Cl (60 mL) and extracted with EtOAc (100 mL*3). The combined organic layers were washed with water (200 mL) and brine (200 mL), dried over Na₂SO₄, filtered and concentrated under reduced pressure to give a residue. The residue was purified by flash silica gel chromatography (ISCO®; 120 g CombiFlash® Silica Flash Column, Eluent of 0˜5% Ethyl acetate/Petroleum ether gradient @ 85 mL/min). The title compound was obtained as a colorless gum (2 batches, 3.8 g, average yield: 78.67%). ¹H NMR (400 MHz, CDCl₃) δ=10.06-9.98 (m, 1H), 7.85 (d, J=1.5 Hz, 1H), 7.73 (d, J=7.3 Hz, 2H), 5.79-5.47 (m, 1H), 1.37 (s, 9H), 1.24-1.19 (m, 2H), 1.05-0.99 (m, 2H). ¹⁹F NMR (376 MHz, CDCl₃) δ=−116.80-−117.03 (m, 2F).

### Example 7

### Preparation of Preparation of 3-[3-tert-butyl-5-(2-hydroxy-1,1-dimethyl-ethyl)phenyl]-4-[1-methyl-5-[2-(5,6,7,8-tetrahydro-1,8-naphthyridin-2-yl)ethoxy]pyrazol-3-yl]butanoic acid

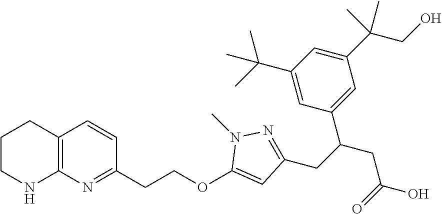 [SMILES: Cn1nc(CC(CC(=O)O)c2cc(C(C)(C)C)cc(C(C)(C)CO)c2)cc1OCCc1ccc2c(n1)NCCC2]

Example 7 was prepared in analogous manner to Example 1, using 3-tert-butyl-5-(1,1-dimethyl-2-triisopropylsilyloxy-ethyl)benzaldehyde (obtained according to Example F) as the required benzaldehyde in the reaction Scheme 4 and reversing the order of the esterification (Step 10) and pyrazole formation (Step 9) steps. The silyl protecting group was removed under the acidic conditions of Step 11. The crude product was purified by prep-HPLC (column: Boston Green ODS 150*30 5μ; mobile phase: [water (0.1% TFA)-ACN]; B %: 25%-55%, 8 min) affording the title compound (30 mg, 43 μmol, 50% yield, 97% purity, TFA) as a white solid. LCMS (m/z 549.1 (M+H)). ¹⁹F NMR (376 MHz, CD₃OD) −77.40 (s, 1F). ¹H NMR (CD₃OD, 400 MHz) δ 7.61 (d, J=7.6 Hz, 1H), 7.25 (t, 7=1.6 Hz, 1H), 7.03 (s, 2H), 6.70 (d, 7=7.2 Hz, 1H), 5.51 (s, 1H), 4.33 (t, 7=6.0 Hz, 2H), 3.49-3.54 (m, 4H), 3.48 (s, 3H), 3.40 (dt, 7=15.2, 7.6 Hz, 1H), 3.16 (t, 7=6.0 Hz, 2H), 2.74-2.90 (m, 4H), 2.58-2.73 (m, 2H), 1.96 (dt, 7=11.6, 6.0 Hz, 2H), 1.24-1.29 (m, 15H).

### Example F

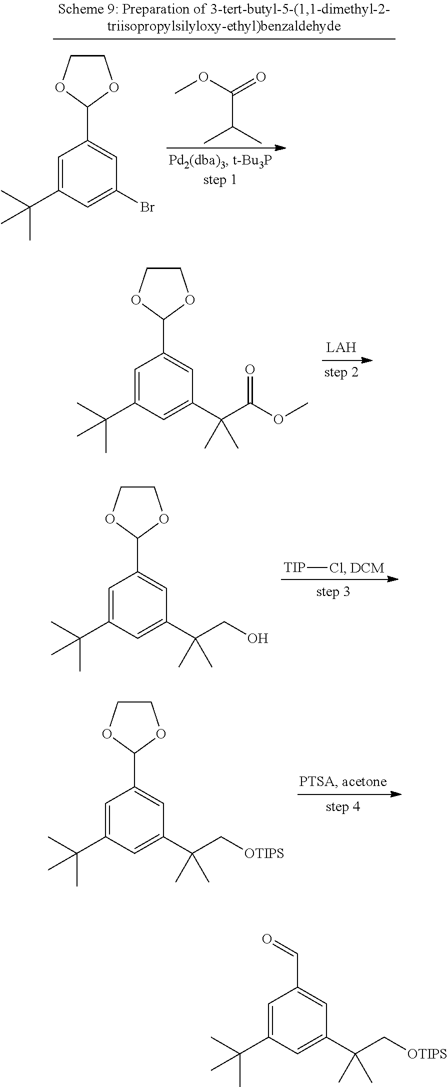

### Step 1. Preparation of methyl 2-(3-(tert-butyl)-5-(1,3-dioxolan-2-yl)phenyl)-2-methylpropanoate

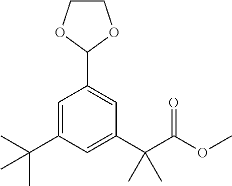 [SMILES: COC(=O)C(C)(C)c1cc(C2OCCO2)cc(C(C)(C)C)c1]

A stirred mixture of dicyclohexylamine (4.13 g, 22.79 mmol, 4.54 mL, 1.3 eq) in toluene (50 mL) at −20° C. under argon was treated with n-BuLi (2.5 M in hexane, 9.12 mL, 1.3 eq) by dropwise addition. The resulting mixture was stirred 15 min at 0° C. and then treated with methyl 2-methylpropanoate (2.15 g, 21.04 mmol, 2.41 mL, 1.2 eq) by dropwise addition. The resulting mixture was allowed to warm to 15° C. and stirred for 30 min. then 2-(3-bromo-5-tert-butyl-phenyl)-1,3-dioxolane (5 g, 17.53 mmol, 1 eq), t-Bu₃P (1.06 g, 526 μmol, 1.23 mL, 10% purity, 0.03 eq) and Pd(dba)₂ (302 mg, 526 μmol, 0.03 eq) were added and the resulting mixture was stirred at 15° C. for 40 hr. The mixture was then diluted with ethyl acetate (100 mL) and combined with aqueous NH₄Cl (100 mL). The mixture was filtered and the organic phase was washed with a saturated NaCl solution (20 mL), dried over Na₂SO₄, filtered, and concentrated under reduced pressure. The residue was purified by flash silica gel chromatography (ISCO®; 80 g SepaFlash® Silica Flash Column, Eluent of 0˜10% Ethyl acetate/Petroleum ether gradient @ 60 mL/min). The title compound (4.4 g, 14.4 mmol, 82% yield) was obtained as a yellow oil. LCMS (m/z 307.0 (M+H)). ¹H NMR (CDCl₃, 400 MHz) 7.39 (s, 1H), 7.35 (t, 7=2.0 Hz, 1H), 7.28 (s, 1H), 5.79 (s, 1H), 4.02-4.19 (m, 4H), 3.65 (s, 3H), 1.59 (s, 6H), 1.32 (s, 9H).

### Step 2. Preparation of 2-(3-(tert-butyl)-5-(1,3-dioxolan-2-yl)phenyl)-2-methylpropan-1-ol

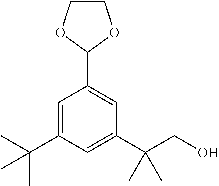 [SMILES: CC(C)(C)c1cc(C2OCCO2)cc(C(C)(C)CO)c1]

A mixture of LAH (495 mg, 13 mmol, 2 eq) in THF (5 mL) was treated with a mixture of methyl 2-[3-tert-butyl-5-(1,3-dioxolan-2-yl)phenyl]-2-methyl-propanoate (2 g, 6.5 mmol, 1 eq) in THF (20 mL) at 0° C. under N₂. The resulting mixture was stirred at 15° C. for 16 hr. The mixture was diluted with ethyl acetate (40 mL) and cooled to 0° C. The excess reagent was quenched with Na₂SO₄.10H₂O and the mixture was stirred for 1 hr and filtered. The organic phase was concentrated under reduced pressure. The residue was used into the next step without further purification. The title compound (1.8 g, 5.83 mmol, 89% yield, 90% purity) was obtained as a white solid. LCMS (m/z 279.0 (M+H)) ¹H NMR (CDCl₃, 400 MHz) 7.41-7.45 (m, 1H), 7.38 (s, 1H), 7.32 (s, 1H), 5.79 (s, 1H), 4.13-4.20 (m, 2H), 4.03-4.08 (m, 2H), 3.62 (s, 2H), 1.36 (s, 6H), 1.34 (s, 9H).

### Step 3. Preparation of [2-[3-tert-butyl-5-(1,3-dioxolan-2-yl)phenyl]-2-methyl-propoxy]-triisopropyl-silane

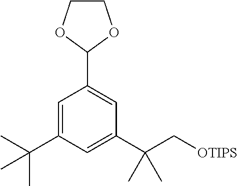

A mixture of 2-[3-tert-butyl-5-(1,3-dioxolan-2-yl)phenyl]-2-methyl-propan-1-ol (1.8 g, 5.83 mmol, 1 eq), imidazole (595 mg, 8.74 mmol, 1.5 eq), and TIPSCl (1.35 g, 6.99 mmol, 1.50 mL, 1.2 eq) in dry DCM (35 mL) and DMF (4 mL) was stirred at 30° C. for 16 hr. The mixture was concentrated under reduced pressure to remove the solvent and 100 mL of water was added. The aqueous mixture was extracted with ethyl acetate (2*100 mL). The combined organic layers were washed with brine (100 mL), dried over anhydrous Na₂SO₄, and concentrated under reduced pressure. The residue was purified by flash silica gel chromatography (ISCO®; 20 g SepaFlash® Silica Flash Column, Eluent of 0˜10% Ethyl acetate/Petroleum ether gradient @35 mL/min). The title compound (1.7 g, 3.88 mmol, 67% yield, 99% purity) was obtained as a colorless oil. LCMS (m/z 435.2 (M+H)) ¹H NMR (CDCl₃, 400 MHz) 7.43 (t, 7=1.6 Hz, 1H), 7.30-7.36 (m, 2H), 5.81 (s, 1H), 4.13-4.18 (m, 2H), 4.01-4.09 (m, 2H), 3.66 (s, 2H), 1.35 (s, 6H), 1.33 (s, 9H), 0.98-1.04 (m, 21H).

### Step 4. Preparation of 3-tert-butyl-5-(1,1-dimethyl-2-triisopropylsilyloxy-ethyl)benzaldehyde

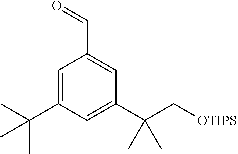

A mixture of [2-[3-tert-butyl-5-(1,3-dioxolan-2-yl)phenyl]-2-methyl-propoxy]-triisopropyl-silane (1.7 g, 3.88 mmol, 1 eq) and PTSA (134 mg, 775 μmol, 0.2 eq) in anhydrous acetone (20 mL) were stirred at 25° C. for 16 hr under nitrogen and then saturated NaHCO₃ (40 mL) was added. The resulting mixture was extracted with EtOAc (50 mL×2), dried over anhydrous Na₂SO₄, filtered and evaporated in vacuo to give the residue. The residue was purified by flash silica gel chromatography (ISCO®; 20 g SepaFlash® Silica Flash Column, Eluent of 0-5% Ethyl acetate/Petroleum ether gradient @ 35 mL/min). The title compound (1.02 g, 2.61 mmol, 67% yield) was obtained as colorless oil. LCMS (m/z 391.2 (M+H)) ¹H NMR (CDCl₃, 400 MHz) 10.01 (s, 1H), 7.70-7.75 (m, 3H), 3.69 (s, 2H), 1.38 (s, 6H), 1.36 (s, 9H), 0.93-1.03 (m, 21H).

### Example 8

### Preparation of 3-(3-chloro-5-(2-cyanopropan-2-yl)phenyl)-4-(1-methyl-5-(2-(5,6,7,8-tetrahydro-1,8-naphthyridin-2-yl)ethoxy)-1H-pyrazol-3-yl)butanoic acid

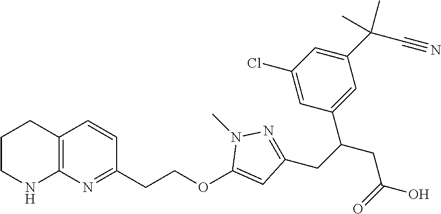 [SMILES: Cn1nc(CC(CC(=O)O)c2cc(Cl)cc(C(C)(C)C#N)c2)cc1OCCc1ccc2c(n1)NCCC2]

Example 8 was prepared in analogous manner to Example 1, using 2-(3-chloro-5-formylphenyl)-2-metliylpropanenitrile (obtained according to Example G) as the required benzaldehyde in the reaction Scheme 4 and replacing Step 12 of Scheme 4 with Step 4 of Scheme 5. The crude product was purified by prep-HPLC (column: DuraShell 150*25 mm*5 um; mobile phase: [water (0.1% TFA)-ACN]; B %: 15%-45%, 8 min) affording the title compound (210 mg, 330 μmol, TFA) as a white solid. LCMS (m/z 522.2 (M+H)⁺). ¹H NMR 5=1.62 (d, J=7.2 Hz, 1H), 7.34 (t, J=1.8 Hz, 1H), 7.26 (t, J=1.6 Hz, 1H), 7.23 (d, J=1.6 Hz, 1H), 6.70 (d, J=7.3 Hz, 1H), 5.53 (s, 1H), 4.35 (dt, J=1.9, 6.0 Hz, 2H), 3.56-3.42 (m, 6H), 3.18 (t, j=6.0 Hz, 2H), 2.96-2.87 (m, 1H), 2.85 (t, J=6.1 Hz, 2H), 2.81-2.73 (m, 2H), 2.72-2.62 (m, 1H), 1.97 (quin, J=6.0 Hz, 2H), 1.68 (s, 3H), 1.67 (s, 3H).

### Example G

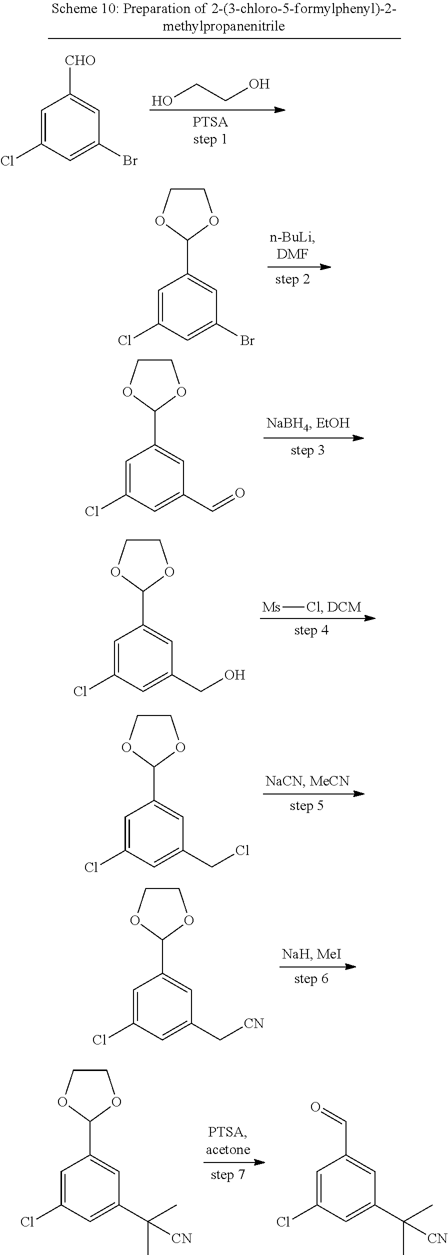

### Step 1. Preparation of 2-(3-bromo-5-chlorophenyl)-1,3-dioxolane

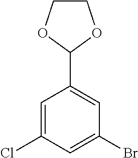 [SMILES: Clc1cc(Br)cc(C2OCCO2)c1]

A mixture of 3-bromo-5-chloro-benzaldehyde (20 g, 91.13 mmol, 1 eq) and ethylene glycol (16.97 g, 273 mmol, 15.3 mL, 3 eq) in toluene (300 mL) was treated with PTSA (314 mg, 1.82 mmol, 0.02 eq) in one portion at 25° C. under N₂. The resulting mixture was then heated to 140° C. and stirred for 3 hours. The mixture was cooled to 25° C. and saturated NaHCO₃ (100 mL) was added, the layers were separated. The aqueous layer was extracted with EtOAc (100 mL*3). The combined organic phase was washed with brine (50 mL*2), dried with anhydrous Na₂SO₄, filtered and concentrated in vacuum to afford the crude product. The crude product 2-(3-bromo-5-chloro-phenyl)-1,3-dioxolane (20 g, 75.9 mmol, 83% yield) was was obtained as colorless liquid and used in the next step without further purification. ¹H NMR (400 MHz, CDCl₃) δ=7.56-7.48 (m, 2H), 7.41 (s, 1H), 5.76 (s, 1H), 4.16-3.93 (m, 4H).

### Step 2. Preparation of 2-(3-bromo-5-chlorophenyl)-1,3-dioxolane

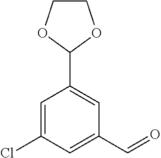 [SMILES: O=Cc1cc(Cl)cc(C2OCCO2)c1]

A stirring mixture of 2-(3-bromo-5-chloro-phenyl)-1,3-dioxolane (17 g, 64.51 mmol, 1 eq) in THF (250 mL) was treated with n-BuLi (2.5 M, 33.55 mL, 1.3 eq) dropwise at −70° C. After addition, the mixture was stirred at −70° C. for 30 min, and then DMF (5.89 g, 80.64 mmol, 6.2 mL, 1.25 eq) was added dropwise at −70° C. The resulting mixture was stirred for 1.5 hr at −70° C. The excess reagent was quenched with saturated NH₄Cl (50 mL) and then warmed to 20° C., and extracted with EtOAc (100 mL*3). The combined organic layer was washed with water (50 mL*3) and brine (50 mL*2), dried over Na₂SO₄, filtered and concentrated in vacuo to give a residue. The residue was purified by flash silica gel chromatography (ISCO®; 80 g SepaFlash® Silica Flash Column, Eluent of 0˜10% Ethyl acetate/Petroleum ether gradient @ 60 mL/min). The title compound (9 g, 42 mmol, 66% yield) was obtained as an colorless oil. ¹H NMR (400 MHz, CDCl₃) δ=9.98 (s, 1H), 7.90-7.82 (m, 2H), 7.73 (t, J=1.6 Hz, 1H), 5.86 (s, 1H), 4.13-4.04 (m, 4H).

### Step 3. Preparation of (3-chloro-5-(1,3-dioxolan-2-yl)phenyl)methanol

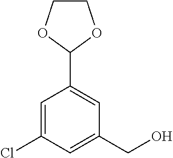 [SMILES: OCc1cc(Cl)cc(C2OCCO2)c1]

A stirring mixture of 3-chloro-5-(1,3-dioxolan-2-yl)benzaldehyde (9 g, 42.3 mmol, 1 eq) in EtOH (100 mL) was treated with NaBH₄ (3.20 g, 84.6 mmol, 2 eq) in one portion. The resulting mixture was stirred at 8° C. for 3 hr. The excess reagent was slowly quenched with saturated NH₄Cl until gas evolution ceased. The mixture was filtered and the cake was washed with CH₂Cl₂ (50 mL). The filtrate was concentrated in vacuo. The residue was taken up with 200 mL of EtOAc, which was washed with water (25 mL*2) and brine (25 mL*2), dried over Na₂SO₄, filtered and concentrated in vacuo to give the crude product. The title compound (8.2 g, 38.2 mmol, 90% yield) was obtained as a colorless oil and was used in the next step without further purification. ¹H NMR (400 MHz, CDCl₃) δ=7.39 (s, 1H), 7.35 (s, 1H), 7.33 (s, 1H), 5.77 (s, 1H), 4.66 (s, 2H), 4.13-4.00 (m, 4H), 2.02 (br s, 1H).

### Step 4. Preparation of 2-(3-chloro-5-(chloromethyl)phenyl)-1,3-dioxolane

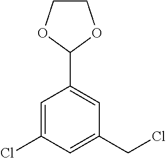 [SMILES: ClCc1cc(Cl)cc(C2OCCO2)c1]

A mixture of [3-chloro-5-(1,3-dioxolan-2-yl)phenyl]methanol (8.2 g, 38.2 mmol, 1 eq) in DCM (100 mL) was treated with TEA (7.73 g, 76.4 mmol, 10.6 mL, 2 eq) and MsCl (6.65 g, 58 mmol, 4.49 mL, 1.52 eq) in one portion. The resulting mixture was stirred at 10° C. for 20 hr and then concentrated in vacuo. The residue was taken up with EtOAc (150 mL) and filtered. The cake was washed with EtOAc (50 mL). The filtrate was washed with water (50 mL*2) and brine (50 mL*2), dried over Na₂SO₄ and concentrated in vacuo. The residue was purified by flash silica gel chromatography (ISCO®; 80 g SepaFlash® Silica Flash Column, Eluent of 0˜10% Ethyl acetate/Petroleum ether gradient @ 60 mL/min). The title compound (5 g, 21.45 mmol, 56% yield) was obtained as a colorless oil. ¹H NMR (400 MHz, CDCl₃) δ=7.43 (s, 1H), 7.39 (d, J=2.0 Hz, 2H), 5.79 (s, 1H), 4.55 (s, 2H), 4.14-4.02 (m, 4H).

### Step 5. Preparation of 2-(3-chloro-5-(1,3-dioxolan-2-yl)phenyl)acetonitrile

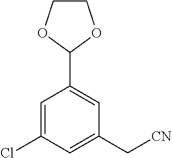 [SMILES: N#CCc1cc(Cl)cc(C2OCCO2)c1]

A mixture of 2-[3-chloro-5-(chloromethyl)phenyl]-1,3-dioxolane (5 g, 21.45 mmol, 1 eq) in EtOH (60 mL) and H₂O (20 mL) was treated with KCN (4.92 g, 75.56 mmol, 3.2 mL, 3.5 eq) in one portion. The resulting mixture was stirred at 60° C. for 20 hr and then diluted with water (35 mL). The mixture was extracted with EtOAc (50 mL*3). The combined organic layer was washed with water (50 mL*2) and brine (50 mL*2), dried over Na₂SO₄ and concentrated in vacuo. The aqueous layer was treated with saturated NaClO solution (with NaOH). The residue was purified by flash silica gel chromatography (ISCO®; 40 g SepaFlash® Silica Flash Column, Eluent of 0˜30% Ethyl acetate/Petroleum ether gradient @ 40 mL/min). The title compound (3.7 g, 16.54 mmol, 77% yield) was obtained as a colorless oil. ¹H NMR (400 MHz, CDCl₃) δ=7.45 (s, 1H), 7.34 (s, 2H), 5.78 (s, 1H), 4.13-4.02 (m, 4H), 3.75 (s, 2H).

### Step 6. Preparation of 2-(3-chloro-5-(1,3-dioxolan-2-yl)phenyl)-2-methylpropanenitrile

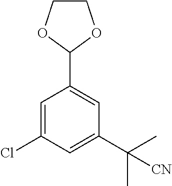 [SMILES: CC(C)(C#N)c1cc(Cl)cc(C2OCCO2)c1]

A mixture of 2-[3-chloro-5-(1,3-dioxolan-2-yl)phenyl]acetonitrile (4 g, 17.88 mmol, 1 eq) in THF (50 mL) was treated with NaH (2.5 g, 62.6 mmol, 60%, 3.5 eq) in portions at 0° C. After addition, CH₃I (14 g, 98.63 mmol, 6.14 mL, 5.51 eq) was added slowly at 0° C. Subsequently, the mixture was stirred at 20° C. for 20 hr. The excess reagent was quenched by slowly adding water (30 mL) at 0° C. to the mixture. The resultant was extracted with EtOAc (100 mL*2). The combined organic layer was washed with water (35 mL*2) and brine (35 mL*2), dried over Na₂SO₄, filtered and concentrated in vacuo. The residue was purified by flash silica gel chromatography (ISCO®; 40 g SepaFlash®Silica Flash Column, Eluent of 0-25% Ethyl acetate/Petroleum ether gradient @ 40 mL/min). The title compound (4 g, 15.89 mmol, 89% yield) was obtained as a light yellow oil. ¹H NMR (400 MHz, CDCl₃) δ=7.50-7.41 (m, 3H), 5.78 (s, 1H), 4.13-4.04 (m, 4H), 1.72 (s, 6H).

### Step 7. Preparation of 2-(3-chloro-5-formylphenyl)-2-methylpropanenitrile

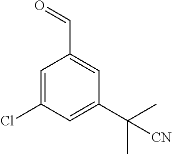

A mixture of 2-[3-chloro-5-(1,3-dioxolan-2-yl)phenyl]-2-methyl-propanenitrile (4 g, 15.9 mmol, 1 eq) in acetone (70 mL) was treated with PPTS (1 g, 3.97 mmol, 0.25 eq) in one portion at 20° C. under N₂. The resulting mixture was then stirred for 3 hours at 20° C. The mixture was concentrated in vacuo. The residue was purified by flash silica gel chromatography (ISCO®; 40 g SepaFlash® Silica Flash Column, Eluent of 0-20% Ethyl acetate/Petroleum ether gradient @ 40 mL/min). The title compound (2.8 g, 13.5 mmol, 85% yield) was obtained as colorless oil. ¹H NMR (400 MHz, CDCl₃) δ=10.00 (s, 1H), 7.87 (t, J=1.6 Hz, 1H), 7.82 (t, J=1.6 Hz, 1H), 7.74 (t, J=2.0 Hz, 1H), 1.78 (s, 6H).

### Example 9

### Preparation of 3-[3-tert-butyl-5-(1-cyano-1-methyl-ethyl)phenyl]-4-[1-methyl-5-[2-(5,6,7,8-tetrahydro-1,8-naphthyridin-2-yl)ethoxy]pyrazol-3-yl]butanoic acid

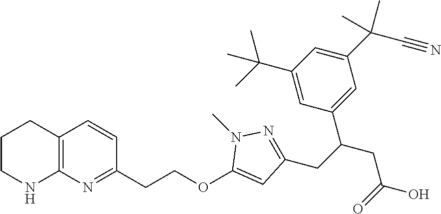 [SMILES: Cn1nc(CC(CC(=O)O)c2cc(C(C)(C)C)cc(C(C)(C)C#N)c2)cc1OCCc1ccc2c(n1)NCCC2]

Example 9 was prepared in analogous manner to Example 1, using 2-(3-(tert-butyl)-5-formylphenyl)-2-methylpropanenitrile (obtained according to Example H) as the required benzaldehyde in the reaction Scheme 4 and replacing Step 12 of Scheme 4 with Step 4 of Scheme 5. The crude product was purified by prep-HPLC (column: Xtimate C18 150*25 mm*5 μm; mobile phase:[water (0.075% TFA)-ACN; B %: 18%-48%, 9 min) affording the title compound (11 mg, 17 μmol, 9.7% yield, 99% purity, TFA) as a white solid. LCMS (m/z 544.3 (M+H)⁺). ¹H NMR (400 MHz, CD₃OD) 7.61 (d, J=7.6 Hz, 1H), 7.34 (s, 1H), 7.20 (s, 1H), 7.10 (s, 1H), 6.70 (d, J=7.6 Hz, 1H), 5.44 (s, 1H), 4.31 (t, J=5.6 Hz, 2H), 3.53-3.48 (m, 2H), 3.46 (s, 4H), 3.15 (t, J=6.0 Hz, 2H), 2.89-2.81 (m, 3H), 2.78-2.61 (m, 3H), 1.96 (quin, J=6.0 Hz, 2H), 1.67 (d, J=4.0 Hz, 6H), 1.29 (s, 9H). ¹⁹F NMR (376 MHz, CD₃OD) −77.32 (br s, 3F). Also, starting material, ethyl 3-[3-tert-butyl-5-(1-cyano-1-methyl-ethyl)phenyl]-4-[1-methyl-5-[2-(5,6,7,8-tetrahydro-1,8-naphthyridin-2-yl)ethoxy]pyrazol-3-yl]butanoate, (20 mg g, 29 μmol, 16% recovered yield, 98% purity, TFA) was obtained as a white solid. LC-MS mass: m/z 572.3 (M+H).

### Example H

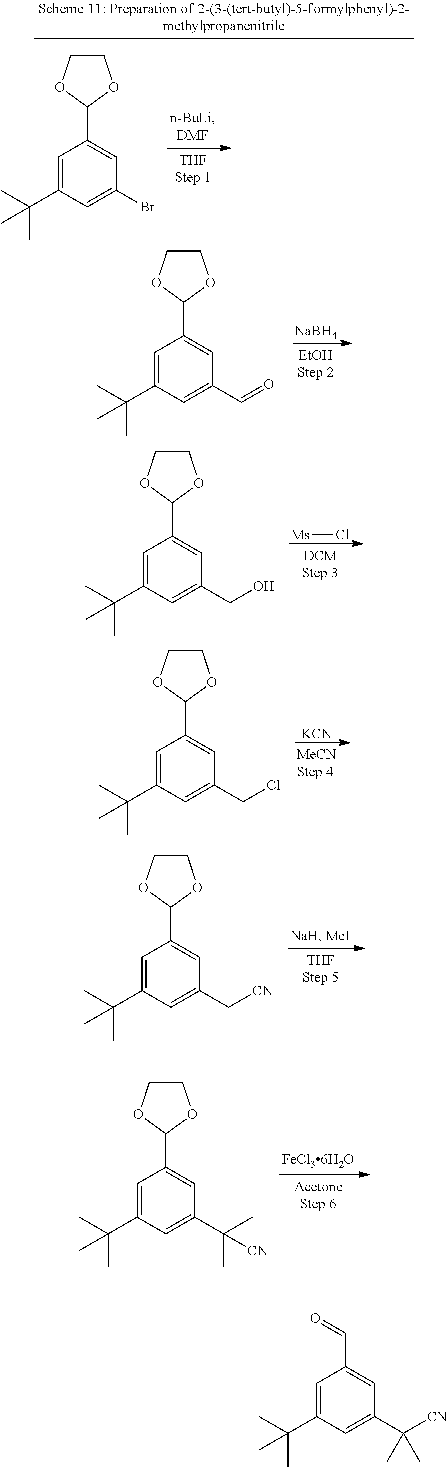

### Step 1. Preparation of 3-(tert-butyl)-5-(1,3-dioxolan-2-yl)benzaldehyde

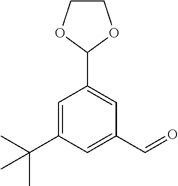 [SMILES: CC(C)(C)c1cc(C=O)cc(C2OCCO2)c1]

A stirring mixture of 2-(3-bromo-5-tert-butyl-phenyl)-1,3-dioxolane (15 g, 52.6 mmol, 1 eq) in anhydrous THF (300 mL) was treated with n-BuLi (2.5 M, 23.14 mL, 1.1 eq) dropwise at −78° C. under N₂. After addition, DMF (3.84 g, 52.60 mmol, 4.05 mL, 1 eq) was added dropwise while keeping the mixture below −78° C. The resulting mixture was stirred for 1.5 h at 78° C. The excess reagent was quenched with saturated NH₄Cl (300 mL) and extracted with Ethyl acetate (300 mL*2). The combined organic layers were washed with water (400 mL*2) and brine (400 mL), dried over Na₂SO₄, filtered and concentrated under reduced pressure to give a residue. The residue was purified by flash silica gel chromatography (ISCO®; 120 g CombiFlash® Silica Flash Column, Eluent of 0˜10% Ethyl acetate/Petroleum ether gradient @ 85 mL/min). The title (10.2 g, 43.5 mmol, 83% yield) was obtained as a yellow liquid. ¹H NMR (400 MHz, CDCl₃) 10.04 (s, 1H), 7.93 (t, J=1.6 Hz, 1H), 7.84 (s, 1H), 7.77 (t, J=1.6 Hz, 1H), 5.87 (s, 1H), 4.22-4.15 (m, 2H), 4.11-4.05 (m, 2H), 1.38 (s, 9H).

### Step 2. Preparation of (3-(tert-butyl)-5-(1,3-dioxolan-2-yl)phenyl)methanol

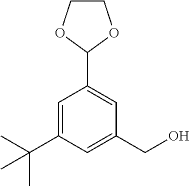 [SMILES: CC(C)(C)c1cc(CO)cc(C2OCCO2)c1]

A stirring mixture of 3-tert-butyl-5-(1,3-dioxolan-2-yl)benzaldehyde (10.2 g, 43.5 mmol, 1 eq) in EtOH (110 mL) was treated with NaBH₄ (8.24 g, 217.7 mmol, 5 eq) and then stirred at 10° C. for 16 hr. The excess reagent was quenched by addition of water (100 mL) to the mixture, and the resultant was extracted with EtOAc (200 mL*3). The combined organic layers were washed with brine (300 mL), dried over Na₂SO₄, filtered and concentrated under reduced pressure to give a residue. The residue was purified by flash silica gel chromatography (ISCO®; 120 g CombiFlash® Silica Flash Column, Eluent of 0˜30% Ethyl acetate/Petroleum ether gradient @ 85 mL/min). The title compound (9.4 g, 39.8 mmol, 91% yield) was obtained as a colorless liquid. ¹H NMR (400 MHz, CDCl₃) 7.42 (br d, J=6.6 Hz, 2H), 7.32 (s, 1H), 5.80 (s, 1H), 4.70 (s, 2H), 4.19-4.13 (m, 2H), 4.08-4.00 (m, 2H), 1.34 (s, 9H).

### Step 3. Preparation of 2-(3-chloro-5-(chloromethyl)phenyl)-1,3-dioxolane

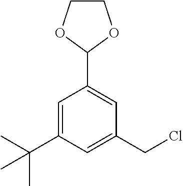 [SMILES: CC(C)(C)c1cc(CCl)cc(C2OCCO2)c1]

2-(3-Chloro-5-(chloromethyl)phenyl)-1,3-dioxolane was prepared in analogous manner to the preparation of 2-(3-chloro-5-(chloromethyl)phenyl)-1,3-dioxolane in Step 4 of Scheme 10 in Example G.

The title compound (10.9 g, crude) was obtained as a black brown liquid. NMR (400 MHz, CDCl₃) 7.46 (s, 1H), 7.41 (s, 1H), 7.35 (s, 1H), 5.81 (s, 1H), 4.67-4.58 (m, 2H), 4.22-4.12 (m, 2H), 4.12-4.02 (m, 2H), 1.34 (s, 9H).

### Step 4. Preparation of 2-(3-(tert-butyl)-5-(1,3-dioxolan-2-yl)phenyl)acetonitrile

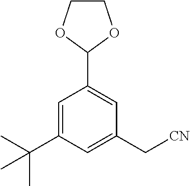 [SMILES: CC(C)(C)c1cc(CC#N)cc(C2OCCO2)c1]

A mixture of 2-[3-tert-butyl-5-(chloromethyl)phenyl]-1,3-dioxolane (10.8 g, 42.39 mmol, 1 eq), 18-CROWN-6 (1.12 g, 4.24 mmol, 0.1 eq) and KCN (13.80 g, 212 mmol, 9.1 mL, 5 eq) in CH₃CN (150 mL) was stirred for 16 hr at 10° C. The solvent was evaporated and the remainder combined with water (200 mL). The aqueous mixture was extracted twice with DCM (300 mL and 200 mL, respectively) The combined organic layers were dried over Na₂SO₄, filtered and concentrated under reduced pressure to give a residue. The residue was purified by flash silica gel chromatography (ISCO®; 120 g CombiFlash® Silica Flash Column, Eluent of 0˜30% Ethyl acetate/Petroleum ether gradient @ 80 mL/min). The title compound (8.74 g, 35.6 mmol, 84% yield) was obtained as a brown liquid. NMR (400 MHz, CDCl₃) 7.46 (s, 1H), 7.35 (s, 1H), 7.29 (s, 1H), 5.79 (s, 1H), 4.18-4.13 (m, 2H), 4.09-4.02 (m, 2H), 3.76 (s, 2H), 1.34 (s, 9H).

### Step 5. Preparation of 2-(3-(tert-butyl)-5-(1,3-dioxolan-2-yl)phenyl)-2-methylpropanenitrile

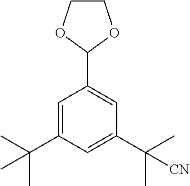 [SMILES: CC(C)(C)c1cc(C2OCCO2)cc(C(C)(C)C#N)c1]

2-(3-(Tert-butyl)-5-(1,3-dioxolan-2-yl)phenyl)-2-methylpropanenitrile was prepared in analogous manner to the preparation of 2-(3-chloro-5-(1,3-dioxolan-2-yl)phenyl)-2-methylpropanenitrile in Step 6 of Scheme 10 in Example G.

The title compound was obtained as as a yellow solid (3 batches, 8.8 g, average yield: 92%). NMR (400 MHz, CDCl₃) 7.54 (t, J=1.8 Hz, 1H), 7.45 (s, 1H), 7.38 (s, 1H), 5.80 (s, 1H), 4.20-4.12 (m, 2H), 4.11-4.03 (m, 2H), 1.74 (s, 6H), 1.35 (s, 9H).

### Step 6. Preparation of 2-(3-(tert-butyl)-5-formylphenyl)-2-methylpropanenitrile

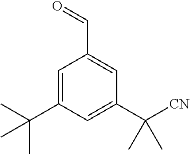 [SMILES: CC(C)(C)c1cc(C=O)cc(C(C)(C)C#N)c1]

A mixture of 2-[3-tert-butyl-5-(1,3-dioxolan-2-yl)phenyl]-2-methyl-propanenitrile (4.4 g, 16.10 mmol, 1 eq) and FeCl₃.6H₂O (870 mg, 3.22 mmol, 0.2 eq) in acetone (70 mL) was degassed and purged with N₂ for 3 times, and then the mixture was stirred at 25° C. for 1 hr under N₂ atmosphere. The mixture was diluted with water (50 mL) and extracted with Ethyl acetate (100 mL*3), the organic layer was washed with brine (200 mL), dried over Na₂SO₄ and concentrated under vacuum to give a residue (3.6 g). The residue was combined with a previous batch and purified by flash chromatography (ISCO®; 120 g CombiFlash® Silica Flash Column, Eluent of 0˜20% Ethyl acetate/Petroleum ether gradient @85 mL/min). The title compound was obtained as a yellow solid (2 batches, 6.6 g, average yield: 94%). ¹H NMR (400 MHz, CDCl₃) 10.04 (s, 1H), 7.87 (t, J=1.5 Hz, 1H), 7.83 (t, J=1.9 Hz, 1H), 7.77 (t, J=1.6 Hz, 1H), 1.78 (s, 6H), 1.39 (s, 9H).

### Example 10

### Preparation of 3-(3,5-di-tert-butyl-4-methoxyphenyl)-4-(1-methyl-5-(2-(5,6,7,8-tetrahydro-1,8-naphthyridin-2-yl)ethoxy)-1H-pyrazol-3-yl)butanoic acid

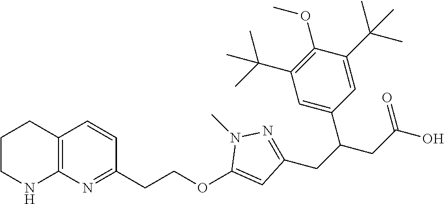 [SMILES: COc1c(C(C)(C)C)cc(C(CC(=O)O)Cc2cc(OCCc3ccc4c(n3)NCCC4)n(C)n2)cc1C(C)(C)C]

Example 10 was prepared in analogous manner to Example 1, using 3,5-di-tert-butyl-4-methoxybenzaldehyde (obtained according to Example I) as the required benzaldehyde in the reaction Scheme 4 and replacing Step 12 of Scheme 4 with Step 4 of Scheme 5. The crude product was purified by prep-HPLC (Boston Green ODS 150*30 5μ; mobile phase: [water (0.1% TFA)-ACN]; B %: 35%-61.25%, 7 min) affording the title compound (23 mg, 33 μmol, 49% yield, 98% purity, TFA) as a white solid. LCMS (m/z 563 (M+H) and m/z 585 (M+Na). ¹H NMR (400 MHz, CD₃OD) δ=7.60 (br d, J=7.2 Hz, 1H), 7.06-6.99 (m, 2H), 6.71-6.66 (m, 1H), 5.53-5.48 (m, 1H), 4.36-4.28 (m, 2H), 3.64-3.57 (m, 3H), 3.52-3.46 (m, 4H), 3.39-3.31 (m, 2H), 3.17 (br t, J=6.0 Hz, 2H), 2.85-2.54 (m, 6H), 1.99-1.89 (m, 2H), 1.54-1.14 (m, 18H).

### Example I

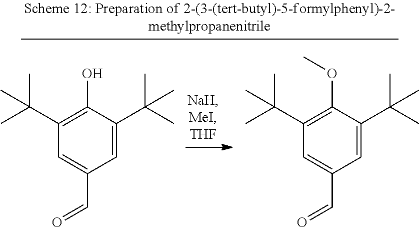 [SMILES: CC(C)(C)c1cc(C=O)cc(C(C)(C)C)c1O ; COc1c(C(C)(C)C)cc(C=O)cc1C(C)(C)C]

A stirring mixture of 3,5-di-tert-butyl-4-hydroxy-benzaldehyde (2 g, 8.53 mmol, 1 eq) in anhydrous THF (25 mL) was treated with NaH (853 mg, 21.34 mmol, 60%, 2.5 eq) at 0° C. under an argon atmosphere. The resulting mixture was stirred at 25° C. for 30 min and then treated with Mel (7.7 g, 54.25 mmol, 3.38 mL, 6.36 eq) by dropwise addition. The resulting mixture was stirred at 85° C. for 16 hr. The excess reagents were quenched by slow addition of brine (50 mL) and then extracted with Ethyl acetate (50 mL*3). The combined organic phase was washed with brine (100 mL), dried over anhydrous Na₂SO₄, filtered and concentrated in vacuo. The residue was purified by column chromatography (SiO₂, Petroleum ether to Petroleum ether/Ethyl acetate=10:1). The title compound (955 mg, 3.85 mmol, 45% yield) was obtained as yellow oil. ¹H NMR (CDCl₃, 400 MHZ) δ=9.91 (s, 1H), 7.79 (s, 2H), 3.73 (s, 3H), 1.45 (s, 18H).

### Example 11

### Preparation of 3-(3,5-di-tert-butyl-2-methoxyphenyl)-4-(1-methyl-5-(2-(5,6,7,8-tetrahydro-1,8-naphthyridin-2-yl)ethoxy)-1H-pyrazol-3-yl)butanoic acid

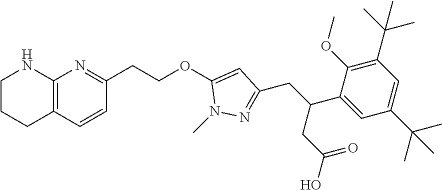 [SMILES: COc1c(C(CC(=O)O)Cc2cc(OCCc3ccc4c(n3)NCCC4)n(C)n2)cc(C(C)(C)C)cc1C(C)(C)C]

Example 11 was prepared in analogous manner to Example 1, using 3,5-di-tert-butyl-2-methoxybenzaldehyde as the required benzaldehyde (obtained according to Example J) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 32%-62%, 8 min). The title compound (87 mg, 128 μmol, 30% yield, TFA) was obtained as a white solid. LCMS (m/z 563 (M+H) and m/z 585 (M+Na). ¹H NMR (400 MHz, CD₃OD) δ=7.57 (d, J=7.3 Hz, 1H), 7.17 (d, J=2.4 Hz, 1H), 7.11 (d, J=2.4 Hz, 1H), 6.65 (d, J=7.3 Hz, 1H), 5.38 (s, 1H), 4.33-4.19 (m, 2H), 3.94-3.82 (m, 1H), 3.79-3.69 (m, 3H), 3.50-3.46 (m, 4H), 3.14 (t, J=6.0 Hz, 2H), 2.90-2.67 (m, 6H), 1.98-1.88 (m, 2H), 1.28 (d, J=19.8 Hz, 18H).

### Example J

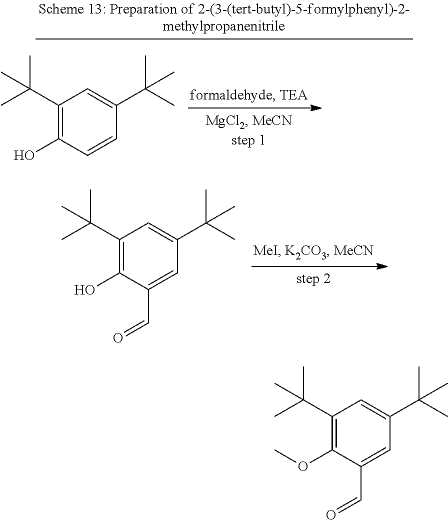

### Step 1. Preparation of 3,5-di-tert-butyl-2-hydroxybenzaldehyde

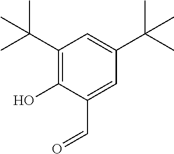 [SMILES: CC(C)(C)c1cc(C=O)c(O)c(C(C)(C)C)c1]

A mixture of 2,4-ditert-butylphenol (20 g, 97 mmol, 1 eq) in THF (300 mL), MgCl₂ (13.84 g, 145.4 mmol, 6 mL, 1.5 eq.) and TEA (20.36 g, 201.17 mmol, 28 mL, 2.1 eq) was stirred at 30° C. for 30 min. Subsequently, MeCHO (6 g, 136 mmol, 7.64 mL, 1.41 eq) was added to the mixture and the resulting mixture was heated at 70° C. for 4 h. After cooling to room temperature, the reaction mixture was quenched by HCl solution (5%, 75 mL) and extracted with ethyl acetate. The organic layer was dried over anhydrous Na₂SO₄ and the solvent was evaporated. The residue was purified by flash chromatography (ISCO®; 220 g SepaFlash® Silica Flash Column, Eluent of 0˜10% Ethyl acetate/Petroleum ether gradient @ 100 mL/min). The title compound (14.32 g, 61 mmol, 63% yield) was obtained as a yellow solid. 1H NMR (400 MHz, CDCl₃): δ ppm=9.88-9.82 (m, 1H), 7.59 (d, J=2.2 Hz, 1H), 7.34 (d, J=2.4 Hz, 1H), 1.47-1.33 (m, 18H).

### Step 2. Preparation of 3,5-di-tert-butyl-2-methoxybenzaldehyde

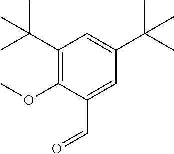 [SMILES: COc1c(C=O)cc(C(C)(C)C)cc1C(C)(C)C]

A mixture of 3,5-ditert-butyl-2-hydroxy-benzaldehyde (17.91 g, 76.43 mmol, 1 eq.) in anhydrous MeCN (85 mL) was treated with K₂CO₃ (11.62 g, 84 mmol, 1.1 eq) and Mel (21.70 g, 153 mmol, 9.52 mL, 2 eq) in a pressure vessel. The vessel was sealed and heated to 85° C. for 18 hrs. The mixture was allowed to cool and concentrated in vacuo. The organic layer was washed with 2N NaOH (150 mL), dried over anhydrous Na₂SO₄ and the solvent evaporated. The residue was purified by flash chromatography (ISCO®; 220 g SepaFlash® Silica Flash Column, Eluent of 0˜5% Ethyl acetate/Petroleum ether gradient @ 100 mL/min). The title compound (18.01 g, 72.5 mmol, 95% yield) was obtained as yellow liquid. 1H NMR (400 MHz, CDCl₃): δ ppm=10.40-10.28 (m, 1H), 7.71 (d, J=2.8 Hz, 1H), 7.65-7.59 (m, 1H), 3.93 (d, J=1.0 Hz, 3H), 1.43 (s, 9H), 1.32 (s, 9H).

### Example 12

### Preparation of 3-(3-bromo-5-(tert-butyl)-2-methoxyphenyl)-4-(1-methyl-5-(2-(5,6,7,8-tetrahydro-1,8-naphthyridin-2-yl)ethoxy)-1H-pyrazol-3-yl)butanoic acid

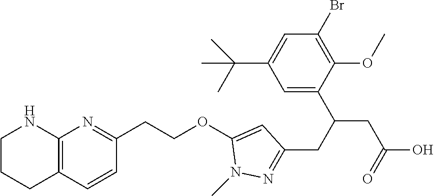 [SMILES: COc1c(Br)cc(C(C)(C)C)cc1C(CC(=O)O)Cc1cc(OCCc2ccc3c(n2)NCCC3)n(C)n1]

Example 12 was prepared in analogous manner to Example 1, using 3-bromo-5-(tert-butyl)-2-methoxybenzaldehyde as the required benzaldehyde (obtained according to Example K) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 25%-55%, 8 min.). The title compound (81 mg, yield 40%) was obtained as a white solid. LCMS (m/z 587 (M+H)). ¹H NMR (400 MHz, CD₃OD) δ=7.60 (d, J=7.3 Hz, 1H), 7.40 (d, J=2.4 Hz, 1H), 7.20 (d, J=2.2 Hz, 1H), 6.67 (d, J=7.5 Hz, 1H), 5.44 (s, 1H), 4.33 (t, J=6.1 Hz, 2H), 3.89-3.80 (m, 4H), 3.53-3.47 (m, 5H), 3.16 (t, J=6.0 Hz, 1H), 3.28-3.06 (m, 1H), 2.86-2.79 (m, 4H), 2.70 (d, 3=1.1 Hz, 2H), 1.99-1.92 (m, 2H), 1.26 (s, 9H).

### Example K

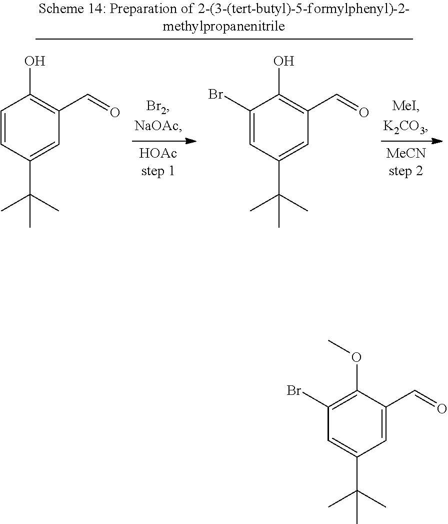

### Step 1. Preparation of 3-bromo-5-(tert-butyl)-2-hydroxybenzaldehyde

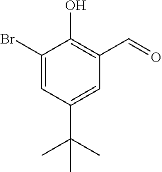

A mixture of 5-tert-butyl-2-hydroxy-benzaldehyde (10 g, 56.1 mmol, 1 eq.) in HO Ac (100 mL) was treated with a solution of Br₂ (13.45 g, 84.16 mmol, 4.34 mL, 1.5 eq.) by dropwise addition. The resulting mixture was stirred for 3 h at 50° C. The mixture was allowed to cool to rt and diluted with DCM (100 mL). The organic layer was washed with saturated sodium bisulfite solution (150 mL), water (100 mL), saturated NaHCO₃ (100 mL) and brine (100 mL), dried over anhydrous Na₂SO₄, filtered and evaporated in vacuum to give a residue (14.1 g, yield 87%). ¹H NMR (400 MHz, CDCl₃) δ=11.40 (s, 1H), 9.83 (s, 1H), 7.79 (d, J=2.2 Hz, 1H), 7.49 (d, J=2.2 Hz, 1H), 1.31 (s, 9H).

### Step 2. Preparation of 3-bromo-5-(tert-butyl)-2-methoxybenzaldehyde

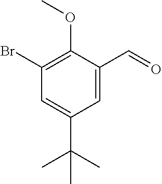

A mixture of 3-bromo-5-tert-butyl-2-hydroxy-benzaldehyde (14 g, 54.45 mmol, 1 eq) in CH₃CN (140 mL) was treated with K₂CO₃ (22.6 g, 163.4 mmol, 3 eq) and CH₃I (19.1 g, 134.6 mmol, 8.4 mL, 2.47 eq.). The resulting mixture was stirred at 85° C. for 16 hr. The reaction was quenched by addition water (120 mL), and the layers separated. The aqueous layer was extracted with Ethyl acetate (250 mL*2). The combined organic layers were dried over Na₂SO₄, filtered and concentrated under reduced pressure to give a residue. The residue was purified by flash chromatography (ISCO®; 120 g CombiFlash® Silica Flash Column, Eluent of 0˜10% Ethyl acetate/Petroleum ether gradient @ 85 mL/min). The title compound was obtained as a yellow liquid (12.1 g, yield 82%). LCMS (m/z 272 (M+H)). 1H NMR (400 MHz, CDCl₃) δ=10.37-10.26 (m, 1H), 7.86-7.70 (m, 1H), 7.86-7.70 (m, 1H), 3.99-3.87 (m, 3H), 1.33-1.25 (m, 9H).

### Example 13

### Preparation of 3-[3-(1-adamantyl)-4-benzyloxy-5-methoxy-phenyl]-4-[1-methyl-5-[2-(5,6,7,8-tetrahydro-1,8-naphthyridin-2-yl)ethoxy]pyrazol-3-yl]butanoic acid

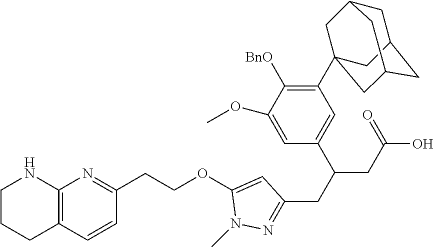 [SMILES: COc1cc(C(CC(=O)O)Cc2cc(OCCc3ccc4c(n3)NCCC4)n(C)n2)cc(C23CC4CC(CC(C4)C2)C3)c1OCc1ccccc1]

Example 13 was prepared in analogous manner to Example 1, using 3-(1-adamantyl)-4-benzyloxy-5-methoxy-benzaldehyde as the required benzaldehyde (obtained according to Example L) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 48%-74.25%, 7 min.). The title compound (10 mg, 14 μmol, 35% yield) was obtained as a white solid. LCMS (m/z 691 (M+H)). ¹H NMR (400 MHz, CD₃OD) δ=7.56 (d, 7=7.3 Hz, 1H), 7.47 (d, 7=7.1 Hz, 2H), 7.39-7.35 (m, 2H), 7.33-7.28 (m, 1H), 6.80 (d, 7=1.8 Hz, 1H), 6.66-6.62 (m, 2H), 5.50 (s, 1H), 5.53-5.47 (m, 1H), 5.02 (s, 2H), 4.34 (t, 7=6.2 Hz, 2H), 3.84 (s, 3H), 3.50 (s, 3H), 3.49-3.46 (m, 2H), 3.38 (br t, 7=7.6 Hz, 1H), 3.15 (t, 7=6.1 Hz, 2H), 2.86-2.63 (m, 6H), 2.00 (s, 6H), 1.93 (br d, 7=2.6 Hz, 5H), 1.72-1.62 (m, 6H).

### Example L

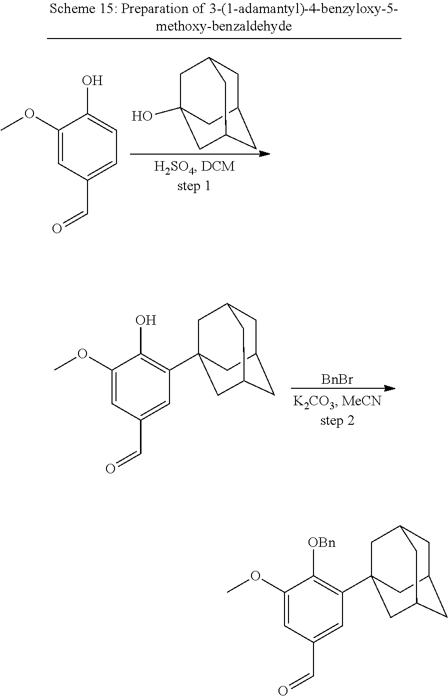

### Step 1. Preparation of 3-(1-adamantyl)-4-hydroxy-5-methoxy-benzaldehyde

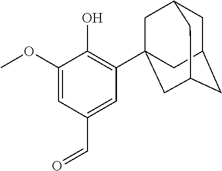 [SMILES: COc1cc(C=O)cc(C23CC4CC(CC(C4)C2)C3)c1O]

A mixture of 4-hydroxy-3-methoxy-benzaldehyde (15 g, 98.59 mmol, 1 eq) in DCM (100 mL) was treated with H₂SO₄ (113.38 mmol, 6.04 mL, 1.15 eq) and adamantan-l-ol (17.26 g, 113 mmol, 1.15 eq). The resulting mixture was stirred at 25° C. for 24 hr. The reaction mixture was concentrated in vacuo to remove the DCM, and then extracted with EtOAc (300 mL) and water (100 mL*3). The organic layer was washed with brine (50 mL*2), dried over Na₂SO₄ and concentrated in vacuo to obtain crude product. The crude product was purified by ISCO (80 g of silica gel column, Petroleum ether/Etheyl acetate is from 100% to 10/1) to obtain the desired product. The title compound (4.5 g, 15.7 mmol, 16% yield) was obtained as a white solid. ¹H NMR (400 MHz, DMSO-d₆) δ=9.78 (s, 1H), 7.39 (d, 7=1.8 Hz, 1H), 7.31 (d, 7=1.5 Hz, 1H), 3.87 (s, 3H), 2.10 (s, 6H), 2.05 (br s, 3H), 1.74 (br s, 6H).

### Step 2. Preparation of 3-(1-adamantyl)-4-benzyloxy-5-methoxy-benzaldehyde

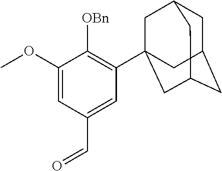 [SMILES: COc1cc(C=O)cc(C23CC4CC(CC(C4)C2)C3)c1OCc1ccccc1]

A mixture of 3-(1-adamantyl)-4-hydroxy-5-methoxy-benzaldehyde (2 g, 6.98 mmol, 1 eq) in MeCN (20 mL) was treated with benzylbromide (1.43 g, 8.38 mmol, 995 μL, 1.2 eq), K₂CO₃ (1.93 g, 13.97 mmol, 2 eq). The resulting mixture was stirred at 80° C. for 2 hr. The mixture was concentrated in vacuo to remove the MeCN, and then extracted with water (100 mL) and EtOAc (80 mL). The organic layer was washed with brine (50 mL*2), dried over Na₂SO₄ and concentrated in vacuo to obtain crude product. The crude product was purified by flash chromatography (ISCO®; 40 g CombiFlash® Silica Flash Column, Eluent of 0˜10% Ethyl acetate/Petroleum ether gradient @ 40 mL/min). The title compound (870 mg, 2.31 mmol, 33% yield) was obtained as a white solid. ¹H NMR (400 MHz, CDCl₃) δ=9.89 (s, 1H), 7.52 (d, 7=7.3 Hz, 2H), 7.45 (d, 7=1.8 Hz, 1H), 7.43-7.41 (m, 1H), 7.40-7.38 (m, 2H), 7.37-7.32 (m, 1H), 5.19 (s, 2H), 3.93 (s, 3H), 2.10 (d, 7=2.4 Hz, 6H), 2.02 (br s, 3H), 1.73-1.64 (m, 6H).

### Example 14

### Preparation of 3-[3-(1-adamantyl)-4,5-dimethoxy-phenyl]-4-[1-methyl-5-[2-(5,6,7,8-tetrahydro-1,8-naphthyridin-2-yl)ethoxy]pyrazol-3-yl]butanoic acid

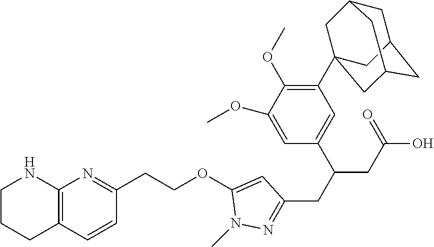 [SMILES: COc1cc(C(CC(=O)O)Cc2cc(OCCc3ccc4c(n3)NCCC4)n(C)n2)cc(C23CC4CC(CC(C4)C2)C3)c1OC]

Example 14 was prepared in analogous manner to Example 1, using 3-(1-adamantyl)-4,5-dimethoxy-benzaldehyde as the required benzaldehyde (obtained according to Example M) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 35%-65%, 8 min.). The title compound (140 mg, 228 μmol, 73% yield) was obtained as a white solid. LCMS (m/z 615 (M+H)). ¹H NMR (400 MHz, CD₃OD) δ=7.60 (d, 7=7.5 Hz, 1H), 6.77 (d, 7=1.5 Hz, 1H), 6.68 (d, 7=7.3 Hz, 1H), 6.61 (d, 7=1.5 Hz, 1H), 5.52 (s, 1H), 4.35 (t, 7=6.0 Hz, 2H), 3.82 (d, 7=10.3 Hz, 6H), 3.51 (s, 3H), 3.42-3.36 (m, 1H), 3.34-3.32 (m, 2H), 3.18 (t, 7=6.0 Hz, 2H), 2.84 (br d, 7=5.3 Hz, 2H), 2.80-2.58 (m, 4H), 2.02 (s, 8H), 2.00-1.91 (m, 3H), 1.79 (br s, 5H), 1.82-1.76 (m, 1H).

### Example M

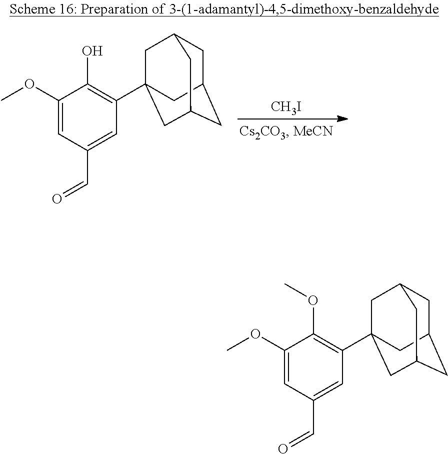 [SMILES: COc1cc(C=O)cc(C23CC4CC(CC(C4)C2)C3)c1O ; "CH3I" ; COc1cc(C=O)cc(C23CC4CC(CC(C4)C2)C3)c1OC]

A mixture of 3-(1-adamantyl)-4-hydroxy-5-methoxy-benzaldehyde (5 g, 17.46 mmol, 1 eq) in anhydrous THF (50 mL) was treated with Cs₂CO₃ (11.38 g, 34.92 mmol, 2 eq) and the resulting mixture was stirred at 40° C. for 0.5 hr and then CH₃I (11.6 g, 81.7 mmol, 5.1 mL, 4.7 eq) was added dropwise to the mixture. Subsequently, the resulting mixture was stirred at 40° C. for 16 hr. The mixture was then concentrated in vacuo to remove the THF, and then combined with water (100 mL) and extracted with EtOAc (80 mL). The organic layer was washed with brine (50 mL*2), dried over Na₂SO₄ and concentrated in vacuo to obtain crude product. The crude product was purified by flash chromatography (ISCO®; 40 g CombiFlash® Silica Flash Column, Eluent of 0˜10% Ethyl acetate/Petroleum ether gradient @ 40 mL/min) to obtain the desired product. The title compound (3.8 g, 12.7 mmol, 72% yield) was obtained as a white solid. LCMS (m/z 301 (M+H)). NMR (400 MHz, CDCl₃) δ=9.91-9.85 (m, 1H), 7.42 (d, J=2.0 Hz, 1H), 7.35 (d, 7=2.0 Hz, 1H), 3.97-3.94 (m, 3H), 3.92 (s, 3H), 2.10 (s, 9H), 1.79 (s, 6H).

### Example 15

### Preparation of 3-[3-(1-adamantyl)-4-benzyloxy-5-methoxy-phenyl]-4-[1-methyl-5-[2-(5,6,7,8-tetrahydro-1,8-naphthyridin-2-yl)ethoxy]pyrazol-3-yl]butanoic acid

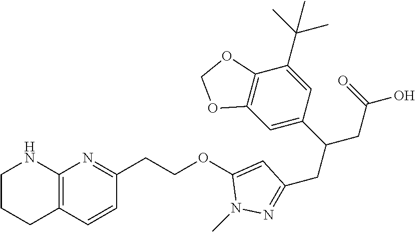 [SMILES: Cn1nc(CC(CC(=O)O)c2cc3c(c(C(C)(C)C)c2)OCO3)cc1OCCc1ccc2c(n1)NCCC2]

Example 15 was prepared in analogous manner to Example 1, using 7-(tert-butyl)benzo[d][1,3]dioxole-5-carbaldehyde as the required benzaldehyde (obtained according to Example N) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 35%-61.25%, 7 min.). The title compound (33 mg, 63 μmol, 34% yield) was obtained as a white solid. LCMS (m/z 521.1 (M+H)). ¹H NMR (400 MHz, CD₃OD) δ=7.60 (d, 7=7.2 Hz, 1H), 6.69 (d, 7=7.2 Hz, 1H), 6.62 (d, 7=2.0 Hz, 1H), 6.58 (d, 7=1.6 Hz, 1H), 5.86 (d, 7=1.2 Hz, 2H), 5.52 (s, 1H), 4.34 (t, 7=6.0 Hz, 2H), 3.53-3.47 (m, 5H), 3.34 (br d, 7=3.2 Hz, 1H), 3.17 (t, 7=6.0 Hz, 2H), 2.86-2.69 (m, 4H), 2.67-2.60 (m, 1H), 2.58-2.50 (m, 1H), 1.95 (quin, 7=6.0 Hz, 2H), 1.29 (s, 9H).

### Example N

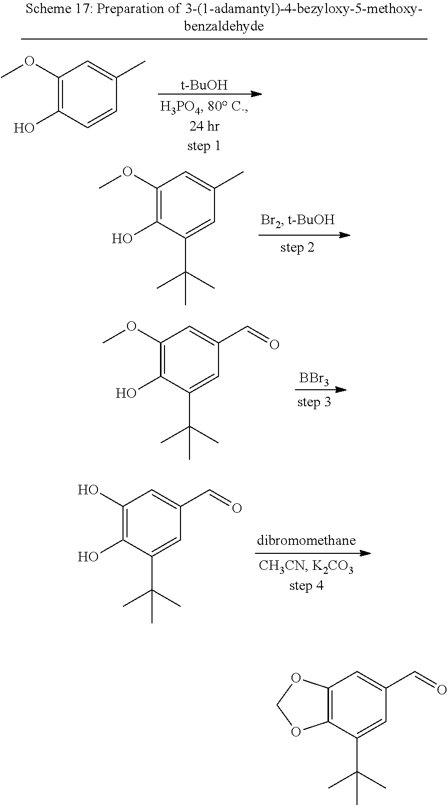

### Step 1. Preparation of 2-(tert-butyl)-6-methoxy-4-methylphenol

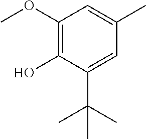

A mixture of 2-methoxy-4-methyl-phenol (20 g, 144.8 mmol, 18.2 mL, 1 eq) and H₃PO₄ (128 g, 1.11 mol, 76 mL, 85%, 7.65 eq) was treated with tert-butanol (11.9 g, 161 mmol, 15.4 mL, 1.11 eq). The resulting mixture was stirred at 80° C. for 36 hr. The reaction mixture was quenched with water (50 mL), extracted with ethyl acetate (3*200 mL) and the combined organic layers were washed with brine (150 mL), dried over anhydrous Na₂SO₄. The solvent was evaporated in vacuo to give a residue. The residue was purified by flash chromatography (ISCO®; 220 g SepaFlash® Silica Flash Column, Eluent of 0˜20% Ethyl acetate/Petroleum ethergradient @ 60 mL/min). The title compound (16 g, 82.4 mmol, 57% yield) was obtained as colorless oil. ¹H NMR (400 MHz, CDCl₃) δ=6.79 (s, 1H), 6.68 (s, 1H), 5.93 (s, 1H), 3.93 (s, 3H), 2.38 (s, 3H), 1.55-1.45 (m, 9H).

### Step 2. Preparation of 3-(tert-butyl)-4-hydroxy-5-methoxybenzaldehyde

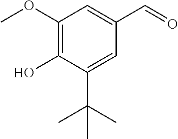

A solution of 2-tert-butyl-6-methoxy-4-methyl-phenol (10 g, 51.47 mmol, 1 eq) in 2-methylpropan-2-ol (160 mL) was treated with Br₂ (24.68 g, 154 mmol, 8 mL, 3.0 eq). The mixture was stirred at 20° C. for 1 hr. The reaction mixture was quenched by addition 10% NaHSO₃ (100 mL) at 28° C., the mixture was extracted with EtOAc 300 mL (100 mL*3). The combined organic layers were washed with brine (50 mL), dried over Na₂SO₄, filtered and concentrated under reduced pressure to give a residue. The residue was purified by flash chromatography (ISCO®; 220 g SepaFlash® Silica Flash Column, Eluent of 0˜30% Ethyl acetate/Petroleum ethergradient @ 40 mL/min). The title compound (11 g, crude) was obtained as a brown solid. ¹H NMR (400 MHz, CDCl₃) δ=9.82 (s, 1H), 7.45 (d, J=2.0 Hz, 1H), 7.33 (d, J=2.0 Hz, 1H), 6.60 (s, 1H), 3.98 (s, 3H), 1.45 (s, 9H).

### Step 3. Preparation of 3-(tert-butyl)-4,5-dihydroxybenzaldehyde

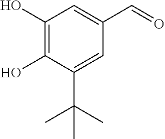

A solution of 3-tert-butyl-4-hydroxy-5-methoxy-benzaldehyde (4 g, 19.21 mmol, 1 eq) in CH₂Cl₂ (120 mL) was treated with BBr₃ (4.81 g, 19.21 mmol, 1.85 mL, 1 eq) in CH₂Cl₂ (15 mL) at −78° C. The resulting mixture was warmed to 28° C. and stirred at this temperature for 2 hr. The mixture was poured into water (50 mL), and the resulting aqueous layer was extracted with CH₂Cl₂ (30 mL*3). The organic extracts were dried with anhydrous Na₂SO₄ and the solvent evaporated under vacuum to afford a residue. The residue was purified by flash chromatography (ISCO®; 20 g SepaFlash® Silica Flash Column, Eluent of 0˜30% Ethyl acetate/Petroleum ethergradient @ 30 mL/min). The title compound (3.2 g, 16.48 mmol, 86% yield) was obtained as a brown solid. ¹H NMR (400 MHz, CDCl₃) δ=9.78 (s, 1H), 7.42 (d, J=7.2 Hz, 2H), 7.27 (s, 1H), 6.49 (s, 1H), 1.45 (s, 9H).

### Step 4. Preparation of 7-(tert-butyl)benzo[d][1,3]dioxole-5-carbaldehyde

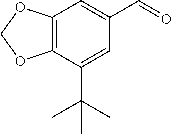

A solution of 3-(tert-butyl)-4,5-dihydroxybenzaldehyde (1.7 g, 8.75 mmol, 1 eq) and dibromomethane (1.83 g, 10.5 mmol, 740 μL, 1.2 eq) in CH₃CN (40 mL) was treated with K₂CO₃ (3.63 g, 26.26 mmol, 3 eq). The resulting mixture was stirred at 90° C. for 12 hr. The mixture was concentrated under reduced pressure to remove CH₃CN and provide a residue. The residue was combined with water (30 mL) and extracted with EtOAc (30 mL*3). The combined organic layers were washed with brine (30 mL), dried over anhydrous Na₂SO₄, filtered and concentrated under reduced pressure to give a residue. The residue was purified by flash chromatography (ISCO®; 40 g SepaFlash® Silica Flash Column, Eluent of 0˜10% Ethyl acetate/Petroleum ethergradient @ 30 mL/min). The title compound (1.1 g, 5.33 mmol, 61% yield) was obtained as a brown oil. ¹H NMR (400 MHz, CDCl₃) δ=9.81 (s, 1H), 7.37 (d, J=1.6 Hz, 1H), 7.23 (d, J=1.6 Hz, 1H), 6.08 (s, 2H), 1.39 (s, 9H).

### Example 16

### Preparation of 3-(8-(tert-butyl)-2,3-dihydrobenzo[b][1,4]dioxin-6-yl)-4-(1-methyl −5-(2-(5,6,7,8-tetrahydro-1,8-naphthyridin-2-yl)ethoxy)-1H-pyrazol-3-yl) butanoic acid

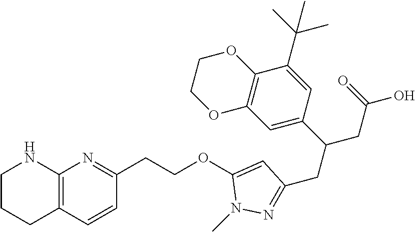 [SMILES: Cn1nc(CC(CC(=O)O)c2cc3c(c(C(C)(C)C)c2)OCCO3)cc1OCCc1ccc2c(n1)NCCC2]

Example 16 was prepared in analogous manner to Example 1, using 8-(tert-butyl)-2,3-dihydrobenzo[b][1,4]dioxine-6-carbaldehyde as the required benzaldehyde (obtained according to Example O) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 30%-60%, 8 min.). The title compound (33 mg, 50 μmol, 40% yield, TFA) was obtained as a white solid. LCMS (m/z 535.0 (M+H)). ¹H NMR (400 MHz, CD₃OD) δ=7.60 (d, J=7.2 Hz, 1H), 6.68 (d, J=7.2 Hz, 1H), 6.59 (dd, j=2.0, 9.2 Hz, 2H), 5.59 (s, 1H), 4.37 (t, j=6.0 Hz, 2H), 4.19 (s, 4H), 3.53-3.48 (m, 5H), 3.30-3.23 (m, 1H), 3.18 (t, J=6.0 Hz, 2H), 2.87-2.46 (m, 6H), 2.00-1.91 (m, 2H), 1.30 (s, 9H). ¹⁹F NMR (376 MHz, CD₃OD)-77.27 (br, s, 1F).

### Example O

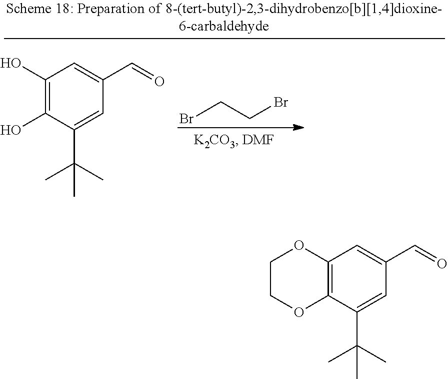

A solution of 3-(tert-butyl)-4,5-dihydroxybenzaldehyde (3 g, 15.45 mmol, 1 eq) and dibromoethane (4.35 g, 23.2 mmol, 1.75 mL, 1.5 eq) in CH₃CN (60 mL) was treated with K₂CO₃ (6.40 g, 46.34 mmol, 3 eq). The resulting mixture was stirred at 90° C. for 24 hr. The mixture was concentrated under reduced pressure to remove CH₃CN and provide a residue. The residue was combined with water (50 mL) and extracted with EtOAc (100 mL*3). The combined organic layers were washed with brine (100 mL), dried over anhydrous Na₂SO₄, filtered and concentrated under reduced pressure to give a residue. The residue was purified by flash chromatography (ISCO®; 40 g SepaFlash® Silica Flash Column, Eluent of 0˜10% Ethyl acetate/Petroleum ethergradient @ 30 mL/min). The title compound (2.2 g, 10 mmol, 65% yield) was obtained as a brown solid. ¹H NMR (400 MHz, CDCl₃) δ=9.81 (s, 1H), 7.43 (d, J=2.0 Hz, 1H), 7.29 (d, J=2.0 Hz, 1H), 4.39-4.35 (m, 2H), 4.33-4.28 (m, 2H), 1.40 (s, 9H).

### Example 17

### Preparation of 3-(2-bromo-5-(tert-butyl)phenyl)-4-(1-methyl-5-(2-(5,6,7,8-tetrahydro-1,8-naphthyridin-2-yl)ethoxy)-1H-pyrazol-3-yl)butanoic acid

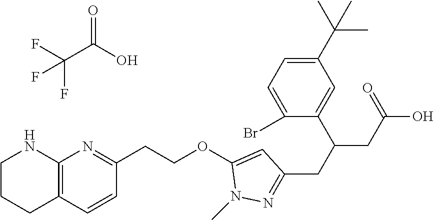 [SMILES: Cn1nc(CC(CC(=O)O)c2cc(C(C)(C)C)ccc2Br)cc1OCCc1ccc2c(n1)NCCC2 ; O=C(O)C(F)(F)F]

Example 17 was prepared in analogous manner to Example 1, using 2-bromo-5-(tert-butyl)benzaldehyde as the required benzaldehyde (obtained according to Example P) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 32%-62%, 8 min.). The title compound (58 mg, 86 μmol, 74% yield, TFA) was obtained as a white solid. LCMS (m/z 557.0 (M+H)). ¹H NMR (400 MHz, CD₃OD) δ=7.60 (d, J=7.2 Hz, 1H), 7.44 (d, J=8.4 Hz, 1H), 7.27 (d, J=2.4 Hz, 1H), 7.13 (dd, J=8.4, 2.4 Hz, 1H), 6.68 (d, J=7.2 Hz, 1H), 5.45 (s, 1H), 4.33 (t, j=6.0 Hz, 2H), 3.95 (quin, j=7.6 Hz, 1H), 3.47-3.53 (m, 5H), 3.15 (t, J=6.0 Hz, 2H), 2.63-2.93 (m, 6H), 1.95 (quin, j=6.0 Hz, 2H), 1.27 (s, 9H); ¹⁹F NMR (CD₃OD, 376 MHz) −77.43 (s, 1F).

### Example P

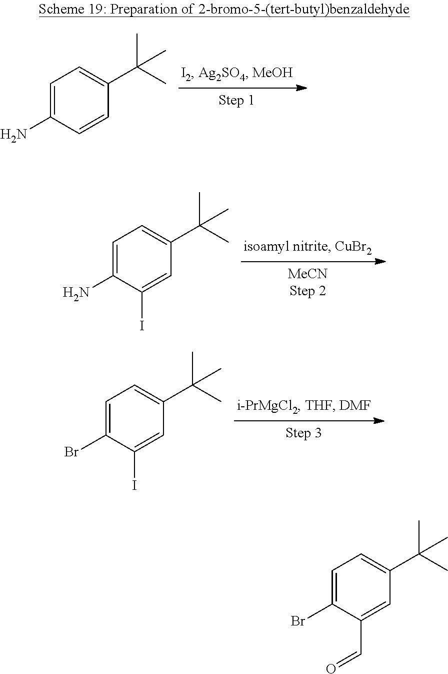

### Step 1. Preparation of 4-(tert-butyl)-2-iodoaniline

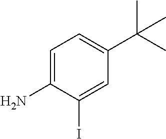

A mixture of 4-tert-butylaniline (9.8 g, 66 mmol, 10.4 mL, 1 eq) and Ag₂SO₄ (19 g, 61 mmol, 10.3 mL) in MeOH (500 mL) was slowly treated with I₂ (16.7 g, 66 mmol, 13.2 mL, 1 eq) at 15° C. The resulting mixture was stirred at 15° C. for 2 hr. The solid was filtered off and the filtrate was quenched with NH₄Cl (aq., 300 mL). The mixture was concentrated. The residue was diluted with EtOAc (300 mL) and the organic layer was washed with NH₄Cl (100 mL*3). The organic layer was dried over Na₂SO₄, filtered and concentrated. The residue was purified by column chromatography (SiO₂, Petroleum ether/Ethyl acetate=I/O to 15/1). The title compound (15 g, 54.5 mmol, 83% yield) was obtained as red oil.

### Step 2. Preparation of 1-bromo-4-(tert-butyl)-2-iodobenzene

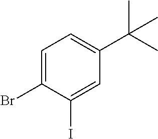

A mixture of 4-tert-butyl-2-iodo-aniline (20 g, 72.7 mmol 1 eq), CuBr₂ (24.35 g, 109 mmol, 5.1 mL, 1.5 eq) and tert-butyl nitrite (14.99 g, 145.39 mmol, 17.29 mL, 2 eq) in CH₃CN (200 mL) was stirred at 65° C. under nitrogen for 2 hr. The mixture was filtered and the filtrate was quenched with water (500 mL) and was concentrated. The residue was extracted with ethyl acetate (300 mL*3). The organic layer was dried over Na₂SO₄, filtered and concentrated. The residue was purified by column chromatography (SiO₂, Petroleum ether: Ethyl acetate=1:0). Compound 1-bromo-4-tert-butyl-2-iodo-benzene (20 g, crude) was obtained as red oil.

### Step 3. Preparation of 2-bromo-5-(tert-butyl)benzaldehyde

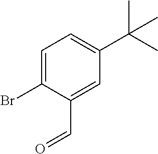

A stirring mixture of 1-bromo-4-tert-butyl-2-iodo-benzene (20 g, 59 mmol, 1 eq) in THE (150 mL) and EtOAc (150 mL) was treated with i-PrMgCl (2 M, 31 mL, 1.05 eq) drop wise at −78° C. The resulting mixture was stirred for 2 h at −78° C. and then treated with N,N-dimethylformamide (8.62 g, 117.99 mmol, 9.08 mL, 2 eq) by drop-wise addition at −78° C. under nitrogen, then warmed slowly to 15° C. over 3 hr. The reaction mixture was quenched with water (300 mL), the organic layer was separated and the aqueous layer was extracted with EtOAc (200 mL*3), the combine organic layers was washed by brine (100 mL*3) and dried with Na₂SO₄, filtered and concentrated to provide a residue. The residue was purified by column chromatography (SiO₂, Petroleum ether/Ethyl acetate=1:0). The title compound (10 g, crude) was obtained as colorless oil. LCMS (m/z 242.0 (M+H)).

### Example 18

### Preparation of 3-(5-(tert-butyl)-2-cyanophenyl)-4-(1-methyl-5-(2-(5,6,7,8-tetrahydro-1,8-naphthyridin-2-yl)ethoxy)-1H-pyrazol-3-yl)butanoic acid

The title compound was prepared according to Scheme 20 where the starting material bromide is the final step starting material from Example 17.

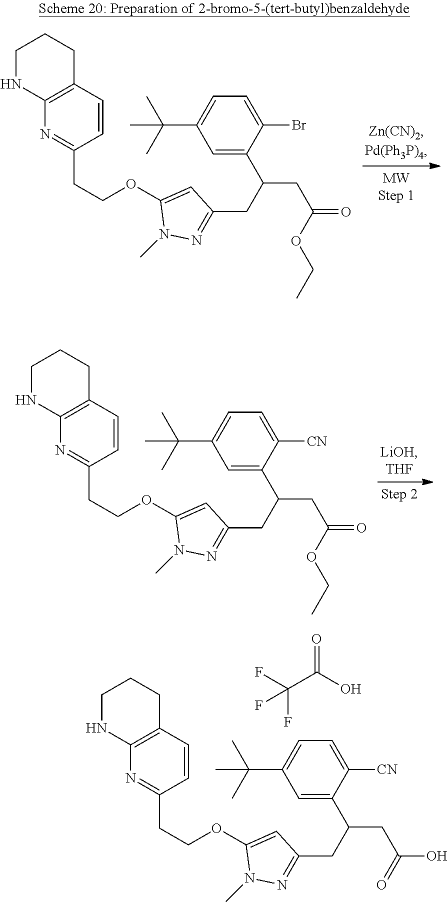

### Step 1. Preparation of ethyl 3-(5-(tert-butyl)-2-cyanophenyl)-4-(1-methyl-5-(2-(5,6,7,8-tetrahydro-1,8-naphthyridin-2-yl)ethoxy)-1H-pyrazol-3-yl)butanoate

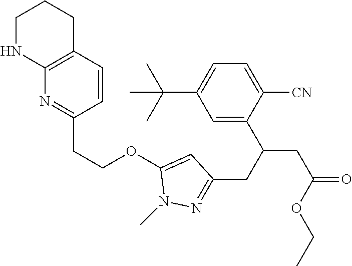 [SMILES: CCOC(=O)CC(Cc1cc(OCCc2ccc3c(n2)NCCC3)n(C)n1)c1cc(C(C)(C)C)ccc1C#N]

A mixture of ethyl 3-(2-bromo-5-tert-butyl-phenyl)-4-[1-methyl-5-[2-(5,6,7,8-tetrahydro-1,8-naphthyridin-2-yl)ethoxy]pyrazol-3-yl]butanoate (330 mg, 522 μmol, 1 eq) and Zn(CN)₂ (184 mg, 1.57 mmol, 100 μL, 3 eq) in DMF (5 mL) in a microwave vial was evacuated and back-filled with N₂ (3×). Subsequently, Pd(PPh₃)₄ (60 mg, 52 μmol, 0.1 eq) was added. The reaction vial was sealed, and the reaction mixture was again degassed and back-filled with N₂ (3×), and then stirred at 120° C. for 1.5 hr under micro-wave irradiation. The reaction mixture was poured into water (80 mL), and extracted with EtOAc (3*50 mL). The combined organic layer was washed with brine (2*50 mL), dried over sodium sulfate, and evaporated to give a residue. The residue was purified by flash chromatography (ISCO®; 12 g SepaFlash® Silica Flash Column, Eluent of 0˜100% Ethyl acetate/Petroleum ether gradient @ 30 mL/min). The title compound (45 mg, 75 μmol, 14% yield, 88% purity) was obtained as colorless oil. The title compound (77 mg, 109 μmol, 21% yield, 75% purity) was obtained as colorless oil. LCMS (m/z 530.1 (M+H)).

### Step 2. Preparation of 3-(5-(tert-butyl)-2-cyanophenyl)-4-(1-methyl-5-(2-(5,6,7,8-tetrahydro-1,8-naphthyridin-2-yl)ethoxy)-1H-pyrazol-3-yl)butanoic acid

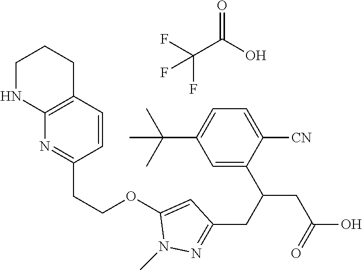

A mixture of ethyl 3-(5-tert-butyl-2-cyano-phenyl)-4-[1-methyl-5-[2-(5,6,7,8-tetrahydro-1,8-naphthyridin-2-yl)ethoxy]pyrazol-3-yl]butanoate (45 mg, 75 μmol, 1 eq) in THF (2 mL) was treated with LiOH (1 M, 2.5 mL, 33 eq). The resulting mixture was stirred at 50° C. for 4 hr. Then the mixture was stirred at 15° C. for 12 hr. The mixture was concentrated under pressure to remove solvent to give a residue, the residue was adjusted pH=5 with AcOH and extracted with ethyl acetate (10 mL*2). The combined organic phase was concentrated in vacuo. The residue was purified by prep-HPLC (column: Boston Green ODS 150*30 5μ; mobile phase: [water (0.1% TFA)-ACN]; B %: 25%-55%, 8 min). The title compound (38 mg, 62 μmol, 82% yield, TFA) was obtained as a white solid. ¹H NMR (CD₃OD, 400 MHz) δ=7.60 (d, J=7.2 Hz, 1H), 7.55 (d, J=8.4 Hz, 1H), 7.46 (d, J=1.2 Hz, 1H), 7.40 (dd, J=8.4, 1.6 Hz, 1H), 6.69 (d, J=7.6 Hz, 1H), 5.47 (s, 1H), 4.33 (t, J=6.0 Hz, 2H), 3.83 (quin, J=7.6 Hz, 1H), 3.47-3.54 (m, 2H), 3.45 (s, 3H), 3.16 (t, J=6.0 Hz, 2H), 2.78-2.94 (m, 6H), 1.95 (quin, J=6.0 Hz, 2H), 1.31 (s, 9H). 19F NMR (CD₃OD, 376 MHz) −77.39 (s, 1F). LCMS (m/z 502.1 (M+H)).

### Example 19

### Preparation of 3-(5-(tert-butyl)-2-fluorophenyl)-4-(1-methyl-5-(2-(5,6,7,8-tetrahydro-1,8-naphthyridin-2-yl)ethoxy)-1H-pyrazol-3-yl)butanoic acid

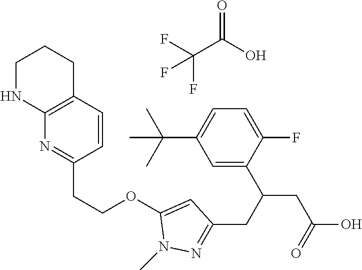

Example 19 was prepared in analogous manner to Example 1, using 5-(tert-butyl)-2-fluorobenzaldehyde as the required benzaldehyde (obtained according to Example Q) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 25%-55%, 8 min.). The title compound (193 mg, 314 μmol, 71% yield, TFA) was obtained as a white solid. LCMS (m/z 495.0 (M+H)). ¹H NMR (400 MHz, CD₃OD) δ=7.60 (d, J=7.2 Hz, 1H), 7.18-7.25 (m, 2H), 6.88-6.96 (m, 1H), 6.68 (d, 7=7.2 Hz, 1H), 5.48 (s, 1H), 4.32 (t, 7=6.0 Hz, 2H), 3.67 (quin, 7=7.6 Hz, 1H), 3.48-3.53 (m, 2H), 3.47 (s, 3H), 3.15 (t, 7=6.0 Hz, 2H), 2.79-2.90 (m, 4H), 2.71 (d, 7=8.0 Hz, 2H), 1.95 (quin, 7=6.0 Hz, 2H), 1.26 (s, 9H). 19F NMR (CD₃OD, 376 MHz) −77.37 (s, 1F), −124.67 (br s, 1F).

### Example Q

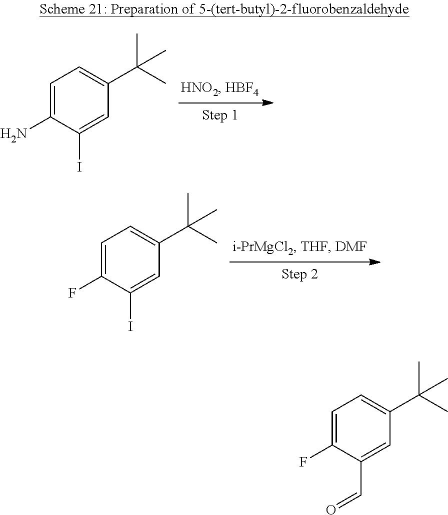

### Step 1. Preparation of 4-tert-butyl-1-fluoro-2-iodo-benzene

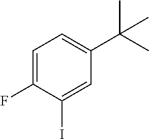

A solution of 4-tert-butyl-2-iodo-aniline (20 g, 73 mmol, 1 eq), CuBr₂ (24.35 g, 109 mmol, 5.1 mL, 1.5 eq) and tert-butyl nitrite (15 g, 145 mmol, 17.3 mL, 2 eq) in CH₃CN (200 mL) was stirred at 65° C. under nitrogen for 2 hr. The mixture was filtered and the filtrate was quenched with water (500 mL) and was concentrated. The residue was extracted with ethyl acetate (300 mL*3). The organic layer was dried over Na₂SO₄, filtered and concentrated. The residue was purified by column chromatography (SiO₂, Petroleum ether: Ethyl acetate=1:0). The title compound (20 g, crude) was obtained as red oil.

### Step 2. Preparation of 5-(tert-butyl)-2-fluorobenzaldehyde

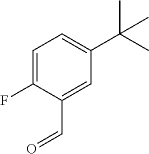

A stirring solution 4-tert-butyl-1-fluoro-2-iodo-benzene (5 g, 18 mmol, 1 eq) in THF (150 mL) and EtOAc (150 mL) was treated with i-PrMgCl (2 M, 9.44 mL, 1.05 eq) drop wise at −78° C. The resulting mixture was stirred for 2 h at −78° C. and then treated drop wise with N,N-dimethylformamide (2.63 g, 36 mmol, 2.8 mL, 2 eq) at −78° C. under nitrogen. The mixture then warmed slowly to 15° C. over 3 hr. The reaction was quenched with water (300 mL), the organic layer was separated and the aqueous layer was extracted with EtOAc (200 mL*3), the combined organic layers were washed by brine (100 mL*3) and dried with Na₂SO₄, filtered and concentrated. The residue was purified by column chromatography (SiO₂, Petroleum ether/Ethyl acetate=1/0). The title compound (2.63 g, 14.6 mmol, 81% yield) was obtained as a colorless oil.

### Example 20

### Preparation of 3-(5-(tert-butyl)-2-(trifluoromethoxy)phenyl)-4-(1-methyl-5-(2-(5, 6, 7, 8-tetrahydro-1,8-naphthyridin-2-yl)ethoxy)-1H-pyrazol-3-yl)butanoic acid

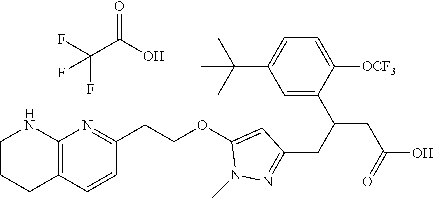

Example 20 was prepared in analogous manner to Example 1, using 5-(tert-butyl)-2-(trifluoromethoxy)benzaldehyde as the required benzaldehyde (obtained according to Example R) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 25%-55%, 8 min.). The title compound (110 mg, 160 μmol, 47% yield, TFA) was obtained as a white solid. LCMS (m/z 561.1 (M+H)). ¹H NMR (400 MHz, CD₃OD) δ=7.60 (d, J=7.2 Hz, 1H), 7.36-7.28 (m, 2H), 7.14 (br d, J=8.8 Hz, 1H), 6.68 (d, J=7.2 Hz, 1H), 5.40 (s, 1H), 4.30 (t, J=6.0 Hz, 2H), 3.82 (quin, J=7.2 Hz, 1H), 3.54-3.45 (m, 5H), 3.15 (t, J=6.0 Hz, 2H), 2.88-2.79 (m, 4H), 2.76-2.60 (m, 2H), 1.95 (quin, J=6.0 Hz, 2H), 1.29 (s, 9H). ¹⁹F NMR (376 MHz, CD₃OD) −58.02 (s, 3F), −77.41 (s, 3F).

### Example R

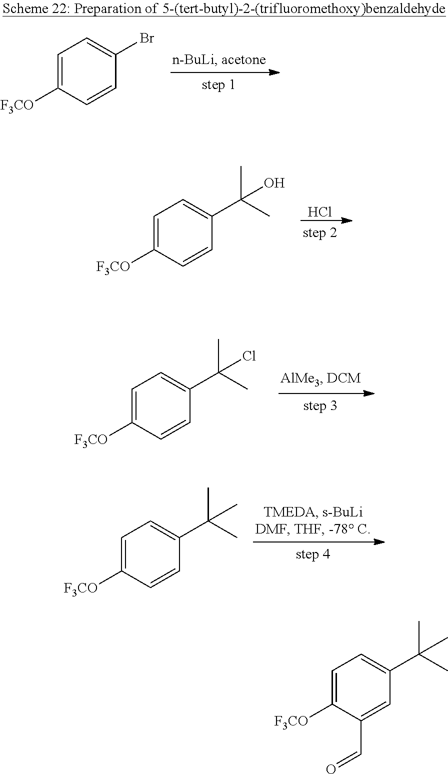 [SMILES: FC(F)(F)Oc1ccc(Br)cc1 ; CC(C)(O)c1ccc(OC(F)(F)F)cc1 ; Cl ; CC(C)(Cl)c1ccc(OC(F)(F)F)cc1 ; CC(C)(C)c1ccc(OC(F)(F)F)cc1 ; CC(C)(C)c1ccc(OC(F)(F)F)c(C=O)c1]

### Step 1. Preparation of 2-(4-(trifluoromethoxy)phenyl)propan-2-ol

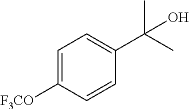

A mixture of 1-bromo-4-(trifluoromethoxy)benzene (20 g, 83 mmol, 12 mL, 1 eq) in THF (150 mL) was degassed and purged with N₂ for 3 times, and then the mixture was cooled to −78° C. under N₂ atmosphere, then n-BuLi (2.5 M, 39.8 mL, 1.2 eq) in hexane was added dropwise at −78° C. The mixture was stirred at −78° C. for 0.5 hr, then propan-2-one (5.3 g, 91.3 mmol, 6.7 mL, 1.1 eq) in THF (40 mL) was added dropwise at −78° C. The mixture was stirred at −78° C. for 1.5 hr. The reaction mixture was pour into water (150 mL) at 0° C., and extracted with EtOAc (100 mL*3). The combined organic layers were washed with brine (100 mL), dried over Na₂SO₄, filtered and concentrated under reduced pressure to give a residue. The residue was purified by flash chromatography (ISCO®; 120 g SepaFlash® Silica Flash Column, Eluent of 0-50% Ethyl acetate/Petroleum ether gradient @ 40 mL/min). The title compound (11 g, 50 mmol, 60% yield) was obtained as colorless liquid. ¹H NMR (400 MHz, CDCl₃) δ=7.57-7.48 (m, 2H), 7.18 (br d, J=8.4 Hz, 2H), 1.94 (br s, 1H), 1.58 (s, 6H). ¹⁹F NMR (376 MHz, CDCl₃) δ=−57.96 (s, 3F).

### Step 2. Preparation of 1-(2-chloropropan-2-yl)-4-(trifluoromethoxy)benzene

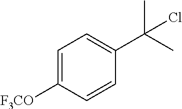

2-[4-(trifluoromethoxy)phenyl]propan-2-ol (6 g, 27.25 mmol, 1 eq) was added to HCl (36% aqueous, 55 mL, 56.1 g, 554 mmol, 20.3 eq). The resulting mixture was stirred at 15° C. for 24 hr. The reaction mixture was quenched by dilution with water (50 mL) at 0° C., and then extracted with EtOAc (30 mL*3). The combined organic layers were washed with brine (30 mL), dried over anhydrous Na₂SO₄, filtered and concentrated under reduced pressure to give a residue. The residue was used into the next step without further purification. The title compound (5.7 g, 23.89 mmol, 87.66% yield) was obtained as a yellow oil and was used to next step without further purification. ¹H NMR (400 MHz, CDCl₃) δ=7.66-7.58 (m, 2H), 7.21 (br d, J=8.4 Hz, 2H), 2.00-2.01 (m, 6H).

### Step 3. Preparation of 1-(tert-butyl)-4-(trifluoromethoxy)benzene

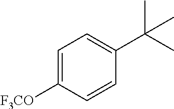

A solution of 1-(1-chloro-1-methyl-ethyl)-4-(trifluoromethoxy)benzene (5.7 g, 23.89 mmol, 1 eq) in DCM (60 mL) was treated with Al(CH₃)₃ (1 M, 47.8 mL, 2 eq) at −78° C. under N₂. The mixture was stirred at −78° C. for 2 hr then warmed to 10° C. and stirred for 10 hr. The reaction mixture was pour into ice water (100 mL) at 0° C., and extracted with EtOAc (50 mL*3). The combined organic layers were washed with brine (50 mL), dried over anhydrous Na₂SO₄, filtered and concentrated under reduced pressure to give a residue. The residue was purified by flash chromatography (ISCO®; 40 g SepaFlash® Silica Flash Column, Eluent of 0˜0% Ethyl acetate/Petroleum ether gradient @ 30 mL/min). The title compound (3.8 g, 17.4 mmol, 73% yield) was obtained as colorless liquid. ¹H NMR (400 MHz, CDCl₃) δ=7.41 (d, J=8.8 Hz, 2H), 7.15 (d, J=8.8 Hz, 2H), 1.34 (s, 9H).

### Step 4. Preparation of 5-(tert-butyl)-2-(trifluoromethoxy)benzaldehyde

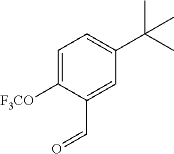 [SMILES: CC(C)(C)c1ccc(OC(F)(F)F)c(C=O)c1]

A solution of 1-tert-butyl-4-(trifluoromethoxy)benzene (2 g, 9.17 mmol, 1 eq) in THF (20 mL) was treated with TMEDA (1.07 g, 9.17 mmol, 1.38 mL, 1 eq) and sec-butyllithium (1.3 M, 14.1 mL, 2 eq) at −78° C. under N₂. The resulting mixture was stirred at −78° C. for 1.5 hr, then DMF (1.34 g, 18.33 mmol, 1.41 mL, 2 eq) was added and the mixture was stirred at −78° C. for 0.5 hr. The reaction was quenched by addition of water (30 mL) at 0° C., and then diluted with EtOAc (30 mL) and extracted with EtOAc (30 mL*3). The combined organic layers were washed with brine (30 mL), dried over Na₂SO₄, filtered and concentrated under reduced pressure to give a residue. The title compound (2 g, 8.12 mmol, 89% yield) was obtained as a yellow liquid used in the next step without further purification. ¹H NMR (400 MHz, CDCl₃) δ=10.37 (s, 1H), 7.98 (d, J=2.4 Hz, 1H), 7.69 (dd, J=2.8, 8.8 Hz, 1H), 7.29 (dd, J=1.6, 8.8 Hz, 1H), 1.35 (s, 9H). ¹⁹F NMR (376 MHz, CDCl₃) −57.79 (d, J=1.5 Hz, 3F).

### Example 21

### Preparation of 3-[5-[1-(difluoromethyl)cyclopropyl]-2-methoxy-phenyl]-4-[1-methyl-5-[2-(5,6,7,8-tetrahydro-1,8-naphthyridin-2-yl)ethoxy]pyrazol-3-yl]butanoic acid

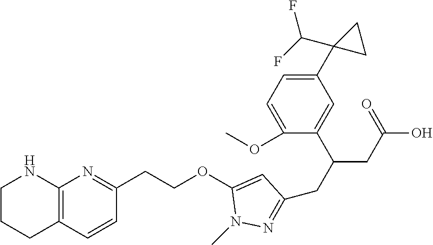 [SMILES: COc1ccc(C2(C(F)F)CC2)cc1C(CC(=O)O)Cc1cc(OCCc2ccc3c(n2)NCCC3)n(C)n1]

Example 21 was prepared in analogous manner to Example 1, using 5-(1-(difluoromethyl)cyclopropyl)-2-methoxybenzaldehyde as the required benzaldehyde (obtained according to Example E starting at Step 2 and using 3-bromo-4-methoxy-benzaldehyde in place of 3-bromo-5-(tert-butyl)benzaldehyde as shown in Scheme 9) as the required benzaldehyde in the reaction Scheme 4 and replacing Step 12 of Scheme 4 with Step 4 of Scheme 5. The crude product was purified by Prep-HPLC (column: Xbridge BEH C18, 250*50 mm, 10 μm; mobile phase: [water (0.1% TFA)-ACN]; B %: 25%-41%, 9 min.). The title compound was obtained as a yellow solid. ¹H NMR (400 MHz, CD₃OD): δ ppm=7.57 (d, J=7.3 Hz, 1H), 7.19 (dd, J=2.1, 8.4 Hz, 1H), 7.07 (d, J=2.0 Hz, 1H), 6.87 (d, J=8.6 Hz, 1H), 6.64 (d, J=7.3 Hz, 1H), 5.68-5.36 (m, 2H), 4.31 (t, J=6.0 Hz, 2H), 3.81 (s, 3H), 3.76-3.67 (m, 1H), 3.50-3.43 (m, 4H), 3.31-3.26 (m, 2H), 3.13 (t, j=5.9 Hz, 2H), 2.98-2.85 (m, 2H), 2.80 (br t, j=6.1 Hz, 2H), 2.73-2.62 (m, 2H), 1.98-1.87 (m, 2H), 1.06-0.98 (m, 2H), 0.82 (br d, J=1.7 Hz, 2H). ¹⁹F NMR (376 MHz, CD₃OD)=−77.34 (s, 1F), −117.73 (br d, J=57.2 Hz, 1F).

### Example 22

### Preparation of 3-(5-(2-hydroxypropan-2-yl)-2-methoxyphenyl)-4-(1-methyl-5-(2-(5,6,7,8-tetrahydro-1,8-naphthyridin-2-yl)ethoxy)-1H-pyrazol-3-yl)butanoic acid

The title compound was prepared according to Scheme 23. PGP-249 C₃

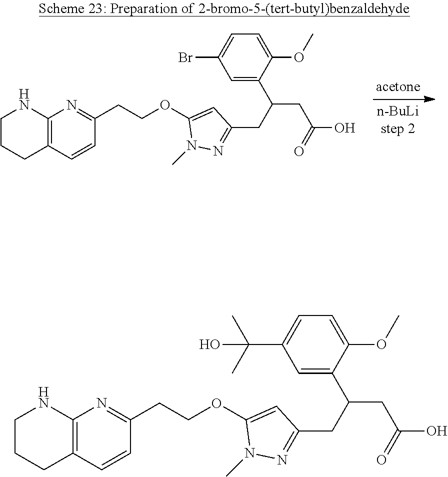 [SMILES: COc1ccc(Br)cc1C(CC(=O)O)Cc1cc(OCCc2ccc3c(n2)NCCC3)n(C)n1 ; COc1ccc(C(C)(C)O)cc1C(CC(=O)O)Cc1cc(OCCc2ccc3c(n2)NCCC3)n(C)n1]

### Step 1. Preparation of 3-(5-bromo-2-methoxyphenyl)-4-(1-methyl-5-(2-(5,6,7,8-tetrahydro-1,8-naphthyridin-2-yl)ethoxy)-1H-pyrazol-3-yl)butanoic acid

The title compound was prepared in analogous manner to Example 1, using 5-bromo-2-methoxy-benzaldehyde as the required benzaldehyde in the in the reaction Scheme 4. The crude product was purified by Prep-HPLC (column: Boston Green ODS 150*30 5μ; mobile phase: [water (0.1% TFA)-ACN]; B %: 20%-50%, 8 min). The title compound (190 mg, 293 μmol, 84% yield, 99.4% purity, TFA) was obtained as a white solid, which was confirmed by LCMS (m/z 529.0 (M+H)), HPLC, ¹H NMR and ¹⁹F NMR. ¹H NMR (CD₃OD, 400 MHz) 7.59 (d, J=7.2 Hz, 1H), 7.29 (dd, J=8.8, 2.4 Hz, 1H), 7.22 (d, J=2.4 Hz, 1H), 6.86 (d, J=8.8 Hz, 1H), 6.66 (d, J=7.2 Hz, 1H), 5.55 (s, 1H), 4.37 (t, J=6.0 Hz, 2H), 3.82 (s, 3H), 3.73 (quin, J=7.6 Hz, 1H), 3.47-3.53 (m, 5H), 3.16 (t, J=6.0 Hz, 2H), 2.85-2.94 (m, 2H), 2.82 (t, J=6.4 Hz, 2H), 2.61-2.73 (m, 2H), 1.95 (quin, J=6.0 Hz, 2H), ¹⁹F NMR (CD₃OD, 376 MHz) −77.32 (br s, 1F).

### Step 2. Preparation of 3-(5-(2-hydroxypropan-2-yl)-2-methoxyphenyl)-4-(1-methyl-5-(2-(5,6,7,8-tetrahydro-1,8-naphthyridin-2-yl)ethoxy)-1H-pyrazol-3-yl)butanoic acid

A solution of 3-(5-bromo-2-methoxy-phenyl)-4-[1-methyl-5-[2-(5,6,7,8-tetrahydro-1,8-naphthyridin-2-yl)ethoxy]pyrazol-3-yl]butanoic acid (100 mg, 189 μmol, 1 eq) in THF (10 mL) was treated with n-BuLi (2.5 M, 300 μL, 4 eq) at −78° C. under N₂ atmosphere. The mixture was stirred for 0.5 hr, then acetone (88 mg, 1.51 mmol, 110 μL, 8 eq) in THF (2 mL) was added dropwise, the mixture was warmed to 10° C. and stirred for 1.5 hr. The mixture was quenched with water (2 mL), and then concentrated under reduced pressure to afford a residue. The residue was purified by prep-HPLC (basic condition: column: Phenomenex Gemini 150*25 mm*10 μm; mobile phase: [water (0.05% ammonia hydroxide v/v)-ACN]; B %: 15%-45%, 8 min.). The title compound (6.4 mg, 12 μmol, 6.5% yield) was obtained as a white solid. ¹H NMR (400 MHz, CD₃OD) δ=7.33-7.21 (m, 3H), 6.90-6.83 (m, 1H), 6.48 (d, J=7.6 Hz, 1H), 5.39 (s, 1H), 4.34-4.21 (m, 2H), 3.86-3.74 (m, 4H), 3.42-3.35 (m, 5H), 3.10-2.91 (m, 2H), 2.89-2.76 (m, 2H), 2.73 (t, J=6.4 Hz, 2H), 2.63 (d, J=7.2 Hz, 2H), 1.95-1.82 (m, 2H), 1.47 (s, 6H). LCMS (m/z 509.0 (M+H)).

### Example 23

### Preparation of 3-[2-methoxy-5-(oxetan-3-yl)phenyl]-4-[1-methyl-5-[2-(5,6,7,8-tetrahydro-1,8-naphthyridin-2-yl)ethoxy]pyrazol-3-yl]butanoic acid

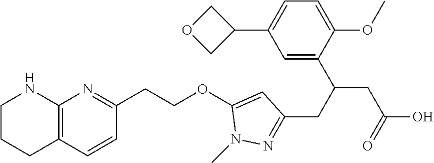 [SMILES: COc1ccc(C2COC2)cc1C(CC(=O)O)Cc1cc(OCCc2ccc3c(n2)NCCC3)n(C)n1]

Example 23 was prepared in analogous manner to Example 1, using 2-methoxy-5-(oxetan-3-yl)benzaldehyde as the required benzaldehyde (obtained according to Example S) as the required benzaldehyde in the reaction Scheme 4 and replacing Step 12 of Scheme 4 with Step 4 of Scheme 5. The crude product was purified by Prep-HPLC (Column: Boston Prime C18 150*30 mm 5 μm; mobile phase: [water (0.05% ammonia hydroxide v/v)-ACN]; B %: 28%-58%, 9 min.). The title compound (2.4 mg, 4.7 μmol, 12% yield) was obtained as a white solid. ¹H NMR (400 MHz, CD₃OD) δ=7.29-7.15 (m, 3H), 6.94 (br d, J=8.3 Hz, 1H), 6.49 (br s, 1H), 5.44 (br s, 1H), 5.10-4.97 (m, 2H), 4.70 (br t, J=6.3 Hz, 2H), 4.30 (br s, 2H), 4.22-4.11 (m, 1H), 3.85 (s, 4H), 3.44-3.38 (m, 4H), 3.00 (br d, J=16.8 Hz, 2H), 2.86 (br s, 2H), 2.75 (br t, J=6.1 Hz, 4H), 1.95-1.84 (m, 2H). ¹⁹F NMR (376 MHZ, MeOD-d₄)=−76.94 (s, 1F).

### Example S

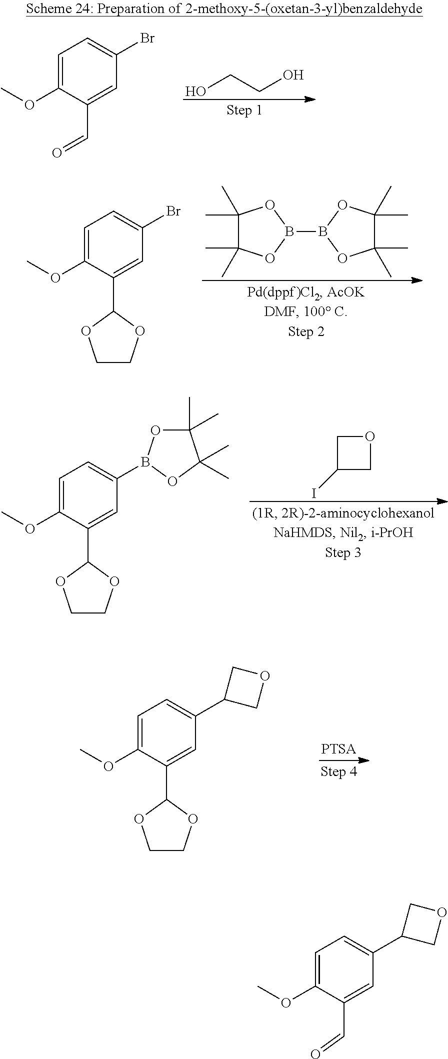

### Step 1. Preparation of 2-(5-bromo-2-methoxy-phenyl)-1,3-dioxolane

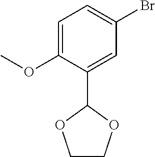

A mixture of 5-bromo-2-methoxy-benzaldehyde (15 g, 69.75 mmol, 1 eq), ethylene glycol (12.99 g, 209 mmol, 11.7 mL, 3 eq), and PTSA (240 mg, 1.40 mmol, 0.02 eq) in toluene (75 mL) was degassed and purged with N₂ for 3 times, and then the mixture was stirred at 140° C. for 2 hr under N₂ atmosphere. The reaction mixture was cooled to rt and washed with saturated NaHCO₃ (100 mL*2), dried over anhydrous Na₂SO₄, filtered and concentrated under reduced pressure to give a residue. The residue was used into the next step without further purification. The title compound (17.6 g, 67.9 mmol, 97% yield) was obtained as a colorless oil. ¹H NMR (400 MHz, CDCl₃): δ ppm=7.62 (d, J=2.4 Hz, 1H), 7.40 (dd, J=2.4, 8.8 Hz, 1H), 6.76 (d, J=8.8 Hz, 1H), 6.09 (s, 1H), 4.15-4.08 (m, 2H), 4.04-3.97 (m, 2H), 3.83 (s, 3H).

### Step 2. Preparation of 2-[3-(1,3-dioxolan-2-yl)-4-methoxy-phenyl]-4,4,5,5-tetramethyl-1,3,2-dioxaborolane

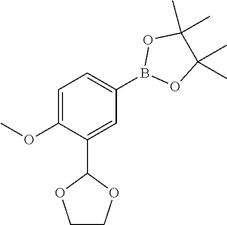

A solution of 2-(5-bromo-2-methoxy-phenyl)-1,3-dioxolane (8 g, 30.88 mmol, 1 eq) in DMF (200 mL) was treated with 4,4,5,5-tetramethyl-2-(4,4,5,5-tetramethyl-1,3,2-dioxaborolan-2-yl)-1,3,2-dioxaborolane (15.68 g, 61.75 mmol, 2 eq), AcOK (9.09 g, 92.6 mmol, 3 eq) and Pd(dppf)Cl₂ (2.26 g, 3.09 mmol, 0.1 eq). The resulting mixture was stirred at 100° C. for 2 h under nitrogen. The mixture was evaporated and the residue was diluted with water (50 mL) and extracted with Ethyl acetate (3×50 mL). The combined organic layers were dried over sodium sulfate and evaporated. The residue was purified by flash chromatography (ISCO®; 80 g SepaFlash® Silica Flash Column, Eluent of 0-15% Ethyl acetate/Petroleum ether gradient @ 40 mL/min). The title compound (6.7 g, 21.9 mmol, 71% yield) was obtained as a colorless oil. ¹H NMR (400 MHz, CDCl₃): δ ppm=7.96 (s, 1H), 7.78 (dd, J=1.0, 8.3 Hz, 1H), 6.88 (d, J=8.3 Hz, 1H), 6.13 (s, 1H), 4.18-4.10 (m, 2H), 4.05-3.96 (m, 2H), 3.86 (s, 3H), 1.30 (s, 12H).

### Step 3. Preparation of 2-[2-methoxy-5-(oxetan-3-yl)phenyl]-1,3-dioxolane

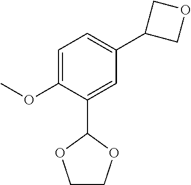 [SMILES: COc1ccc(C2COC2)cc1C1OCCO1]

A mixture of 2-[3-(1,3-dioxolan-2-yl)-4-methoxy-phenyl]-4,4,5,5-tetramethyl-1,3,2-dioxaborolane (6.2 g, 20.25 mmol, 1 eq), (1S,2S)-2-aminocyclohexanol hydrochloride (153.54 mg, 1.01 mmol, 0.05 eq), nickel (II) iodide (316 mg, 1.01 mmol, 54 μL, 0.05 eq), and [bis(trimethylsilyl)amino]sodium (2 M, 10.13 mL, 1 eq) was combined with a suspension of 3-iodooxetane (4.10 g, 22.28 mmol, 1.1 eq) in i-PrOH (40 mL) in a round bottom flask. The flask was purged with N₂ for 30 min while stirring the mixture at 15° C., then purged with N₂ for an additional 15 min while ramping up to 120° C. The mixture was stirred at 120° C. for 3 h and then allowed to cool, quenched with water (100 mL), extracted with EtOAc (100 mL) and brine (100 mL). The combined organic layers were dried over anhydrous Na₂SO₄, filtered, and concentrated to dryness. The residue was purified by flash chromatography (ISCO®; 80 g SepaFlash® Silica Flash Column, Eluent of 0-20% Ethyl acetate/Petroleum ether gradient @ 40 mL/min). The title compound (1.4 g, 5.93 mmol, 29% yield) was obtained as a white solid. ¹H NMR (400 MHz, CDCl₃): δ ppm=7.56 (d, J=2.2 Hz, 1H), 7.38 (dd, J=2.3, 8.4 Hz, 1H), 6.91 (d, J=8.3 Hz, 1H), 6.14 (s, 1H), 5.04 (dd, J=6.0, 8.4 Hz, 2H), 4.76 (t, J=6.5 Hz, 2H), 4.20-3.99 (m, 5H), 3.87 (s, 3H).

### Step 4. Preparation of 2-methoxy-5-(oxetan-3-yl)benzaldehyde

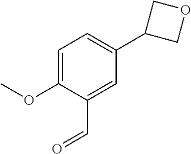

A mixture of 2-[2-methoxy-5-(oxetan-3-yl)phenyl]-1,3-dioxolane (0.85 g, 3.6 mmol, 1 eq) and 4-methylbenzenesulfonic acid hydrate (68 mg, 360 μmol, 0.1 eq) in acetone (12 mL) in a dried flask under nitrogen was stirred at 15° C. for 12 hrs. The mixture was treated with saturated NaHCO₃ (10 mL) and then the resulting mixture was extracted with EtOAc (10 mL×2). The combined organics were dried over anhydrous Na₂SO₄, filtered and evaporated under vacuum to afford a residue. The residue was purified by flash chromatography (ISCO®; 12 g SepaFlash® Silica Flash Column, Eluent of 0-25% Ethyl acetate/Petroleum ether gradient @ 30 mL/min). The title compound (170 mg, 884 μmol, 25% yield) was obtained as a colorless oil.

### Example 24

### Preparation of 3-[2-methoxy-5-(pentafluoro-sulfanyl)phenyl]-4-[1-methyl-5-[2-(5,6,7,8-tetrahydro-1,8-naphthyridin-2-yl)ethoxy]pyrazol-3-yl]butanoic acid

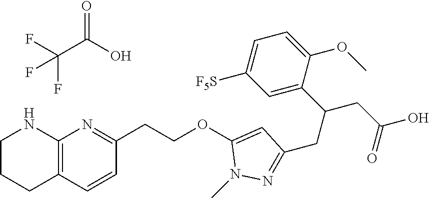

Example 24 was prepared in analogous manner to Example 1, using 2-methoxy-5-(pentafluoro-sulfanyl)benzaldehyde as the required benzaldehyde (obtained according to Example T) as the required benzaldehyde in the reaction Scheme 4 and replacing Step 12 of Scheme 4 with Step 4 of Scheme 5. The crude product was purified by Prep-HPLC (Column: Boston Green ODS 150*30 5μ; mobile phase: [water (0.1% TFA)-ACN]; B %: 25%-55%, 8 min.). The title compound (35 mg, 51 μmol, 44% yield, TFA) was obtained as a white solid. ¹H NMR (400 MHz, CD₃OD) δ=7.69-7.54 (m, 2H), 7.44 (s, 1H), 7.06 (d, J=9.2 Hz, 1H), 6.68 (d, J=7.6 Hz, 1H), 5.53-5.43 (m, 1H), 4.37-4.25 (m, 2H), 3.93 (s, 3H), 3.79 (quin, J=7.2 Hz, 1H), 3.54-3.43 (m, 5H), 3.16 (t, J=6.0 Hz, 2H), 2.94-2.86 (m, 2H), 2.82 (br t, J=6.0 Hz, 2H), 2.77-2.65 (m, 2H), 1.95 (quin, J=6.0 Hz, 2H). ¹⁹F NMR (376 MHz, CD₃OD) δ5.26 (quin, J=150.2 Hz, 1F), 64.76-60.48 (m, 4F), −73.26-−83.02 (m, 3F). LCMS (m/z 577.1 (M+H)).

### Example T

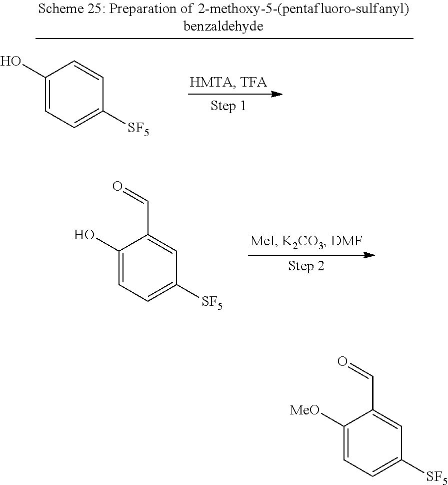

### Step 1. Preparation of 2-hydroxy-5-(pentafluoro-sulfanyl)benzaldehyde

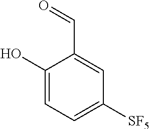

A mixture of 4-(pentafluoro-sulfanyl)phenol (1 g, 4.54 mmol, 1 eq) in TFA (10 mL) was treated with 6,7,8,9-tetrazatricyclodecane (1.27 g, 9.08 mmol, 1.7 mL, 2 eq). The resulting mixture was stirred at 80° C. for 4 hr. The reaction mixture was cooled at room temperature and neutralized with NaHCO₃. The aqueous layer was extracted with EtOAc (30 mL*3) and dried over anhydrous Na₂SO₄ and the combined organic layers were concentrated. The residue was purified by flash chromatography (ISCO®; 12 g SepaFlash® Silica Flash Column, Eluent of 0˜10% Ethyl acetate/Petroleum ether gradient @ 30 mL/min). The title compound (0.26 g, 1.05 mmol, 23% yield) was obtained as yellow oil. ¹H NMR (400 MHz, CDCl₃) δ=11.32 (s, 1H), 10.05-9.82 (m, 1H), 8.00 (d, J=2.8 Hz, 1H), 7.91 (dd, J=2.8, 9.2 Hz, 1H), 7.08 (d, J=9.6 Hz, 1H).

### Step 2. Preparation of 2-methoxy-5-(pentafluoro-sulfanyl)benzaldehyde

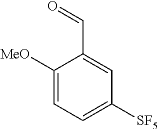

A solution of 2-hydroxy-5-(pentafluoro-sulfanyl)benzaldehyde (0.26 g, 1.05 mmol, 1 eq) in THF (10 mL) was treated with Cs₂CO₃ (683 mg, 2.1 mmol, 2 eq) and CH₃I (595 mg, 4.2 mmol, 260 μL, 4eg). The resulting mixture was stirred at 30° C. for 12 hr. The mixture was concentrated under reduced pressure to remove THF. The residue was diluted with water (10 mL) and extracted with EtOAc (10 mL*3). The combined organic layers were washed with brine (10 mL), dried over Na₂SO₄, filtered and concentrated under reduced pressure to give a residue. The residue was used in the next step without further purification. The title compound (0.26 g, 992 μmol, 95% yield) was obtained as yellow solid. ¹H NMR (400 MHz, CDCl₃) δ=10.46 (s, 1H), 8.23 (d, J=2.8 Hz, 1H), 7.94 (dd, J=2.8, 9.2 Hz, 1H), 7.06 (d, J=9.6 Hz, 1H), 4.02 (s, 3H).

### Example 25

### Preparation of 3-(3,5-diisopropyl-4-methoxy-phenyl)-4-[1-methyl-5-[2-(5,6,7,8-tetrahydro-1,8-naphthyridin-2-yl)ethoxy]pyrazol-3-yl]butanoic acid

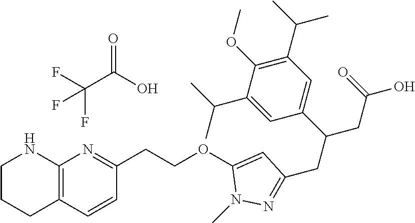

Example 25 was prepared in analogous manner to Example 1, using 4-(3,5-diisopropyl-4-methoxy-phenyl)tetrahydropyran-2,6-dione (obtained according to Example U) as the required anhydride in the reaction Scheme 4 and replacing Step 12 of Scheme 4 with Step 4 of Scheme 5. The crude product was purified by prep-HPLC (Boston Green ODS 150*30 5μ; mobile phase: [water (0.1% TFA)-ACN]; B %: 30%-56.25%, 7 min) affording the title compound (0.039 g, 59 μmol, 55% yield, 98% purity, TFA) as a white solid. LCMS (m/z 535.1 (M+H)). ¹H NMR (400 MHz, CD₃OD) δ=7.60 (d, J=7.6 Hz, 1H), 6.91 (s, 2H), 6.68 (d, J=7.6 Hz, 1H), 5.55 (s, 1H), 4.41-4.28 (m, 2H), 3.67 (s, 3H), 3.54-3.48 (m, 5H), 3.43-3.35 (m, 1H), 3.30-3.23 (m, 2H), 3.17 (t, J=6.0 Hz, 2H), 2.92-2.55 (m, 6H), 1.96 (quin, J=6.0 Hz, 2H), 1.17 (dd, J=6.8, 14.4 Hz, 12H). ¹⁹F NMR (376 MHz, CD₃OD) δ=−77.35 (br s, 3F).

### Example U

### Scheme 26: Preparation of 3,5-diisopropyl-4-methoxybenzaldehyde

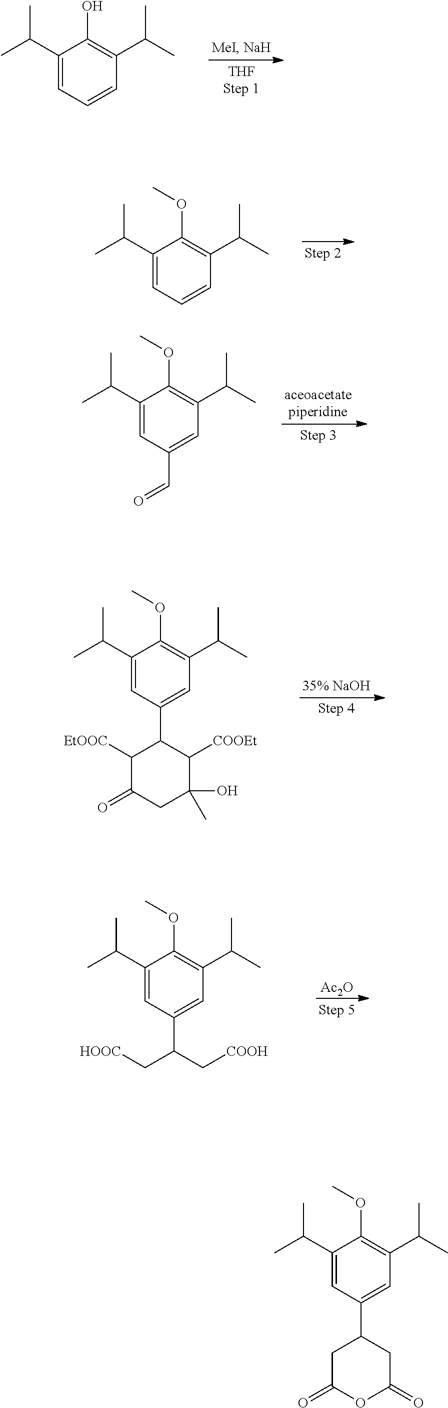

### Step 1. Preparation of 1,3-diisopropyl-2-methoxy-benzene

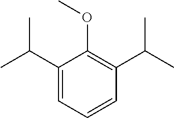

A mixture of 2,6-diisopropylphenol (10 g, 56.09 mmol, 10.40 mL, 1 eq) in anhydrous THF (120 mL) was treated with NaH (5.61 g, 140 mmol, 60%, 2.5 eq) at 0° C. The resulting mixture was stirred at 10° C. for 0.5 hr, then CH₃I (31.85 g, 224.38 mmol, 13.97 mL, 4 eq) was added dropwise to the mixture and the mixture was stirred at 10° C. for 1.5 hr. The reaction mixture was quenched with water (300 mL, slow addition) and then extracted with ethyl acetate (100 mL*3). The combined organic phase was washed with brine (100 mL), dried over anhydrous Na₂SO₄, filtered and concentrated in vacuo. The residue was purified by flash chromatography (ISCO®; 80 g SepaFlash® Silica Flash Column, Eluent of 0˜5% Ethyl acetate/Petroleum ether gradient @ 60 mL/min). The title compound (5.95 g, 31 mmol, 55% yield) was obtained as a colorless liquid. ¹H NMR (400 MHz, CDCl₃) δ=7.22-7.05 (m, 3H), 3.78 (s, 3H), 3.39 (spt, J=7.2 Hz, 2H), 1.28 (d, J=7.2 Hz, 12H).

### Step 2. Preparation of 3,5-diisopropyl-4-methoxybenzaldehyde

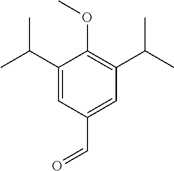 [SMILES: COc1c(C(C)C)cc(C=O)cc1C(C)C]

A solution of 1,3-diisopropyl-2-methoxybenzene (0.92 g, 4.78 mmol, 1 eq) in TFA (10 mL) was treated with 6,7,8,9-tetrazatricyclodecane (1.34 g, 9.6 mmol, 1.8 mL, 2 eq). The resulting mixture was stirred at 80° C. for 5 hr. The reaction mixture was cooled at room temperature and neutralized with NaHCO₃. The aqueous layer was extracted with EtOAc 450 mL (150 mL*3) and dried over anhydrous Na₂SO₄, filtered and concentrated in vacuo. The residue was used in the next step without further purification. The title compound (1 g, 4.54 mmol, 95% yield) was obtained as a yellow oil. ¹H NMR (400 MHz, CDCl₃) δ=10.00-9.88 (m, 1H), 7.66 (s, 2H), 3.85-3.74 (m, 3H), 3.43-3.31 (m, 2H), 1.28 (d, J=6.8 Hz, 12H).

### Step 3. Preparation of diethyl 2-(3,5-diisopropyl-4-methoxy-phenyl)-4-hydroxy-4-methyl-6-oxo-cyclohexane-1,3-dicarboxylate

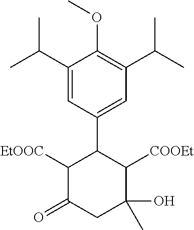 [SMILES: CCOC(=O)C1C(=O)CC(C)(O)C(C(=O)OCC)C1c1cc(C(C)C)c(OC)c(C(C)C)c1]

A solution of 3,5-diisopropyl-4-methoxy-benzaldehyde (1 g, 4.54 mmol, 1 eq) was treated with ethyl acetoacetate (2.07 g, 15.9 mmol, 2 mL, 3.5 eq) and piperidine (135 mg, 1.6 mmol, 160 μL, 0.35 eq). The resulting mixture was stirred at 10° C. for 12 hr. The mixture was combined with petroleum ether and stirred 1 hr to give a residue. The residue was dry under vacuum. The residue was used into the next step without further purification. The title compound (2 g, 4.32 mmol, 95% yield) was obtained as a white solid. ¹H NMR (400 MHz, CDCl₃) δ=6.92 (s, 2H), 4.08-3.96 (m, 2H), 3.91-3.85 (m, 1H), 3.82-3.75 (m, 2H), 3.72-3.59 (m, 4H), 3.34-3.24 (m, 2H), 3.01 (d, J=12.4 Hz, 1H), 2.72 (d, J=14.4 Hz, 1H), 2.50 (dd, J=2.8, 14.4 Hz, 1H), 1.35 (s, 3H), 1.20 (dd, J=5.2, 6.8 Hz, 12H), 1.02 (t, J=7.2 Hz, 3H), 0.76 (t, J=7.2 Hz, 3H).

### Step 4. Preparation of 3-(3,5-diisopropyl-4-methoxy-phenyl)pentanedioic acid

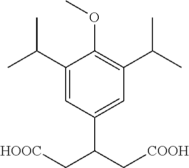 [SMILES: COc1c(C(C)C)cc(C(CC(=O)O)CC(=O)O)cc1C(C)C]

A solution of diethyl 2-(3,5-diisopropyl-4-methoxy-phenyl)-4-hydroxy-4-methyl-6-oxo-cyclohexane-1,3-dicarboxylate (2 g, 4.32 mmol, 1 eq) in water (4 mL) was treated with NaOH (12 mL, 35% aq.) and EtOH (16 mL). The resulting mixture was stirred at 110° C. for 3 hr. The mixture was concentrated under reduced pressure to remove EtOH. The residue was diluted with water (30 mL) and extracted with EtOAc (30 mL). The aqueous layer was adjusted by HCl (20%) to pH=1, then extracted with EtOAc (150 mL*3). The combined organic layers were dried over anhydrous Na₂SO₄, filtered, and the filtrate was concentrated in vacuo to give a residue. The residue was used into the next step without further purification. The title compound (1 g, 3.10 mmol, 72% yield) was obtained as a brown solid. ¹H NMR (400 MHz, CD₃OD) δ=7.00 (s, 2H), 3.68 (s, 3H), 3.53 (quin, J=7.6 Hz, 1H), 3.31-3.23 (m, 2H), 2.76-2.66 (m, 2H), 2.62-2.52 (m, 2H), 1.21 (d, J=6.8 Hz, 12H).

### Step 5. Preparation of 4-(3,5-diisopropyl-4-methoxy-phenyl)tetrahydropyran-2,6-dione

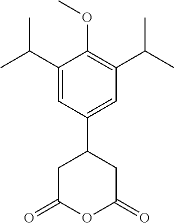 [SMILES: COc1c(C(C)C)cc(C2CC(=O)OC(=O)C2)cc1C(C)C]

A solution of 3-(3,5-diisopropyl-4-methoxy-phenyl)pentanedioic acid (1 g, 3.1 mmol, 1 eq) was treated with Ac₂O (24 mL). The resulting mixture was stirred at 140° C. for 2.5 hr. The reaction mixture was concentrated under reduced pressure to give a residue. The residue was used into the next step without further purification. The title compound (0.9 g, 3.0 mmol, 95% yield) was obtained as a brown solid. ¹H NMR (400 MHz, CDCl₃) δ=6.83 (s, 2H), 3.74-3.60 (m, 3H), 3.36-3.30 (m, 1H), 3.29-3.19 (m, 1H), 3.30-3.19 (m, 1H), 3.12-2.96 (m, 2H), 2.89-2.76 (m, 2H), 1.15 (br d, J=7.2 Hz, 12H).

### Example 26

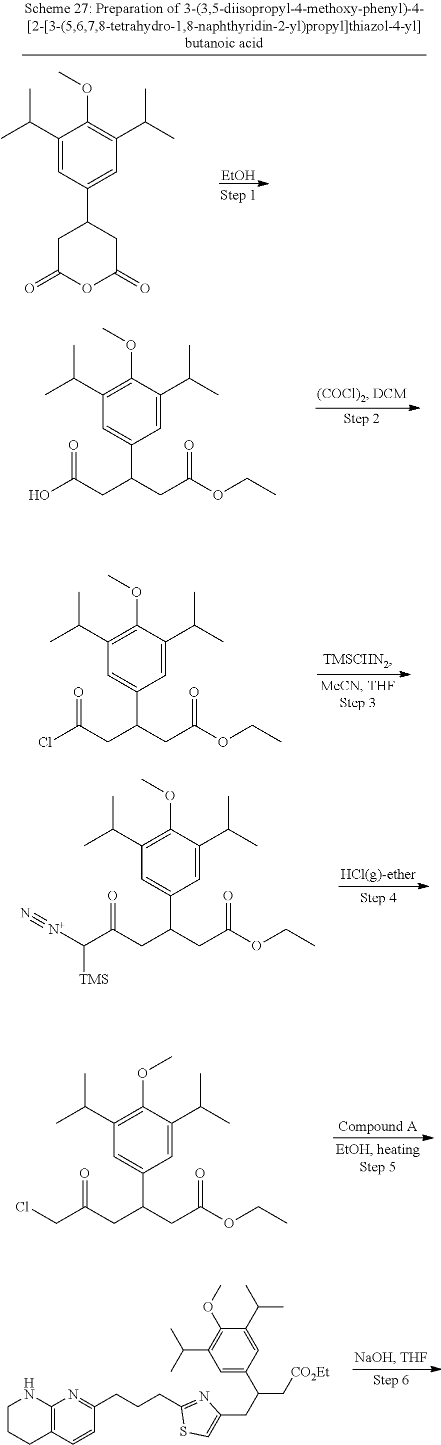

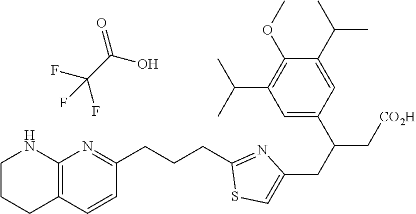

### Step 1. Preparation of 3-(3,5-diisopropyl-4-methoxy-phenyl)-5-ethoxy-5-oxo-pentanoic acid

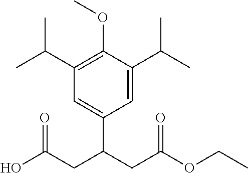 [SMILES: CCOC(=O)CC(CC(=O)O)c1cc(C(C)C)c(OC)c(C(C)C)c1]

A solution of 4-(3,5-diisopropyl-4-methoxy-phenyl)tetrahydropyran-2,6-dione (1.44 g, 4.73 mmol, 1 eq) was treated with EtOH (23.26 g, 505 mmol, 29.5 mL, 107 eq). The resulting mixture was stirred at 90° C. for 16 hr. The mixture was concentrated under reduced pressure to give a residue. The residue was purified by flash chromatography (ISCO®; 12 g SepaFlash® Silica Flash Column, Eluent of 0˜30% Ethyl acetate/Petroleum ether gradient @ 35 mL/min). LCMS (m/z 373.0 (M+Na)). The residue was purified by prep-HPLC (HCl condition: column: Phenomenex Synergi Max-RP 250*50 mm*10 μm; mobile phase: [water (0.225% FA)-ACN]; B %: 20%-75%, 24 min). The title compound (1.2 g, 3.42 mmol, 72% yield) was obtained as a colorless gum. ¹H NMR (400 MHz, CDCl₃) δ=6.92 (s, 2H), 4.04 (q, J=7.2 Hz, 2H), 3.71 (s, 3H), 3.65-3.54 (m, 1H), 3.29 (quin, J=6.8 Hz, 2H), 2.83-2.58 (m, 4H), 1.21 (br d, J=6.8 Hz, 12H), 1.18-1.11 (m, 3H).

### Step 2. Preparation of ethyl 5-chloro-3-(3,5-diisopropyl-4-methoxy-phenyl)-5-oxo-pentanoate

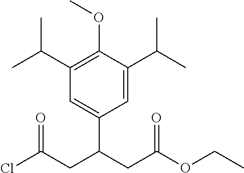 [SMILES: CCOC(=O)CC(CC(=O)Cl)c1cc(C(C)C)c(OC)c(C(C)C)c1]

A solution of 3-(3,5-diisopropyl-4-methoxy-phenyl)-5-ethoxy-5-oxo-pentanoic acid (0.6 g, 1.71 mmol, 1 eq) in DCM (7 mL), was treated with DMF (0.1 mL). The resulting mixture was treated with oxalyl chloride (652 mg, 5.14 mmol, 450 μL, 3 eq) via dropwise addition, and the resulting mixture was stirred for 3 hr at 10° C. The mixture was concentrated under reduced pressure to give a residue. The residue was used into the next step without further purification. The title compound (0.6 g, 1.63 mmol, 95% yield) was obtained as brown solid.

### Step 3. Preparation of 4-(3,5-diisopropyl-4-methoxy-phenyl)-6-ethoxy-2,6-dioxo-1-trimethylsilyl-hexane-1-diazonium

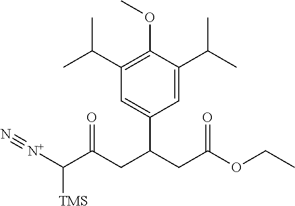 [SMILES: CCOC(=O)CC(CC(=O)C([N+]#N)[Si](C)(C)C)c1cc(C(C)C)c(OC)c(C(C)C)c1]

Ethyl 5-chloro-3-(3,5-diisopropyl-4-methoxy-phenyl)-5-oxo-pentanoate (0.6 g, 1.63 mmol, 1 eq) in a mixture of THF (5 mL) and CH₃CN (5 mL) was treated with TMSCHN₂ (2 M, 1.63 mL, 2 eq) at 0° C. The resulting mixture was warmed to 10° C. and stirred for 12 hr. The mixture was concentrated under reduced pressure to give the residue. The residue was used into the next step without further purification. The title compound (0.7 g, 1.56 mmol, 96% yield) was obtained as black brown oil. LCMS (m/z 447.3 (M)).

### Step 4. Preparation of ethyl 6-chloro-3-(3,5-diisopropyl-4-methoxy-phenyl)-5-oxo-hexanoate

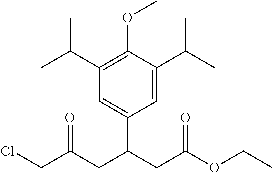 [SMILES: CCOC(=O)CC(CC(=O)CCl)c1cc(C(C)C)c(OC)c(C(C)C)c1]

A solution of 4-(3,5-diisopropyl-4-methoxy-phenyl)-6-ethoxy-2,6-dioxo-1-trimethylsilyl-hexane-1-diazonium (0.7 g, 1.56 mmol, 1 eq) was treated with HCl/EtOAc (4 M, 15 mL, 38.37 eq). The resulting mixture was stirred at 10° C. for 15 hr.

The mixture was concentrated under reduced pressure to give a residue. The residue was purified by flash chromatography (ISCO®; 12 g SepaFlash® Silica Flash Column, Eluent of 0˜30% Ethyl acetate/Petroleum ether gradient @ 30 mL/min). The title compound (0.36 g, 940 μmol, 60% yield) was obtained as colorless oil. LCMS (m/z 405.0 (M+Na)). ¹H NMR (400 MHz, CDCl₃) δ=6.90 (s, 2H), 4.06 (q, J=7.6 Hz, 2H), 3.98-3.85 (m, 2H), 3.74-3.63 (m, 4H), 3.30 (spt, J=6.8 Hz, 2H), 3.02-2.88 (m, 2H), 2.72-2.57 (m, 2H), 1.24-1.14 (m, 15H).

### Step 5. Preparation of ethyl 3-(3,5-diisopropyl-4-methoxyphenyl)-4-(2-(3-(5,6,7,8-tetrahydro-1,8-naphthyridin-2-yl)propyl)thiazol-4-yl)butanoate

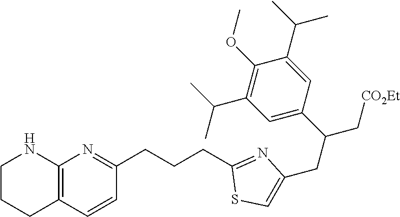 [SMILES: CCOC(=O)CC(Cc1csc(CCCc2ccc3c(n2)NCCC3)n1)c1cc(C(C)C)c(OC)c(C(C)C)c1]

A solution of ethyl 6-chloro-3-(3,5-diisopropyl-4-methoxy-phenyl)-5-oxo-hexanoate (0.16 g, 417.84 umol, 1.2 eq) in EtOH (10 mL) was treated with 4-(5,6,7,8-tetrahydro-1,8-naphthyridin-2-yl)butanethioamide (Compound A, 82 mg, 348 μmol, 1 eq). The resulting mixture was stirred at 90° C. for 12 hr. The mixture was concentrated under reduced pressure to give a residue. The residue was purified by flash chromatography (ISCO®; 4 g SepaFlash® Silica Flash Column, Eluent of 0˜3% Methanol/Dichloromethane gradient @ 25 mL/min). The title compound (0.15 g, 266 μmol, 76% yield) was obtained as a yellow gum. LCMS (m/z 564.6 (M+Na)). ¹H NMR (400 MHz, CDCl₃) δ=7.30 (d, J=7.6 Hz, 1H), 6.86 (s, 2H), 6.57 (s, 1H), 6.41 (d, J=7.6 Hz, 1H), 3.96 (q, J=7.2 Hz, 2H), 3.69 (s, 3H), 3.61-3.53 (m, 1H), 3.52-3.46 (m, 2H), 3.33-3.23 (m, 2H), 3.07-2.96 (m, 4H), 2.82 (t, J=7.6 Hz, 2H), 2.75 (t, J=6.4 Hz, 2H), 2.70-2.55 (m, 2H), 2.31-2.20 (m, 2H), 1.94 (quin, 7=6.0 Hz, 2H), 1.18 (t, 7=7.2 Hz, 12H), 1.08 (t, 7=7.2 Hz, 3H).

### Step 6. Preparation of 3-(3,5-diisopropyl-4-methoxy-phenyl)-4-[2-[3-(5,6,7,8-tetrahydro-1,8-naphthyridin-2-yl)propyl]thiazol-4-yl]butanoic acid

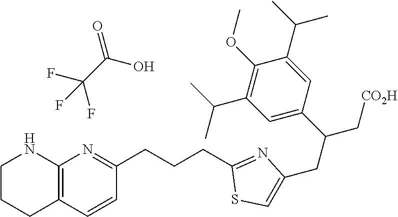

A solution of ethyl 3-(3,5-diisopropyl-4-methoxy-phenyl)-4-[2-[3-(5,6,7,8-tetrahydro-1,8-naphthyridin-2-yl)propyl]thiazol-4-yl]butanoate (0.15 g, 266 μmol, 1 eq) in THF (3 mL) was treated with NaOH (1 M, 3 mL, 11.3 eg). The resulting mixture was stirred at 60° C. for 12 hr. The reaction mixture was quenched by HO Ac (240 mg) at 10° C. and then concentrated under reduced pressure to give a residue. The residue was purified by prep-HPLC (column: Boston Green ODS 150*30 5μ; mobile phase: [water (0.1% TFA)-ACN]; B %: 37%-67%, 8 min). The title compound (90 mg, 135 μmol, 51% yield, 97% purity, TFA) was obtained as a white solid. LCMS (m/z 536.0 (M+H)). ¹H NMR (400 MHz, CD₃OD) δ=7.58 (d, 7=7.6 Hz, 1H), 6.93-6.81 (m, 3H), 6.63 (d, 7=7.6 Hz, 1H), 3.66 (s, 3H), 3.58-3.47 (m, 3H), 3.29-3.21 (m, 2H), 3.14-3.04 (m, 3H), 3.02-2.92 (m, 1H), 2.85-2.74 (m, 4H), 2.72-2.58 (m, 2H), 2.14 (quin, J=7.6 Hz, 2H), 1.95 (td, 7=6.4, 11.6 Hz, 2H), 1.17 (dd, 7=6.8, 12.4 Hz, 12H). ¹⁹F NMR (376 MHz, CD₃OD) −77.40 (s, 3F).

### Example V

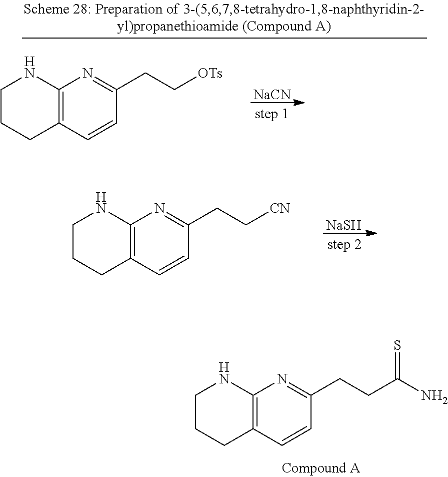

### Step 1. Preparation of 3-(5,6,7,8-tetrahydro-1,8-naphthyridin-2-yl)propanenitrile

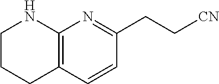 [SMILES: N#CCCc1ccc2c(n1)NCCC2]

A solution of 2-(5,6,7,8-tetrahydro-1,8-naphthyridin-2-yl)ethyl 4-methylbenzenesulfonate (1 g, 3.01 mmol, 1 eq) in DMF (10 mL) was treated with NaCN (0.39 g, 8.0 mmol, 2.65 eq). The resulting mixture was stirred at 80° C. for 6 hr. The reaction mixture was poured into water (50 mL), and extracted with EtOAc (3*50 mL). The combined organic layer was washed with brine (50 mL), dried over sodium sulfate, filtered and evaporated to give a residue (0.6 g) as a yellow solid used into the next step without further purification. LCMS (m/z 188.0 (M+H)). ¹H NMR (CDCl₃, 400 MHz) δ=7.09 (d, 7=7.2 Hz, 1H), 6.39 (d, 7=7.2 Hz, 1H), 4.81 (br s, 1H), 3.41 (td, J=5.6, 2.4 Hz, 2H), 2.83-2.89 (m, 2H), 2.68-2.78 (m, 4H), 1.87-1.97 (m, 2H)).

### Step 2. Preparation of 3-(5,6,7,8-tetrahydro-1,8-naphthyridin-2-yl)propanethioamide (Compound A)

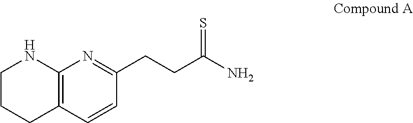 [SMILES: NC(=S)CCc1ccc2c(n1)NCCC2]

A mixture of Compound A, diethylamine hydrochloride (1.71 g, 15.59 mmol, 9.73 eq) and sulfanylsodium hydrate (1.16 g, 15.67 mmol, 9.78 eq) in DMF (15 mL) was stirred at 55° C. for 20 hr. The reaction mixture was poured into water (50 mL), then extracted with Ethyl acetate (50 mL*3). The combined organic phase was washed with brine (100 mL), dried with anhydrous Na₂SO₄, filtered and concentrated under vacuum. The residue was purified by flash chromatography (ISCO®; 4 g SepaFlash® Silica Flash Column, Eluent of 0˜10% Dichloromethane/Methanol gradient @ 30 mL/min). Compound A (310 mg, 1.4 mmol, 87% yield) was obtained as a yellow solid. LCMS (m/z 222.0 (M+H)).

### Example 27

### Preparation of 3-(3,5-di-tert-butylphenyl)-4-(2-(3-(5,6,7,8-tetrahydro-1,8-naphthyridin-2-yl)propyl)thiazol-4-yl)butanoic acid

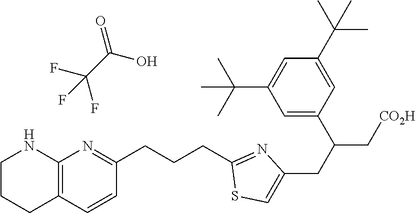

Example 27 was prepared in analogous manner to Example 26, using 4-(3,5-di-tert-butylphenyl)dihydro-2H-pyran-2,6(3H)-dione as the required anhydride in the reaction Scheme 27 where the anhydride is prepared in analogous manner to Example 1 using 3,5-ditert-butylbenzaldehyde as the required benzaldehyde in the reaction Scheme 4. The crude product was purified by prep-HPLC (Boston Green ODS 150*30 5μ; mobile phase: [water (0.1% TFA)-ACN]; B %: 42%-72%, 8 min) affording the title compound (151 mg, 234 umol, 58% yield, TFA) as a white solid. LCMS (m/z 534.1 (M+H)). ¹H NMR (400 MHz, CD₃OD) δ=7.58 (br d, 7=7.2 Hz, 1H), 7.24 (s, 1H), 7.01 (d, 7=1.2 Hz, 2H), 6.81-6.90 (m, 1H), 6.63 (br d, 7=7.2 Hz, 1H), 3.57 (quin, 7=7.6 Hz, 1H), 3.46-3.52 (m, 2H), 2.95-3.16 (m, 4H), 2.74-2.86 (m, 4H), 2.62-2.73 (m, 2H), 2.14 (quin, 7=7.2 Hz, 2H), 1.95 (quin, 7=6.0 Hz, 2H), 1.27 (s, 18H), ¹⁹F NMR (CD₃OD, 376 MHz) δ=−77.45-−77.32 (m, 1F).

### Example 28

### Preparation of 3-(5-(tert-butyl)-2-methoxyphenyl)-4-(2-(3-(5,6,7,8-tetrahydro-1,8-naphthyridin-2-yl)propyl)thiazol-4-yl)butanoic acid

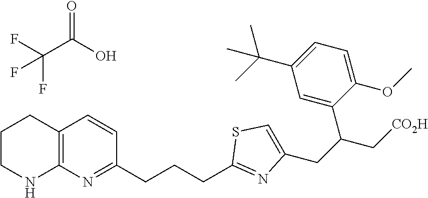

Example 28 was prepared in analogous manner to Example 26, using 4-(5-(tert-butyl)-2-methoxyphenyl)dihydro-2H-pyran-2,6(3H)-dione as the required anhydride in the reaction Scheme 27. The crude product was purified by prep-HPLC (Boston Green ODS 150*30 5μ; mobile phase: [water (0.1% TFA)-ACN]; B %: 35%-65%, 8 min) affording the title compound (131 mg, 204 μmol, 48% yield, TFA) as a white solid. LCMS (m/z 508.0 (M+H)). ¹H NMR (400 MHz, CD₃OD) δ=7.58 (d, 7=7.2 Hz, 1H), 7.15 (dd, 7=8.8, 2.4 Hz, 1H), 7.08 (d, 7=2.4 Hz, 1H), 6.87 (s, 1H), 6.80 (d, 7=8.8 Hz, 1H), 6.61 (d, 7=7.2 Hz, 1H), 3.87 (quin, 7=7.6 Hz, 1H), 3.77 (s, 3H), 3.46-3.54 (m, 2H), 3.15 (d, 7=7.6 Hz, 2H), 3.06 (t, 7=7.6 Hz, 2H), 2.82 (t, 7=6.0 Hz, 2H), 2.64-2.78 (m, 4H), 2.10 (quin, 7=7.6 Hz, 2H), 1.96 (quin, 7=6.0 Hz, 2H), 1.22 (s, 9H). ¹⁹F NMR (CD₃OD, 376 MHz) δ=−77.43 (s, 1F).

### Example 29

### Preparation of 3-(3,5-di-tert-butyl-4-methoxyphenyl)-4-(2-(3-(5,6,7,8-tetrahydro-1,8-naphthyridin-2-yl)propyl)thiazol-4-yl)butanoic acid

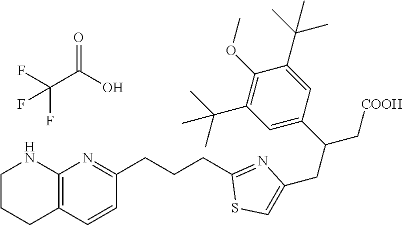

Example 29 was prepared in analogous manner to Example 26, using 4-(3,5-di-tert-butyl-4-methoxyphenyl)dihydro-2H-pyran-2,6(3H)-dione as the required anhydride in the reaction Scheme 27 where the anhydride is prepared in analogous manner to Example 1 using 3,5-di-tert-butyl-4-methoxybenzaldehyde as the required benzaldehyde in the reaction Scheme 4. The crude product was purified by prep-HPLC (Boston Green ODS 150*30 5μ; mobile phase: [water (0.1% TFA)-ACN]; B %: 44%-74%, 8 min) affording the title compound (88 mg, 130 umol, 63% yield, TFA) as a white solid. LCMS (m/z 564.3 (M+H)). ¹H NMR (400 MHz, CD₃OD) δ=7.58 (d, 7=7.2 Hz, 1H), 7.03 (s, 2H), 6.85 (s, 1H), 6.63 (d, 7=7.6 Hz, 1H), 3.60 (s, 3H), 3.47-3.54 (m, 3H), 3.04-3.12 (m, 3H), 2.92-2.99 (m, 1H), 2.75-2.85 (m, 4H), 2.57-2.71 (m, 2H), 2.14 (quin, 7=7.6 Hz, 2H), 1.92-1.99 (m, 2H), 1.36 (s, 18H). ¹⁹F NMR (376 MHz, CD₃OD) δ=−77.36 (s, 1F).

### Example 30

### Preparation of 3-[3-tert-butyl-5-[1-(difluoromethyl)cyclopropyl]phenyl]-4-[2-[3-(5,6,7,8-tetrahydro-1,8-naphthyridin-2-yl)propyl]thiazol-4-yl]butanoic acid

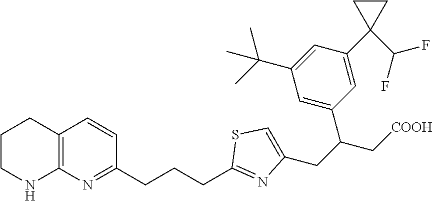 [SMILES: CC(C)(C)c1cc(C(CC(=O)O)Cc2csc(CCCc3ccc4c(n3)NCCC4)n2)cc(C2(C(F)F)CC2)c1]

Example 30 was prepared in analogous manner to Example 26, using 4-[3-tert-butyl-5-[1-(difluoromethyl)cyclopropyl]phenyl]tetrahydropyran-2,6-dione as the required anhydride in the reaction Scheme 27 where the anhydride is prepared in analogous manner to Example 1 using 3-(tert-butyl)-5-(1-(difluoromethyl)cyclopropyl) benzaldehyde as the required benzaldehyde in the reaction Scheme 4. The crude product was purified by prep-HPLC (Boston Prime C18 150*30 mm 5 μm; mobile phase: [water (0.05% ammonia hydroxide v/v)-ACN]; B %: 25%-55%, 9 min) affording the title compound (29 mg, 50 μmol, 44% yield) as a white solid. LCMS (m/z 568.3 (M+H)). ¹H NMR (400 MHz, CD₃OD) δ=7.38 (d, J=7.2 Hz, 1H), 7.23 (d, J=12.4 Hz, 2H), 7.11 (s, 1H), 6.87 (s, 1H), 6.51 (d, 7=7.2 Hz, 1H), 5.84-5.50 (m, 1H), 3.69 (quin, 7=7.2 Hz, 1H), 3.43-3.36 (m, 2H), 3.06-2.91 (m, 4H), 2.82-2.66 (m, 4H), 2.64-2.50 (m, 2H), 2.31-2.18 (m, 1H), 2.13-1.99 (m, 1H), 1.89 (quin, 7=6.0 Hz, 2H), 1.28 (s, 9H), 1.13-1.06 (m, 2H), 0.90 (br d, 7=2.4 Hz, 2H). ¹⁹F NMR (376 MHz, CD₃OD) δ=−118.73 (br d, J=57.2 Hz, 2F).

### Example 31

### Preparation of 3-[3-tert-butyl-5-(2,2,2-trifluoro-1,1-dimethyl-ethyl)phenyl]-4-[2-[3-(5,6,7,8-tetrahydro-1,8-naphthyridin-2-yl)propyl]thiazol-4-yl]butanoic acid

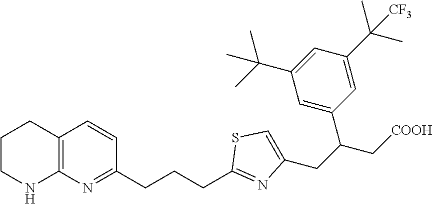 [SMILES: CC(C)(C)c1cc(C(CC(=O)O)Cc2csc(CCCc3ccc4c(n3)NCCC4)n2)cc(C(C)(C)C(F)(F)F)c1]

Example 31 was prepared in analogous manner to Example 26, using 4-[3-tert-butyl-5-(2,2,2-trifluoro-1,1-dimethyl-ethyl)phenyl]tetrahydropyran-2,6-dione as the required anhydride in the reaction Scheme 27 where the anhydride is prepared in analogous manner to Example 1 using 3-tert-butyl-5-(2,2,2-trifluoro-1,1-dimethyl-ethyl)benzaldehyde as the required benzaldehyde in the reaction Scheme 4. The crude product was purified by prep-HPLC (Boston Prime C18 150*30 mm 5 μm; mobile phase: [water (0.05% ammonia hydroxide v/v)-ACN]; B %: 35%-65%, 9 min) affording the title compound (21 mg, 35 μmol, 14% yield) as a white solid. LCMS (m/z 588.1 (M+H)). ¹H NMR (400 MHz, CD₃OD) δ=7.34 (s, 1H), 7.30 (br d, J=7.6 Hz, 1H), 7.23 (br d, J=11.6 Hz, 2H), 6.85 (d, J=2.8 Hz, 1H), 6.47 (d, J=7.6 Hz, 1H), 3.73-3.66 (m, 1H), 3.42-3.36 (m, 2H), 3.06-2.92 (m, 4H), 2.77-2.65 (m, 4H), 2.60-2.50 (m, 2H), 2.19 (br d, J=7.6 Hz, 1H), 2.10-1.98 (m, 1H), 1.92-1.84 (m, 2H), 1.54 (s, 6H), 1.29 (s, 9H). ¹⁹F NMR (376 MHz, CD₃OD) δ=−77.14 (br d, J=143.1 Hz, 3F).

### Example 32

### Preparation of 3-[3-tert-butyl-5-[4-(methoxymethyl)tetrahydropyran-4-yl]phenyl]-4-[2-[3-(5,6,7,8-tetrahydro-1,8-naphthyridin-2-yl)propyl]thiazol-4-yl]butanoic acid

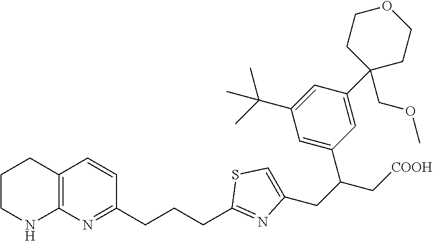 [SMILES: COCC1(c2cc(C(CC(=O)O)Cc3csc(CCCc4ccc5c(n4)NCCC5)n3)cc(C(C)(C)C)c2)CCOCC1]

Example 32 was prepared in analogous manner to Example 26, using 4-[3-tert-butyl-5-[4-(methoxymethyl)tetrahydropyran-4-yl]phenyl]tetrahydropyran-2,6-dione as the required anhydride in the reaction Scheme 27 where the anhydride is prepared in analogous manner to Example 1 using 3-tert-butyl-5-[4-(methoxymethyl)tetrahydropyran-4-yl]benzaldehyde in the reaction Scheme 4. The crude product was purified by prep-HPLC (Boston Green ODS 150*30 5μ; mobile phase: [water (0.1% TFA)-ACN]; B %: 35%-65%, 8 min) affording the title compound (10 mg, 14 μmol, 15% yield, TFA) as a white solid. ¹H NMR (400 MHz, CD₃OD) δ=7.58 (d, J=7.3 Hz, 1H), 7.21 (s, 1H), 7.09 (s, 1H), 6.99 (s, 1H), 6.83 (s, 1H), 6.63 (d, J=7.3 Hz, 1H), 3.77-3.70 (m, 2H), 3.58 (br td, J=7.6, 15.2 Hz, 1H), 3.53-3.47 (m, 2H), 3.45-3.38 (m, 2H), 3.34-3.31 (m, 2H), 3.16 (s, 3H), 3.14-3.07 (m, 1H), 2.97-2.97 (m, 1H), 3.08-2.97 (m, 2H), 2.85-2.66 (m, 5H), 2.19-2.11 (m, 2H), 2.10-2.03 (m, 2H), 2.00-1.91 (m, 4H), 1.27 (s, 9H). ¹⁹F NMR (376 MHz, CD₃OD) δ=−77.44 (br s, 1F).

### Example 33

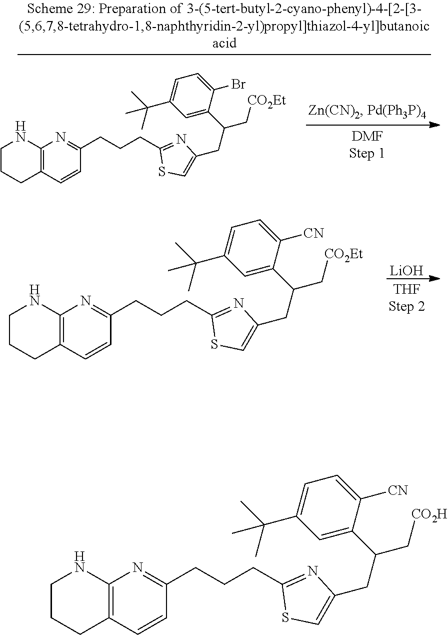 [SMILES: CCOC(=O)CC(Cc1csc(CCCc2ccc3c(n2)NCCC3)n1)c1cc(C(C)(C)C)ccc1Br ; CCOC(=O)CC(Cc1csc(CCCc2ccc3c(n2)NCCC3)n1)c1cc(C(C)(C)C)ccc1C#N ; "LiOH" ; CC(C)(C)c1ccc(C#N)c(C(CC(=O)O)Cc2csc(CCCc3ccc4c(n3)NCCC4)n2)c1]

### Step 1. Preparation of ethyl 3-(5-tert-butyl-2-cyano-phenyl)-4-[2-[3-(5,6,7,8-tetrahydro-1,8-naphthyridin-2-yl)propyl]thiazol-4-yl]butanoate

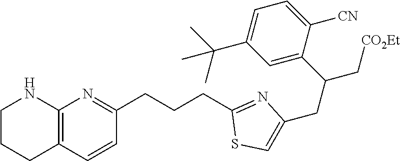 [SMILES: CCOC(=O)CC(Cc1csc(CCCc2ccc3c(n2)NCCC3)n1)c1cc(C(C)(C)C)ccc1C#N]

A mixture of ethyl 3-(2-bromo-5-tert-butyl-phenyl)-4-[2-[3-(5,6,7,8-tetrahydro-1,8-naphthyridin-2-yl)propyl]thiazol-4-yl]butanoate (100 mg, 171 μmol, 1 eq., prepared according to Example W) and Zn(CN)₂ (60 mg, 513 μmol, 33 μL, 3 eq) in DMF (5 mL) in a microwave vial was evacuated and back-filled with N₂ (3×). Pd(PPh₃)₄ (19.77 mg, 17.11 umol, 0.1 eq) was then added to the vial. The reaction vial was sealed, and the reaction mixture was again degassed and back-filled with N₂ (3×), and then stirred at 120° C. for 1.5 hr under micro-wave irradiation. The reaction mixture was poured into water (80 mL), and extracted with EtOAc (3*50 mL). The combined organic layer was washed with brine (2*50 mL), dried over sodium sulfate, filtered and evaporated to give a residue. The residue was purified by column chromatography (SiO₂, DCM/MeOH=1/0 to 20:1). The title compound (40 mg, crude) was obtained as a colorless oil. LC-MS (m/z 531.3 (M+H⁺)).

### Step 2. Preparation of 3-(5-tert-butyl-2-cyano-phenyl)-4-[2-[3-(5,6,7,8-tetrahydro-1,8-naphthyridin-2-yl)propyl]thiazol-4-yl]butanoic acid

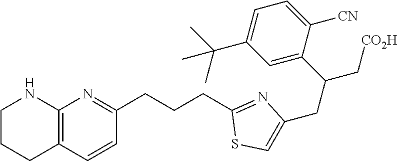 [SMILES: CC(C)(C)c1ccc(C#N)c(C(CC(=O)O)Cc2csc(CCCc3ccc4c(n3)NCCC4)n2)c1]

A mixture of ethyl 3-(5-tert-butyl-2-cyano-phenyl)-4-[2-[3-(5,6,7,8-tetrahydro-1,8-naphthyridin-2-yl)propyl]thiazol-4-yl]butanoate (40 mg, 75 μmol, 1 eq), LiOHH₂O (16 mg, 377 μmol, 5 eq) in EtOH (5 mL) and water (0.5 mL) was degassed and purged with N₂ for 3 times, and then the mixture was stirred at 25° C. for 16 hr under N₂ atmosphere. The reaction mixture was concentrated under reduced pressure to give the residue, the residue was adjusted pH=5 with AcOH and extracted with ethyl acetate (20 mL*2). The combined organic phase was concentrated under vacuum. The residue was purified by prep-HPLC (TFA condition, column: Boston Green ODS 150*30 5μ; mobile phase: [water (0.1% TFA)-ACN]; B %: 35%-65%, 8 min). The title compound (30 mg, 49 μmol, 65% yield, TFA) was obtained as a white solid. ¹H NMR (400 MHz, CD₃OD) δ=1.32 (s, 9H), 1.96 (quin, J=6.0 Hz, 2H), 2.14 (quin, J=7.6 Hz, 2H), 2.72-2.90 (m, 6H), 3.00-3.14 (m, 3H), 3.17-3.28 (m, 1H), 3.44-3.55 (m, 2H), 3.93-4.06 (m, 1H), 6.64 (d, j=7.6 Hz, 1H), 6.75 (s, 1H), 7.37 (d, j=8.4, 1H), 7.45-7.53 (m, 2H), 7.59 (d, J=7.6 Hz, 1H). ¹⁹F NMR (376 MHz, CD₃OD) δ=−77.30 (br s, 1 F).

### Example W

### Preparation of ethyl 3-(2-bromo-5-tert-butyl-phenyl)-4-[2-[3-(5,6,7,8-tetrahydro-1,8-naphthyridin-2-yl)propyl]thiazol-4-yl]butanoate

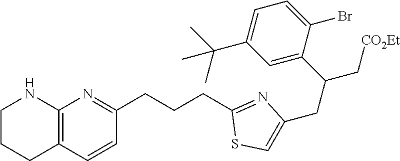 [SMILES: CCOC(=O)CC(Cc1csc(CCCc2ccc3c(n2)NCCC3)n1)c1cc(C(C)(C)C)ccc1Br]

Example W was prepared in analogous manner to Example 26 (corresponding to penultimate ester compound in the reaction Scheme 27), using 4-(2-bromo-5-tert-butyl-phenyl) tetrahydropyran-2,6-dione as the required anhydride in the reaction Scheme 27 where the anhydride is prepared in analogous manner to Example 1 using 2-bromo-5-tert-butyl-benzaldehyde (prepared according to Example P) in the reaction Scheme 4. The crude product was purified by column chromatography (SiO₂, Ethyl acetate/MeOH=1/0 to 10:1). The title compound (300 mg, 513 μmol, 73% yield) was obtained as a yellow oil. ¹H NMR (400 MHz, CDCl₃) δ=1.10 (t, J=7.2 Hz, 4H), 1.25 (s, 10H), 1.92 (dt, J=11.6, 6.0 Hz, 2H), 2.12-2.24 (m, 2H), 2.64-2.73 (m, 4H), 2.77 (d, J=7.6 Hz, 2H), 3.00 (t, J=7.6 Hz, 2H), 3.10 (dd, J=16.0, 7.2 Hz, 2H), 3.44 (br t, J=4.4 Hz, 2H), 3.92-4.04 (m, 2H), 6.38 (d, J=7.2 Hz, 1H), 6.59 (s, 1H) 7.05 (dd, J=8.4, 2.38 Hz, 1H), 7.12-7.20 (m, 2H), 7.42 (d, J=8.4 Hz, 1H).

### Example 34

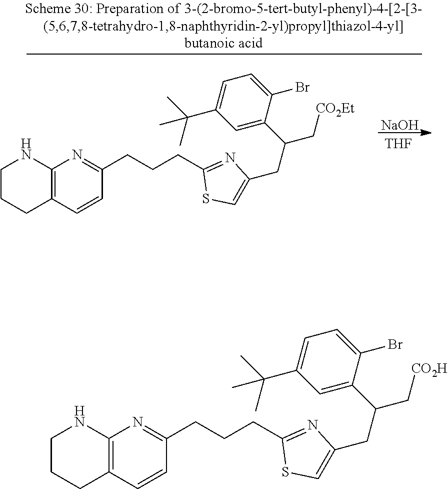 [SMILES: CCOC(=O)CC(Cc1csc(CCCc2ccc3c(n2)NCCC3)n1)c1cc(C(C)(C)C)ccc1Br ; "NaOH" ; CC(C)(C)c1ccc(Br)c(C(CC(=O)O)Cc2csc(CCCc3ccc4c(n3)NCCC4)n2)c1 ; "THF"]

A mixture of ethyl 3-(2-bromo-5-tert-butyl-phenyl)-4-[2-[3-(5,6,7,8-tetrahydro-1,8-naphthyridin-2-yl)propyl]thiazol-4-yl]butanoate (100 mg, 171 μmol, 1 eq., prepared according to Example P), NaOH (1 M, 5 mL, 29 eq) in THF (5 mL) was degassed and purged with N₂ for 3 times, and then the mixture was stirred at 65° C. for 12 hr under N₂ atmosphere. The reaction mixture was concentrated under reduced pressure to give a residue, the residue was adjusted pH=5 with AcOH and extracted with ethyl acetate (10 mL*2). The solvent was removed and the crude material was purified by prep-HPLC (Boston Green ODS 150*30 5μ; mobile phase: [water (0.1% TFA)-ACN]; B %: 35%-65%, 8 min). The title compound (45 mg, 67 μmol, 39% yield, TFA) was obtained as a white solid. ¹H NMR (CD₃OD, 400 MHz) δ=7.59 (d, J=7.2 Hz, 1H), 7.39 (d, J=8.4 Hz, 1H), 7.29 (d, J=2.0 Hz, 1H), 7.11 (dd, J=8.4, 2.4 Hz, 1H), 6.81 (s, 1H), 6.63 (d, J=7.2 Hz, 1H), 4.12 (quin, J=7.2 Hz, 1H), 3.46-3.54 (m, 2H), 3.14 (d, J=7.2 Hz, 2H), 3.03 (t, J=7.2 Hz, 2H), 2.83 (t, J=6.0 Hz, 2H), 2.69-2.77 (m, 4H), 2.12 (quin, J=7.2 Hz, 2H), 1.96 (quin, J=6.0 Hz, 2H), 1.27 (s, 9H). ¹⁹F NMR (376 MHz, CD₃OD) δ=−77.37 (s, 1 F).

### Example 35

### Preparation of 3-[2-methoxy-5-(trifluoromethyl)phenyl]-4-[2-[3-(5,6,7,8-tetrahydro-1,8-naphthyridin-2-yl)propyl]thiazol-4-yl]butanoic acid

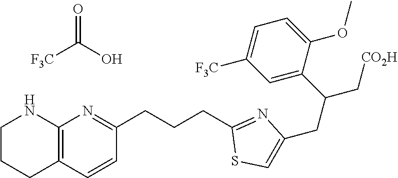 [SMILES: COc1ccc(C(F)(F)F)cc1C(CC(=O)O)Cc1csc(CCCc2ccc3c(n2)NCCC3)n1 ; O=C(O)C(F)(F)F]

Example 35 was prepared in analogous manner to Example 26, using 4-[2-methoxy-5-(trifluoromethyl)phenyl]tetrahydropyran-2,6-dione as the required anhydride in the reaction Scheme 27 where the anhydride is prepared in analogous manner to Example 1 using 2-methoxy-5-(trifluoromethyl)benzaldehyde as the required benzaldehyde in the reaction Scheme 4. The crude product was purified by prep-HPLC (Boston Green ODS 150*30 5μ; mobile phase: [water (0.1% TFA)-ACN]; B %: 28%-58%, 8 min) affording the title compound (99 mg, 156 μmol, 86% yield, TFA) as a white solid. LCMS (m/z 520.0 (M+H)). ¹H NMR (400 MHz, CD₃OD) δ=7.58 (d, J=7.6 Hz, 1H), 7.45 (br d, j=8.8 Hz, 1H), 7.35 (s, 1H), 7.06 (d, j=8.8 Hz, 1H), 6.92 (s, 1H), 6.61 (d, J=7.6 Hz, 1H), 4.00-3.85 (m, 4H), 3.54-3.46 (m, 2H), 3.16 (d, J=7.6 Hz, 2H), 3.05 (t, J=7.6 Hz, 2H), 2.85-2.79 (m, 2H), 2.79-2.68 (m, 4H), 2.10 (quin, J=7.6 Hz, 2H), 1.96 (td, J=6.0, 11.6 Hz, 2H). ¹⁹F NMR (376 MHz, CD₃OD) δ=−62.83 (s, 3F), −77.40 (br s, 3F).

### Example 36

### Preparation of 3-(5-(1-(difluoromethyl)cyclopropyl)-2-methoxyphenyl)-4-(2-(3-(5,6,7,8-tetrahydro-1,8-naphthyridin-2-yl)propyl)thiazol-4-yl)butanoic acid

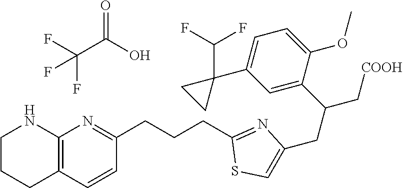

Example 36 was prepared in analogous manner to Example 26, using 4-(5-(1-(difluoromethyl)cyclopropyl)-2-methoxyphenyl)dihydro-2H-pyran-2,6(3H)-dione as the required anhydride in the reaction Scheme 27 where the anhydride is prepared in analogous manner to Example 1 using 5-[1-(difluoromethyl)cyclopropyl]-2-methoxy-benzaldehyde (obtained according to Example E starting at Step 2 and using 3-bromo-4-methoxy-benzaldehyde in place of 3-bromo-5-(tert-butyl)benzaldehyde as shown in Scheme 9) as the required benzaldehyde in the reaction Scheme 4 and replacing Step 12 of Scheme 4 with Step 2 of Scheme 29. The crude product was purified by prep-HPLC (Boston Green ODS 150*30 5μ; mobile phase: [water (0.1% TFA)-ACN]; B %: 30%-60%, 8 min) affording the title compound (21 mg, 31 μmol, 11% yield, TFA) as colorless gum. LCMS (m/z 542.2 (M+H)). ¹H NMR (400 MHz, CD₃OD) δ=7.56 (d, J=7.2 Hz, 1H), 7.18 (dd, 7=8.4, 2.0 Hz, 1H), 7.10 (d, 7=2.0 Hz, 1H), 6.91 (s, 1H), 6.85 (d, J=8.8 Hz, 1H), 6.60 (d, 7=7.2 Hz, 1H), 5.39-5.73 (m, 1H), 3.88 (quin, 7=7.6 Hz, 1H), 3.79 (s, 3H), 3.46-3.53 (m, 2H), 3.16 (d, 7=7.6 Hz, 2H), 3.08 (t, 7=7.6 Hz, 2H), 2.81 (t, 7=6.0 Hz, 2H), 2.65-2.77 (m, 4H), 2.11 (quin, 7=7.6 Hz, 2H), 1.95 (quin, 7=6.0 Hz, 2H), 1.00-1.06 (m, 2H), 0.82 (br d, 7=2.0 Hz, 2H). ¹⁹F NMR (CD₃OD, 376 MHz) δ=−77.33 (s, 1F), −118.09 (brs, 1F),-118.24 (brs, 1F).

### Example 37

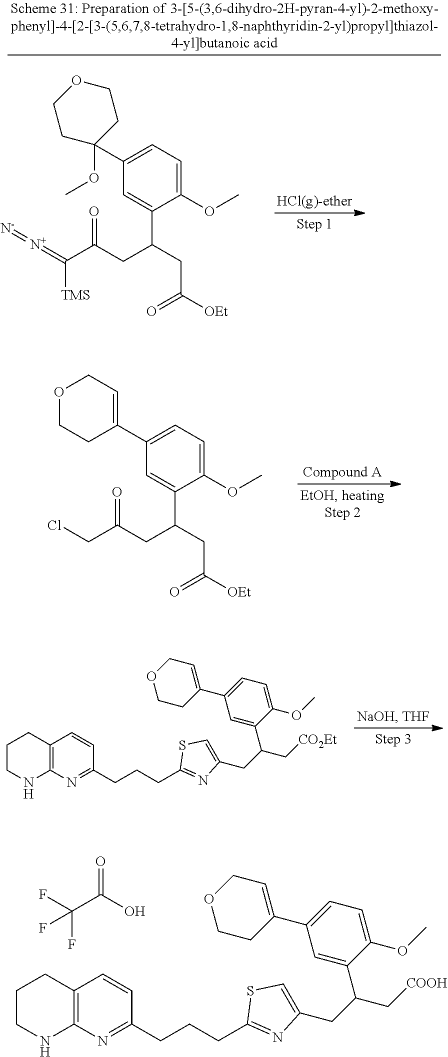

### Step 1. Preparation of ethyl 6-chloro-3-[5-(3,6-dihydro-2H-pyran-4-yl)-2-methoxy-phenyl]-5-oxo-hexanoate

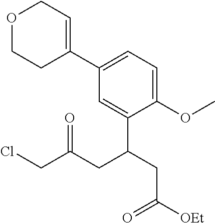 [SMILES: CCOC(=O)CC(CC(=O)CCl)c1cc(C2=CCOCC2)ccc1OC]

A solution of ethyl 6-diazo-3-(2-methoxy-5-(4-methoxytetrahydro-2H-pyran-4-yl)phenyl)-5-oxo-6-(trimethylsilyl)hexanoate (1 g, 2.1 mmol, 1 eq) was treated with HCl/EtOAc (4 M, 25 mL, 50.8 eq). The resulting mixture was stirred at 15° C. for 12 hr. The reaction mixture was concentrated under reduced pressure to give a residue. The residue was purified by flash chromatography (ISCO®; 12 g SepaFlash® Silica Flash Column, Eluent of 0-20% Ethyl acetate/Petroleum ether gradient @ 30 mL/min). The title compound (0.13 g, 341 μmol) was obtained as a yellow oil. LCMS (m/z 381.0 (M+H)).

### Step 2. Preparation of ethyl 3-[5-(3,6-dihydro-2H-pyran-4-yl)-2-methoxy-phenyl]-4-[2-[3-(5,6,7,8-tetrahydro-1,8-naphthyridin-2-yl)propyl]thiazol-4-yl]butanoate

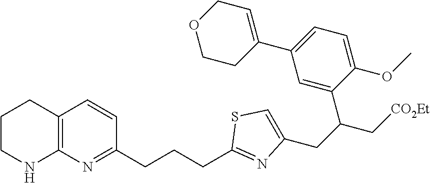 [SMILES: CCOC(=O)CC(Cc1csc(CCCc2ccc3c(n2)NCCC3)n1)c1cc(C2=CCOCC2)ccc1OC]

A solution of ethyl 6-chloro-3-[5-(3,6-dihydro-2H-pyran-4-yl)-2-methoxy-phenyl]-5-oxo-hexanoate (0.13 g, 341 μmol, 1.2 eq) in EtOH (10 mL) was treated with Compound A (67 mg, 284 μmol, 1 eq). The resulting mixture was stirred at 90° C. for 12 hr. The reaction mixture was concentrated under reduced pressure to give a residue. The residue was purified by flash chromatography (ISCO®; 4 g SepaFlash® Silica Flash Column, Eluent of 0-2% Methanol/Dichloromethane gradient @ 25 mL/min). The title compound (0.09 g, 160 μmol, 56% yield) was obtained as a brown solid. LCMS (m/z 562.1 (M+H)).

### Step 3. Preparation of 3-[5-(3,6-dihydro-2H-pyran-4-yl)-2-methoxy-phenyl]-4-[2-[3-(5,6,7,8-tetrahydro-1,8-naphthyridin-2-yl)propyl]thiazol-4-yl]butanoic acid

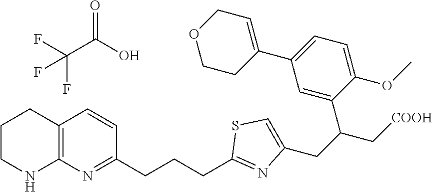

A solution of ethyl 3-[5-(3,6-dihydro-2H-pyran-4-yl)-2-methoxy-phenyl]-4-[2-[3-(5,6,7,8-tetrahydro-1,8-naphthyridin-2-yl)propyl]thiazol-4-yl]butanoate (0.09 g, 160 μmol, 1 eq) in THF (2 mL) was treated with NaOH (1 M, 2 mL, 12.5 eq). The resulting mixture was stirred at 60° C. for 16 hr. The reaction mixture was quenched by HO Ac to pH=5 at 10° C. and then the mixture was concentrated under reduced pressure to give a residue. The residue was purified by prep-HPLC (Boston Green ODS 150*30 5μ; mobile phase: [water (0.1% TFA)-ACN]; B %: 25%-55%, 8 min). The title compound (34 mg, 52 μmol, 32% yield, TFA) was obtained as a white solid. LCMS (m/z 534.2 (M+H)). (M+H); Clacd for: C₃₀H₃₅O₄N₃S: 533.23. ¹H NMR (CD₃OD, 400 MHz) δ=7.55 (br d, J=6.0 Hz, 1H), 7.16-7.22 (m, 2H), 6.99 (br s, 1H), 6.86 (d, J=8.4 Hz, 1H), 6.55-6.60 (m, 1H), 5.96-6.01 (m, 1H), 4.22 (br s, 2H), 3.82-3.95 (m, 3H), 3.80 (s, 3H), 3.49 (br d, J=4.4 Hz, 2H), 3.18 (d, J=7.6 Hz, 2H), 3.02-3.11 (m, 2H), 2.81 (br s, 2H), 2.64-2.77 (m, 4H), 2.39 (br s, 2H), 2.08 (quin, J=7.6 Hz, 2H), 1.95 (br s, 2H). ¹⁹F NMR (376 MHz, CD₃OD) δ=−77.29 (br d, 7=33.0 Hz, 3F).

### Example X

### Preparation of ethyl 6-diazo-3-(2-methoxy-5-(4-methoxytetrahydro-2H-pyran-4-yl)phenyl)-5-oxo-6-(trimethylsilyl)hexanoate

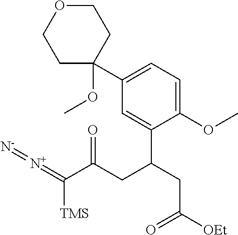 [SMILES: CCOC(=O)CC(CC(=O)C(=[N+]=[N-])[Si](C)(C)C)c1cc(C2(OC)CCOCC2)ccc1OC]

Example X was prepared in analogous manner to Example 26 (corresponding to trimethylsilyl-hexane-1-diazonium compound in the reaction Scheme 27), using 4-[2-methoxy-5-(4-methoxytetrahydropyran-4-yl)phenyl]tetrahydropyran-2,6-dione as the required anhydride in the reaction Scheme 27 where the anhydride is prepared in analogous manner to Example 1 using 2-methoxy-5-(4-methoxytetrahydropyran-4-yl)benzaldehyde (prepared according to Example Y) in the reaction Scheme 4. The crude product (1 g, 2.1 mmol, 84% yield) was obtained as brown oil.

### Example Y

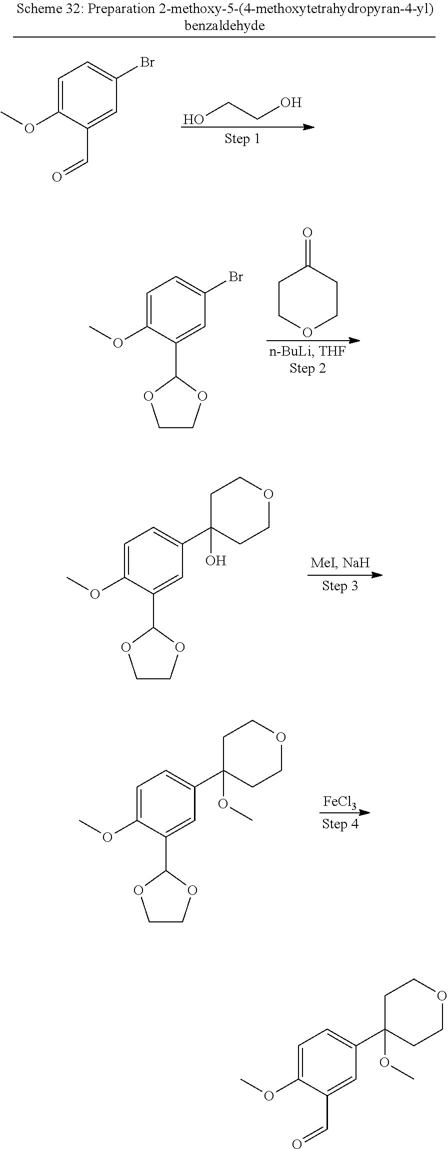

### Step 1. Preparation of 2-(5-bromo-2-methoxy-phenyl)-1,3-dioxolane

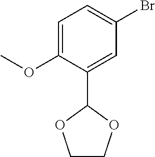

A solution of 5-bromo-2-methoxy-benzaldehyde (10 g, 46.5 mmol, 1 eq) in toluene (200 mL) was treated with p-TsOH (801 mg, 4.65 mmol, 0.1 eq) and ethylene glycol (5.77 g, 93 mmol, 5.2 mL, 2 eq), the mixture was stirred at 145° C. for 3 hr. The reaction mixture was cooled to room temperature and quenched by addition of saturated NaHCO₃ (400 mL), and extracted with toluene (100 mL*2). The combined organic layers were dried over Na₂SO₄, filtered and concentrated under reduced pressure to give a residue. The title compound (9 g, 34.7 mmol, 75% yield) was obtained as a yellow oil, and used in the next step without further purification.

### Step 2. Preparation of 4-[3-(1,3-dioxolan-2-yl)-4-methoxy-phenyl]tetrahydropyran-4-ol

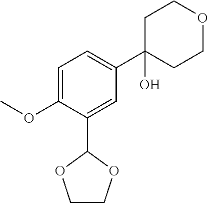 [SMILES: COc1ccc(C2(O)CCOCC2)cc1C1OCCO1]

A solution of 2-(5-bromo-2-methoxy-phenyl)-1,3-dioxolane (9 g, 34.74 mmol, 1 eq) in THF (50 mL) was treated with n-BuLi (2.5 M, 34.20 mL, 2.46 eq) dropwise at −78° C., the mixture was stirred at −78° C. for 0.5 hr. Subsequently, tetrahydropyran-4-one (3.83 g, 38.2 mmol, 3.5 mL, 1.1 eq) was added to the mixture at −78° C. and stirred at this temperature for 1 hr, then the mixture was stirred at 15° C. for 2 hr. The reaction mixture was quenched by addition water (100 mL) at 15° C., and concentrated under reduced pressure to remove THF. The remainder was diluted with ethyl acetate (200 mL) and extracted with water (100 mL*2). The combined organic layers were dried over Na₂SO₄, filtered and concentrated under reduced pressure to give a residue. The residue was purified by column chromatography (SiO₂, Petroleum ether/Ethyl acetate=20/l to 1:2). Compound 4-[3-(1,3-dioxolan-2-yl)-4-methoxy-phenyl]tetrahydropyran-4-ol (5.1 g, 18.19 mmol, 52.38% yield) was obtained as a white solid. ¹H NMR (400 MHz, CDCl₃) δ=1.67 (br d, J=13.6 Hz, 2H), 2.14 (br t, J=12.4 Hz, 2H), 3.70-3.95 (m, 7H), 4.03 (br s, 2H), 4.13 (br s, 2H), 6.12 (s, 1H), 6.89 (br d, J=8.8 Hz, 1H), 7.44 (br d, J=8.8 Hz, 1H), 7.67 (br s, 1H).

### Step 3. Preparation of 4-[3-(1,3-dioxolan-2-yl)-4-methoxy-phenyl]-4-methoxy-tetrahydropyran

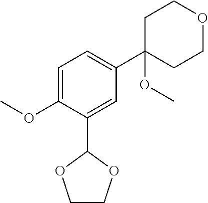 [SMILES: COc1ccc(C2(OC)CCOCC2)cc1C1OCCO1]

A mixture of NaH (3.25 g, 81 mmol, 60%, 3 eq) in THF (90 mL) was treated with 4-[3-(1,3-dioxolan-2-yl)-4-methoxyphenyl]tetrahydropyran-4-ol (7.6 g, 27 mmol, 1 eq) in THF (10 mL) under N₂ at 0° C. and stirred for 0.5 hr, then CH₃I (23.1 g, 162.7 mmol, 10.1 mL, 6 eq) was added and the mixture was stirred at 15° C. for 2.5 hr. The reaction mixture was quenched by addition of water (60 mL), and then extracted with ethyl acetate (100 mL*3). The combined organic layers were washed with brine, dried over anhydrous Na₂SO₄, filtered and concentrated under reduced pressure to give a residue. The residue was purified by flash chromatography (ISCO®; 80 g SepaFlash® Silica Flash Column, Eluent of 0-50% Ethyl acetate/Petroleum ether gradient @ 60 mL/min). The title compound (6.8 g, 23.1 mmol, 85% yield) was obtained as a white solid. ¹H NMR (400 MHz, CDCl₃) δ=7.56 (d, J=2.8 Hz, 1H), 7.36 (dd, J=2.4, 8.8 Hz, 1H), 6.92 (d, J=8.8 Hz, 1H), 6.14 (s, 1H), 4.18-4.12 (m, 2H), 4.08-4.01 (m, 2H), 3.90-3.76 (m, 7H), 2.95 (s, 3H), 2.05-1.96 (m, 4H).

### Step 4. Preparation of 2-methoxy-5-(4-methoxytetrahydropyran-4-yl)benzaldehyde

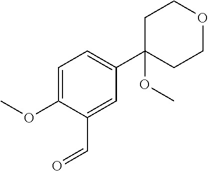 [SMILES: COc1ccc(C2(OC)CCOCC2)cc1C=O]

A solution of 4-[3-(1,3-dioxolan-2-yl)-4-methoxy-phenyl]-4-methoxy-tetrahydropyran (6.8 g, 23.1 mmol, 1 eq) in acetone (150 mL) was treated with FeCl₃-6H₂O (1.25 g, 4.62 mmol, 0.2 eq) under N₂ and stirred at 15° C. for 5 min. The reaction mixture was concentrated under reduced pressure to give a residue. The residue was purified by flash chromatography (ISCO®; 40 g SepaFlash® Silica Flash Column, Eluent of 0-40% Ethyl acetate/Petroleum ether gradient @ 40 mL/min). The title compound (3.8 g, 15.2 mmol, 65.7% yield) was obtained as a yellow oil. ¹H NMR (400 MHz, CDCl₃) δ=10.46 (s, 1H), 7.80 (d, J=2.4 Hz, 1H), 7.62 (dd, J=2.8, 8.8 Hz, 1H), 7.01 (d, J=8.8 Hz, 1H), 3.95-3.92 (m, 3H), 3.85-3.76 (m, 4H), 2.95-2.89 (m, 3H), 2.02-1.93 (m, 4H).

### Example 37A and Example 37B

### Step 1. Preparation of (S)-3-(5-(3,6-dihydro-2H-pyran-4-yl)-2-methoxyphenyl)-4-(2-(3-(5,6,7,8-tetrahydro-1,8-naphthyridin-2-yl)propyl)thiazol-4-yl)butanoic acid & (R)-3-(5-(3,6-dihydro-2H-pyran-4-yl)-2-methoxyphenyl)-4-(2-(3-(5,6,7,8-tetrahydro-1,8-naphthyridin-2-yl)propyl)thiazol-4-yl)butanoic acid

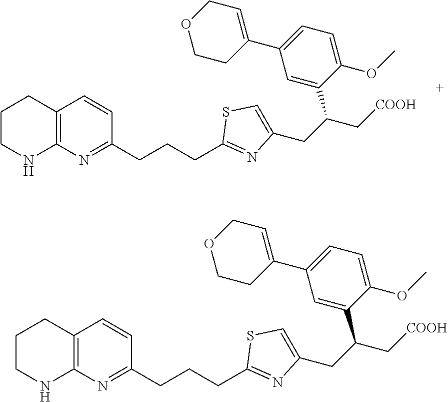 [SMILES: COc1ccc(C2=CCOCC2)cc1[C@H](CC(=O)O)Cc1csc(CCCc2ccc3c(n2)NCCC3)n1 ; COc1ccc(C2=CCOCC2)cc1[C@@H](CC(=O)O)Cc1csc(CCCc2ccc3c(n2)NCCC3)n1]

3-[5-(3,6-dihydro-2H-pyran-4-yl)-2-methoxy-phenyl]-4-[2-[3-(5,6,7,8-tetrahydro-1,8-naphthyridin-2-yl)propyl]thiazol-4-yl]butanoic acid (2.5 g, 4.68 mmol, 1 eq) was separated by SFC (column: DAICEL CHIRALPAK AD (250 mm*50 mm, 10 μm); mobile phase: [0.1% NH₃H₂O CH₃OH]; B %: 40%-40%, 17.3 (S isomer) and 23.2 min (R isomer)). (3S)-3-[5-(3,6-dihydro-2H-pyran-4-yl)-2-methoxy-phenyl]-4-[2-[3-(5,6,7,8-tetrahydro-1,8-naphthyridin-2-yl)propyl]thiazol-4-yl]butanoic acid (1.1 g, 2.06 mmol, 44% yield) and (3R)-3-[5-(3,6-dihydro-2H-pyran-4-yl)-2-methoxyphenyl]-4-[2-[3-(5,6,7,8-tetrahydro-1,8-naphthyridin-2-yl)propyl]thiazol-4-yl]butanoic acid (1.2 g, 2.25 mmol, 48% yield) was obtained as off-white solid.

### Example 37A

### Preparation of (S)-3-(5-(3,6-dihydro-2H-pyran-4-yl)-2-methoxyphenyl)-4-(2-(3-(5,6,7,8-tetrahydro-1,8-naphthyridin-2-yl)propyl)thiazol-4-yl)butanoic acid trifluoroacetate

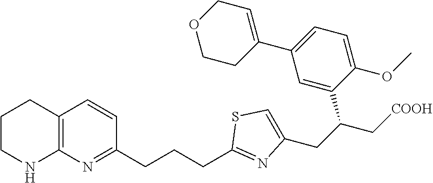 [SMILES: COc1ccc(C2=CCOCC2)cc1[C@H](CC(=O)O)Cc1csc(CCCc2ccc3c(n2)NCCC3)n1]

(3S)-3-[5-(3,6-dihydro-2H-pyran-4-yl)-2-methoxy-phenyl]-4-[2-[3-(5,6,7,8-tetrahydro-1,8-naphthyridin-2-yl)propyl]thiazol-4-yl]butanoic acid (1.1 g, 2.06 mmol, 1 eq) was purified by preparative HPLC (TFA condition:column: YMC-Triart Prep C18 150*40 mm*7 μm; mobile phase: [water (0.1% TFA)-ACN]; B %: 25%-55%, 10 min). The title compound (1.08 g, 1.67 mmol, 81% yield, TFA) was obtained as a white solid. LC-MS mass: m/z 534.3 (M+H)⁺. ¹H NMR (400 MHz, CD₃OD) δ=7.58 (d, J=7.2 Hz, 1H), 7.24-7.17 (m, 2H), 6.97 (s, 1H), 6.88 (d, J=8.4 Hz, 1H), 6.60 (d, J=7.6 Hz, 1H), 6.00 (br s, 1H), 4.24 (q, J=2.4 Hz, 2H), 3.97-3.85 (m, 3H), 3.82 (s, 3H), 3.54-3.49 (m, 2H), 3.19 (d, J=7.6 Hz, 2H), 3.08 (t, J=7.34 Hz, 2H), 2.83 (t, J=6.0 Hz, 2H), 2.79-2.66 (m, 4H), 2.41 (br d, J=2.0 Hz, 2H), 2.14-2.04 (m, 2H), 1.97 (quin, J=6.0 Hz, 2H).

### Example 37B

### Preparation of (R)-3-(5-(3,6-dihydro-2H-pyran-4-yl)-2-methoxyphenyl)-4-(2-(3-(5,6,7,8-tetrahydro-1,8-naphthyridin-2-yl)propyl)thiazol-4-yl)butanoic acid trifluoroacetate

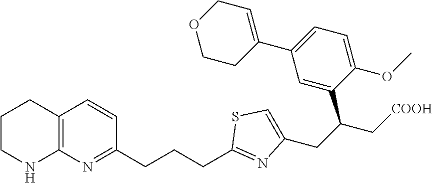 [SMILES: COc1ccc(C2=CCOCC2)cc1[C@@H](CC(=O)O)Cc1csc(CCCc2ccc3c(n2)NCCC3)n1]

(3R)-3-[5-(3,6-dihydro-2H-pyran-4-yl)-2-methoxy-phenyl]-4-[2-[3-(5,6,7,8-tetrahydro-1,8-naphthyridin-2-yl)propyl]thiazol-4-yl]butanoic acid (1.20 g, 2.25 mmol, 1 eq) was purified by preparative HPLC (TFA condition:column: YMC-Triart Prep C18 150*40 mm*7 μm; mobile phase: [water (0.1% TFA)-ACN]; B %: 25%-55%, 10 min). The title compound (1.25 g, 1.93 mmol, 86% yield, TFA) was obtained as a white solid. LC-MS mass: m/z 534.3 (M+H)⁺. ¹H NMR (400 MHz, CD₃OD) δ=7.58 (d, J=7.2 Hz, 1H), 7.24-7.16 (m, 2H), 6.95 (s, 1H), 6.88 (d, J=8.4 Hz, 1H), 6.60 (d, J=7.2 Hz, 1H), 6.00 (br s, 1H), 4.24 (br d, J=2.4 Hz, 2H), 3.96-3.85 (m, 3H), 3.82 (s, 3H), 3.52 (t, J=5.6 Hz, 2H), 3.19 (d, J=7.6 Hz, 2H), 3.07 (t, J=7.6 Hz, 2H), 2.84 (br t, J=6.0 Hz, 2H), 2.77-2.65 (m, 4H), 2.41 (br s, 2H), 2.09 (quin, J=7.6 Hz, 2H), 1.97 (quin, J=6.0 Hz, 2H).

### Example 38

### Preparation of 3-(3-(3,5-dimethyl-1H-pyrazol-1-yl)phenyl)-4-(2-(2-(5,6,7,8-tetrahydro-1,8-naphthyridin-2-yl)ethyl)thiazol-4-yl)butanoic acid

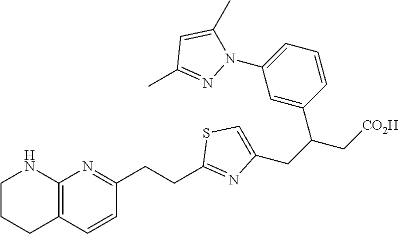 [SMILES: Cc1cc(C)n(-c2cccc(C(CC(=O)O)Cc3csc(CCc4ccc5c(n4)NCCC5)n3)c2)n1]

Example 38 was prepared in analogous manner to Example 26, using 4-(3-(3,5-dimethyl-1H-pyrazol-1-yl)phenyl)dihydro-2H-pyran-2,6(3H)-dione as the required anhydride in the reaction Scheme 27 where the anhydride is prepared in analogous manner to Example 1 using 3-(3,5-dimethyl-1H-pyrazol-1-yl)benzaldehyde (obtained according to Example Z) as the required benzaldehyde in the reaction Scheme. The crude product was purified by prep-HPLC (Boston Green ODS 150*30 5μ; mobile phase: [water (0.1% TFA)-ACN]; B %: 40, 6 min) affording the title compound (17 mg, 27 μmol, 38% yield, TFA) as brown oil. LCMS (m/z 502.2 (M+H)). ¹H NMR (400 MHz, CD₃OD) δ=7.48 (d, J=7.2 Hz, 1H), 7.39-7.45 (m, 1H), 7.27 (dd, J=12.4, 8.0 Hz, 2H), 7.21 (s, 1H), 6.91 (s, 1H), 6.52 (d, J=7.2 Hz, 1H), 6.14 (s, 1H), 3.62 (quin, J=7.6 Hz, 1H), 3.49 (t, J=5.6 Hz, 2H), 3.35-3.42 (m, 2H), 3.00-3.20 (m, 4H), 2.78 (br t, j=6.0 Hz, 2H), 2.69 (d, J=7.6 Hz, 2H), 2.27 (s, 3H), 2.19 (s, 3H), 1.92 (quin, J=6.0 Hz, 2H).

### Example Z

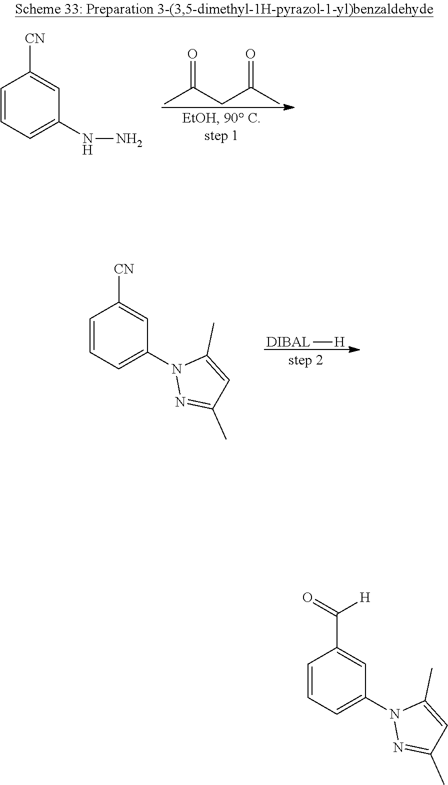

### Step 1. Preparation of 3-(3,5-dimethyl-1H-pyrazol-1-yl)benzonitrile

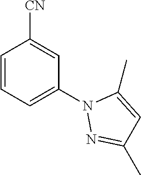

A suspension of 3-hydrazinobenzonitrile (25.4 g, 150 mmol, 1 eq, HCl) in EtOH (300 mL) was treated with pentane-2,4-dione (15 g, 150 mmol, 15.4 mL, 1 eq). The resulting mixture was refluxed at 90° C. for 16 hr. The reaction mixture was concentrated under reduced pressure to remove solvent. The crude product was used into the next step without further purification. The title compound (28.9 g, crude) was obtained as brown solid. ¹H NMR (CDCl₃, 400 MHz) δ=7.74-7.88 (m, 3H), 7.65-7.72 (m, 1H), 6.25 (s, 1H), 2.48 (s, 3H), 2.36 (s, 3H).

### Step 2. Preparation of 3-(3,5-dimethyl-1H-pyrazol-1-yl)benzaldehyde

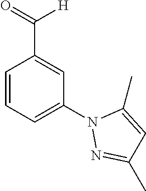

A solution of 3-(3,5-dimethylpyrazol-1-yl)benzonitrile (10 g, 50.7 mmol, 1 eq) in toluene (100 mL) was treated with DIBAL-H (1 M, 101.4 mL, 2 eq) slowly under N₂ at 0° C. The reaction mixture was stirred at 0° C. for 30 min. The reaction mixture was cooled to 25° C. and quenched with water (120 mL) slowly, then extracted with Ethyl acetate (80 mL*3). The combined organic phase was washed with brine (150 mL), dried over anhydrous Na₂SO₄, filtered and concentrated in vacuo. The residue was purified by flash chromatography (ISCO®; 80 g SepaLlash® Silica Llash Column, Eluent of 0-40% Ethyl acetate/Petroleum ethergradient @ 60 mL/min). The title compound (5.7 g, 28.5 mmol, 56% yield) was obtained as yellow oil. ¹H NMR (CDCl₃, 400 MHz) δ=10.04 (s, 1H), 7.95 (t, 7=2.0 Hz, 1H), 7.83 (dd, 7=7.6, 1.2 Hz, 1H), 7.70-7.77 (m, 1H), 7.57-7.65 (m, 1H), 6.02 (s, 1H), 2.34 (s, 3H), 2.29 (s, 3H).

### Example 39

### Preparation of 3-(5-(tert-butyl)-2-methoxyphenyl)-4-(3-(2-(5,6,7,8-tetrahydro-1,8-naphthyridin-2-yl)ethoxy)-1-(2,2,2-trifluoroethyl)-1H-pyrazol-5-yl)butanoic acid trifluoroacetate

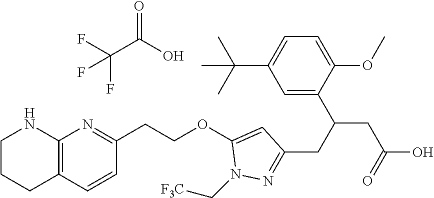

Example 39 was prepared in analogous manner to Example 1 using 5-(tert-butyl)-2-methoxybenzaldehyde as the required benzaldehyde and 2,2,2-trifluoroethylhydrazine as the required hydrazine in the reaction Scheme 4. The crude product was purified by prep-HPLC (column: Agela DuraShell ODS 150*25 5μ; mobile phase: [water (0.1% TFA)-ACN]; B %: 30%-60%, 8.5 min) affording the title compound (270 mg, 470 μmol, 57% yield, TFA) as a white solid. LCMS (m/z 601.3 (M+H)). ¹H NMR (CD₃OD, 400 MHZ) δ=7.60 (d, J=7.5 Hz, 1H), 7.18 (dd, J=2.3, 8.5 Hz, 1H), 7.11 (d, J=2.3 Hz, 1H), 6.84 (d, J=8.5 Hz, 1H), 6.68 (d, J=7.3 Hz, 1H), 5.39 (s, 1H), 4.52 (q, j=8.7 Hz, 2H), 4.31 (t, j=5.9 Hz, 2H), 3.82 (s, 3H), 3.78-3.68 (m, 1H), 3.55-3.48 (m, 2H), 3.17 (t, J=5.9 Hz, 2H), 2.95-2.80 (m, 4H), 2.78-2.61 (m, 2H), 1.96 (quin, J=6.0 Hz, 2H), 1.24 (s, 9H).

### Example 40

### Preparation of 3-(3,5-di-tert-butylphenyl)-4-(5-(2-(5,6,7,8-tetrahydro-1,8-naphthyridin-2-yl)ethoxy)-1-(2,2,2-trifluoroethyl)-1H-pyrazol-3-yl)butanoic acid trifluoroacetate

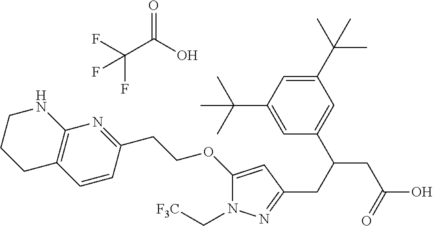

Example 40 was prepared in analogous manner to Example 1 using 3,5-ditert-butylbenzaldehyde as the required benzaldehyde and 2,2,2-trifluoroethylhydrazine as the required hydrazine in the reaction Scheme 4. The crude product was purified by prep-HPLC (column: Agela DuraShell ODS 150*25 5μ; mobile phase: [water (0.1% TFA)-ACN]; B %: 58%, 8.5 min) affording the title compound (14 mg, 19 μmol, 2% yield, 97% purity, TFA) as a yellow solid. ¹H NMR (CD₃OD, 400 MHz) δ=7.64-7.54 (m, 1H), 7.28-7.20 (m, 1H), 7.03 (d, J=1.6 Hz, 2H), 6.74-6.63 (m, 1H), 5.44 (s, 1H), 4.57-4.44 (m, 2H), 4.37-4.25 (m, 2H), 3.55-3.47 (m, 2H), 3.45-3.35 (m, 1H), 3.21-3.11 (m, 2H), 2.90-2.56 (m, 6H), 2.03-1.89 (m, 2H), 1.27 (s, 18H). ¹⁹F NMR (376 MHz, CD₃OD) δ=−72.68 (t, J=8.8 Hz, 3F), −77.22 (br s, 3F).

### Example 41

### Preparation of 3-(3,5-bis(2-cyanopropan-2-yl)phenyl)-4-(1-methyl-5-(2-(5,6,7,8-tetrahydro-1,8-naphthyridin-2-yl)ethoxy)-1H-pyrazol-3-yl)butanoic acid trifluoroacetate

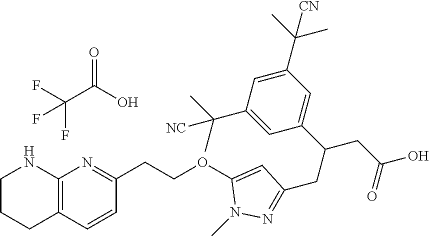

Example 41 was prepared in analogous manner to Example 1 using 3-(3,5-bis(2-cyanopropan-2-yl)phenyl)pentanedioic acid (obtained according to Example A A) as the required diacid and LiOH instead of NaOH in the final step in the reaction Scheme 4. The crude product was purified by prep-HPLC (column: Boston Green ODS 150*30 5μ; mobile phase: [water (0.075% TFA)-ACN]; B %: 30%-50%, 11 min) affording the title compound (3 mg, 4.5 μmol, 5% yield, TFA) as a white solid. LC-MS mass: m/z 555.3 (M+H). ¹H NMR (CD₃OD, 400 MHz) δ=7.64-7.57 (m, 1H), 7.43 (s, 1H), 7.28 (d, J=1.6 Hz, 2H), 6.72-6.66 (m, 1H), 5.46-5.38 (m, 1H), 4.35-4.23 (m, 2H), 3.54-3.47 (m, 3H), 3.43 (s, 3H), 3.14 (s, 2H), 2.92-2.64 (m, 6H), 2.00-1.91 (m, 2H), 1.69 (d, J=3.2 Hz, 12H). ¹⁹F NMR (376 MHz, CD₃OD) δ=−77.29 (s, 3F).

### Example AA

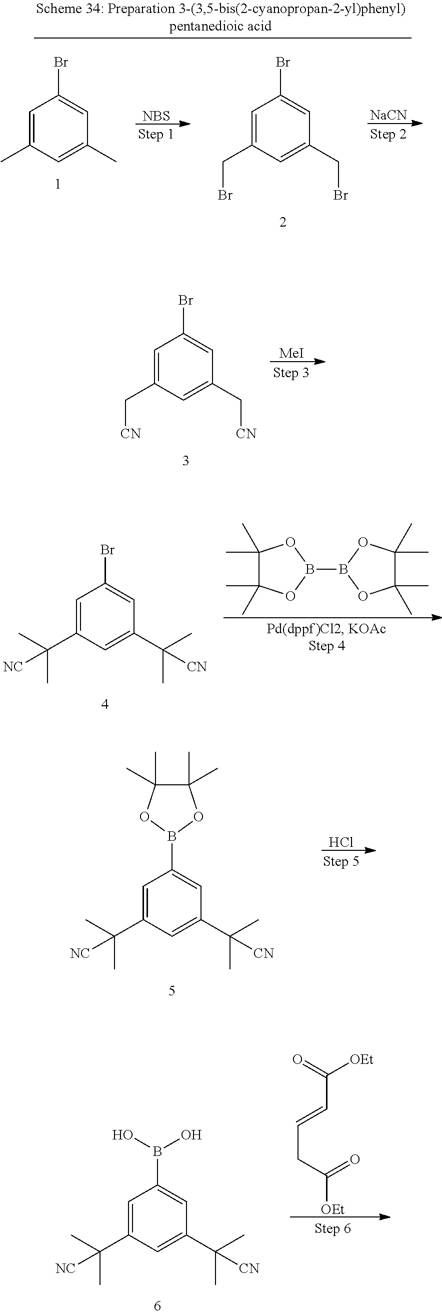

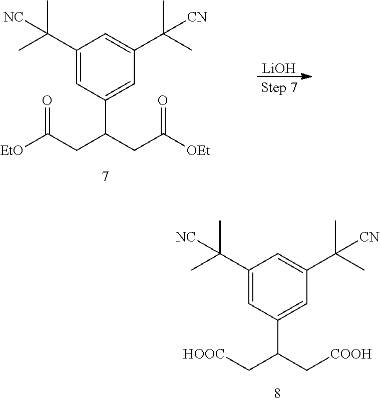 [SMILES: CCOC(=O)CC(CC(=O)OCC)c1cc(C(C)(C)C#N)cc(C(C)(C)C#N)c1 ; "LiOH" ; CC(C)(C#N)c1cc(C(CC(=O)O)CC(=O)O)cc(C(C)(C)C#N)c1]

### Step 1. Preparation of 1-bromo-3,5-bis(bromomethyl)benzene

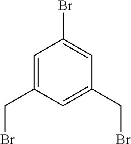 [SMILES: BrCc1cc(Br)cc(CBr)c1]

A mixture of 1-bromo-3,5-dimethyl-benzene (10 g, 54 mmol, 7.4 mL, 1 eq) and NBS (20.2 g, 113 mmol, 2.1 eq) in CH₃CN (200 mL) was treated with AIBN (887 mg, 5.4 mmol, 0.1 eq), and the resulting mixture was stirred at 90° C. for 3 hr under N₂ atmosphere. The mixture was combined with 10% Na₂SO₃ (100 mL) and concentrated under reduced pressure to remove solvent. The residue was diluted with ethyl acetate (200 mL) and washed with H₂O (150 mL*3). The organic layer was dried over Na₂SO₄, filtered and concentrated under reduced pressure to give a residue. The residue was purified by crystallization from EtOH. The title compound (7.2 g, 21 mmol, 39% yield) was obtained as a white solid. NMR (CDCl₃, 400 MHz) δ ppm=7.48 (d, J=1.6 Hz, 2H), 7.35 (s, 1H), 4.41 (s, 4H).

### Step 2. Preparation of 2,2′-(5-bromo-1,3-phenylene)bis(2-methylpropanenitrile)

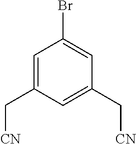 [SMILES: N#CCc1cc(Br)cc(CC#N)c1]

A solution of 1-bromo-3,5-bis(bromomethyl)benzene (7.2 g, 21.00 mmol, 1 eq) in CH₃CN (100 mL) was treated with KCN (3.14 g, 48 mmol, 2.1 mL, 2.3 eq), 18-Crown-6 (1.11 g, 4.2 mmol, 0.2 eq), KI (349 mg, 2.1 mmol, 0.1 eq), and H₂O (8.5 mL). The resulting mixture was stirred at 20° C. for 24 hr. The mixture was concentrated under reduced pressure to give a residue. The residue was combined with EtOAc (100 mL). The organic mixture was washed with water (150 mL*3) and brine (100 mL), dried over Na₂SO₄, filtered, and concentrated in vacuo to afford a residue. The residue was purified by flash silica gel chromatography (ISCO®; 40 g SepaFlash® Silica Flash Column, Eluent of 0˜60% Ethyl acetate/Petroleum ether gradient @ 40 mL/min). The title compound (1.75 g, 7.44 mmol, 35% yield) was obtained as a white solid. ¹H NMR (CDCl₃, 400 MHZ) δ ppm=7.50 (s, 2H), 7.27 (s, 1H), 3.77 (s, 4H).

### Step 3. Preparation of 2,2′-(5-bromo-1,3-phenylene)bis(2-methylpropanenitrile)

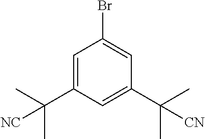 [SMILES: CC(C)(C#N)c1cc(Br)cc(C(C)(C)C#N)c1]

A mixture of 2-[3-bromo-5-(cyanomethyl)phenyl]acetonitrile (2.5 g, 10.63 mmol, 1 eq) in THF (25 mL) was treated with NaH (2.98 g, 74 mmol, 60% purity, 7 eq) at 0° C. under N₂. The resulting mixture was stirred at 0° C. for 30 min, then Mel (13.2 g, 93 mmol, 5.8 mL, 8.74 eq) was added dropwise at 0° C. The resulting mixture was stirred at 16° C. for 16 hr and then combined with water (100 mL). The aqueous mixture was extracted with EtOAc (150 mL*2). The combined organic component was washed with brine (150 mL), dried over Na₂SO₄ and concentrated under vacuum to give a residue. The title compound (3 g, crude) was obtained as a yellow solid. ¹H NMR (400 MHz, CDCl₃) δ=7.58 (d, J=1.6 Hz, 2H), 7.51 (t, J=1.6 Hz, 1H), 1.75 (s, 12H).

### Step 4. Preparation of 2,2′-(5-(4,4,5,5-tetramethyl-1,3,2-dioxaborolan-2-yl)-1,3-phenylene)bis(2-methylpropanenitrile)

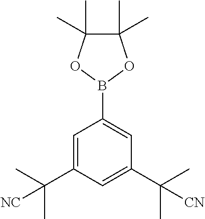 [SMILES: CC(C)(C#N)c1cc(B2OC(C)(C)C(C)(C)O2)cc(C(C)(C)C#N)c1]

A mixture of 2-[3-bromo-5-(1-cyano-1-methyl-ethyl)phenyl]-2-methyl-propanenitrile (4 g, 13.7 mmol, 1 eq), bis(pinacolato)diboron (5.23 g, 20.6 mmol, 1.5 eq), Pd(dppf)Cl₂ (302 mg, 412 μmol, 0.03 eq) and KOAc (4.04 g, 41.2 mmol, 3 eq) in DMSO (100 mL) was de-gassed and then heated to 90° C. for 16 hours under N₂. The mixture was poured into water (250 mL). The mixture was extracted with ethyl acetate (150 mL*2). The combined organic phase was washed with brine (150 mL), dried over anhydrous Na₂SO₄, concentrated under vacuum to give a residue. The residue was purified by flash silica gel chromatography (ISCO®; 40 g SepaFlash® Silica Flash Column, eluent of 0˜6% Ethyl acetate/Petroleum ethergradient @ 40 mL/min). The title compound (2.29 g, 6.8 mmol, 49% yield) was obtained as a white solid. ¹H NMR (400 MHz, CDCl₃) δ=7.83 (d, J=1.6 Hz, 2H), 7.69 (d, J=2.0 Hz, 1H), 1.77 (s, 12H), 1.36 (s, 12H).

### Step 5. Preparation of (3,5-bis(2-cyanopropan-2-yl)phenyl)boronic acid

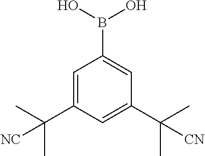 [SMILES: CC(C)(C#N)c1cc(B(O)O)cc(C(C)(C)C#N)c1]

A mixture of 2-[3-(1-cyano-1-methyl-ethyl)-5-(4,4,5,5-tetramethyl-1,3,2-dioxaborolan-2-yl)phenyl]-2-methyl-propanenitrile (2.7 g, 8 mmol, 1 eq), and ammonium acetate (1.85 g, 24 mmol, 3 eq) in a mixture of acetone (50 mL) and water (50 mL) was treated with sodium periodate (5.12 g, 24 mmol, 1.3 mL, 3 eq). The resulting mixture was stirred at 18° C. for 16 h. The mixture was concentrated under reduced pressure to remove acetone and afford a residue. The residue was diluted with water (150 mL) and extracted with EtOAc (100 mL*3). The combined organic layers were washed with brine (150 mL), dried over Na₂SO₄ and concentrated under vacuum to give a residue. The title compound was obtained as a light yellow solid. ¹H NMR (400 MHz, DMSO-d₆) δ=7.88 (d, J=2.0 Hz, 2H), 7.60 (s, 1H), 1.68 (s, 12H).

### Step 6. Preparation of diethyl 3-(3,5-bis(2-cyanopropan-2-yl)phenyl)pentanedioate

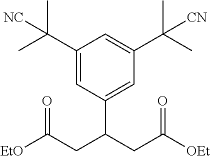 [SMILES: CCOC(=O)CC(CC(=O)OCC)c1cc(C(C)(C)C#N)cc(C(C)(C)C#N)c1]

A solution of [3,5-bis(1-cyano-1-methyl-ethyl)phenyl]boronic acid (1.4 g, 5.5 mmol, 1.5 eq) and chloro(1,5-cyclooctadiene)rhodium(I) dimer (54 mg, 109 μmol, 0.03 eq) in mixture of dioxane (5 mL) and water (1 mL) was treated with TEA (369 mg, 3.6 mmol, 507 μL, 1 eq) and (E)-diethyl pent-2-enedioate (679 mg, 3.6 mmol, 1 eq). The resulting mixture was stirred at 50° C. for 16 h. The mixture was concentrated under reduced pressure to remove dioxane affording a residue. The residue was diluted with water (100 mL) and extracted with EtOAc (100 mL*3). The combined organic layers were washed with brine (100 mL), dried over Na₂SO₄ and concentrated under vacuum to give a residue. The residue was purified by flash silica gel chromatography (ISCO®; 12 g SepaLlash® Silica Llash Column, Eluent of 0-20% Ethyl acetate/Petroleum ethergradient @ 25 mL/min). The title compound (380 mg, 591 μmol, 16% yield, 62% purity) was obtained as a yellow oil.

### Step 7. Preparation of 3-(3,5-bis(2-cyanopropan-2-yl)phenyl)pentanedioic acid

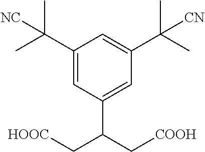 [SMILES: CC(C)(C#N)c1cc(C(CC(=O)O)CC(=O)O)cc(C(C)(C)C#N)c1]

A mixture of diethyl 3-[3,5-bis(1-cyano-1-methyl-ethyl)phenyl]pentanedioate (380 mg, 591 μmol, 1 eq) and LiOHH₂O (74 mg, 1.77 mmol, 3 eq) in EtOH (5 mL) and water (1 mL) was stirred at 18° C. for 16 h. The mixture was concentrated under reduced pressure to remove EtOH and then water (50 mL) was added. The aqueous mixture was extracted with ethyl acetate (50 mL*3) and the aqueous phase was acidified to pH=5 with 1M HCl and the layers partitioned. The aqueous phase was extracted with ethyl acetate (50 mL*3). The combined organic phase was washed with brine (50 mL), dried over Na₂SO₄, filtered and concentrated under vacuum. The title compound (200 mg, crude) was obtained as a yellow solid.

### Example 42

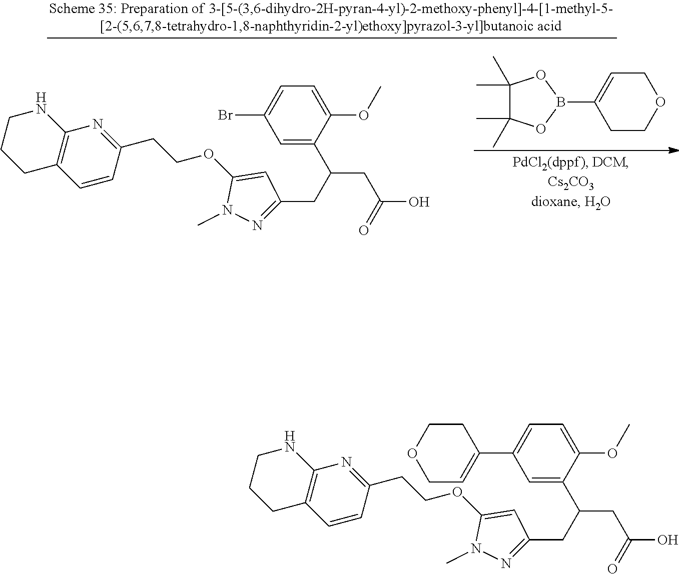 [SMILES: COc1ccc(Br)cc1C(CC(=O)O)Cc1cc(OCCc2ccc3c(n2)NCCC3)n(C)n1 ; CC1(C)OB(C2=CCOCC2)OC1(C)C ; COc1ccc(C2=CCOCC2)cc1C(CC(=O)O)Cc1cc(OCCc2ccc3c(n2)NCCC3)n(C)n1]

A mixture of 3-(5-bromo-2-methoxy-phenyl)-4-[1-methyl-5-[2-(5,6,7,8-tetrahydro-1,8-naphthyridin-2-yl)ethoxy]pyrazol-3-yl]butanoic acid (140 mg, 264 μmol, 1 eq), 2-(3,6-dihydro-2H-pyran-4-yl)-4,4,5,5-tetramethyl-1,3,2-dioxaborolane (111 mg, 529 μmol, 2 eq), Cs₂CO₃ (172 mg, 529 μmol, 2 eq) and Pd(dppf)Cl₂—CH₂Cl₂ (22 mg, 26 μmol, 0.1 eq) in a mixture of dioxane (5 mL) and water (1 mL) was stirred for 20 h at 100° C. under N₂. The mixture was filtered through celite and the filtrate was concentrated in vacuo to afford a residue. The residue was purified by prep-HPLC (TFA condition: column: Boston Green ODS 150*30 5μ; mobile phase: [water (0.075% TFA)-ACN]; B %: 26%-46%, 9 min). The title compound (40 mg, 75 μmol, 28% yield) was obtained as a white solid. ¹H NMR (400 MHz, CD₃OD) δ=7.61 (d, J=7.3 Hz, 1H), 7.27 (dd, J=2.3, 8.5 Hz, 1H), 7.22 (d, J=2.3 Hz, 1H), 6.93 (d, J=8.5 Hz, 1H), 6.66 (d, J=7.3 Hz, 1H), 6.08-6.01 (m, 1H), 5.52 (s, 1H), 4.36 (t, J=6.0 Hz, 2H), 4.27 (q, J=2.6 Hz, 2H), 3.90 (t, J=5.5 Hz, 2H), 3.86 (s, 3H), 3.82-3.73 (m, 1H), 3.54-3.44 (m, 5H), 3.16 (t, J=6.0 Hz, 2H), 2.99-2.87 (m, 2H), 2.84 (t, J=6.4 Hz, 2H), 2.78-2.63 (m, 2H), 2.46 (td, J=2.4, 4.5 Hz, 2H), 1.97 (td, J=6.0, 11.9 Hz, 2H).

### Example 43

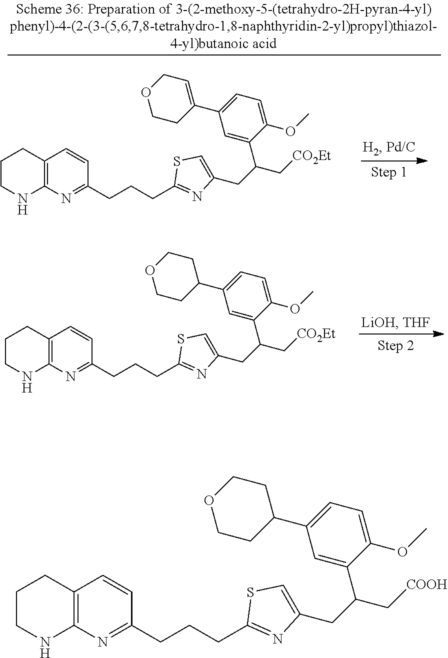 [SMILES: CCOC(=O)CC(Cc1csc(CCCc2ccc3c(n2)NCCC3)n1)c1cc(C2=CCOCC2)ccc1OC ; CCOC(=O)CC(Cc1csc(CCCc2ccc3c(n2)NCCC3)n1)c1cc(C2CCOCC2)ccc1OC ; COc1ccc(C2CCOCC2)cc1C(CC(=O)O)Cc1csc(CCCc2ccc3c(n2)NCCC3)n1]

### Step 1. Preparation of ethyl 3-(2-methoxy-5-(tetrahydro-2H-pyran-4-yl)phenyl)-4-(2-(3-(5,6,7,8-tetrahydro-1,8-naphthyridin-2-yl)propyl)thiazol-4-yl)butanoate

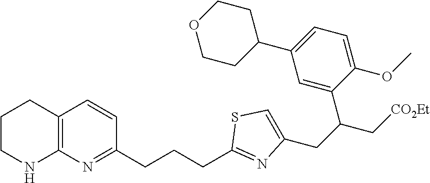 [SMILES: CCOC(=O)CC(Cc1csc(CCCc2ccc3c(n2)NCCC3)n1)c1cc(C2CCOCC2)ccc1OC]

A solution of ethyl 3-[5-(3,6-dihydro-2H-pyran-4-yl)-2-methoxy-phenyl]-4-[2-[3-(5,6,7,8-tetrahydro-1,8-naphthyridin-2-yl)propyl]thiazol-4-yl]butanoate (300 mg, 534 μmol, 1 eq) in EtOH (40 mL) treated with Pd/C (500 mg, 470 μmol, 10%, 0.88 eq) under N₂ atmosphere. The suspension was degassed and purged with H₂ for three times. The mixture was stirred under H₂ (45 psi) at 25° C. for 48 hr. The mixture was filtered through celite, and the filter cake was washed with EtOH (20 mL). The filtrate was concentrated in vacuo to afford the title compound (301 mg, crude) as light yellow oil, which was used into the next step without further purification.

### Step 2. Preparation of 3-(2-methoxy-5-(tetrahydro-2H-pyran-4-yl)phenyl)-4-(2-(3-(5,6,7,8-tetrahydro-1,8-naphthyridin-2-yl)propyl)thiazol-4-yl)butanoic acid

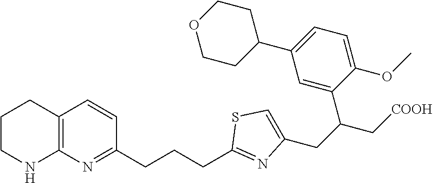 [SMILES: COc1ccc(C2CCOCC2)cc1C(CC(=O)O)Cc1csc(CCCc2ccc3c(n2)NCCC3)n1]

A solution of the compound from step 1 (301 mg, 534 μmol, 1 eq) in a mixture of EtOH (15 mL) and H₂O (2 mL) was treated with LiOHH₂O (112 mg, 2.67 mmol, 5 eq). The resulting mixture was stirred at 25° C. for 24 hr. The solution was concentrated in vacuo to afford a residue which was taken up with water (2 mL) and acidified with 2 N HCl to pH=6. The solvent was removed under vacuum to afford a residue. The residue was purified by prep-HPLC (TFA condition: column: Boston Green ODS 150*30 5μ; mobile phase: [water (0.075% TFA)-ACN]; B %: 34%-54%, 9 min). The title compound (70 mg, 108 μmol, 20% yield, TFA) was obtained as a white solid. LC-MS mass: m/z 536.4 (M+H). ¹H NMR (400 MHz, CD₃OD) δ=7.60 (d, J=7.2 Hz, 1H), 7.03 (br d, J=8.4 Hz, 1H), 6.97 (s, 1H), 6.90-6.82 (m, 2H), 6.64 (d, J=7.5 Hz, 1H), 4.00 (br d, J=11.2 Hz, 2H), 3.94-3.85 (m, 1H), 3.79 (s, 3H), 3.56-3.48 (m, 4H), 3.16 (d, J=7.6 Hz, 2H), 3.07 (t, J=7.6 Hz, 2H), 2.84 (br t, J=6.0 Hz, 2H), 2.78-2.70 (m, 4H), 2.69-2.62 (m, 1H), 2.19-2.08 (m, 2H), 1.98 (td, 7=6.0, 11.6 Hz, 2H), 1.70-1.63 (m, 4H).

### Example 44

### Preparation of 3-(3-fluoro-4-methoxyphenyl)-4-(1-methyl-5-(2-(5,6,7,8-tetrahydro-1,8-naphthyridin-2-yl)ethoxy)-1H-pyrazol-3-yl)butanoic acid

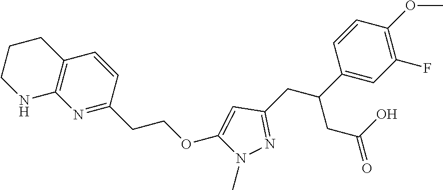 [SMILES: COc1ccc(C(CC(=O)O)Cc2cc(OCCc3ccc4c(n3)NCCC4)n(C)n2)cc1F]

Example 44 was prepared in analogous manner to Example 1, using 3-fluoro-4-methoxybenzaldehyde as the required benzaldehyde in the reaction Scheme 4. The crude product was purified by reverse-phase preparative HPLC and after lyophilization of the fractions afforded the title compound as a cream powder (26.6 mg). LC-MS analysis of the solid showed the desired product at rt 1.76 min and the desired product's mass: m/z 469 (M+H), and m/z 491 (M+Na); Calculated for C₂₅H₂₉FN₄O₄: 468.52. ¹H NMR (400 MHz, DMSO-d₆): δ 1.77-1.87 (m, 2H), 2.54-2.69 (m, 3H), 2.70-2.78 (m, 2H), 3.09 (t, J=6.09 Hz, 2H), 3.17-3.29 (m, 1H), 3.39-3.47 (m, 3H), 3.78 (s, 4H), 4.25 (t, J=6.09 Hz, 2H), 5.43 (s, 1H), 6.69 (d, J=7.28 Hz, 1H), 6.95-7.06 (m, 2H), 7.09 (dd, J=12.86, 1.82 Hz, 1H), 7.58-7.70 (m, 1H), 8.26 (brs, 1H).

### Example 44A and Example 44B

### Step 1. Preparation of (S)-3-(3-fluoro-4-methoxyphenyl)-4-(1-methyl-5-(2-(5,6,7,8-tetrahydro-1,8-naphthyridin-2-yl)ethoxy)-1H-pyrazol-3-yl)butanoic acid and (R)-3-(3-fluoro-4-methoxyphenyl)-4-(1-methyl-5-(2-(5,6,7,8-tetrahydro-1,8-naphthyridin-2-yl)ethoxy)-1H-pyrazol-3-yl)butanoic acid

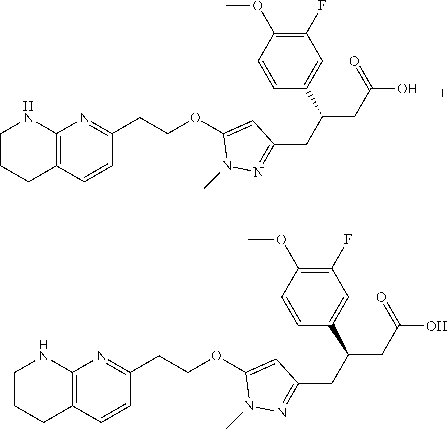 [SMILES: COc1ccc([C@H](CC(=O)O)Cc2cc(OCCc3ccc4c(n3)NCCC4)n(C)n2)cc1F ; COc1ccc([C@@H](CC(=O)O)Cc2cc(OCCc3ccc4c(n3)NCCC4)n(C)n2)cc1F]

3-(3-fluoro-4-methoxy-phenyl)-4-[1-methyl-5-[2-(5,6,7,8-tetrahydro-1,8-naphthyridin-2-yl)ethoxy]pyrazol-3-yl]butanoic acid (2.3 g, 4.91 mmol, 1 eq) was separated by SFC (column: DAICEL CHIRALPAK AD (250 mm*50 mm, 10 μm); mobile phase: [0.1% NH₃H₂O CH₃CH₂OH]; B %: 40%-40%, 7.5 min (S isomer) and 10.4 min (R isomer)). (35)-3-(3-fluoro-4-methoxy-phenyl)-4-[1-methyl-5-[2-(5,6,7,8-tetrahydro-1,8-naphthyridin-2-yl)ethoxy]pyrazol-3-yl]butanoic acid (1.1 g, 2.35 mmol, 48% yield) and (3R)-3-(3-fluoro-4-methoxy-phenyl)-4-[1-methyl-5-[2-(5,6,7,8-tetrahydro-1,8-naphthyridin-2-yl)ethoxy]pyrazol-3-yl]butanoic acid (1.1 g, 2.35 mmol, 48% yield) as off-white solid.

### Example 44A

### Preparation of (S)-3-(5-(3,6-dihydro-2H-pyran-4-yl)-2-methoxyphenyl)-4-(2-(3-(5,6,7,8-tetrahydro-1,8-naphthyridin-2-yl)propyl)thiazol-4-yl)butanoic acid trifluoroacetate

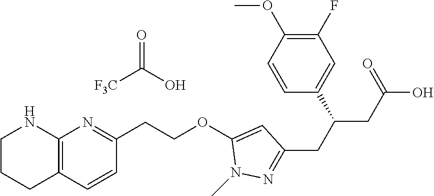

(35)-3-(3-fluoro-4-methoxy-phenyl)-4-[1-methyl-5-[2-(5,6,7,8-tetrahydro-1,8-naphthyridin-2-yl)ethoxy] pyrazol-3-yl]butanoic acid (1.10 g, 2.35 mmol, 1 eq) was purified by preparative HPLC (TFA conditiom:column: YMC-Triart Prep C18 150*40 mm*7 μm; mobile phase: [water (0.1% TFA)-ACN]; B %: 17%-47%, 10 min). The title compound (1.2 g, 2.06 mmol, 88% yield, TFA) was obtained as a white solid. LC-MS mass: m/z 469.3 (M+H)⁺. ¹H NMR (400 MHz, CD₃OD) δ=7.58 (d, J=7.5 Hz, 1H), 7.04-6.91 (m, 3H), 6.67 (d, j=7.3 Hz, 1H), 5.62 (s, 1H), 4.39 (t, j=6.0 Hz, 2H), 3.82 (s, 3H), 3.55-3.45 (m, 5H), 3.43-3.34 (m, 1H), 3.18 (t, J=6.0 Hz, 2H), 2.93-2.76 (m, 4H), 2.73-2.64 (m, 1H), 2.61-2.53 (m, 1H), 1.99-1.90 (m, 2H).

### Example 44B

### Preparation of R)-3-(3-fluoro-4-methoxyphenyl)-4-(1-methyl-5-(2-(5,6,7,8-tetrahydro-1,8-naphthyridin-2-yl)ethoxy)-1H-pyrazol-3-yl)butanoic acid trifluoroacetate

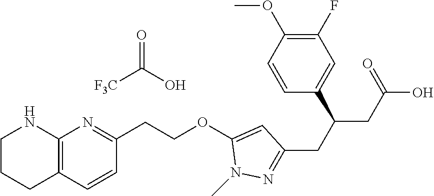

(3R)-3-(3-fluoro-4-methoxy-phenyl)-4-[1-methyl-5-[2-(5,6,7,8-tetrahydro-1,8-naphthyridin-2-yl)ethoxy] pyrazol-3-yl]butanoicacid (1.10 g, 2.35 mmol, 1 eq) was purified by preparative HPLC (TFA conditiom:column: YMC-Triart Prep C18 150*40 mm*7 μm; mobile phase: [water (0.1% TFA)-ACN]; B %: 17%-47%, 10 min). The title compound (1.2 g, 2.06 mmol, 88% yield, TFA) was obtained as a white solid. LC-MS mass: m/z 469.3 (M+H)⁺. ¹H NMR (400 MHz, CD₃OD) δ=7.58 (d, J=7.3 Hz, 1H), 7.02-6.89 (m, 3H), 6.66 (d, J=7.3 Hz, 1H), 5.53 (s, 1H), 4.35 (t, J=6.1 Hz, 2H), 3.81 (s, 3H), 3.54-3.43 (m, 5H), 3.40-3.32 (m, 1H), 3.15 (t, J=6.1 Hz, 2H), 2.90-2.72 (m, 4H), 2.69-2.61 (m, 1H), 2.59-2.49 (m, 1H), 1.94 (td, J=6.1, 11.7 Hz, 2H).

### Example 45

### Preparation of 3-(2-methoxy-5-(4-(methoxymethyl)tetrahydro-2H-pyran-4-yl)phenyl)-4-(1-methyl-5-(2-(5,6,7,8-tetrahydro-1,8-naphthyridin-2-yl)ethoxy)-1H-pyrazol-3-yl)butanoic acid

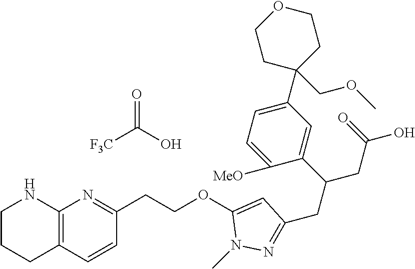 [SMILES: COCC1(c2ccc(OC)c(C(CC(=O)O)Cc3cc(OCCc4ccc5c(n4)NCCC5)n(C)n3)c2)CCOCC1 ; O=C(O)C(F)(F)F]

Example 45 was prepared in analogous manner to Example 1, using 2-methoxy-5-[4-(methoxymethyl)tetrahydropyran-4-yl]benzaldehyde as the required benzaldehyde (obtained according to Example BB) as the required benzaldehyde in the reaction Scheme 4, except performing Step 10 of Scheme 4 before Step 9 of Scheme 4. The title compound was purified by preparatory-HPLC (TFA condition: column: Boston Green ODS 150*30 5u; mobile phase: [water (0.075% TFA)-ACN]; B %: 24%-54%, 7 min). Compound 45, namely 3-[2-methoxy-5-[4-(methoxymethyl)tetrahydropyran-4-yl]phenyl]-4-[1-methyl-5-[2-(5,6,7,8-tetrahydro-1,8-naphthyridin-2-yl)ethoxy]pyrazol-3-yl]butanoic acid (70 mg, 101.05 umol, 40.88% yield, TFA) was obtained as a white solid. ¹H NMR (400 MHz, METHANOL-d4) δ=7.57 (d, 7=7.2 Hz, 1H), 7.15 (dd, 7=2.4, 8.6 Hz, 1H), 7.00 (d, 7=2.4 Hz, 1H), 6.90 (d, 7=8.8 Hz, 1H), 6.66 (d, 7=7.2 Hz, 1H), 5.47 (s, 1H), 4.30 (t, 7=6.0 Hz, 2H), 3.73-3.59 (m, 3H), 3.51-3.42 (m, 4H), 3.35-3.29 (m, 3H), 3.24-3.19 (m, 2H), 3.18-3.08 (m, 5H), 2.97-2.84 (m, 2H), 2.82-2.76 (m, 2H), 2.75-2.63 (m, 2H), 2.04-1.82 (m, 6H).

### Example BB

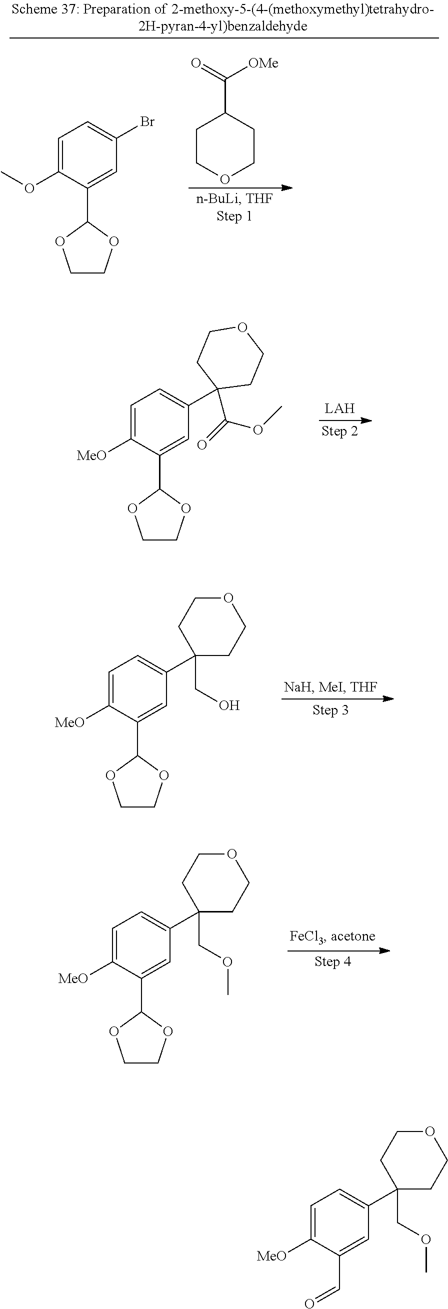 [SMILES: COc1ccc(Br)cc1C1OCCO1 ; COC(=O)C1CCOCC1 ; COC(=O)C1(c2ccc(OC)c(C3OCCO3)c2)CCOCC1 ; COc1ccc(C2(CO)CCOCC2)cc1C1OCCO1 ; COCC1(c2ccc(OC)c(C3OCCO3)c2)CCOCC1 ; COCC1(c2ccc(OC)c(C=O)c2)CCOCC1]

### Step 1. Preparation of methyl 4-(3-(1,3-dioxolan-2-yl)-4-methoxyphenyl)tetrahydro-2-pyran-4-carboxylate

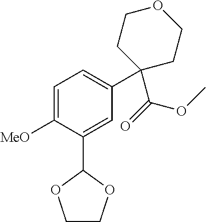 [SMILES: COC(=O)C1(c2ccc(OC)c(C3OCCO3)c2)CCOCC1]

To a mixture of N-cyclohexylcyclohexanamine (7.28 g, 40.14 mmol, 8.00 mL, 1.3 eq) in toluene (150 mL) was added n-BuLi (2.5 M, 16.06 mL, 1.3 eq) at −20° C. under N₂. The mixture warm to 0° C. and stirred for 20 min, and methyl tetrahydropyran-4-carboxylate (4.45 g, 30.88 mmol, 4.12 mL, 1 eq) was added and stirred at 28° C. for 10 min. Then 2-(5-bromo-2-methoxy-phenyl)-1,3-dioxolane (8 g, 30.88 mmol, 1 eq), Pd₂(dba)₃ (848.23 mg, 926.30 umol, 0.03 eq) and t-Bu₃P (1.87 g, 926.30 umol, 2.17 mL, 10% purity, 0.03 eq) was added. The mixture was stirred at 25° C. for 16 hr. LC-MS showed ˜90% desired compound was detected. TLC (PE:EA=3:1) indicated starting material remained and three major new spots formed. The reaction mixture was diluted with NaHCO₃ 60 mL and extracted with ethyl acetate 150 mL (50 mL*3). The combined organic layers were washed with brine 100 mL (50 mL*2), dried over Na₂SO₄, filtered and concentrated under reduced pressure to give a residue. The residue was purified by flash silica gel chromatography (ISCO®; 120 g SepaFlash® Silica Flash Column, Eluent of 0˜30% Ethyl acetate/Petroleum ether gradient @ 40 mL/min). Compound methyl 4-(3-(1,3-dioxolan-2-yl)-4-methoxyphenyl)tetrahydro-2H-pyran-4-carboxylate (20 g, 62.04 mmol, 66.98% yield) was obtained as a yellow solid. The average yield was ˜67% for three batches. ¹H NMR (400 MHz, CHLOROFORM-d) 8=7.53 (br s, 1H), 7.31 (br d, J=8.8 Hz, 1H), 6.86 (br d, J=8.8 Hz, 1H), 6.09 (s, 1H), 4.15-4.09 (m, 2H), 4.05-3.99 (m, 2H), 3.90 (br d, J=11.6 Hz, 2H), 3.84 (s, 3H), 3.63 (s, 3H), 3.57-3.47 (m, 2H), 2.50 (br d, J=13.2 Hz, 2H), 2.01-1.89 (m, 2H).

### Step 2. Preparation of [4-[3-(1,3-dioxolan-2-yl)-4-methoxy-phenyl]tetrahydropyran-4-yl]methanol

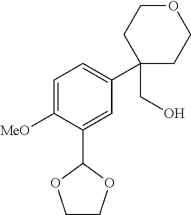 [SMILES: COc1ccc(C2(CO)CCOCC2)cc1C1OCCO1]

To a solution of LAH (2.94 g, 77.55 mmol, 2.5 eq) in THF (60 mL) was added methyl 4-[3-(1,3-dioxolan-2-yl)-4-methoxy-phenyl]tetrahydropyran-4-carboxylate (10 g, 31.02 mmol, 1 eq) slowly at 0° C. Then the mixture was stirred at 25° C. for 2 h. TLC (PE:EA=2:1) indicated started material was consumed completely, and one new major spot with larger polarity was detected. The mixture was taken up by Na₂SO₄.10H₂O, and then the mixture was filtered and the cake was washed with EtOAc (300 mL), after that the organic phase was concentrated in vacuo to get the crude product as a yellow oil. The crude product was used for the next step directly. Compound [4-[3-(1,3-dioxolan-2-yl)-4-methoxy-phenyl]tetrahydropyran-4-yl]methanol (9.13 g, crude) was obtained as a yellow oil. ¹H NMR (400 MHz, CHLOROFORM-d) 8=7.48 (d, J=2.4 Hz, 1H), 7.29-7.24 (m, 1H), 6.90 (d, J=8.8 Hz, 1H), 6.09 (s, 1H), 4.14-4.05 (m, 3H), 4.04-3.98 (m, 2H), 3.85 (s, 3H), 3.79-3.70 (m, 2H), 3.58-3.48 (m, 4H), 2.09 (br d, J=14.4 Hz, 2H), 1.92-1.78 (m, 3H).

### Step 3. Preparation of 4-[3-(1,3-dioxolan-2-yl)-4-methoxy-phenyl]-4-(methoxymethyl)tetrahydropyran

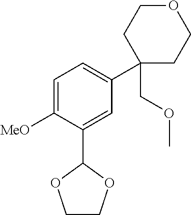 [SMILES: COCC1(c2ccc(OC)c(C3OCCO3)c2)CCOCC1]

To a solution of [4-[3-(1,3-dioxolan-2-yl)-4-methoxy-phenyl]tetrahydropyran-4-yl]methanol (16 g, 54.36 mmol, 1 eq) in THF (150 mL) was added NaH (5.44 g, 135.90 mmol, 60% purity, 2.5 eq) at 0° C. and the mixture was stirred at 0° C. for 30 min. After that, Mel (115.39 g, 812.95 mmol, 50.61 mL, 14.96 eq) was added drop wise to the reaction solution at 0° C. At last, the mixture was stirred at 25° C. for 16 hr. LC-MS indicated ˜66% desired compound was formed. TLC (PE:EA=2:1) indicated started material was consumed completely, and one new major spot with lower polarity was detected. The reaction mixture was quenched with H₂O (150 mL) slowly and then extracted with Ethyl acetate 300 mL (100 mL*3). The combined organic phases were washed with brine 200 mL (100 mL*2), dried over anhydrous Na₂SO₄, filtered and concentrated in vacuo to obtain a residue. The residue was purified by flash silica gel chromatography (ISCO®; 80 g SepaFlash® Silica Flash Column, Eluent of 0-35% Ethyl acetate/Petroleum ether gradient @ 50 mL/min). Compound 4-[3-(1,3-dioxolan-2-yl)-4-methoxy-phenyl]-4-(methoxymethyl) tetrahydropyran (13.45 g, 43.62 mmol, 80.24% yield) was obtained as a white solid. ¹H NMR (400 MHz, CHLOROFORM-d) δ=7.48 (br d, J=2.0 Hz, 1H), 7.32-7.19 (m, 1H), 6.87 (br d, J=8.8 Hz, 1H), 6.11 (s, 1H), 4.16-4.06 (m, 2H), 4.06-3.97 (m, 2H), 3.84 (s, 3H), 3.80-3.70 (m, 2H), 3.58-3.47 (m, 2H), 3.30 (s, 2H), 3.19 (s, 2H), 3.25-3.13 (m, 1H), 2.10-1.91 (m, 4H).

### Step 4. Preparation of 2-methoxy-5-[4-(methoxymethyl)tetrahydropyran-4-yl]benzaldehyde

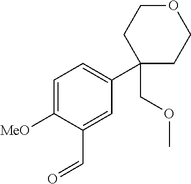 [SMILES: COCC1(c2ccc(OC)c(C=O)c2)CCOCC1]

A mixture of 4-[3-(1,3-dioxolan-2-yl)-4-methoxy-phenyl]-4-(methoxymethyl)tetrahydropyran (2 g, 6.49 mmol, 1 eq) and FeCl₃ (315.60 mg, 1.95 mmol, 112.72 uL, 0.3 eq) in acetone (40 mL) was degassed and purged with N₂ for 3 times, and then the mixture was stirred at 25° C. for 30 min under N₂ atmosphere. TLC (PE:EA=2:1) indicated started material was consumed completely, and three new spots with lower polarity was detected. The reaction mixture concentrated under reduced pressure to give a residue. Then the residue was purified by flash silica gel chromatography (ISCO®; 24 g SepaFlash® Silica Flash Column, Eluent of 0˜30% Ethyl acetate/Petroleum ether gradient @ 35 mL/min). Compound 2-methoxy-5-[4-(methoxymethyl)tetrahydropyran-4-yl]benzaldehyde (1.35 g, 5.10 mmol, 78.58% yield) was obtained as a yellow oil. ¹H NMR (400 MHz, CHLOROFORM-d) 8=10.44 (s, 1H), 7.77 (d, J=2.8 Hz, 1H), 7.52 (dd, J=2.8, 8.8 Hz, 1H), 6.96 (d, J=8.8 Hz, 1H), 3.90 (s, 3H), 3.74 (td, J=4.4, 11.9 Hz, 2H), 3.49 (ddd, J=2.8, 9.2, 11.6 Hz, 2H), 3.31 (s, 2H), 3.17 (s, 3H), 2.10-2.02 (m, 2H), 1.98-1.89 (m, 2H).

### Example 46

### Preparation of 3-(2-methoxy-5-(tetrahydro-2H-pyran-4-yl)phenyl)-4-(1-methyl-5-(2-(5,6,7,8-tetrahydro-1,8-naphthyridin-2-yl)ethoxy)-1H-pyrazol-3-yl)butanoic acid

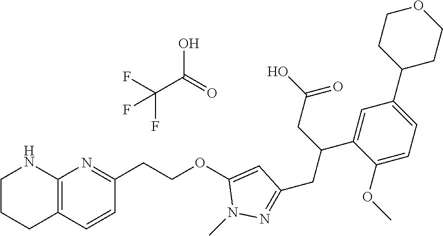

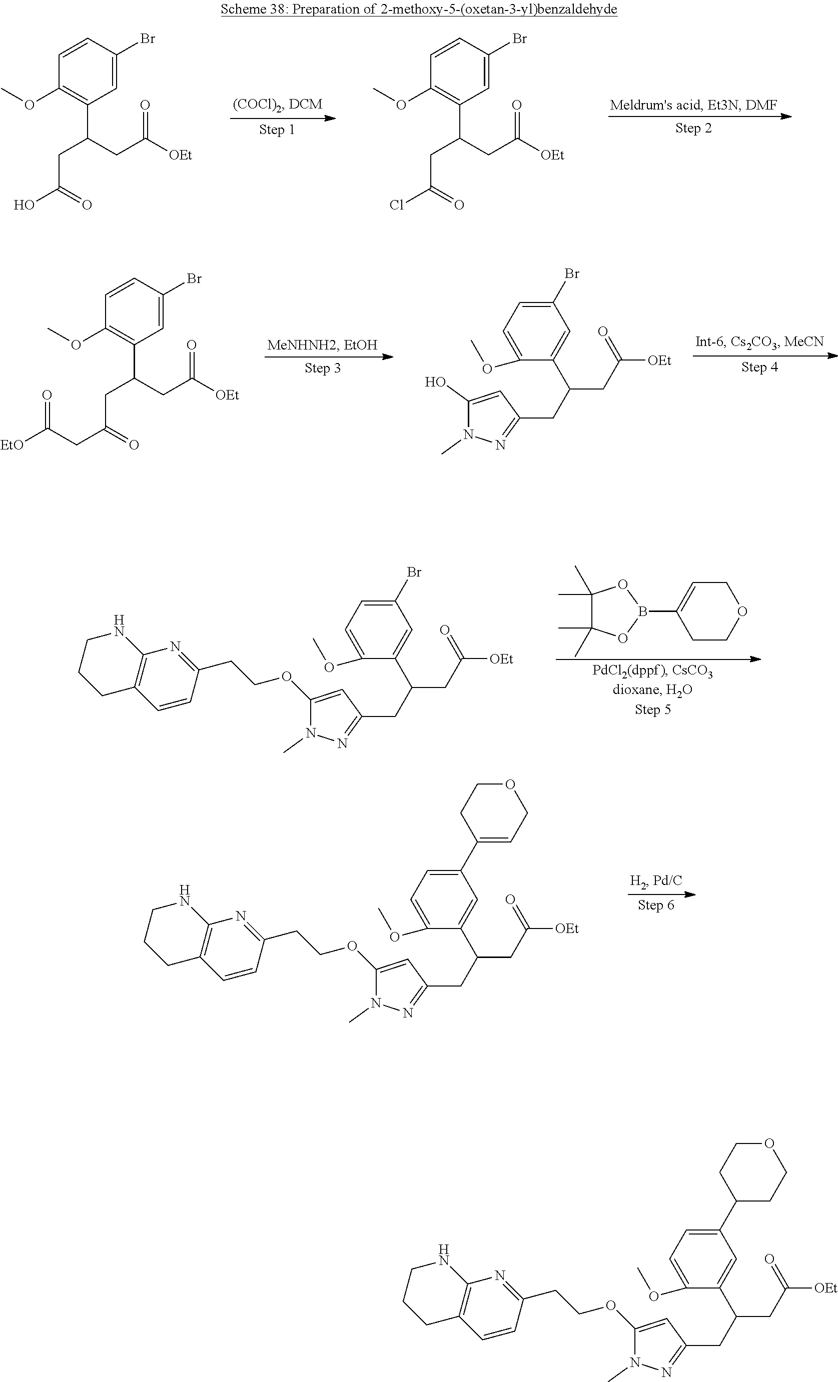

### Step 1. Preparation of ethyl 3-(5-bromo-2-methoxyphenyl)-5-chloro-5-oxopentanoate

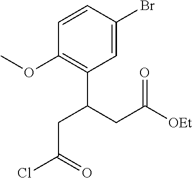 [SMILES: CCOC(=O)CC(CC(=O)Cl)c1cc(Br)ccc1OC]

To a mixture of 3-(5-bromo-2-methoxy-phenyl)-5-ethoxy-5-oxo-pentanoic acid (5.0 g, 14.48 mmol, 1 eq) in DCM (50 mL) was added oxalyl dichloride (4.60 g, 36.21 mmol, 3.17 mL, 2.5 eq) at 0° C., the mixture was stirred at 26° C. for 3 h. LC-MS showed 3-(5-bromo-2-methoxy-phenyl)-5-ethoxy-5-oxo-pentanoic acid was consumed completely and one main peak with was detected. The mixture was concentrated under vacuum to give the residue. The crude product was used without purification for the next step directly. Compound ethyl 3-(5-bromo-2-methoxy-phenyl)-5-chloro-5-oxo-pentanoate (5.27 g, 14.49 mmol, 100.00% yield) was obtained as a yellow oil.

### Step 2. Preparation of diethyl 3-(5-bromo-2-methoxyphenyl)-5-oxoheptanedioate

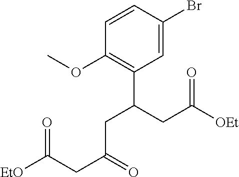 [SMILES: CCOC(=O)CC(=O)CC(CC(=O)OCC)c1cc(Br)ccc1OC]

To a mixture of ethyl 3-(5-bromo-2-methoxy-phenyl)-5-chloro-5-oxo-pentanoate (5.27 g, 14.49 mmol, 1 eq) in DCM (50 mL) was added DMAP (1.95 g, 15.94 mmol, 1.1 eq) and 2,2-dimethyl-1,3-dioxane-4,6-dione (2.51 g, 17.39 mmol, 1.2 eq) at 0° C., the mixture was stirred at 26° C. for 2 hr, the mixture was concentrated in vacuo to give a residue, EtOH (50 mL) was added and the mixture was stirred at 80° C. for 2 h. LC-MS showed ethyl 3-(5-bromo-2-methoxy-phenyl)-5-chloro-5-oxo-pentanoate was consumed completely and one main peak with desired mass was detected. The reaction mixture was concentrated under reduced pressure to give a residue. The residue was purified by flash silica gel chromatography (ISCO®; 80 g SepaFlash® Silica Flash Column, Eluent of 0˜30% Ethyl acetate/Petroleum ether gradient @ 60 mL/min). Compound diethyl 3-(5-bromo-2-methoxy-phenyl)-5-oxo-heptanedioate (4 g, 9.63 mmol, 66.46% yield) was obtained as a yellow oil.

### Step 3. Preparation of ethyl 3-(5-bromo-2-methoxyphenyl)-4-(5-hydroxy-1-methyl-1H-pyrazol-3-yl)butanoate

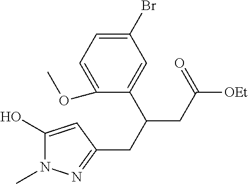 [SMILES: CCOC(=O)CC(Cc1cc(O)n(C)n1)c1cc(Br)ccc1OC]

To a mixture of diethyl 3-(5-bromo-2-methoxy-phenyl)-5-oxo-heptanedioate (4.0 g, 9.63 mmol, 1 eq) in EtOH (500 mL) was added AcOH (578.41 mg, 9.63 mmol, 550.87 uL, 1 eq) and methylhydrazine (2.81 g, 24.40 mmol, 3.21 mL, 2.53 eq), the mixture was stirred at 30° C. for 1.5 h, then warm to 60° C. and stirred for 2 hr. LC-MS showed diethyl 3-(5-bromo-2-methoxy-phenyl)-5-oxo-heptanedioate was consumed completely and one main peak with desired mass was detected. The mixture was concentrated under vacuum to give the residue. The residue was purified by flash silica gel chromatography (ISCO®; 40 g SepaFlash® Silica Flash Column, Eluent of 0˜100% Ethyl acetate/Petroleum ether gradient @ 40 mL/min). Compound ethyl 3-(5-bromo-2-methoxy-phenyl)-4-(5-hydroxy-1-methyl-pyrazol-3-yl)butanoate (700 mg, 1.68 mmol, 17.40% yield, 95.115% purity) was obtained as a yellow oil.

### Step 4. Preparation of ethyl 3-(5-bromo-2-methoxyphenyl)-4-(1-methyl-5-(2-(5,6,7,8-tetrahydro-1,8-naphthyridin-2-yl)ethoxy)-1H-pyrazol-3-yl)butanoate

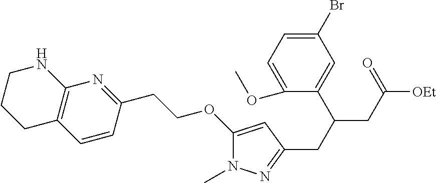 [SMILES: CCOC(=O)CC(Cc1cc(OCCc2ccc3c(n2)NCCC3)n(C)n1)c1cc(Br)ccc1OC]

To a mixture of ethyl 3-(5-bromo-2-methoxy-phenyl)-4-(5-hydroxy-1-methyl-pyrazol-3-yl)butanoate (700 mg, 1.76 mmol, 1 eq), 2-(5,6,7,8-tetrahydro-1,8-naphthyridin-2-yl)ethyl 4-methylbenzenesulfonate (585.74 mg, 1.76 mmol, 1.0 eq) and Cs₂CO₃ (1.15 g, 3.52 mmol, 2 eq) in CH₃CN (150 mL) was stirred at 80° C. for 16 hr. LC-MS showed ethyl 3-(5-bromo-2-methoxy-phenyl)-4-(5-hydroxy-1-methyl-pyrazol-3-yl)butanoate was consumed completely and one main peak with desired mass was detected. The reaction mixture was concentrated under reduced pressure to remove MeCN. The residue was diluted with water 50 mL and extracted with EtOAc (50 mL*3). The combined organic layers were washed with brine 50 mL, dried over anhydrous Na₂SO₄, filtered and concentrated under reduced pressure to give a residue. The residue was purified by flash silica gel chromatography (ISCO®; 4 g SepaFlash® Silica Flash Column, Eluent of 0˜100% Ethyl acetate/Petroleum ether gradient @ 25 mL/min). Compound ethyl 3-(5-bromo-2-methoxy-phenyl)-4-[1-methyl-5-[2-(5,6,7,8-tetrahydro-1,8-naphthyridin-2-yl)ethoxy]pyrazol-3-yl]butanoate (460 mg, 825.14 umol, 46.83% yield) was obtained as a yellow oil.

### Step 5. Preparation of ethyl 3-(5-(3,6-dihydro-2H-pyran-4-yl)-2-methoxyphenyl)-4-(1-methyl-5-(2-(5,6,7,8-tetrahydro-1,8-naphthyridin-2-yl)ethoxy)-1H-pyrazol-3-yl)butanoate

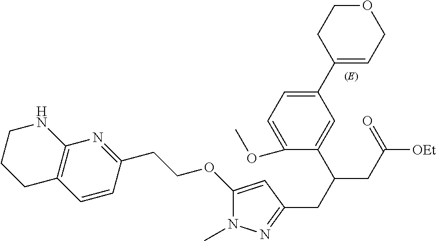 [SMILES: CCOC(=O)CC(Cc1cc(OCCc2ccc3c(n2)NCCC3)n(C)n1)c1cc(C2=CCOCC2)ccc1OC]

Ethyl 3-(5-bromo-2-methoxy-phenyl)-4-[1-methyl-5-[2-(5,6,7,8-tetrahydro-1,8-naphthyridin-2-yl)ethoxy]pyrazol-3-yl]butanoate (400 mg, 717.52 umol, 1 eq), 2-(3,6-dihydro-2H-pyran-4-yl)-4,4,5,5-tetramethyl-1,3,2-dioxaborolane (452.20 mg, 2.15 mmol, 3 eq), Pd(dppf)Cl₂.CH₂Cl₂ (11.72 mg, 14.35 umol, 0.02 eq) and Cs₂CO₃ (467.56 mg, 1.44 mmol, 2 eq) in dioxane (10 mL) and H₂O (1 mL) was de-gassed and then heated to 100° C. for 16 hours under N₂. LC-MS showed the starting material was consumed completely. The mixture was concentrated in vacuo to give crude compound. Ethyl acetate (100 mL) was added. The organic phase was washed with H₂O (100 mL), brine (100 mL), dried over anhydrous Na₂SO₄, concentrated in vacuum to give a residue. The residue was purified by flash silica gel chromatography (ISCO®; 4 g SepaLlash® Silica Flash Column, Eluent of 0˜100% Ethyl acetate/Petroleum ether gradient @ 25 mL/min). Compound ethyl 3-[5-(3,6-dihydro-2H-pyran-4-yl)-2-methoxy-phenyl]-4-[1-methyl-5-[2-(5,6,7,8-tetrahydro-1,8-naphthyridin-2-yl)ethoxy]pyrazol-3-yl]butanoate (335 mg, 597.49 umol, 83.27% yield) was obtained as a yellow oil.

### Step 6. Preparation of ethyl 3-(2-methoxy-5-(tetrahydro-2H-pyran-4-yl)phenyl)-4-(1-methyl-5-(2-(5,6,7,8-tetrahydro-1,8-naphthyridin-2-yl)ethoxy)-1H-pyrazol-3-yl)butanoate

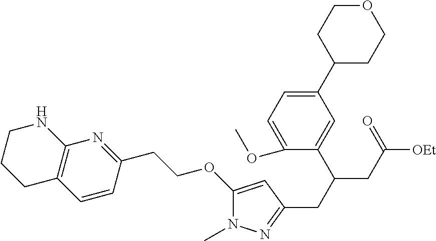 [SMILES: CCOC(=O)CC(Cc1cc(OCCc2ccc3c(n2)NCCC3)n(C)n1)c1cc(C2CCOCC2)ccc1OC]

To a mixture of ethyl 3-[5-(3,6-dihydro-2H-pyran-4-yl)-2-methoxy-phenyl]-4-[1-methyl-5-[2-(5,6,7,8-tetrahydro-1,8-naphthyridin-2-yl)ethoxy]pyrazol-3-yl]butanoate (370 mg, 659.91 umol, 1 eq) in EtOH (50 mL) was added Pd/C (200 mg, 659.91 umol, 10% purity, 1 eq), the mixture was stirred at 26° C. for 16 h under H₂ (45 Psi). LC-MS showed ethyl 3-[5-(3,6-dihydro-2H-pyran-4-yl)-2-methoxy-phenyl]-4-[1-methyl-5-[2-(5,6,7,8-tetrahydro-1,8-naphthyridin-2-yl)ethoxy]pyrazol-3-yl]butanoate was consumed completely and one main peak with desired mass was detected. The reaction mixture was filtered and the filter was concentrated. Compound ethyl 3-(2-methoxy-5-tetrahydropyran-4-yl-phenyl)-4-[1-methyl-5-[2-(5,6,7,8-tetrahydro-1,8-naphthyridin-2-yl)ethoxy]pyrazol-3-yl]butanoate (350 mg, crude) was obtained as a yellow oil.

### Step 7. Preparation of 3-(2-methoxy-5-(tetrahydro-2H-pyran-4-yl)phenyl)-4-(1-methyl-5-(2-(5,6,7,8-tetrahydro-1,8-naphthyridin-2-yl)ethoxy)-1H-pyrazol-3-yl)butanoic acid trifluoroacetate

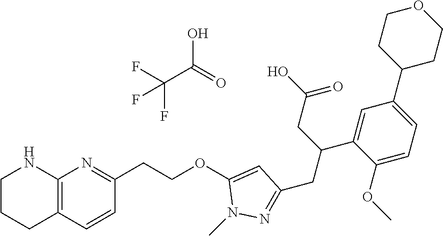 [SMILES: COc1ccc(C2CCOCC2)cc1C(CC(=O)O)Cc1cc(OCCc2ccc3c(n2)NCCC3)n(C)n1 ; O=C(O)C(F)(F)F]

A mixture of ethyl 3-(2-methoxy-5-tetrahydropyran-4-yl-phenyl)-4-[1-methyl-5-[2-(5,6,7,8-tetrahydro-1,8-naphthyridin-2-yl)ethoxy]pyrazol-3-yl]butanoate (350 mg, 622.00 umol, 1 eq) and LiOH.H₂O (78.30 mg, 1.87 mmol, 3 eq) in EtOH (50 mL) and H₂O (10 mL) was stirred at 26° C. for 16 h. LCMS showed methyl ethyl 3-(2-methoxy-5-tetrahydropyran-4-yl-phenyl)-4-[1-methyl-5-[2-(5,6,7,8-tetrahydro-1,8-naphthyridin-2-yl)ethoxy]pyrazol-3-yl]butanoate was consumed completely and one main peak with desired mass was detected. The mixture was extracted with Ethyl acetate (50 mL), the aqueous layer was adjusted the pH to 5 with HCl then extracted with Ethyl acetate (50 mL*2), washed with brine (50 mL), dried over Na₂SO₄ and concentrated under vacuum to give the residue. The residue was purified by prep-HPLC (column: Boston Green ODS 150*30 5u; mobile phase: [water (0.075% TFA)-ACN]; B %: 28%-48%, 7 min). Compound 3-(2-methoxy-5-tetrahydropyran-4-yl-phenyl)-4-[1-methyl-5-[2-(5,6,7,8-tetrahydro-1,8-naphthyridin-2-yl)ethoxy]pyrazol-3-yl]butanoic acid (15 mg, 23.12 umol, 3.72% yield, TFA) was obtained as a yellow gum. ¹H NMR (400 MHz, MeOD) δ=7.64-7.53 (m, 1H), 7.09-7.00 (m, 1H), 7.00-6.97 (m, 1H), 6.89-6.83 (m, 1H), 6.70-6.64 (m, 1H), 5.56-5.46 (m, 1H), 4.39-4.28 (m, 2H), 4.04-3.96 (m, 2H), 3.81 (s, 3H), 3.77-3.68 (m, 1H), 3.57-3.46 (m, 7H), 3.19-3.13 (m, 2H), 2.93-2.79 (m, 4H), 2.74-2.60 (m, 3H), 2.00-1.89 (m, 2H), 1.74-1.62 (m, 4H). LC-MS mass: m/z 535.4 (M+H).

### Example 47

### Preparation of (5)-3-(7-(tert-butyl)benzo[d][1,3]dioxol-5-yl)-4-(1-methyl-5-(2-(5,6,7,8-tetrahydro-1,8-naphthyridin-2-yl)ethoxy)-1H-pyrazol-3-yl)butanoic acid

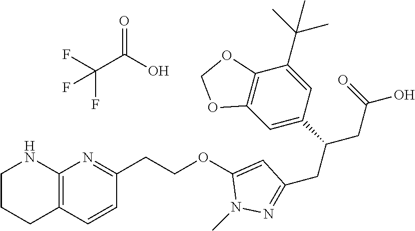 [SMILES: Cn1nc(C[C@@H](CC(=O)O)c2cc3c(c(C(C)(C)C)c2)OCO3)cc1OCCc1ccc2c(n1)NCCC2 ; O=C(O)C(F)(F)F]

A mixture of 3-(7-tert-butyl-1,3-benzodioxol-5-yl)-4-[1-methyl-5-[2-(5,6,7,8-tetrahydro-1,8-naphthyridin-2-yl)ethoxy]pyrazol-3-yl]butanoic acid (2.5 g, 4.80 mmol, 1 eq) was purified by prep-SFC. (column: DAICEL CHIRALPAK AD (250 mm*50 mm, 10 um); mobile phase: [0.1% NH₃H₂O ETOH]; B %: 30%-30%, min). Compound (5)-3-(7-(tert-butyl)benzo[d][1,3]dioxol-5-yl)-4-(1-methyl-5-(2-(5,6,7,8-tetrahydro-1,8-naphthyridin-2-yl)ethoxy)-1H-pyrazol-3-yl (butanoic acid (1.1 g, 2.11 mmol, 44.00% yield) was obtained as a yellow solid. (S)-3-(7-(tert-butyl)benzo[d][1,3]dioxol-5-yl)-4-(1-methyl-5-(2-(5,6,7,8-tetrahydro-1,8-naphthyridin-2-yl)ethoxy)-1H-pyrazol-3-yl)butanoic acid (1.0 g, 2.11 mmol, 1 eq) was purified by prep-HPLC (column: Phenomenex Synergi C18 250*21.2 mm*4 um; mobile phase: [water (0.1% TFA)-ACN]; B %: 25%-55%, 18 min). Compound (S)-3-(7-(tert-butyl)benzo[d][1,3]dioxol-5-yl)-4-(1-methyl-5-(2-(5,6,7,8-tetrahydro-1,8-naphthyridin-2-yl)ethoxy)-1H-pyrazol-3-yl)butanoic acid (1.1 g, 1.73 mmol, 90.24% yield, TFA) was obtained as a yellow gum. ¹H NMR (400 MHz, MeOD) δ=7.60 (d, J=7.2 Hz, 1H), 6.69 (d, J=7.2 Hz, 1H), 6.63-6.60 (m, 1H), 6.59-6.56 (m, 1H), 5.89-5.82 (m, 2H), 5.56-5.50 (m, 1H), 4.40-4.30 (m, 2H), 3.49 (s, 5H), 3.37-3.32 (m, 1H), 3.21-3.12 (m, 2H), 2.86-2.49 (m, 6H), 1.99-1.89 (m, 2H), 1.29 (s, 9H). ¹⁹F NMR (376 MHz, MeOD) −77.410 (s, 3F). LC-MS mass: m/z 521.3 (M+H).

### Example 48

### Step 1. Preparation of (S)-3-(2-methoxy-5-(4-(methoxymethyl)tetrahydro-2H-pyran-4-yl)phenyl)-4-(1-methyl-5-(2-(5,6,7,8-tetrahydro-1,8-naphthyridin-2-yl)ethoxy)-1H-pyrazol-3-yl)butanoic acid

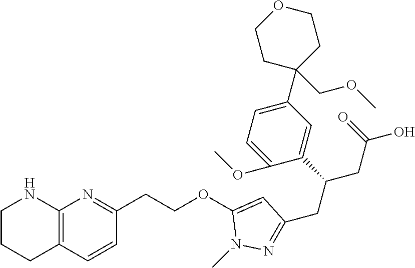 [SMILES: COCC1(c2ccc(OC)c([C@H](CC(=O)O)Cc3cc(OCCc4ccc5c(n4)NCCC5)n(C)n3)c2)CCOCC1]

3-[2-methoxy-5-[4-(methoxymethyl)tetrahydropyran-4-yl]phenyl]-4-[1-methyl-5-[2-(5,6,7,8-tetrahydro-1,8-naphthyridin-2-yl)ethoxy]pyrazol-3-yl]butanoic acid (61.7 mg, 89.07 umol, 1 eq, TFA) was separated by SFC (column: DAICEL CHIRALPAK AD (250 mm*50 mm, 10 um); mobile phase: [0.1% NH₃H₂O IPA]; B %: 35%-35%, min). The sample was checked by chiral HPLC. The sample was lyophilized. (5)-3-(2-methoxy-5-(4-(methoxymethyl)tetrahydro-2H-pyran-4-yl)phenyl)-4-(1-methyl-5-(2-(5,6,7,8-tetrahydro-1,8-naphthyridin-2-yl)ethoxy)-1H-pyrazol-3-yl)butanoic acid (13.7 mg, 23.67 umol, 26.58% yield) was obtained as a white solid. LC-MS: m/z 579.4 (M+H). ¹H NMR (400 MHz, CD₃Cl) δ=8.64 (br s, 1H), 8.55-8.73 (m, 1H), 7.09-7.22 (m, 3H), 6.84 (d, J=8.8 Hz, 1H), 6.36 (d, J=7.2 Hz, 1H), 5.55 (s, 1H), 4.24-4.40 (m, 2H), 3.81-3.93 (m, 4H), 3.74 (br d, J=11.2 Hz, 2H), 3.49 (br t, J=10.8 Hz, 2H), 3.38 (s, 5H), 3.29 (s, 2H), 3.10-3.23 (m, 4H), 2.82-2.99 (m, 3H), 2.58-2.79 (m, 4H), 1.81-2.11 (m, 6H).

### I. Biological Assay Results

The integrin inhibitory activities of the compounds of the present disclosure are shown in Table 6 along with data from a Comparator Compounds 1 and 2 (CC1 and CC2), which are depicted in Table 5.

The methods for conducting each assay are described below.

TABLE 6
Integrin Inhibition Assay Data

| | α_{V}β₁ | α_{V}β₃ | α_{V}β₅ | α_{V}β₆ | α_{V}β₈ | α₅β₁ |
|---|---|---|---|---|---|---|
| | SPRA | SPRA | SPRA | SPRA | SPRA | SPRA |
| Example | IC₅₀ (nM) | IC₅₀ (nM) | IC₅₀ (nM) | IC₅₀ (nM) | IC₅₀ (nM) | IC₅₀ (nM) |
| Example 1 | 6 | 48 | 44 | 200 | 27 | 2 |
| Example 2 | 9 | 6 | 0.3 | 20 | 7 | 7 |
| Example 3 | 12 | 8 | 2 | 54 | 13 | 31 |
| Example 4 | 7 | 15 | 6 | 84 | 18 | 4 |
| Example 5 | 4 | 140 | 160 | 460 | 20 | 2 |
| Example 6 | 5 | 120 | 54 | 140 | 15 | 6 |
| Example 7 | 3 | 17 | 27 | 300 | 18 | 1 |
| Example 8 | 2 | 3 | 0.3 | 36 | 3 | 3 |
| Example 9 | 2 | 38 | 32 | 150 | 8 | 1 |
| Example 10 | 4 | 410 | 84 | 130 | 92 | 15 |
| Example 11 | 800 | 13000 | 17000 | 11000 | 740 | 15000 |
| Example 12 | 13 | 90 | 160 | 180 | 17 | 180 |
| Example 13 | 21 | 31 | 140 | 170 | 540 | 210 |
| Example 14 | 9 | 9 | 9 | 170 | 170 | 47 |
| Example 15 | 3 | 9 | 0.2 | 60 | 120 | 28 |
| Example 16 | 5 | 7 | 0.7 | 19 | 41 | 18 |
| Example 17 | 14 | 930 | 230 | 1900 | 110 | 120 |
| Example 18 | 5 | 56 | 4 | 12 | 3 | 4 |
| Example 19 | 7 | 6 | 0.6 | 180 | 58 | 10 |
| Example 20 | 6 | 880 | 590 | 220 | 67 | 9 |
| Example 21 | 10 | 340 | 290 | 320 | 140 | 29 |
| Example 22 | 3 | 5 | 10 | 160 | 79 | 2 |
| Example 23 | 8 | 26 | 29 | 140 | 190 | 36 |
| Example 24 | 9 | 130 | 96 | 48 | 33 | 25 |
| Example 25 | 6 | 37 | 78 | 160 | 18 | 2 |
| Example 26 | 8 | 530 | 610 | 330 | 38 | 61 |
| Example 27 | 11 | 2900 | 1100 | 1000 | 55 | 43 |
| Example 28 | 6 | 1500 | 440 | 310 | 31 | 25 |
| Example 29 | 7 | 1600 | 800 | 450 | 87 | 40 |
| Example 30 | 5 | 720 | 260 | 270 | 25 | 30 |
| Example 31 | 8 | 2600 | 1500 | 1700 | 76 | 66 |
| Example 32 | 7 | 600 | 240 | 130 | 14 | 37 |
| Example 33 | 2 | 38 | 32 | 150 | 8 | 0.7 |
| Example 34 | 4 | 2500 | 360 | 3400 | 140 | 69 |
| Example 35 | 5 | 78 | 69 | 200 | 51 | 88 |
| Example 36 | 2 | 840 | 150 | 120 | 29 | 7 |
| Example 37 | 3 | 230 | 37 | 81 | 83 | 25 |
| Example 37A | 0.9 | 160 | 31 | 31 | 43 | 17 |
| Example 37B | 190 | 940 | 62 | 3000 | 5600 | 2800 |
| Example 38 | 15 | 9 | 0.7 | 240 | 2700 | 420 |
| Example 39 | 4 | 520 | 93 | 370 | 55 | 33 |
| Example 40 | 3 | 740 | 180 | 790 | 30 | 6 |
| Example 41 | 1 | 9 | 5 | 37 | 1 | 0.3 |
| Example 42 | 2 | 58 | 14 | 17 | 41 | 10 |
| Example 43 | 3 | 520 | 61 | 95 | 210 | 65 |
| Example 44 | 3.2 | 2.3 | 0.22 | 44 | 80 | 47 |
| Example 45 | 2.2 | 1200 | 1200 | 4100 | 1000 | 13 |
| Example 46 | 2.8 | 310 | 87 | 260 | 250 | 16 |
| Example 47 | 0.92 | 1.8 | 0.067 | 15 | 34 | 6.2 |
| Example 48 | 2.4 | 690 | 770 | 2800 | 540 | 9.7 |
| CC1 | 7 | 3 | 0.3 | 132 | 100 | 79 |
| CC2 | 19 | 3.1 | 0.2 | 54 | 680 | 770 |

A. Solid Phase Receptor Assay (SPRA) for α5β1 Function

Purified human fibronectin (R&D Systems, 1918-FN) diluted to 2 μg/mL in TBS+ buffer (25 mM Tris pH 7.4, 137 mM NaCl, 2.7 mM KCl, 1 mM CaCl₂, 1 mM MgCl₂, 1 mM MnCl₂) was added to wells (50 μL/well) of a 96-well half-well transparent microtiter plate (Costar 3690) and incubated overnight at 4° C. Wells were washed 3 times with 150 μL TBS+ and then 150 μL of blocking buffer (TBS+ with 1% bovine serum albumin, Sigma A7906) was added. The plate was incubated for 1 hr at 37° C. and then washed 3× with TBS+ buffer. Recombinant human integrin α5β1 (R&D Systems, 3230-A5) was diluted to 0.1 μg/mL in TBS+/0.1% bovine serum albumin, and 49 μL was added to each well. Compounds were diluted to 20 μM and then 1 μL was added to each well of the plate according to a standard template with each sample repeated in triplicate. After incubation for two hours at room temperature, the plate was washed 3× with 150 μL of TBS+ buffer. To each well, 50 μL of biotinylated anti-α5 antibody (R&D Systems, BAF1864) at 0.5 μg/mL in TBS+/0.1% BSA were added and the plate covered and incubated for 1 hr at room temperature. After washing the plate 3× with 150 μL of TBS+ buffer, 50 μL of streptavidin-conjugated horseradish peroxidase (R&D Systems, DY998) diluted in TBS+ blocking buffer were added to the wells and the plate incubated for 20 min at room temperature. The plate was washed 3× with TBS+ buffer followed by 50 μL of room temperature TMB substrate (Sigma, T4444) added to each well in the dark and the plate incubated for 25 min at room temperature. 25 μL of 1.0 M phosphoric acid was added as a stop solution and the plates were read at 450 nm using a Spectramax plate reader. Concentration-response curves were constructed by non-linear regression (best fit) analysis, and IC₅₀ values were calculated for each compound.

B. Solid Phase Receptor Assay (SPRA) for α_{V}β1 Function

Purified human fibronectin (R&D Systems, 1918-FN) diluted to 5 μg/mL in TBS+ buffer (25 mM Tris pH 7.4, 137 mM NaCl, 2.7 mM KCl, 1 mM CaCl₂, 1 mM MgCl₂, 1 mM MnCl₂) was added to wells (50 μL/well) of a 96-well half-well transparent microtiter plate (Costar 3690) and incubated overnight at 4° C. Wells were washed 3 times with 150 μL TBS+ and then 150 μL of blocking buffer (TBS+ with 1% bovine serum albumin, Sigma A7906) was added. The plate was incubated for 1 hr at 37° C. and then washed 3× with TBS+ buffer. Recombinant human integrin αvβ1 (R&D Systems. 6579-AV) was diluted to 2.0 μg/mL in TBS+/0.1% bovine serum albumin, and 49 μL was added to each well. Compounds were diluted to 20 μM and 1 μL was added to each well of the plate according to a standard template with each sample repeated in triplicate. After incubation for two hours at room temperature, the plate was washed 3× with 150 μL of TBS+ buffer. To each well, 50 μL of biotinylated anti-α_{V} antibody (R&D Systems, BAF1219) at 1 μg/mL in TBS+/0.1% BSA were added and the plate covered and incubated for 1 hr at room temperature. After washing the plate 3× with 150 μL of TBS+ buffer, 50 μL of streptavidin-conjugated horseradish peroxidase (R&D Systems, DY998) diluted in TBS+ blocking buffer were added to the wells and the plate incubated for 20 min at room temperature. The plate was washed 3× with TBS+ buffer followed by 50 μL of TMB substrate (Sigma, T4444) added to each well in the dark and the plate incubated for 25 min at room temperature. 25 μL of 1.0 M phosphoric acid was added as a stop solution and the plates were read at 450 nm using a Spectramax plate reader. Concentration-response curves were constructed by non-linear regression (best fit) analysis, and IC₅₀ values were calculated for each compound.

C. Solid Phase Receptor Assay (SFRA) for α_{V}β3 Function

Recombinant human vitronectin (R&D Systems, 2308-VN) diluted to 1 μg/mL in TBS+ buffer (25 mM Tris pH 7.4, 137 mM NaCl, 2.7 mM KCl, 1 mM CaCl₂, 1 mM MgCl₂, 1 mM MnCl₂) was added to wells (50 μL/well) of a 96-well half-well transparent microtiter plate (Costar 3690) and incubated overnight at 4° C. Wells were washed 3 times with 150 μL TBS+ and then 150 μL of blocking buffer (TBS+ with 1% bovine serum albumin, Sigma A7906) was added. The plate was incubated for 1 hr at 37° C. and then washed 3× with TBS+ buffer. Recombinant human integrin α_{V}β3 (R&D Systems, 3050-AV) was diluted to 1 μg/mL in TBS+/0.1% bovine serum albumin, and 49 μL was added to each well. Compounds were diluted to 20 μM and then 1 μL was added to each well of the plate according to a standard template with each sample repeated in triplicate. After incubation for two hours at room temperature, the plate was washed 3× with 150 μL of TBS+ buffer. To each well, 50 μL of biotinylated anti-α_{V} antibody (R&D Systems, BAF1219) at 0.5 μg/mL in TBS+/0.1% BSA were added and the plate covered and incubated for 1 hr at room temperature. After washing the plate 3× with 150 μL of TBS+ buffer, 50 μL of streptavidin-conjugated horseradish peroxidase (R&D Systems, DY998) diluted in TBS+ blocking buffer were added to the wells and the plate incubated for 20 min at room temperature. The plate was washed 3× with TBS+ buffer followed by 50 μL of TMB substrate (Sigma, T4444) added to each well in the dark and the plate was incubated for 25 min at room temperature. 25 μL of 1.0 M phosphoric acid was added as a stop solution and the plates were read at 450 nm using a Spectramax plate reader. Concentration-response curves were constructed by non-linear regression (best fit) analysis, and IC₅₀ values were calculated for each compound.

D. Solid Phase Receptor Assay (SPRA) for α_{V}β5 Function

Recombinant human vitronectin (R& D Systems, 2308-VN) at 0.25 μg/mL in TBS+ buffer (25 mM Tris pH 7.4, 137 mM NaCl, 2.7 mM KCl, 1 mM CaCl₂, 1 mM MgCl₂, 1 mM MnCl₂) was added to wells (50 μL/well) of a 96-well half-well transparent microtiter plate (Costar 3690) and incubated overnight at 4° C. Wells were washed 3 times with 150 μL TBS+ and then 150 μL of blocking buffer (TBS+ with 1% bovine serum albumin, Sigma A7906) was added. The plate was incubated for 1 hr at 37° C. and then washed 3× with TBS+ buffer. Recombinant human integrin α_{V}β5 (R&D Systems, 2528-AV) was diluted to 0.1 μg/mL in TBS+/0.1% bovine serum albumin, and 49 μL was added to each well. Compounds were diluted to 20 μM and then 1 μL was added to each well of the plate according to a standard template with each sample repeated in triplicate. After incubation for two hours at room temperature, the plate was washed 3× with 150 μL of TBS+ buffer. To each well, 50 μl of biotinylated anti-α_{V} antibody (R&D Systems, BAF1219) at 0.5 μg/mL in TBS+/0.1% BSA at 0.5 μg/mL were added and the plate covered and incubated for 1 hr at room temperature. After washing the plate 3× with 150 μL of TBS+ buffer, 50 μL of streptavidin-conjugated horseradish peroxidase (R&D Systems, DY998) diluted in TBS+ blocking buffer were added to the wells and the plate incubated for 20 min at room temperature. The plate was washed 3× with TBS+ buffer followed by 50 μL of TMB substrate (Sigma T4444) added to each well in the dark and the plate incubated for 5 min at room temperature. 25 μL of 1.0 M phosphoric acid was added as a stop solution and the plates were read at 450 nm using a Spectramax plate reader. Concentration-response curves were constructed by non-linear regression (best fit) analysis, and IC₅₀ values were calculated for each compound.

E. Solid Phase Receptor Assay (SPRA) for α_{V}β6 Function

Recombinant human LAP (R&D Systems, 246-LP) diluted to 0.25 μg/mL in TBS+ buffer (25 mM Tris pH 7.4, 137 mM NaCl, 2.7 mM KCl, 1 mM CaCl₂, 1 mM MgCl₂, 1 mM MnCl₂) was added to wells (50 μL/well) of a 96-well half-well transparent microtiter plate (Costar 3690) and incubated overnight at 4° C. Wells were washed 3 times with 150 μL TBS+, and then 150 μL of blocking buffer (TBS+ with 1% bovine serum albumin, Sigma A7906) was added. The plate was incubated for 1 hr at 37° C., and then washed 3× with TBS+ buffer. Recombinant human integrin α_{V}β6 (R&D Systems, 3817-AV) was diluted to 0.1 μg/mL in TBS+/0.1% bovine serum albumin, and 49 μL was added to each well. Compounds were diluted to 20 μM and then 1 μL was added to each well of the plate according to a standard template with each sample repeated in triplicate. After incubation for two hours at room temperature, the plate was washed 3× with 150 μL of TBS+ buffer. To each well, 50 μL of biotinylated anti-α_{V} antibody (R&D Systems, BAF1219) at 0.5 μg/mL in TBS+/0.1% BSA were added and the plate was covered and incubated for 1 hr at room temperature. After washing the plate 3× with 150 μL of TBS+ buffer, 50 μL of streptavidin-conjugated horseradish peroxidase (R&D Systems, DY998) diluted in TBS+ blocking buffer were added to the wells and the plate incubated for 20 min at room temperature. The plate was washed 3× with TBS+ buffer followed by 50 μL of TMB substrate (Sigma T4444) added to each well in the dark and the plate incubated for 10 min at room temperature. 25 μL of 1.0 M phosphoric acid was added as a stop solution and the plates were read at 450 nm using a Spectramax plate reader. Concentration-response curves were constructed by non-linear regression (best fit) analysis, and IC₅₀ values were calculated for each compound.

F. Solid Phase Receptor Assay (SFRA) for α_{V}β8 Function

Recombinant human LAP protein (R&D Systems, Inc, 246-LP) diluted to 0.5 μg/mL in TBS+ buffer (25 mM Tris pH 7.4, 137 mM NaCl, 2.7 mM KCl, 1 mM CaCl₂, 1 mM MgCl₂, 1 mM MnCl₂) was added to wells (50 μl/well) of a 96-well half-well transparent microtiter plate (Costar 3690), and incubated overnight at 4° C. Wells were washed 3 times with 150 μL TBS+ and then 150 μL of blocking buffer (TBS+ with 1% bovine serum albumin, Sigma A7906) was added. The plate was incubated for 1 hr at 37° C. and then washed 3× with TBS+. Recombinant human integrin α_{V}β8 (R&D Systems, 4135-AV) was diluted to 0.1 μg/mL in TBS+/0.1% bovine serum albumin, and 49 μL was added to each well. Compounds were diluted to 20 μM and 1 μL was added to each well of the plate according to a standard template with each sample repeated in triplicate. After incubation for two hours at room temperature, the plate was washed 3× with 150 μL of TBS+. To each well, 50 μL of biotinylated anti-α_{V} antibody (R&D Systems, BAF1219) at 1 μg/mL in TBS+/0.1% BSA were added and the plate was covered and incubated for 1 hr at room temperature. After washing the plate 3× with 150 μL of TBS+ buffer, 50 μL of streptavidin-conjugated horseradish peroxidase (R&D Systems, DY998) diluted in TBS+ blocking buffer were added to the wells and the plate incubated for 20 min at room temperature. The plate was washed 3× with TBS+ followed by 50 μL of TMB substrate (Sigma T4444) added to each well in the dark and the plate incubated for 10 min at room temperature. 25 μL of 1.0 M phosphoric acid was added as a stop solution and the plates were read at 450 nm using a Spectramax plate reader. Concentration-response curves were constructed by non-linear regression (best fit) analysis, and IC₅₀ values were calculated for each compound.

While the disclosure may have focused on several embodiments or may have been described in terms of preferred embodiments, it will be apparent to those of skill in the art that variations and modifications may be applied to the compounds, compositions, and methods without departing from the spirit, scope, and concept of the invention. All variations and modifications apparent to those skilled in the art are deemed to be within the spirit, scope, and concept of the invention as defined by the appended claims.

### REFERENCES

The following references to the extent that they provide exemplary procedural or other details supplementary to those set forth herein, are specifically incorporated herein by reference.
Adachi et al., Clin. Cancer Res., 6(1):96-101, 2000.
Asano et al., J. Immunol., 175(11):7708-7718, 2005a.
Asano, et al., Arthritis Rheum., 52(9):2897-2905, 2005b.
Avraamides et al., Nat. Rev. Cancer, 8(8):604-617, 2008.
Bax et al., J. Biol. Chem., 278(36):34605-34616, 2003.
Becker et al., Tetrahedron, 39:4189-4192, 1983.
Bennet, J. Clin. Invest., 115(12):3363-69, 2005.
Bhaskar et al., J. Transl. Med., 5:61, 2007.
Blase et al., Int. J. Cancer, 60(6):860-866, 1995.
Bouzeghrane, et al., J. Mol. Cell. Cardiology, 36:343-353, 2004.
Clark, et al., Organic Process Research & Development, 8:51-61, 2004.
Clark, et al., 8:571-575, 2004.
Collo, J. Cell Sci., 112(Pt 4):569-578, 1999.
Cook et al., JPET, 281:677-89, 1997.
Danen et al., Histopathology, 24(3):249-256, 1994.
Edward, Curr. Opin. Oncol., 7(2): 185-191, 1995.
Engleman et al, J. Clin. Invest., 99(9):2284-2292, 1997.
Faulconbridge et al., Tetrahedron Lett., 41:2679-2681, 2000.
Ferrari et al., Proc. Natl. Acad. Sci. USA, 103(46):17260-17265, 2006.
Gao and Brigstock, Gut, 55:856-862, 2006.
Gerber, et al., Nature, 503(7474):126-130, 2013.
Girsch et al., J. Med. Chem., 50:1658-1667, 2007.
Girsch et al., J. Med. Chem., 51:6752-6760, 2008.
Giordano et al., Curr. Drug Metab., 17(2): 194-203, 2016.
Greene & Wuts, Protective Groups in Organic Synthesis, 3^{rd} Ed., John Wiley, 1999. Handbook of Pharmaceutical Salts: Properties, and Use, Stahl and Wermuth (Eds.), Verlag Helvetica Chimica Acta, 2002.
Henderson, et al., Nat. Med, 19(12): 1617-1624, 2013.
Herlt, et al., Austr. J. Chem., 34(6):1319-1324, 1981.
Horan et al., Am. J. Respir. Grit. Care Med., 177(1):56-65, 2008.
Huang, et al., J. Cell Biol., 133(4):921-928, 1996.
Jørgensen, et al., J. Am. Chem. Soc., 124(42):12557-12565, 2002.
Kim et al., Am. J. Pathol., 156(4):1345-1362, 2000.
King et al., Vascular Pharmacology, 78:10-16, 2016.
Lacy-Hulbert, et al., Proc. Natl. Acad Sci. USA, 104(40): 15823-15828, 2007.
Landis et al., Organic Process Research &Development, 6:539-546, 2002.
Levine, et al., Am. J. Pathol., 156:1927-1935, 2000.
Li et al., Invest. Ophthalmol. Vis. Sci., 50(12):5988-5996, 2009.
Livant et al., J. Clin. Invest., 105(11): 1537-1545, 2000.
Lobert et al., Dev. Cell, 19(1): 148-159, 2010.
Lu, et al., J. Cell Sci., 115:4641-4648, 2002.
Luzina, et al., Arthritis Rheum., 60(5):1530-1539, 2009. March's Advanced Organic Chemistry: Reactions, Mechanisms, and Structure, 2007.
Melton et al., J. Clin. Invest., 120(12):4436-4444, 2010.
Millard et al., Theranostics, 1:154-88, 2011.
Mu et al., Cell Biol., 157(3):493-507, 2002.
Munger et al., Cell., 96(3):319-328, 1999.
Munger et al., Mol Biol. Ceil, 9:2627-2638, 1998.
Nakamura, et al., J. Bone Miner. Metab., 25(6):337-334, 2007.
Nishimura, Am. J. Pathol., 175(4):1362-1370, 2009.
Patsenker, et al., J. Hepatol., 46(5):878-887, 2007.
Penning, et al., Bioorg. Med. Chem. Lett., 16:3156-3161, 2006.
Perdih, Curr. Med Chem., 17(22):2371-2392, 2010.
Popov et al., J. Hepatol., 48(3):453-464, 2008.
Reagan-Shaw et al., FASEB J., 22(3):659-661, 2008
Reed, et al., Sci. Transl. Med, 7(288):288ra79, 2015.
Schneider, et al., Bone, 48(l):54-65, 2011.
Scotton et al., J. Clin. Invest., 119(9):2550-2563, 2009.
Suehiro et al., J. Biochem., 128(4):705-710, 2000.
Sun, et al., Am. J. Therapeutics, 2014.
Tatler, et al, J. Immunol., 187(11):6094-6107, 2011.
Travis, et al, Nature, 449(7160):361-365, 2007.
Wipff et al., J. Cell Biol., 179(6): 1311-1323, 2007.
Worthington, et al., Immunity, 42(5):903-915, 2015.
Yang et al, Development, 119(4): 1093-1105, 1993.
Yoshimura, Curr. Top. Microbiol. Immunol., 350:127-147, 2011.
Zahn et al, Arch. Ophthalmol., 127(10): 1329-1335, 2009.
Zahn et al., Invest. Ophthalmol. Vis. Sci., 51(2):1028-1035, 2010.

What is claimed is:

1. A compound of the formula: 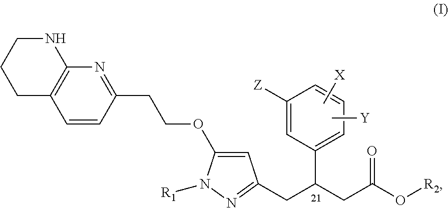
or a pharmaceutically acceptable salt or tautomer thereof,
wherein:
R₁ is hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, or substituted C₇₋₁₂aralkyl;
R₂ is hydrogen, unsubstituted C₁₋₈alkyl, or substituted C₁₋₈alkyl;
i) X and Y are each independently —OH, —OR^{X}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryl-CH₂O—, substituted C_{6 or 10}aryl-CH₂O—, unsubstituted C_{6 or 10}aryloxy, substituted C_{6 or 10}aryloxy, unsubstituted C₂₋₁₂acyloxy, substituted C₂₋₁₂acyloxy, 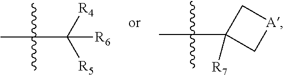
R₄ and R₅ are each independently unsubstituted C₁₋₈alkyl or substituted C₁₋₈alkyl, and
R⁶ is hydrogen, —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
A′ is —CF₂—, —O—, C₁₋₆alkanediyl, C₁₋₈alkoxydiyl, C₂₋₈alkylalkyoxy, or a covalent bond, thereby forming a cyclopropane ring, and
R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and
R^{X} is —(CH₂CH₂O)ᵣH or —(CH₂CH₂O)ᵣC₁₋₆alkyl, where r is an integer from 1-6; or
ii) X is —OR^{A} and Y is —OR^{B}, where R^{A} and R^{B} together are —(CR₁₂)ₙ—,
each R₁₂ is independently hydrogen or unsubstituted C₁₋₆alkyl, and
n is 1 or 2; and
Z is —OR^{Z}, t-butyl, unsubstituted 3-12 membered cycloalkyl, substituted 3-12 membered cycloalkyl, or 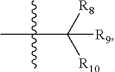
R₈ and R₉ are each independently unsubstituted C₁₋₈alkyl or substituted C₁₋₈alkyl,
R₁₀ is hydrogen, —OH, —CN, —NH₂, —CF₃, —CF₂H, —CFH₂, —CO₂H, —CO₂—C₁₋₈alkyl, —C(═O)NH₂, —CH₂OH, CH₂O—C₁₋₈alkyl, or C₁₋₈alkoxy, and
R^{Z} is —(CH₂CH₂O)ₛH or —(CH₂CH₂O)ₛC₁₋₆alkyl, where s is an integer from 1-6,
or
Z is 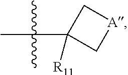
where A″ is —CF₂—, —O—, or C₁₋₈alkoxydiyl; and
R₁₁ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy.

2. The compound of claim 1 further defined as: 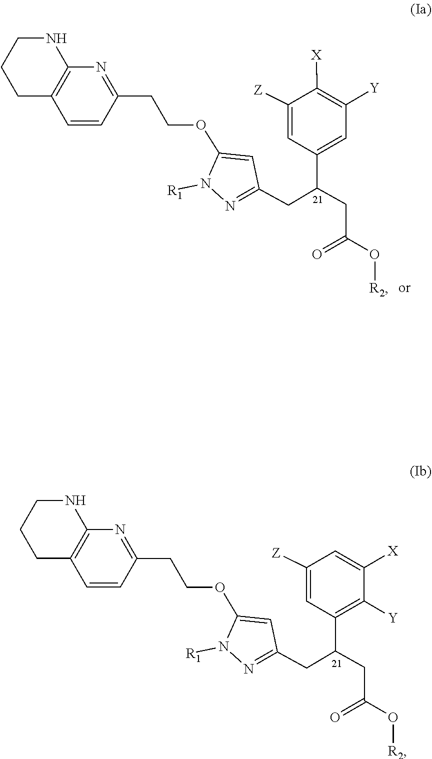
or a pharmaceutically acceptable salt or tautomer thereof.

3. The compound of claim 1 further defined as: 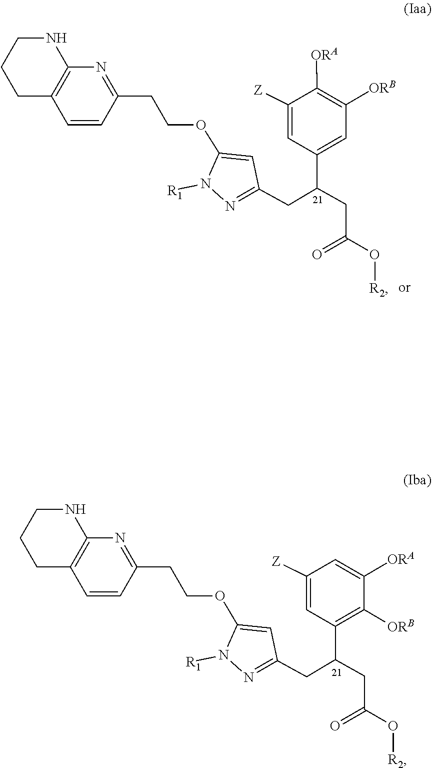
or a pharmaceutically acceptable salt or tautomer thereof.

4. The compound according to any one of claims 1-3,
wherein
R₁ is unsubstituted C₁₋₈alkyl, substituted C₁₋₈alkyl, unsubstituted C_{6 or 10}aryl, substituted C_{6 or 10}aryl, unsubstituted C₇₋₁₀aralkyl, or substituted C₇₋₁₀aralkyl; and
R₂ is hydrogen, unsubstituted C₁₋₆alkyl, or substituted C₁₋₆alkyl.

5. The compound according to any one of claims 1-4, wherein R₁ is unsubstituted C₁₋₈alkyl.

6. The compound of claim 4, wherein R₁ is methyl.

7. The compound according to any one of claims 1-6, wherein R₂ is hydrogen.

8. The compound according to any one of claims 1 and 4-7, wherein X is halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl-CH₂O—, substituted C_{6 or 10}aryl-CH₂O—, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, or substituted C₂₋₁₂acyloxy.

9. The compound of claim 1 or 2, wherein X is halo, cyano, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, unsubstituted C_{6 or 10}aryl-CH₂O—, substituted C_{6 or 10}aryl-CH₂O—, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy or

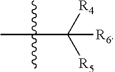

10. The compound of claim 8, wherein X is halo.

11. The compound of claim 10, wherein X is bromo, fluoro, or chloro.

12. The compound of claim 8, wherein X is —CF₃.

13. The compound of claim 8, wherein X is —OH or cyano.

14. The compound of claim 8, wherein X is unsubstituted C₁₋₈alkyl.

15. The compound of claim 14, wherein X is unsubstituted C₃₋₆alkyl.

16. The compound of claim 15, wherein X is t-butyl.

17. The compound of claim 8, wherein X is unsubstituted C₁₋₈alkoxy.

18. The compound of claim 17, wherein X is methoxy or isopropoxy.

19. The compound according to any one of claims 1, 2 and 4-18, wherein Y is t-butyl.

20. The compound according to any one of claims 1, 2 and 4-18, wherein Y is

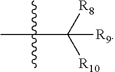

21. The compound of claim 20, wherein R₈ and R₉ are each independently unsubstituted C₂₋₈alkyl.

22. The compound of claim 20, wherein R₈ is methyl and R₉ is unsubstituted C₂₋₈alkyl.

23. The compound of claim 20, wherein R₈ and R₉ are each —CH₃.

24. The compound according to any one of claims 20-23, wherein R₁₀ is —CF₃, —CF₂H, or —CFH₂.

25. The compound of claim 24, wherein R₁₀ is —CF₃.

26. The compound of claim 20, wherein R₁₀ is hydrogen or —CH₃.

27. The compound according to any one of claims 1, 2 and 4-18, wherein Y is

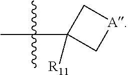

28. The compound of claim 27, wherein A″ is C₁₋₃alkanediyl, C₁₋₄alkoxydiyl, or a covalent bond, thereby forming a cyclopropane ring.

29. The compound of claim 27, wherein A″ is a covalent bond, thereby forming a cyclopropane ring.

30. The compound according to any one of claims 27-29, wherein R₁₁ is —CF₃, —CF₂H, —CH₂F, —CH₂O—C₁₋₆alkyl, C₁₋₆alkyl or C₁₋₈alkoxy.

31. The compound according to any one of claims 27-30, wherein R₁₁ is —CF₃, —CF₂H, —CH₂F, C₁₋₆alkyl or C₁₋₆alkoxy.

32. The compound of claim 31, wherein R₁₁ is —CF₃, —CF₂H or methoxy.

33. The compound of claim 32, wherein R₁₁ is —CF₃ or —CF₂H.

34. The compound of claim 32, wherein R₁₁ is —CH₂O—CH₃.

35. The compound according to any one of claims 1 and 4-18, wherein Y is halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl-CH₂O—, substituted C_{6 or 10}aryl-CH₂O—, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, or substituted C₂₋₁₂acyloxy.

36. The compound of claim 1 or 2, wherein Y is halo, cyano, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, unsubstituted C_{6 or 10}aryl-CH₂O—, substituted C_{6 or 10}aryl-CH₂O—, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy or

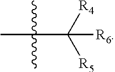

37. The compound of claim 36, wherein Y is halo.

38. The compound of claim 37, wherein Y is bromo, fluoro, or chloro.

39. The compound of claim 35, wherein Y is —CF₃.

40. The compound of claim 36, wherein Y is —OH or cyano.

41. The compound of claim 35, wherein Y is unsubstituted C₁₋₈alkyl.

42. The compound of claim 41, wherein Y is unsubstituted C₃₋₆alkyl.

43. The compound of claim 42, wherein Y is t-butyl.

44. The compound of claim 36, wherein Y is unsubstituted C₁₋₈alkoxy.

45. The compound of claim 44, wherein X is methoxy or isopropoxy.

46. The compound of claim 1, wherein X is —OR^{A} and Y is —OR^{B} and R^{A} and R^{B} together are —(CH₂)— or —(CH₂CH₂)—.

47. The compound according to any one of claims 1-46, wherein Z is t-butyl or adamantyl.

48. The compound according to any one of claims 1-46, wherein Z is t-butyl.

49. The compound according to any one of claims 1-46, wherein Z is adamantyl.

50. A compound of the formula: 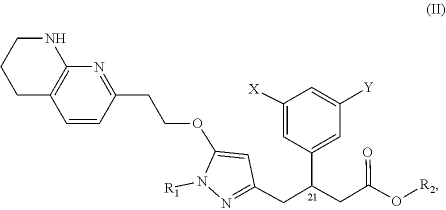
or a pharmaceutically acceptable salt or tautomer thereof,
wherein:
R₁ is hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, or substituted C₇₋₁₂aralkyl;
R₂ is hydrogen, unsubstituted C₁₋₈alkyl, or substituted C₁₋₈alkyl; and
i) X is —OR^{X} or halo,
Y is —OH, —OR^{Z}, —SF₅, 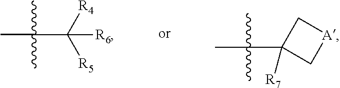
R₄ and R₅ are each independently unsubstituted C₁₋₈alkyl or substituted C₁₋₈alkyl, and
R₆ is —OH, —CN, —NH₂, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
A′ is —CF₂—, —O—, C₁₋₆alkanediyl, or a covalent bond, thereby forming a cyclopropane ring,
R₇ is —OH, —CN, —NH₂, —CO₂H, —CO₂—C₁₋₈alkyl, —C(═O)NH₂, —CF₃, —CH₂F, —CH₂OH, —CH₂O—C₁₋₈alkyl, C₁₋₈alkyl or C₁₋₈alkoxy,
where R^{X} is —(CH₂CH₂O)ᵣH or —(CH₂CH₂O)ᵣC₁₋₆alkyl, where r is an integer from 1-6, and
where R^{Z} is —(CH₂CH₂O)ₛH or —(CH₂CH₂O)ₛC₁₋₆alkyl, where s is an integer from 1-6;
ii) X is i-propyl, t-butyl, or 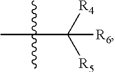
Y is i-propyl, 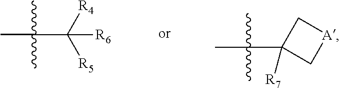
each R₄ and each R₅ are independently unsubstituted C₁₋₈alkyl or substituted C₁₋₈alkyl, and
each R₆ is independently —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
A′ is —CF₂—, —O—, C₁₋₆alkanediyl, C₁₋₈alkoxydiyl, C₂₋₈alkylalkoxydiyl, or a covalent bond, thereby forming a cyclopropane ring, and
R₇ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈ alkyl, C₁₋₈alkyl or C₁₋₈alkoxy; or
iii) X is cyano,
Y is —OR^{Z}, —SF₅, 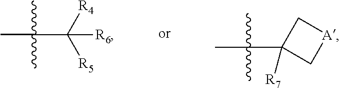
R₄ and R₅ are each independently unsubstituted C₁₋₈alkyl or substituted C₁₋₈alkyl, and
R₆ is —H, —OH, —CN, —NH₂, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
A′ is —CF₂—, —O—, C₁₋₆alkanediyl, C₁₋₈alkoxydiyl, or a covalent bond, thereby forming a cyclopropane ring,
R₇ is —OH, —CN, —NH₂, —CO₂H, —CO₂—C₁₋₈alkyl, —C(═O)NH₂, —CF₃, —CH₂F, —CH₂OH, —CH₂O—C₁₋₈alkyl, C₁₋₈alkyl or C₁₋₈alkoxy,
where R^{X} is —(CH₂CH₂O)ᵣH or —(CH₂CH₂O)ᵣC₁₋₆alkyl, where r is an integer from 1-6, and
where R^{Z} is —(CH₂CH₂O)ₛH or —(CH₂CH₂O)ₛC₁₋₆alkyl, where s is an integer from 1-6.

51. The compound of claim 50, wherein R₁ is unsubstituted C₁₋₈alkyl.

52. The compound of claim 51, wherein R₁ is methyl.

53. The compound according to any one of claims 50-52, wherein R₂ is hydrogen.

54. The compound according to any one of claims 50-53, wherein Y is —OR^{Z}.

55. The compound according to any one of claims 50-53, wherein Y is —SF₅.

56. The compound of claims 50-53, wherein Y is i-propyl.

57. The compound according to any one of claims 50-53, wherein Y is —OH.

58. The compound according to any one of claims 50-53, wherein i-propyl, or

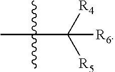

59. The compound of claim 59, wherein Y is

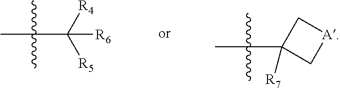

60. The compound according to any one of claims 50-53, wherein Y is

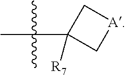

61. A compound of the formula: 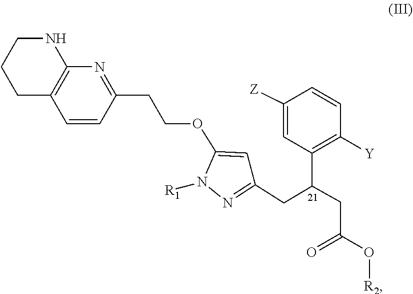
or a pharmaceutically acceptable salt or tautomer thereof,
wherein:
R₁ is hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, or substituted C₇₋₁₂aralkyl;
R₂ is hydrogen, unsubstituted C₁₋₈alkyl, or substituted C₁₋₈alkyl; and
i) Y is bromo, fluoro, cyano or substituted C₁₋₁₂alkoxy,
Z is t-butyl, —SF₅, 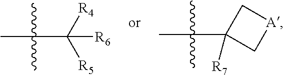
R₄ and R₅ are each independently unsubstituted C₁₋₈alkyl or substituted C₁₋₈alkyl, and
R₆ is —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
A′ is —CF₂—, —O—, C₁₋₆alkanediyl, C₁₋₈alkoxydiyl, or a covalent bond, thereby forming a cyclopropane ring, and
R₇ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈ alkyl, C₁₋₈alkyl or C₁₋₈alkoxy; or
ii) Y is —OR^{X} or C₁₋₈alkoxy,
Z is —OR^{Z}, —SF₅, unsubstituted 3-10 membered heterocycloalkyl, 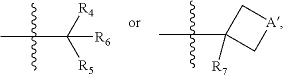
R₄ and R₅ are each independently unsubstituted C₁₋₈alkyl or substituted C₁₋₈alkyl, and
R₆ is —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
A′ is —CF₂—, —O—, C₁₋₆alkanediyl, or a covalent bond, thereby forming a cyclopropane ring,
R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and
R^{X} is —(CH₂CH₂O)ᵣH or —(CH₂CH₂O)ᵣC₁₋₆alkyl, where r is an integer from 1-6, and
R^{Z} is —(CH₂CH₂O)ₛH or —(CH₂CH₂O)ₛC₁₋₆alkyl, where s is an integer from 1-6.

62. The compound of claim 61, wherein R₁ is unsubstituted C₁₋₈alkyl.

63. The compound of claim 62, wherein R₁ is methyl.

64. The compound according to any one of claims 61-63, wherein R₂ is hydrogen.

65. The compound according to any one of claims 61-64, wherein Y is bromo, fluoro, cyano or substituted C₁₋₂alkoxy.

66. The compound of claim 65, wherein Y is —OCF₃.

67. The compound according to any one of claims 61-63, wherein Y is C₁₋₃lkoxy.

68. The compound of claim 67, wherein Y is —OCH₃.

69. The compound according to any one of claims 61-66, wherein Z is t-butyl.

70. The compound according to any one of claims 61, 67 and 68, wherein Z is 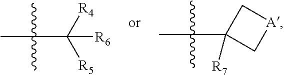
R₄ and R₅ are each —CH₃, and
R₆ is —OH, —CN, —CF₃, —CF₂H, —CH₂F, —CH₂OH or C₁₋₈alkoxy,
A′ is —CF₂—, —O—, or a covalent bond, thereby forming a cyclopropane ring, and
R₇ is hydrogen, —OH, —CN, —CF₃, —CF₂H, —CH₂F, —CH₂OH, —CH₂O—C₁₋₈alkyl, C₁₋₃alkyl or C₁₋₃alkoxy.

71. The compound of claim 70, wherein Z is 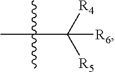
where R₆ is —OH, or —CH₂OH.

72. The compound of claim 70, wherein Z is 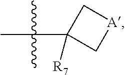
A′ is —O—, or a covalent bond, thereby forming a cyclopropane ring, and
R₇ is hydrogen, —CF₃, —CF₂H, —CH₂F or —OCH₃.

73. The compound according to any one of claims 1-72, wherein the carbon atom 21 is in the S configuration.

74. The compound of claim 1-73, wherein the compound is an integrin antagonist.

75. The compound of claim 74, wherein the integrin is an α₅β₁ integrin antagonist.

76. The compound of claim 75, wherein the compound exhibits an IC₅₀ value for the α₅β₁ integrin of less than 50 nM, 40 nM, 30 nM, 20 nM, 15 nm or 1 nM, or a range defined by any of the preceding as measured by a solid phase receptor assay for α₅β₁ integrin function.

77. The compound of any one of claims 74-76, wherein the integrin is an α_{V}β₁ integrin antagonist.

78. The compound according to any one of claims 1-75, wherein the compound exhibits an IC₅₀ value for the α_{V}β₁ integrin of less than 15 nM as measured by a solid phase receptor assay for α_{V}β₁ integrin function.

79. The compound according to any one of claims 1-78 wherein the compound exhibits an IC₅₀ value for an α_{V}β₃ integrin of less than 10 nM as measured by a solid phase receptor assay for α_{V}β₃ integrin function.

80. The compound according to any one of claims 1-79, wherein the compound exhibits an IC₅₀ value for an α_{V}β₅ integrin of less than 10 nM as measured by a solid phase receptor assay for α_{V}β₅ integrin function.

81. The compound according to any one of claims 1-80, wherein the compound exhibits an IC₅₀ value for the α_{V}β₁, α_{V}β₃, and α_{V}β₅ integrins of less than 10 nM as measured by a solid phase receptor assays for α_{V}β₁, α_{V}β₃, and α_{V}β₅ integrin function.

82. The compound according to any one of claims 1-81, wherein the compound exhibits an IC₅₀ value for an α_{V}β₆ integrin of greater than 10 nM as measured by a solid phase receptor assay for α_{V}β₆ integrin function.

83. The compound according to any one of claims 1-82, wherein the compound exhibits an IC₅₀ value for an α_{V}β₆ integrin of greater than 12 nM as measured by a solid phase receptor assay for α_{V}β₆ integrin function.

84. The compound according to any one of claims 1-83, wherein the compound exhibits an IC₅₀ value for the α_{V}β₆ and α_{V}β₈ integrins of greater than 10 nM as measured by solid phase receptor assays for α_{V}β₆ and α_{V}β₈ integrin function.

85. The compound of claim 1, wherein the compound is further defined as: 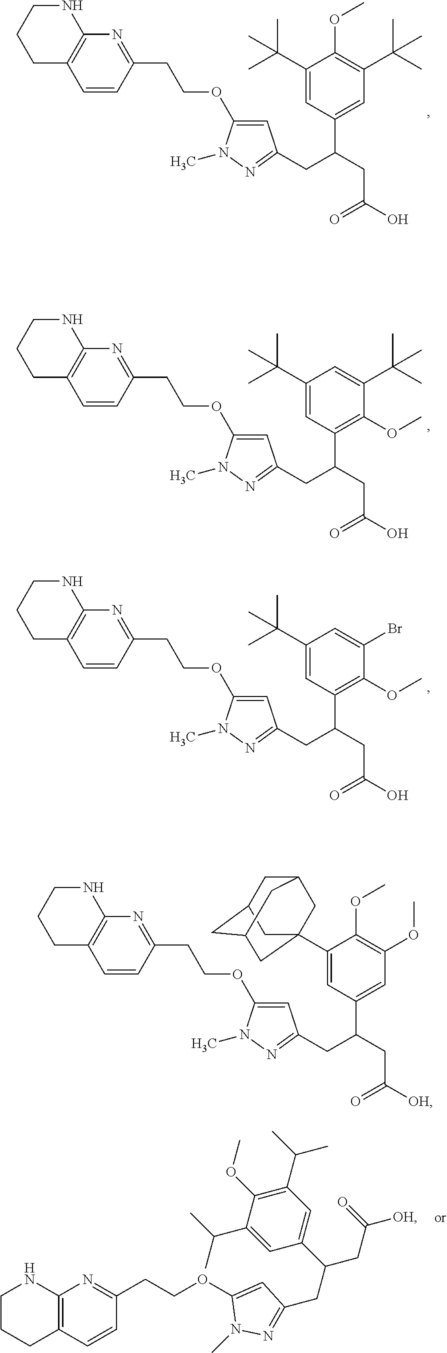 [SMILES: COc1c(C(C)(C)C)cc(C(CC(=O)O)Cc2cc(OCCc3ccc4c(n3)NCCC4)n(C)n2)cc1C(C)(C)C ; COc1c(C(CC(=O)O)Cc2cc(OCCc3ccc4c(n3)NCCC4)n(C)n2)cc(C(C)(C)C)cc1C(C)(C)C ; COc1c(Br)cc(C(C)(C)C)cc1C(CC(=O)O)Cc1cc(OCCc2ccc3c(n2)NCCC3)n(C)n1 ; COc1cc(C(CC(=O)O)Cc2cc(OCCc3ccc4c(n3)NCCC4)n(C)n2)cc(C23CC4CC(CC(C4)C2)C3)c1OC ; COc1c(C(C)C)cc(C(CC(=O)O)Cc2cc(OCCc3ccc4c(n3)NCCC4)n(C)n2)cc1C(C)C] 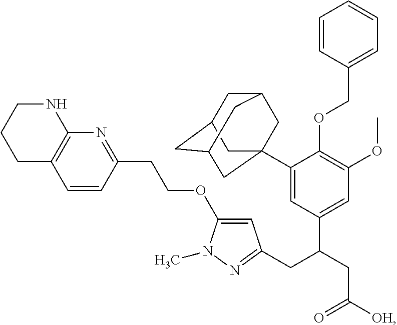 [SMILES: COc1cc(C(CC(=O)O)Cc2cc(OCCc3ccc4c(n3)NCCC4)n(C)n2)cc(C23CC4CC(CC(C4)C2)C3)c1OCc1ccccc1]
or a pharmaceutically acceptable salt thereof.

86. The compound of claim 1 further defined as: 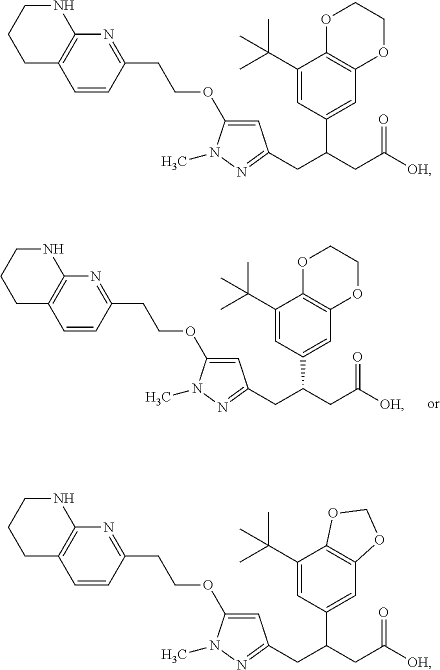 [SMILES: Cn1nc(CC(CC(=O)O)c2cc3c(c(C(C)(C)C)c2)OCCO3)cc1OCCc1ccc2c(n1)NCCC2 ; Cn1nc(C[C@@H](CC(=O)O)c2cc3c(c(C(C)(C)C)c2)OCCO3)cc1OCCc1ccc2c(n1)NCCC2 ; Cn1nc(CC(CC(=O)O)c2cc3c(c(C(C)(C)C)c2)OCO3)cc1OCCc1ccc2c(n1)NCCC2]
or a pharmaceutically acceptable salt thereof.

87. The compound of claim 50, wherein the compound is further defined as: 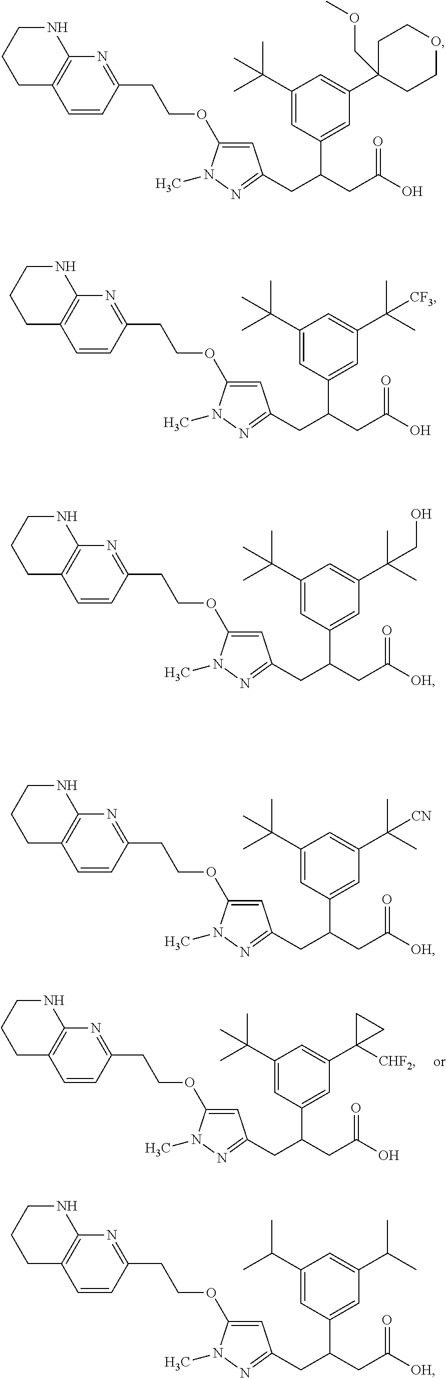 [SMILES: COCC1(c2cc(C(CC(=O)O)Cc3cc(OCCc4ccc5c(n4)NCCC5)n(C)n3)cc(C(C)(C)C)c2)CCOCC1 ; Cn1nc(CC(CC(=O)O)c2cc(C(C)(C)C)cc(C(C)(C)C(F)(F)F)c2)cc1OCCc1ccc2c(n1)NCCC2 ; Cn1nc(CC(CC(=O)O)c2cc(C(C)(C)C)cc(C(C)(C)CO)c2)cc1OCCc1ccc2c(n1)NCCC2 ; Cn1nc(CC(CC(=O)O)c2cc(C(C)(C)C)cc(C(C)(C)C#N)c2)cc1OCCc1ccc2c(n1)NCCC2 ; Cn1nc(CC(CC(=O)O)c2cc(C(C)(C)C)cc(C3(C(F)F)CC3)c2)cc1OCCc1ccc2c(n1)NCCC2 ; CC(C)c1cc(C(C)C)cc(C(CC(=O)O)Cc2cc(OCCc3ccc4c(n3)NCCC4)n(C)n2)c1]
or a pharmaceutically acceptable salt thereof.

88. The compound of claim 50 further defined as: 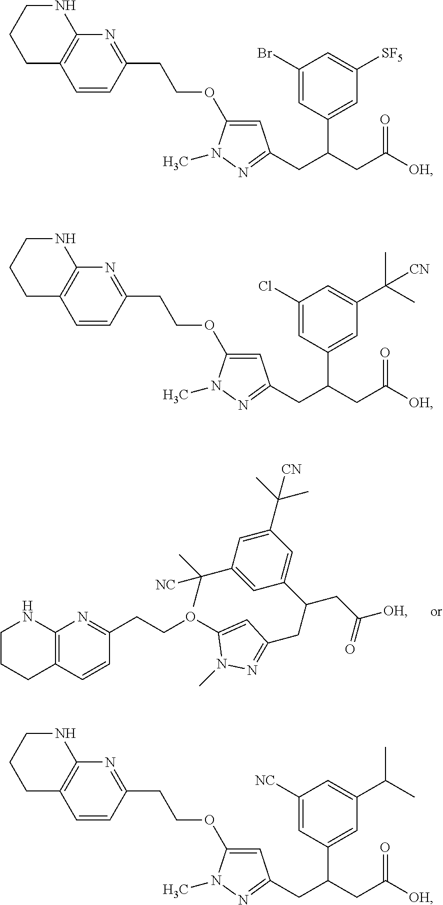
or a pharmaceutically acceptable salt thereof.

89. The compound of claim 61, wherein the compound is further defined as: 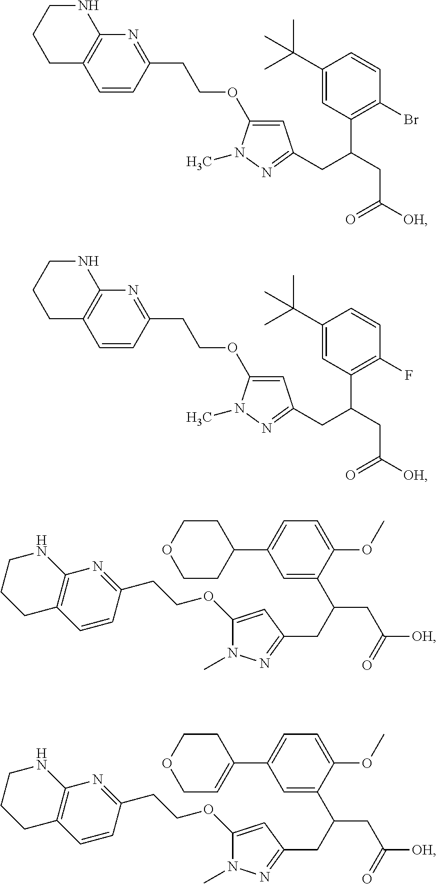 [SMILES: Cn1nc(CC(CC(=O)O)c2cc(C(C)(C)C)ccc2Br)cc1OCCc1ccc2c(n1)NCCC2 ; Cn1nc(CC(CC(=O)O)c2cc(C(C)(C)C)ccc2F)cc1OCCc1ccc2c(n1)NCCC2 ; COc1ccc(C2CCOCC2)cc1C(CC(=O)O)Cc1cc(OCCc2ccc3c(n2)NCCC3)n(C)n1 ; COc1ccc(C2=CCOCC2)cc1C(CC(=O)O)Cc1cc(OCCc2ccc3c(n2)NCCC3)n(C)n1] 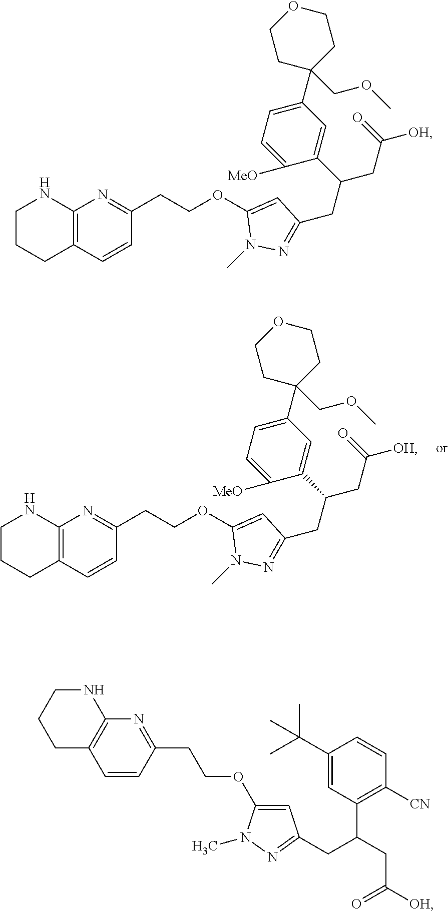 [SMILES: COCC1(c2ccc(OC)c(C(CC(=O)O)Cc3cc(OCCc4ccc5c(n4)NCCC5)n(C)n3)c2)CCOCC1 ; COCC1(c2ccc(OC)c([C@H](CC(=O)O)Cc3cc(OCCc4ccc5c(n4)NCCC5)n(C)n3)c2)CCOCC1 ; Cn1nc(CC(CC(=O)O)c2cc(C(C)(C)C)ccc2C#N)cc1OCCc1ccc2c(n1)NCCC2]
or a pharmaceutically acceptable salt thereof.

90. The compound of claim 61 further defined as: 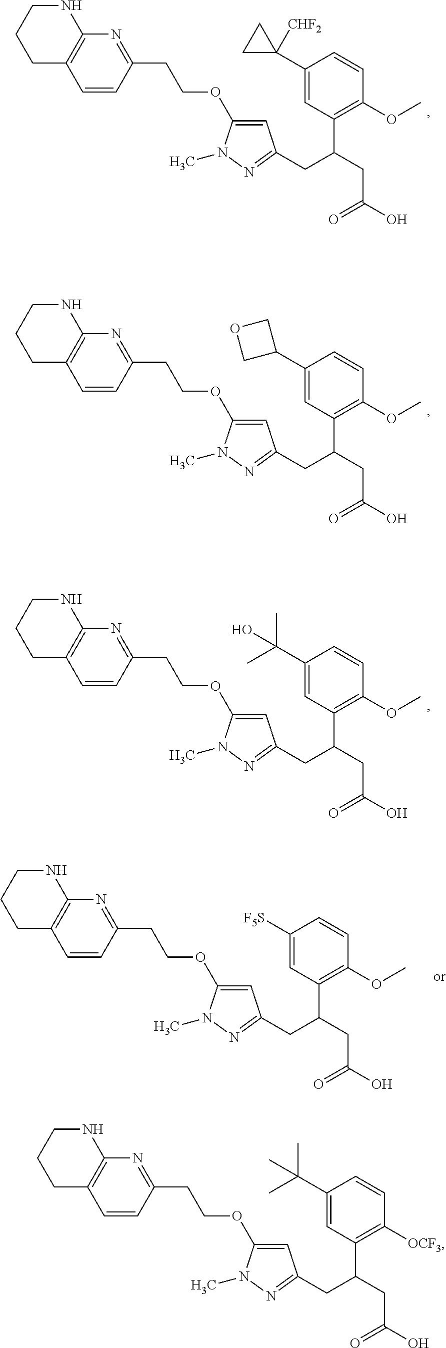
or a pharmaceutically acceptable salt thereof.

91. The compound according to any one of claims 85-90, wherein the depicted chiral center in the S configuration.

92. A compound of the formula: 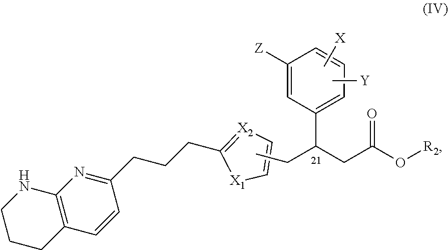
or a pharmaceutically acceptable salt or tautomer thereof,
wherein:
R₂ is hydrogen, unsubstituted C₁₋₈alkyl, or substituted C₁₋₈alkyl;
X₁ is O (oxygen), S (sulfur), or —NR^{1A}—;
R^{1A} is hydrogen, unsubstituted C₁₋₈alkyl or substituted C₁₋₈alkyl;
X₂ is N (nitrogen);
i) X and Y are each independently hydrogen, —OR^{X}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryl-CH₂O—, substituted C_{6 or 10}aryl-CH₂O—, unsubstituted C_{6 or 10}aryloxy, substituted C_{6 or 10}aryloxy, unsubstituted C₂₋₁₂acyloxy, substituted C₂₋₁₂acyloxy, 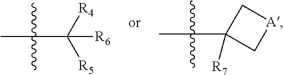
where R₄ and R₅ are each independently unsubstituted C₁₋₈alkyl or substituted C₁₋₈alkyl, and
R₆ is hydrogen, —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
where A′ is —CF₂—, —O—, C₁₋₆alkanediyl, C₁₋₈alkoxydiyl, C₂₋₈alkylalkoxydiyl, or a covalent bond, thereby forming a cyclopropane ring, and
R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and
R^{X} is —(CH₂CH₂O)ᵣH or —(CH₂CH₂O)ᵣC₁₋₆alkyl, where r is an integer from 1-6; or
ii) X is —OR^{A} and Y is —OR^{B}, where R^{A} and R^{B} together are —(CR₁₂)ₙ—,
each R₁₂ is independently hydrogen or unsubstituted C₁₋₆alkyl, and
n is 1 or 2; and
Z is —OR^{Z}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy, 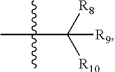
where R₈ and R₉ are each independently unsubstituted C₁₋₈alkyl or substituted C₁₋₈alkyl,
R₁₀ is hydrogen, —OH, —CN, —NH₂, —CF₃, —CF₂H, —CFH₂, —CO₂H, —CO₂— C₁₋₈alkyl, —C(═O)NH₂, —CH₂OH, CH₂O—C₁₋₈alkyl, or C₁₋₈alkoxy, and
R^{Z} is —(CH₂CH₂O)ₛH or —(CH₂CH₂O)ₛC₁₋₆alkyl, where s is an integer from 1-6, or
Z is 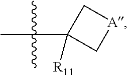
where A″ is —CF₂—, —O—, C₁₋₆alkanediyl, C₁₋₈alkoxydiyl, or C₂₋₈alkylalkoxydiyl, where R₁₁ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy.

93. The compound of claim 92 further defined as: 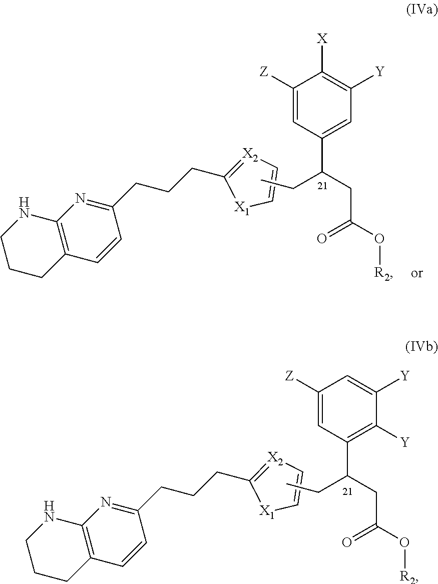
or a pharmaceutically acceptable salt or tautomer thereof.

94. The compound of claim 92 further defined as: 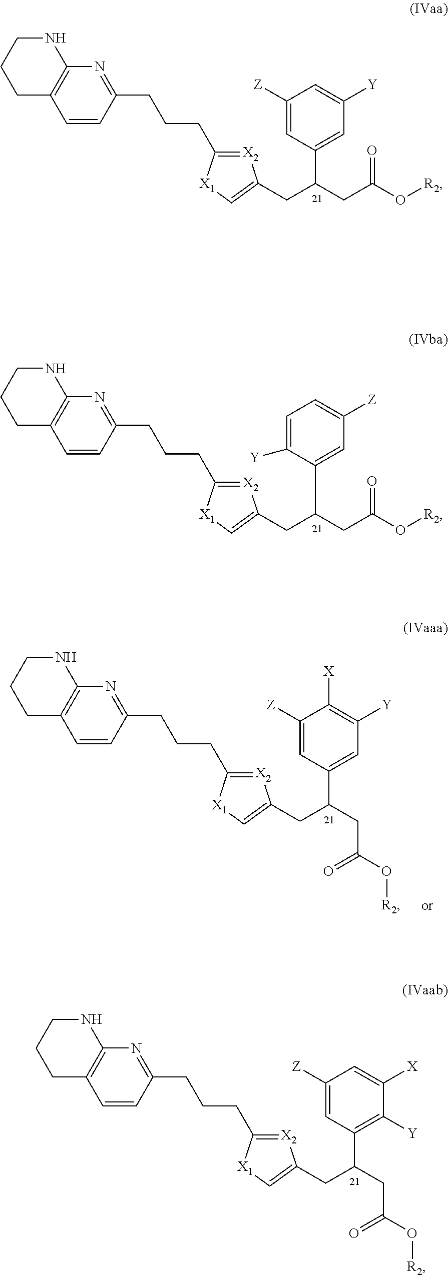
or a pharmaceutically acceptable salt or tautomer thereof.

95. The compound of claim 92 further defined as:
a compound of Table 4, or a pharmaceutically acceptable salt or tautomer thereof.

96. A compound of the formula: 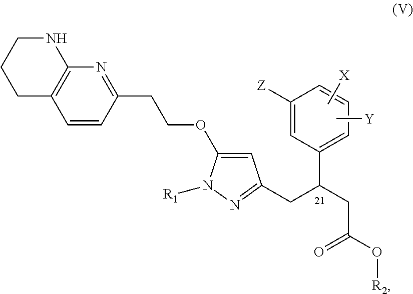
or a pharmaceutically acceptable salt or tautomer thereof,
wherein:
R₁ is unsubstituted C₂₋₈alkyl, substituted C₁₋₈alkyl, unsubstituted C_{6 or 10}aryl, substituted C_{6 or 10}aryl, unsubstituted C₇₋₁₂aralkyl, or substituted C₇₋₁₂aralkyl;
R₂ is hydrogen, unsubstituted C₁₋₈alkyl, or substituted C₁₋₈alkyl;
i) X and Y are each independently hydrogen, —OR^{X}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryl-CH₂O—, substituted C_{6 or 10}aryl-CH₂O—, unsubstituted C_{6 or 10}aryloxy, substituted C_{6 or 10}aryloxy, unsubstituted C₂₋₁₂acyloxy, substituted C₂₋₁₂acyloxy, 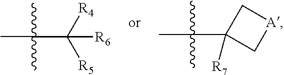
where R₄ and R₅ are each independently unsubstituted C₁₋₈alkyl or substituted C₁₋₈alkyl, and
R₆ is hydrogen, —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
where A′ is —CF₂—, —O—, C₁₋₆alkanediyl, C₁₋₈alkoxydiyl, C₂₋₈alkylalkoxydiyl, or a covalent bond, thereby forming a cyclopropane ring, and
R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and
R^{X} is —(CH₂CH₂O)ᵣH or —(CH₂CH₂O)ᵣC₁₋₆alkyl, where r is an integer from 1-6; or
ii) X is —OR^{A} and Y is —OR^{B}, where R^{A} and R^{B} together are —(CR₁₂)ₙ—,
each R₁₂ is independently hydrogen or unsubstituted C₁₋₆alkyl, and
n is 1 or 2; and
Z is —OR^{Z}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy, or 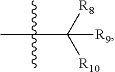
where R₈ and R₉ are each independently unsubstituted C₁₋₈alkyl or substituted C₁₋₈alkyl,
R₁₀ is hydrogen, —OH, —CN, —NH₂, —CF₃, —CF₂H, —CFH₂, —CO₂H, —CO₂— C₁₋₈alkyl, —C(═O)NH₂, —CH₂OH, CH₂O—C₁₋₈alkyl, or C₁₋₈alkoxy, and
R^{Z} is —(CH₂CH₂O)ₛH or —(CH₂CH₂O)ₛC₁₋₆alkyl, where s is an integer from 1-6, or
Z is 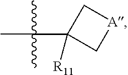
where A″ is —CF₂—, —O—, C₁₋₆alkanediyl, C₁₋₈alkoxydiyl, or C₂₋₈alkylalkoxydiyl, where R₁₁ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy.

97. The compound of claim 96 further defined as: 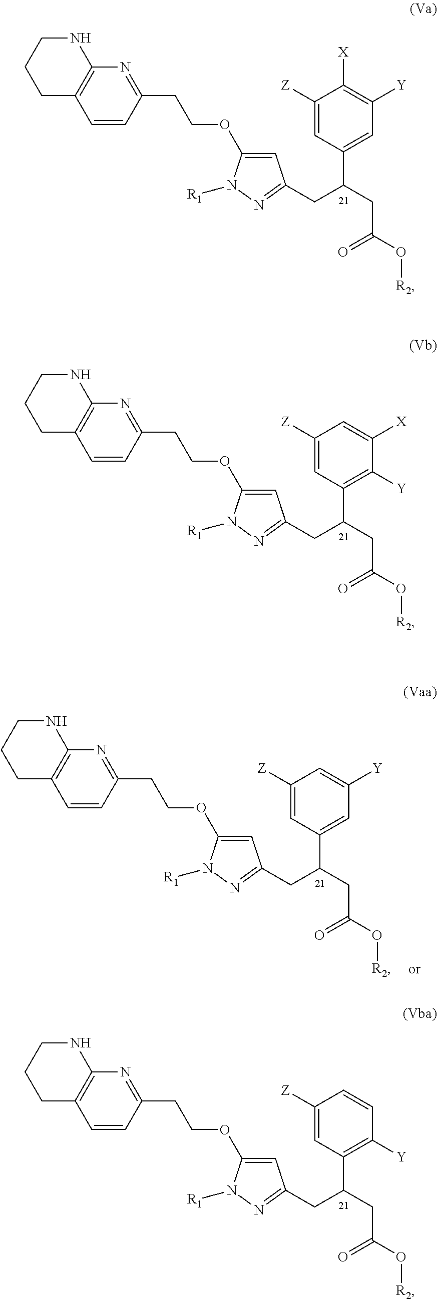
or a pharmaceutically acceptable salt or tautomer thereof.

98. The compound of claim 96 further defined as: 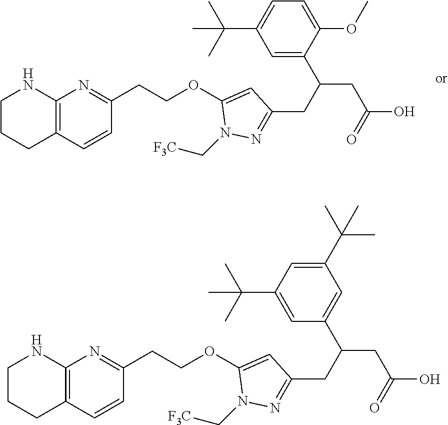 [SMILES: COc1ccc(C(C)(C)C)cc1C(CC(=O)O)Cc1cc(OCCc2ccc3c(n2)NCCC3)n(CC(F)(F)F)n1 ; CC(C)(C)c1cc(C(CC(=O)O)Cc2cc(OCCc3ccc4c(n3)NCCC4)n(CC(F)(F)F)n2)cc(C(C)(C)C)c1]
or a pharmaceutically acceptable salt or tautomer thereof.

99. A compound of the formula: 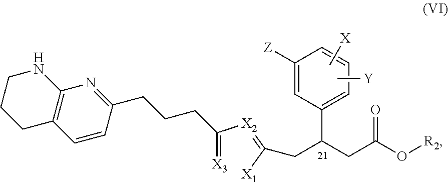
or a pharmaceutically acceptable salt or tautomer thereof,
wherein:
R₂ is hydrogen, unsubstituted C₁₋₈alkyl, or substituted C₁₋₈alkyl;
X₁ is O (oxygen), S (sulfur), or —NR^{1A}—;
X₂ is CR^{1A} or N (nitrogen);
X₃ is CR^{1A} or N (nitrogen);
each R^{1A} is independently hydrogen, unsubstituted C₁₋₈alkyl or substituted C₁₋₈alkyl;
i) X and Y are each independently hydrogen, —OR^{X}, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryl-CH₂O—, substituted C_{6 or 10}aryl-CH₂O—, unsubstituted C_{6 or 10}aryloxy, substituted C_{6 or 10}aryloxy, unsubstituted C₂₋₁₂acyloxy, substituted C₂₋₁₂acyloxy, 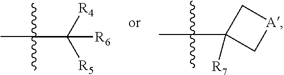
where R₄ and R₅ are each independently unsubstituted C₁₋₈alkyl or substituted C₁₋₈alkyl, and
R₆ is hydrogen, —OH, —CN, —NH₂, —CF₃, —CF₂H, —CH₂F, —CO₂H, —CO₂—C₁₋₈alkyl, —C(═O)NH₂, —CH₂OH, —CH₂O—C₁₋₈alkyl, or C₁₋₈alkoxy,
where A′ is —CF₂—, —O—, C₁₋₆alkanediyl, C₁₋₈alkoxydiyl, C₂₋₈alkylalkoxydiyl, or a covalent bond, thereby forming a cyclopropane ring, and
R₇ is hydrogen, —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy, and
R^{X} is —(CH₂CH₂O)ᵣH or —(CH₂CH₂O)ᵣC₁₋₆alkyl, where r is an integer from 1-6; or
ii) X is —OR^{A} and Y is —OR^{B}, where R^{A} and R^{B} together are —(CR₁₂)ₙ—,
each R₁₂ is independently hydrogen or unsubstituted C₁₋₆alkyl, and
n is 1 or 2; and
Z is —OR^{Z}, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy, or 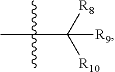
where R₈ and R₉ are each independently unsubstituted C₁₋₈alkyl or substituted C₁₋₈alkyl,
R₁₀ is hydrogen, —OH, —CN, —NH₂, —CF₃, —CF₂H, —CFH₂, —CO₂H, —CO₂— C₁₋₈alkyl, —C(═O)NH₂, —CH₂OH, CH₂O—C₁₋₈alkyl, or C₁₋₈alkoxy, and
R^{Z} is —(CH₂CH₂O)ₛH or —(CH₂CH₂O)ₛC₁₋₆alkyl, where s is an integer from 1-6, or
Z is 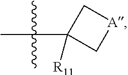
where A″ is —CF₂—, —O—, C₁₋₆alkanediyl, C₁₋₈alkoxydiyl, or C₂₋₈alkylalkoxydiyl, where R₁₁ is —OH, —CN, —NH₂, —CO₂H, —CO₂—C₁₋₈alkyl, —C(═O)NH₂, —CF₃, —CF₂H, —CH₂F, —CH₂OH, —CH₂O—C₁₋₈alkyl, C₁₋₈alkyl or C₁₋₈alkoxy.

100. The compound of claim 99, wherein X₂ is N (nitrogen).

101. The compound of claim 99 or 100, wherein X₃ is N (nitrogen).

102. The compound according to any one of claims 92-94 and 99-101, wherein X₁ is O (oxygen), or S (sulfur).

103. The compound according to any one of claims 92-94 and 99-101, wherein X₁ is S (sulfur).

104. The compound according to any one of claims 92-103, wherein R₂ is hydrogen.

105. The compound according to any one of claims 92-104, wherein X is hydrogen, halo, cyano, unsubstituted C₁₋₁₂alkyl, substituted C₁₋₁₂alkyl, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, or substituted C₂₋₁₂acyloxy.

106. The compound according to any one of claims 92-104, wherein X is hydrogen, halo, cyano, unsubstituted C₁₋₁₂alkoxy, substituted C₁₋₁₂alkoxy, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocycloalkyl, substituted 3-10 membered heterocycloalkyl, unsubstituted C_{6 or 10}aryloxy, substituted C_{6 or 10}aryloxy, unsubstituted C₂₋₁₂acyloxy, substituted C₂₋₁₂acyloxy or

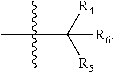

107. The compound according to any one of claims 92-104, wherein X is halo.

108. The compound of claim 100, wherein X is bromo, fluoro, or chloro.

109. The compound according to any one of claims 92-104, wherein X is —CF₃.

110. The compound according to any one of claims 92-104, wherein X is —OH or cyano.

111. The compound according to any one of claims 92-104, wherein X is unsubstituted C₁₋₈alkyl.

112. The compound of claim 111, wherein X is unsubstituted C₃₋₆alkyl.

113. The compound of claim 112, wherein X is t-butyl.

114. The compound according to any one of claims 92-101, wherein X is unsubstituted C₁₋₈alkoxy.

115. The compound according to any one of claims 92-94 and 96-115, wherein Z is unsubstituted 3-10 membered heterocycloalkyl, or substituted 3-10 membered heterocycloalkyl.

116. The compound according to any one of claims 92-94 and 96-115, wherein Z is t-butyl.

117. The compound according to any one of claims 92-94 and 96-115, wherein Z is

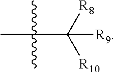

118. The compound of claim 117, wherein R₈ and R₉ are each independently unsubstituted C₂₋₈alkyl.

119. The compound of claim 117, wherein R₈ is methyl and R₉ is unsubstituted C₂₋₈alkyl.

120. The compound of claim 117, wherein R₈ and R₉ are each —CH₃.

121. The compound according to any one of claims 20-23, wherein R₁₀ is —CF₃, —CF₂H, or —CFH₂.

122. The compound of claim 121, wherein R₁₀ is —CF₃.

123. The compound of claim 117, wherein R₁₀ is hydrogen or —CH₃.

124. The compound according to any one of claims 92-94 and 96-115, wherein Z is

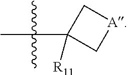

125. The compound of claim 123, wherein A″ is C₁₋₃alkanediyl, C₁₋₄alkoxydiyl, or a covalent bond, thereby forming a cyclopropane ring.

126. The compound of claim 123, wherein A″ is a covalent bond, thereby forming a cyclopropane ring.

127. The compound according to any one of claims 123-126, wherein R₁₁ is —CF₃, —CF₂H, —CH₂F, —CH₂O—C₁₋₆alkyl, C₁₋₆alkyl or C₁₋₈alkoxy.

128. The compound according to any one of claims 123-127, wherein R₁₁ is —CF₃, —CF₂H, —CH₂F, C₁₋₆alkyl or C₁₋₆alkoxy.

129. The compound of claim 128, wherein R₁₁ is —CF₃, —CF₂H or methoxy.

130. The compound of claim 129, wherein R₁₁ is —CF₃ or —CF₂H.

131. The compound of claim 129, wherein R₁₁ is —CH₂O—CH₃.

132. The compound of claim 115, wherein Z is unsubstituted 3-10 membered heterocycloalkyl where the unsubstituted 3-10 membered heterocycloalkyl is pyran or unsaturated pyran.

133. The compound of claim 115, wherein Z is substituted 3-10 membered heterocycloalkyl where the substituted 3-10 membered heterocycloalkyl is substituted pyran or substituted unsaturated pyran.

134. The compound according to any one of claims 92-133, wherein the carbon atom 21 is in the S configuration.

135. A pharmaceutical composition comprising:
a) the compound according to any one of claims 1-134; and
b) an excipient.

136. The pharmaceutical composition of claim 135, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

137. The pharmaceutical composition of claim 135 or 136, wherein the pharmaceutical composition is formulated for oral, topical, intravenous, or intravitreal administration.

138. The pharmaceutical composition according to any one of claims 135-137, wherein the pharmaceutical composition is formulated as a unit dose.

139. A method of treating and/or preventing a disease or a disorder in a patient in need thereof, comprising administering to the patient a compound or composition according to any one of claims 1-138 in an amount sufficient to treat and/or prevent the disease or disorder.

140. The method of claim 139, wherein the disease or disorder is associated with fibrosis.

141. The method of either claim 139 or 140, wherein the disease or disorder is scleroderma or fibrosis of the lungs, liver, kidneys, heart, skin, or pancreas.

142. The method of claim 141, wherein the disease or disorder is fibrosis of the lungs.

143. The method of claim 141, wherein the disease or disorder is fibrosis of the liver.

144. The method of claim 141, wherein the disease or disorder is fibrosis of the heart.

145. The method of claim 141, wherein the disease or disorder is fibrosis of the kidneys.

146. The method of claim 141, wherein the disease or disorder is fibrosis of the pancreas.

147. The method of claim 141, wherein the disease or disorder is fibrosis of the skin.

148. The method of claim 141, wherein the disease or disorder is scleroderma.

149. The method of claim 139-148, wherein the patient is a human, monkey, cow, horse, sheep, goat, dog, cat, mouse, rat, guinea pig, or transgenic species thereof.

150. The method of claim 149, wherein the patient is a monkey, cow, horse, sheep, goat, dog, cat, mouse, rat, or guinea pig.

151. The method of claim 149, wherein the patient is a human.

152. A method of inhibiting the binding of an integrin comprising contacting the integrin with a compound or composition according to any one of claims 1-138.

153. The method of claim 152, wherein the integrin is α₅β₁, α_{V}β₁, α_{V}β₃, or α_{V}β₅.

154. The method of claim 153, wherein the integrin is α_{V}β₁.

155. The method of claim 153, wherein the integrin is α₅β₁.

156. The method according to any one of claims 152-155, wherein the method is performed in vitro.

157. The method according to any one of claims 152-155, wherein the method is performed ex vivo or in vivo.

158. The method according to any one of claims 152-155 and 157, wherein the inhibition of binding is sufficient to treat or prevent a disease or disorder in a patient.
